(19) United States
(12) Patent Application Publication
(10) Pub. No.: US 2024/0110242 A1
(43) Pub. Date: Apr. 4, 2024

(54) SYSTEMS AND METHODS OF DIAGNOSING IDIOPATHIC PULMONARY FIBROSIS

(71) Applicant: Veracyte, Inc., South San Francisco, CA (US)

(72) Inventors: Giulia C. Kennedy, San Francisco, CA (US); James Diggans, South San Francisco, CA (US); Jing Huang, South San Francisco, CA (US); Yoonha Choi, South San Francisco, CA (US); Su Yeon Kim, South San Francisco, CA (US); Daniel Pankratz, Los Gatos, CA (US); Moraima Pagan, San Francisco, CA (US)

(21) Appl. No.: 18/181,535

(22) Filed: Mar. 9, 2023

Related U.S. Application Data
(63) Continuation of application No. 17/218,125, filed on Mar. 30, 2021, now Pat. No. 11,639,527.
Application No. 17/218,125 is a continuation of application No. 16/840,009, filed on Apr. 3, 2020, now abandoned.
Application No. 16/840,009 is a continuation of application No. 16/551,645, filed on Aug. 26, 2019, now abandoned.
Application No. 16/551,645 is a continuation of application No. 15/523,654, filed on May 1, 2017, now abandoned.
(60) Provisional application No. 62/130,800, filed on Mar. 10, 2015.
(60) Provisional application No. 62/075,328, filed on Nov. 5, 2014.

(51) Int. Cl. C12Q 1/6883; C12Q 1/6806; C12Q 1/686; C12Q 1/6874; C12Q 1/6876

(52) U.S. Cl. CPC C12Q 1/6883; C12Q 1/6806; C12Q 1/686; C12Q 1/6874; C12Q 1/6876; C12Q 2600/112; C12Q 2600/156; C12Q 2600/158

(57) ABSTRACT

The present invention provides methods for sequencing and analysis of nucleic acids and determining that a subject is positive for a non-usual interstitial pneumonia subtype.

### RELATED PATENT APPLICATIONS

This application is a continuation of U.S. patent application Ser. No. 17/218,125, filed Mar. 30, 2021, which is a continuation of U.S. patent application Ser. No. 16/840,009, filed Apr. 3, 2020, which is a continuation of U.S. patent application Ser. No. 16/551,645, filed Aug. 26, 2019, which is a continuation of U.S. patent application Ser. No. 15/523,654, filed May, 2017, which is a U.S. National Stage Application pursuant to 35 U.S.C. § 371 of PCT/US2015/059309, filed Nov. 5, 2015, which claims benefit of U.S. Provisional Application No. 62/130,800, filed Mar. 10, 2015 and claims benefit of U.S. Provisional Application No. 62/075,328, filed on Nov. 5, 2014, each incorporated in its entirety by reference herein.

### SEQUENCE LISTING

The instant application contains a Sequence Listing which has been submitted electronically in XML format and is hereby incorporated by reference in its entirety. Said XML copy, created on Mar. 9, 2023, is named 36024-739.304 SL.xml and is 67,863 bytes in size.

### INTRODUCTION

Interstitial lung diseases (ILD) are a heterogeneous group of acute and chronic bilateral parenchymal pulmonary disorders with similar clinical manifestations, but a wide spectrum of severity and outcome^{1,2}. Among these, idiopathic pulmonary fibrosis (IPF) is one of the most common and severe ILD, characterized by progressive fibrosis, worsening lung function and death³⁻⁶. Most patients diagnosed with IPF die within five years of their initial diagnosis^{7,8}. However, the recent availability of two new drugs and other therapeutics in development may change this picture⁹⁻¹¹, and accurate diagnosis is critical for appropriate therapeutic intervention^{5,12}.

IPF can be challenging to diagnose. The diagnostic approach to IPF requires exclusion of other interstitial pneumonias, as well as connective tissue disease and environmental and occupational exposures³⁻⁶. Patients suspected of having IPF usually undergo high-resolution computed tomography (HRCT), which confirms the disease with high specificity only if the pattern of usual interstitial pneumonia (UIP) is clearly evident^{5,13}. Yet, for a large number of patients, diagnosis necessitates an invasive surgical lung biopsy (SLB) to clarify the histopathologic features of interstitial pneumonia and/or UIP pattern^{5,14} and the typical length of time to diagnose IPF from the onset of symptoms may be 1-2 years¹⁵. Discordance between pathologists occurs, and a correct diagnosis can be dependent on individual experience¹⁶. Despite histopathologic evaluation, a definitive diagnosis may remain elusive. Diagnostic accuracy has been shown to increase when multidisciplinary teams (MDT) of pulmonologists, radiologists, and pathologists confer¹⁷; unfortunately not all patients and their physicians have access to this level of expert review by an experienced MDT. Such reviews are time consuming and require patients to be seen at regional centers of recognized expertise.

Accordingly, more effective methods of diagnosing IPF are required. In addition, methods of differentiating UIP from non-UIP are required.

### SUMMARY OF THE INVENTION

Disclosed herein is a method for nucleic acid sequencing comprising (a) obtaining a nucleic acid sample, wherein said nucleic acid sample comprises a plurality of messenger ribonucleic acid molecules; (b) subjecting said plurality of messenger ribonucleic acid molecules to reverse transcription to yield a plurality of complementary deoxyribonucleic acid molecules; and (c) subjecting the plurality of messenger ribonucleic acid molecules or derivatives thereof to sequencing. The messenger ribonucleic acid molecules can be derived from a tissue sample of the subject. Sequencing can comprise PCR. Subjecting can comprise hybridizing a plurality of probes to said plurality of messenger ribonucleic acid molecules. The plurality of probes can be labeled with a molecular marker.

Herein we describe methods of and systems used for differentiating between samples as usual interstitial pneumonia (UIP) or non-UIP using classifiers whose accuracy was confirmed using expert pathology diagnoses as truth labels. While gene expression profiling studies in the scientific literature have reported differential expression between IPF and other ILD subtypes^{18,19}, none have attempted to classify UIP in datasets containing other subtypes frequently present as part of the clinician's differential diagnosis.

In some embodiments, the present invention provides a method and/or system for detecting whether a lung tissue sample is positive for usual interstitial pneumonia (UIP) or non-usual interstitial pneumonia (non-UIP). In some embodiments a method is provided for: assaying the expression level of each of a first group of transcripts and a second group of transcripts in a test sample of a subject, wherein the first group of transcripts includes any one or more of the genes overexpressed in UIP and listed in any of Tables 5, 7, 9, 10, 11, and 12 and the second group of transcripts includes any one or more of the genes under-expressed in UIP and listed in any of Tables 5, 8, 9, 10, 11 or 12. In some embodiment, the method further provides for comparing the expression level of each of the first group of transcripts and the second group of transcripts with reference expression levels of the corresponding transcripts to (1) classify said lung tissue as usual interstitial pneumonia (UIP) if there is (a) an increase in an expression level corresponding to the first group or (b) a decrease in an expression level corresponding to the second group as compared to the reference expression levels, or (2) classify the lung tissue as non-usual interstitial pneumonia (non-UIP) if there is (c) an increase in the expression level corresponding to the second group or (d) a decrease in the expression level corresponding to the first group as compared to the reference expression levels. In some embodiments, the method further provides for determining and/or comparing sequence variants for any of the one or more genes listed in tables 5, 8, 9, 11, and/or 12.

In some embodiments, the present invention provides a method and/or system for detecting whether a lung tissue sample is positive for usual interstitial pneumonia (UIP) or non-usual interstitial pneumonia (non-UIP). In some embodiments, the method and/or system is used to assay by sequencing, array hybridization, or nucleic acid amplification the expression level of each of a first group of transcripts and a second group of transcripts in a test sample from a lung tissue of a subject, wherein the first group of transcripts includes any one or more of the genes over-expressed in UIP and listed in Tables 5, 7, 9, 10, 11 or 12 and the second group of transcripts includes any one or more of the genes under-expressed in UIP and listed in Tables 5, 8, 9, 10, 11 or 12. In certain embodiments, the method and/or system further compares the expression level of each of the first group of transcripts and the second group of transcripts with reference expression levels of the corresponding transcripts to (1) classify said lung tissue as usual interstitial pneumonia (UIP) if there is (a) an increase in an expression level corresponding to the first group or (b) a decrease in an expression level corresponding to the second group as compared to the reference expression levels, or (2) classify the lung tissue as non-usual interstitial pneumonia (non-UIP) if there is (c) an increase in the expression level corresponding to the second group or (d) a decrease in the expression level corresponding to the first group as compared to the reference expression levels.

In some embodiments, the present invention provides a method and/or system for detecting whether a test sample is positive for UIP or non-UIP by
measuring the expression level of two or more transcripts expressed and/or determining sequence variants for one or more transcripts expressed in the sample;
using a computer generated classifier to distinguish between UIP and non-UIP;
wherein the classifier is built using a spectrum of Non-UIP pathology subtypes comprising HP, NSIP, sarcoidosis, RB, bronchiolitis, and organizing pneumonia (OP).

In some embodiments, the test sample is a biopsy sample or a bronchoalveolar lavage sample. In some embodiments, the test sample is fresh-frozen or fixed.

In some embodiments, the transcript expression levels are determined by RT-PCR, DNA microarray hybridization, RNASeq, or a combination thereof. In some embodiments, one or more of the transcripts is labeled.

In some embodiments, the method comprises detecting cDNA produced from RNA expressed in the test sample, wherein, optionally, the cDNA is amplified from a plurality of cDNA transcripts prior to the detecting step.

In some embodiments, the methods of the present invention further comprise measuring the expression level of at least one control nucleic acid in the test sample.

In some embodiments, the methods of the present invention classify the lung tissue as any one of interstitial lung diseases (ILD), a particular type of ILD, a non-ILD, or non-diagnostic. In particular embodiments, methods of the present invention classify the lung tissue as either idiopathic pulmonary fibrosis (IPF) or Nonspecific interstitial pneumonia (NSIP).

In some embodiments, the method and/or system of the present invention comprises assaying the test sample for the expression level of one or more transcripts of any one of SEQ ID NOS: 1-22. In some embodiments, the method further comprises assaying the test sample for the expression level of from 1 to 20 other genes. In some embodiments, the other genes comprise one or more, or optionally all of HMCN2, ADAMTSL1, CD79B, KEL, KLHL14, MPP2, NMNAT2, PLXDC1, CAPN9, TALDO1, PLK4, IGHV3-72, IGKV1-9, and CNTN4.

In some embodiments, the method and/or systems of the present invention further comprise using smoking status as a covariate during training of a UIP vs. non-UIP classifier disclosed herein, wherein, optionally, the smoking status is determined by detecting an expression profile indicative of the subject's smoker status. In some embodiments, such a classifier is used to determine whether a test sample is UIP or non-UIP.

In some embodiments, the method and/or systems of the present invention comprises training a UIP vs. non-UIP classifier, wherein genes that are susceptible to smoker-status bias are excluded or weighed differently than genes that are not susceptible to smoker-status bias during the classifier training.

In some embodiments, the present invention provides a method and/or system for detecting whether a lung tissue sample is positive for usual interstitial pneumonia (UIP) or non-usual interstitial pneumonia (non-UIP), as described herein, wherein the method comprises a first classification of a test sample as smoker or non-smoker using a first classifier trained to recognize gene signatures that distinguish smokers from non-smokers; and wherein the method further comprises a second classification of the test sample a UIP or non-UIP, wherein the second classification step uses a second or third classifier, which second and third classifiers are trained to distinguish UIP vs. non-UIP in smokers (smoker-specific classifier) and non-smokers (non-smoker-specific classifier), respectively, and wherein the second classification uses either (i) the smoker-specific classifier if the test sample is classified as smoker in the first classification or (ii) the non-smoker-specific classifier if the test sample is classified as non-smoker in the first classification.

In some embodiments, the present invention provides a method and/or system for detecting whether a lung tissue sample is positive for usual interstitial pneumonia (UIP) or non-usual interstitial pneumonia (non-UIP), wherein the methods comprise implementing a classifier trained using one or more feature selected from gene expression, variants, mutations, fusions, loss of heterozygoxity (LOH), and biological pathway effect. In some embodiments, the classifier is trained using features comprising gene expression, sequence variants, mutations, fusions, loss of heterozygoxity (LOH), and biological pathway effect.

In some embodiments, the present invention provides for assaying 2 or more different transcripts, or 3 or more, 4 or more, 5 or more, 10 or more, 15 or more, 20 or more, or more than 20 different transcripts in the first group and/or 2 or more different transcripts, or 3 or more, 4 or more, 5 or more, 10 or more, 15 or more, 20 or more, or more than 20 different transcripts in the second group.

In some embodiments, the method provides for detecting 2 or more different transcripts of any one of SEQ ID NOS:1-22, or 3 or more, 4 or more, 5 or more, 10 or more, 15 or more, 20 or more, or more than 20 different transcripts of any one of SEQ ID NOS:1-22. In particular embodiments, the current methods provide for assaying the test sample for the expression level of all of the transcripts of SEQ ID NOS: 1-22. In some embodiments, the method further comprises assaying the test sample for the expression level of from 1 to 20 other genes. In some embodiments, the method provides for assaying one or more of HMCN2, ADAMTSL1, CD79B, KEL, KLHL14, MPP2, NMNAT2, PLXDC1, CAPN9, TALDO1, PLK4, IGHV3-72, IGKV1-9, and CNTN4.

Disclosed herein is a method for determining that a subject is at risk for a non-usual interstitial pneumonia (non-UIP) subtype of a plurality of non-UIP subtypes, comprising:(a) obtaining a biological sample of said subject; (b) assaying nucleic acid molecules derived from said biological sample to identify a level of expression of at least one gene associated with said non-UIP subtype; and (c)processing said level of expression to generate a classification of said biological sample as being at risk for said non-UIP subtype. The non-UIP subtype can be hypersensitivity pneumonitis (HP), non-specific interstitial pneumonia (NSIP), sarcoidosis, respiratory bronchiolitis (RB), bronchiolitis, diffuse alveolar damage (DAD) or organizing pneumonia (OP). The biological sample can be a transbronchial biopsy sample or a bronchoalveolar lavage sample. Step (b) can comprise sequencing. Assaying can further comprise identifying a level of expression of at least one control nucleic acid molecule in said biological sample. The plurality of non-UIP subtypes can comprise hypersensitivity pneumonitis (HP), non-specific interstitial pneumonia (NSIP), sarcoidosis, respiratory bronchiolitis (RB), bronchiolitis, diffuse alveolar damage (DAD) or organizing pneumonia (OP). Step (c) can be performed using a machine learning algorithm that is trained to identify said non-UIP subtype of said plurality of non-UIP subtypes. The machine learning algorithm can be trained using features comprising gene expression variants, gene fusions, loss of heterozygosity, or biological pathway effect. The gene expression variants can be alternative splice variants. The machine learning algorithm can be trained with a training set that is independent of said biological sample. The biological sample can be fresh-frozen or fixed. The nucleic acid molecules can be ribonucleic acids (RNA) molecules, and said assaying can comprise generating complementary deoxyribonucleic acid (cDNA) molecules from said RNA molecules. The subject can be suspected of having an interstitial lung disease based at least in part on one or more clinical signs or one or more symptoms. The one or more symptoms can comprise shortness of breath or dry cough. The one or more clinical signs can comprise a result of an imaging test, a pulmonary function test, or a lung tissue analysis. The imaging test can be chest X-ray or computerized tomography. The computerized tomography can be high-resolution computerized tomography. The pulmonary function test can be spirometry, oximetry, or an exercise stress test. The lung tissue analysis can comprise histological or cytological analysis of a lung tissue sample of said subject. The method can further comprise providing a therapeutic intervention to said subject based at least in part on said classification generated in (c).

### BRIEF DESCRIPTION OF THE FIGURES

FIG. 1. Pairwise correlation on explant samples obtained from three patients diagnosed with IPF (Patients P1, P2, and P3). Locations (upper or lower, central or peripheral) are indicated for each sample. The top 200 differentially expressed genes separating IPF samples from normal lung samples were used to compute pairwise Pearson correlation coefficients and plotted as a heatmap with higher correlation represented in magenta color, and lower correlation represented in green color. Correlation between and with normal lung samples are in the 0.7 range (not shown).

FIGS. 2A-2D. Performance of a classifier built using microarray data. ROC curves were used to characterize performance in the training set using leave-one-patient-out (LOPO) cross-validation (FIG. 2A) and in the independent test set by scoring the samples with a fixed model (FIG. 2C). Scores for individual samples are shown across patients in the training set (FIG. 2B), and across patients in an independent test set (FIG. 2D). Patient-level pathology diagnosis is shown on the x-axis. Samples with UIP pathology labels are indicated by closed circles, and non-UIP samples by pathology are shown in open triangles. A dotted horizontal line is drawn to indicate the threshold that corresponds to 92% specificity and 64% sensitivity (FIG. 2B) and 92% specificity and 82% sensitivity (FIG. 2D).

FIGS. 3A-3D. Performance of classifiers built using RNASeq (FIG. 3A and FIG. 3B) and microarray on the matched set (FIG. 3C and FIG. 3D). Leave-one-patient-out (LOPO) cross-validation was performed and receiver operator characteristic (ROC) curves were produced for RNASeq (FIG. 3A) and microarray (FIG. 3C) classifiers. Scores for individual samples in the training sets are shown for RNASeq (FIG. 3B), and microarray (FIG. 3D) classification. Patient-level pathology diagnosis is shown on the x-axis. Samples with UIP pathology labels are indicated by closed circles, and non-UIP samples by pathology are shown in open triangles. A score threshold corresponding to 95% specificity is indicated as a horizontal line in FIG. 3B and FIG. 3D.

FIG. 4. Simulation study assessing the impact of mislabeling on the classification performance. The array training set (n=77) was used for this study. At a given proportion of swapped labels in the data set (x-axis), individual samples’ classification labels were swapped to another class label with a weight accounting for the disagreement level of three expert pathology diagnoses. Each boxplot was drawn using LOPO CV performances (AUC) from 100 repeated simulations. The finer dotted horizontal line at AUC=0.5 represents random performance, i.e., no classification, and the coarser dotted line corresponds to the classifier performance shown in FIG. 2A.

FIG. 5. Central pathology diagnostic process for a hypothetical patient with two samples (sample A and sample B). Three expert pathologists participate in the review process. For sample-level diagnosis, the glass slides for each sample are reviewed by each pathologist (Pathologist is abbreviated as Path). For patient-level diagnosis, glass slides from all samples (two in this exercise) are gathered and reviewed together by each pathologist. Both sample-level and patient-level diagnoses go through the same review process. A majority vote is used as the final diagnosis, unless expert pathologists disagree even after the conferral, in which case, the sample is omitted due to lack of confidence in the diagnosis. Only a single such case was observed among all banked tissues (n=128).

FIG. 6. Location of lung samplings from three normal organ donors (top) and three patients diagnosed with IPF (bottom). Donors N1-N3 and P3 were female. Donors P1 and P2 were male.

FIG. 7A. Illustration of a computer system usable for implementing aspects disclosed herein.

FIG. 7B. Detailed illustration of the processor of the computer system of FIG. 7A.

FIG. 7C. Detailed illustration of one non-limiting method of the present invention, wherein gene product expression data for known UIP and non-UIP samples are used to train a classifier (e.g., using a classifier training module) for differentiating UIP vs. non-UIP, wherein the classifier optionally considers smoker status as a covariant, and wherein gene product expression data from unknown samples are input into the trained classifier to identify the unknown samples as either UIP or non-UIP, and wherein the results of the classification via the classifier are defined and output via a report.

FIG. 8. Differential gene expression in UIP and Non-UIP samples between smokers and non-smokers. The number of genes differentially expressed between UIP and Non UIP samples differs drastically between smokers and non-smokers.

FIG. 9. Shows differential gene expression between UIP and Non-UIP samples is susceptible to smoker-status bias. Direction (i.e., over- vs. under-expression) and magnitude (circle size) of differential gene expression is confounded by smoking status.

FIGS. 10A-10D. Examples of genes that are differentially expressed in UIP vs. Non-UIP and the effect of smoking status on expression levels. FIG. 10A: differential expression of IGHV3-72 in UIP vs Non-UIP smokers vs. non-smokers. FIG. 10B: differential expression of CPXM1 in UIP vs Non-UIP smokers vs. non-smokers. FIG. 10C: differential expression of BPIFA1 in UIP vs Non-UIP smokers vs. non-smokers. FIG. 10D: differential expression of HLA-U in UIP vs Non-UIP smokers vs. non-smokers.

### DEFINITIONS

“Interstitial lung disease” or “ILD” (also known as diffuse parenchymal lung disease (DPLD)) as used herein refers to a group of lung diseases affecting the interstitium (the tissue and space around the air sacs of the lungs). ILD can be classified according to a suspected or known cause, or can be idiopathic. For example, ILD can be classified as caused by inhaled substances (inorganic or organic), drug induced (e.g., antibiotics, chemotherapeutic drugs, antiarrhythmic agents, statins), associated with connective tissue disease (e.g., systemic sclerosis, polymyositis, dermatomyositis, systemic lupus erythematous, rheumatoid arthritis), associated with pulmonary infection (e.g., atypical pneumonia, Pneumocystis pneumonia (PCP), tuberculosis, Chlamydia trachomatis, Respiratory Syncytial Virus), associated with a malignancy (e.g., Lymphangitic carcinomatosis), or can be idiopathic (e.g., sarcoidosis, idiopathic pulmonary fibrosis, Hamman-Rich syndrome, antisynthetase syndrome).

“ILD Inflammation” as used herein refers to an analytical grouping of inflammatory ILD subtypes characterized by underlying inflammation. These subtypes can be used collectively as a comparator against IPF and/or any other non-inflammation lung disease subtype. “ILD inflammation” can include HP, NSIP, sarcoidosis, and/or organizing pneumonia.

“Idiopathic interstitial pneumonia” or “IIP” (also referred to as noninfectious pneumonia” refers to a class of ILDs which includes, for example, desquamative interstitial pneumonia, nonspecific interstitial pneumonia, lymphoid interstitial pneumonia, cryptogenic organizing pneumonia, and idiopathic pulmonary fibrosis.

“Idiopathic pulmonary fibrosis” or “IPF” as used herein refers to a chronic, progressive form of lung disease characterized by fibrosis of the supporting framework (interstitium) of the lungs. By definition, the term is used when the cause of the pulmonary fibrosis is unknown (“idiopathic”). Microscopically, lung tissue from patients having IPF shows a characteristic set of histologic/pathologic features known as usual interstitial pneumonia (UIP), which is a pathologic counterpart of IPF.

“Nonspecific interstitial pneumonia” or “NSIP” is a form of idiopathic interstitial pneumonia generally characterized by a cellular pattern defined by chronic inflammatory cells with collagen deposition that is consistent or patchy, and a fibrosing pattern defined by a diffuse patchy fibrosis. In contrast to UIP, there is no honeycomb appearance nor fibroblast foci that characterize usual interstitial pneumonia.

“Hypersensitivity pneumonitis” or “HP” refers to also called extrinsic allergic alveolitis, (EAA) refers to an inflammation of the alveoli within the lung caused by an exaggerated immune response and hypersensitivity to as a result of an inhaled antigen (e.g., organic dust).

“Pulmonary sarcoidosis” or “PS” refers to a syndrome involving abnormal collections of chronic inflammatory cells (granulomas) that can form as nodules. The inflammatory process for HP generally involves the alveoli, small bronchi, and small blood vessels. In acute and subacute cases of HP, physical examination usually reveals dry rales.

The term “microarray” refers to an ordered arrangement of hybridizable array elements, preferably polynucleotide probes, on a substrate.

The term “polynucleotide,” when used in singular or plural, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” can also include DNAs (e.g., cDNAs) and RNAs that contain one or more modified bases (e.g., to provide a detectable signal, such as a fluorophore). Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide (e.g., 100, 50, 20 or fewer nucleotides) including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The terms “gene product” or “expression product” are used herein interchangeably to refer to the RNA transcription products (RNA transcript) of a gene, including mRNA, and the polypeptide translation product of such RNA transcripts. A gene product can be, for example, a polynucleotide gene expression product (e.g., an unspliced RNA, an mRNA, a splice variant mRNA, a microRNA, a fragmented RNA, and the like) or a protein expression product (e.g., a mature polypeptide, a post-translationally modified polypeptide, a splice variant polypeptide, and the like). In some embodiments the gene expression product may be a sequence variant including mutations, fusions, loss of heterozygoxity (LOH), and/or biological pathway effects.

The term “normalized expression level” as applied to a gene expression product refers to a level of the gene product normalized relative to one or more reference (or control) gene expression products.

A “reference expression level” as applied to a gene expression product refers to an expression level for one or more reference (or control) gene expression products. A “reference normalized expression level” as applied to a gene expression product refers to a normalized expression level value for one or more reference (or control) gene expression products (i.e., a normalized reference expression level). In some embodiments, a reference expression level is an expression level for one or more gene product in normal sample, as described herein. In some embodiments, a reference expression level is determined experimentally. In some embodiments, a reference expression level is a historical expression level, e.g., a database value of a reference expression level in a normal sample, which sample indicates a single reference expression level, or a summary of a plurality of reference expression levels (such as, e.g., (i) an average of two or more, preferably three or more reference expression levels from replicate analysis of the reference expression level from a single sample; (ii) an average of two or more, preferably three or more reference expression levels from analysis of the reference expression level from a plurality of different samples (e.g., normal samples); (iii) and a combination of the above mentioned steps (i) and (ii) (i.e., average of reference expression levels analyzed from a plurality of samples, wherein at least one of the reference expression levels are analyzed in replicate). In some embodiments, the “reference expression level” is an expression level of sequence variants, for example, in a sample that has been definitively determined to be UIP or non-UIP by other means (i.e. confirmed pathological diagnosis).

A “reference expression level value” as applied to a gene expression product refers to an expression level value for one or more reference (or control) gene expression products. A “reference normalized expression level value” as applied to a gene expression product refers to a normalized expression level value for one or more reference (or control) gene expression products.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, (Wiley Interscience, 1995).

“Stringent conditions” or “high stringency conditions”, as defined herein, typically: (1) employ low ionic strength solutions and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual (Cold Spring Harbor Press, 1989), and include the use of washing solution and hybridization conditions (e.g., temperature, ionic strength and % SDS) less stringent that those described above. An example of moderately stringent condition is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

“Sensitivity” as used herein refers to the proportion of true positives of the total number tested that actually have the target disorder (i.e., the proportion of patients with the target disorder who have a positive test result). “Specificity” as used herein refers to the proportion of true negatives of all the patients tested who actually do not have the target disorder (i.e., the proportion of patients without the target disorder who have a negative test result).

In the context of the present invention, reference to “at least one,” “at least two,” “at least five,” etc. of the genes listed in any particular gene set means any one or any and all combinations of the genes listed.

The terms “splicing” and “RNA splicing” are used interchangeably and refer to RNA processing that removes introns and joins exons to produce mature mRNA with continuous coding sequence that moves into the cytoplasm of a eukaryotic cell.

The term “exon” refers to any segment of an interrupted gene that is represented in a mature RNA product (B. Lewin,Genes 7V(Cell Press, 1990)). In theory the term “intron” refers to any segment of DNA that is transcribed but removed from within the transcript by splicing together the exons on either side of it. Operationally, exon sequences occur in the mRNA sequence of a gene as defined by Ref. SEQ ID numbers. Operationally, intron sequences are the intervening sequences within the genomic DNA of a gene, bracketed by exon sequences and usually having GT and AG splice consensus sequences at their 5′ and 3′ boundaries.

A “computer-based system” refers to a system of hardware, software, and data storage medium used to analyze information. Hardware of a patient computer-based system can include a central processing unit (CPU), and hardware for data input, data output (e.g., display), and data storage. The data storage medium can include any manufacture comprising a recording of the present information as described above, or a memory access device that can access such a manufacture.

As used herein the term “module” refers to any assembly and/or set of operatively-coupled electrical components that can include, for example, a memory, a processor, electrical traces, optical connectors, software (executing in hardware), and/or the like. For example, a module executed in the processor can be any combination of hardware-based module (e.g., a field-programmable gate array (FPGA), an application specific integrated circuit (ASIC), a digital signal processor (DSP)) and/or software-based module (e.g., a module of computer code stored in memory and/or executed at the processor) capable of performing one or more specific functions associated with that module.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” or “computing means” references any hardware and/or software combination that will perform the functions required of it. For example, a suitable processor may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

A “test sample” is a sample of one or more cells, preferable a tissue sample (e.g., a lung tissue sample such as a transbronchial biopsy (TBB) sample) obtained from a subject. In some embodiments, a test sample is a biopsy sample obtained by any means known in the art. In particular embodiments, the test sample is a sample obtained by a video-assisted thoracoscopic surgery (VATS); a bronchoalveolar lavage (BAL); a transbronchial biopsy (TBB); or a cryo-transbronchial biopsy. In some embodiments the test sample is obtained from a patient suspected of having a lung disease, e.g., an ILD, based on clinical signs and symptoms with which the patient presents (e.g., shortness of breath (generally aggravated by exertion), dry cough), and, optionally the results of one or more of an imaging test (e.g., chest X-ray, computerized tomography (CT)), a pulmonary function test (e.g., spirometry, oximetry, exercise stress test), lung tissue analysis (e.g., histological and/or cytological analysis of samples obtained by bronchoscopy, bronchoalveolar lavage, surgical biopsy).

A “gene signature” is a gene expression pattern (i.e., expression level of one or more gene, or fragments thereof), which is indicative of some characteristic or phenotype. In some embodiments, gene signature refers to the expression (and/or lack of expression) of a gene, a plurality of genes, a fragment of a gene or a plurality fragments of one or more genes, which expression and/or lack of expression is indicative of UIP, Non-UIP, smoker-status, or Non-smoker-status.

As used herein, “is a smoker” is meant to refer to a subject who currently smokes cigarettes or a person who has smoked cigarettes in the past or a person who has the gene signature of a person who currently smokes cigarettes or has smoked cigarettes in the past.

As used herein, “variant”, when used to describe a feature used during training of a classifier of the present invention, refers to an alternative splice variant.

As used herein, “mutation”, when used to describe a feature used during training of a classifier of the present invention, refers to a sequence deviation from a known normal reference sequence. In some embodiments, the deviation is a deviation from an accepted native gene sequence according to a publically accessible database such as the UniGene database (Pontius J U, Wagner L, Schuler G D. UniGene: a unified view of the transcriptome. In: The NCBI Handbook. Bethesda (MD): National Center for Biotechnology Information; 2003, incorporated herein), RefSeq (The NCBI handbook [Internet]. Bethesda (MD): National Library of Medicine (US), National Center for Biotechnology Information; 2002 October. Chapter 18, The Reference Sequence (RefSeq) Project, available at the world wide web address: ncbi.nlm.nih.gov/refseq/), Ensembl (EMBL, available at the world wide web address: ensembl.org/index.html), and the like. In some embodiments, the mutation includes an addition, deletion, or substitution of a sequence residue present in the reference sequence.

Abbreviations include: HRCT, high-resolution computed tomography; VATS, video-assisted thorascopic surgery; SLB, surgical lung biopsy; TBB, transbronchial biopsy; RB, respiratory bronchiolitis; OP, organizing pneumonia, DAD, diffuse alveolar damage, CIF/NOC, chronic interstitial fibrosis not otherwise classified; MDT, multidisciplinary team; CV, cross-validation; LOPO, leave-one-patient-out; ROC, receiver operator characteristic; AUC, area under the curve; RNASeq, RNA sequencing by next-generation sequencing technology; NGS, next-generation sequencing technology; H&E, hematoxylin and eosin; FDR, false discovery rate; IRB, Institutional Review Board; ATS, American Thoracic Society; COPD, chronic obstructive pulmonary disease; KEGG, Kyoto Encyclopedia of Genes and Genomes; CI, confidence interval

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. As used herein, “about” means plus or minus 10% of the indicated value.

### DETAILED DESCRIPTION OF THE INVENTION

Disclosed herein are methods of and/or systems for using a molecular signature to differentiate UIP from other ILD subtypes. The accurate diagnosis of UIP from samples where expert pathology is not available stands to benefit ILD patients by accelerating diagnosis, thus facilitating treatment decisions and reducing surgical risk to patients and costs to the healthcare system.

Also disclosed herein are methods of and/or systems for using the smoker or non-smoker status of a subject to improve differentiation of UIP from other ILD subtypes using a molecular signature.

Thus, the methods and/or systems disclosed herein provide classifiers which can differentiate UIP from non-UIP patterns based on high-dimensional transcriptional data without prior knowledge of clinical or demographic information.

In some embodiments, the present invention provides methods for differentiating UIP from non-UIP using a classifier that comprises or consists of one or more sequences or fragments thereof presented in any of Tables 5, 7, 8, 9, 10, 11, or 12 or at least one sequence or fragment thereof from each of Tables 5, 7, 8, 9, 10, 11 and 12. In some embodiments, the present invention provides such methods that use a classifier comprising or consisting of at least 1, 2, 3, 4, 5, 6, 7, 8, 9, 10 or more of the sequences provided in any one or more or all of Tables 5, 7, 8, 9, 10, 11 and 12. For example, in some embodiments, the present invention provides such methods that use classifiers comprising or consisting of at least 11, 12, 13, 14, 15, 20, 30, 50, 100, 150, 200, 250, 300, or more sequences provided in any one or more or all of Tables 5, 7, 8, 9, 10, 11 and 12, including all integers (e.g., 16, 17, 18, 19, 21, 22, 23, 24, 25 sequences, etc.) and ranges (e.g., from about 1-10 sequences from any one or more or all of Tables 5, 7, 8, 9, 10, 11, and 12, from about 10-15 sequences, 10-20 sequences, 5-30 sequences, 5-50 sequences, 10-100 sequences, 50-200 sequences, etc.) between.

In some particular embodiments, the present invention provides methods and/or systems for differentiating UIP from non-UIP using a classifier that comprises or consists of one or more of the following sequences or fragments thereof: 1) HLA-F (SEQ ID NO.:1), 2) CDKL2 (SEQ ID NO.:2), 3) GPR98 (SEQ ID NO.:3) 4) PRKCQ (SEQ ID NO.:4) 5) HLA-G (SEQ ID NO.:5), 6) PFKFB3 (SEQ ID NO.:6), 7) CEACAM1 (SEQ ID NO.:7), 8) RABGAP1L (SEQ ID NO.:8), 9) CD274 (SEQ ID NO.:9), 10) PRUNE2 (SEQ ID NO.:10), 11) ARAP2 (SEQ ID NO.:11), 12) DZIP1 (SEQ ID NO.:12), 13) MXRA7 (SEQ ID NO.:13), 14) PTCHD4 (SEQ ID NO.:14), 15) PDLIM3 (SEQ ID NO.:15), 16) CNN1 (SEQ ID NO.:16), 17) NIPSNAP3B (SEQ ID NO.:17), 18) PAQR7 (SEQ ID NO.:18), 19) ACTG2 (SEQ ID NO.:19), 20) NA (SEQ ID NO.:20), 21) TIMP2 (SEQ ID NO.:21), and 22) DES (SEQ ID NO.:22). In particular aspects, the classifier may contain 1, 2, 3, 4, 5, 6, 7, 8, or more additional genes. In other aspects, the classifier may omit 1, 2, 3, 4, 5, 6, 7, 8, or more, of these genes, while optionally including other genes.

In some embodiments, the present invention provides a method and/or system for differentiating UIP from non-UIP using a classifier that comprises or consists of 2; 3; 4; 5; 6; 7; 8; 9; 10; 11; 12; 13; 14; 15; 16; 17; 18; 19; 20; or 21 of the following sequences: 1) HLA-F (SEQ ID NO.:1), 2) CDKL2 (SEQ ID NO.:2), 3) GPR98 (SEQ ID NO.:3), 4) PRKCQ (SEQ ID NO.:4), 5) HLA-G (SEQ ID NO.:5), 6) PFKFB3 (SEQ ID NO.:6), 7) CEACAM1 (SEQ ID NO.:7), 8) RABGAP1L (SEQ ID NO.:8), 9) CD274 (SEQ ID NO.:9), 10) PRUNE2 (SEQ ID NO.:10), 11) ARAP2 (SEQ ID NO.:11), 12) DZIP1 (SEQ ID NO.:12), 13) MXRA7 (SEQ ID NO.:13), 14) PTCHD4 (SEQ ID NO.:14), 15) PDLIM3 (SEQ ID NO.:15), 16) CNN1 (SEQ ID NO.:16), 17) NIPSNAP3B (SEQ ID NO.:17), 18) PAQR7 (SEQ ID NO.:18), 19) ACTG2 (SEQ ID NO.:19), 20) NA (SEQ ID NO.:20), 21) TIMP2 (SEQ ID NO.:21), and 22) DES (SEQ ID NO.:22) in any combination. In particular aspects, such a classifier contains 1, 2, 3, 4, 5, 6, 7, 8, or more additional genes. In other aspects, the classifier may omit 1, 2, 3, 4, 5, 6, 7, 8, or more, of these genes, while optionally including other genes.

In some embodiments, the present invention provides a method and/or system for differentiating UIP from non-UIP using a classifier that comprises or consists of all of the following sequences: 1) HLA-F (SEQ ID NO.:1), 2) CDKL2 (SEQ ID NO.:2), 3) GPR98 (SEQ ID NO.:3), 4) PRKCQ (SEQ ID NO.:4), 5) HLA-G (SEQ ID NO.:5), 6) PFKFB3 (SEQ ID NO.:6), 7) CEACAM1 (SEQ ID NO.:7), 8) RABGAP1L (SEQ ID NO.:8), 9) CD274 (SEQ ID NO.:9), 10) PRUNE2 (SEQ ID NO.:10), 11) ARAP2 (SEQ ID NO.:11), 12) DZIP1 (SEQ ID NO.:12), 13) MXRA7 (SEQ ID NO.:13), 14) PTCHD4 (SEQ ID NO.:14), 15) PDLIM3 (SEQ ID NO.:15), 16) CNN1 (SEQ ID NO.:16), 17) NIPSNAP3B (SEQ ID NO.:17), 18) PAQR7 (SEQ ID NO.:18), 19) ACTG2 (SEQ ID NO.:19), 20) NA (SEQ ID NO.:20), 21) TIMP2 (SEQ ID NO.:21), and 22) DES (SEQ ID NO.:22). In particular aspects, the classifier contains 1, 2, 3, 4, 5, 6, 7, 8, or more additional genes.

In some particular embodiments, the present invention provides methods and/or systems for differentiating UIP from non-UIP using a classifier that comprises or consists of one or more of the following sequences or fragments thereof: 1) HLA-F (SEQ ID NO.:1), 2) HMCN2, 3) ADAMTSL1, 4) CD79B, 5) KEL, 6) KLHL14, 7) MPP2, 8) NMNAT2, 9) PLXDC1, 10) CAPN9, 11) TALDO1, 12) PLK4, 13) IGHV3-72, 14) IGKV1-9, and 15) CNTN4. In particular aspects, the classifier may contain 1, 2, 3, 4, 5, 6, 7, 8, or more additional genes. In other aspects, the classifier may omit 1, 2, 3, 4, 5, 6, 7, 8, or more, of these genes, while optionally including other genes.

In some embodiments, the present invention provides a method and/or system for differentiating UIP from non-UIP using a classifier that comprises or consists of 2; 3; 4; 5; 6; 7; 8; 9; 10; 11; 12; 13; or 14 of the following sequences: 1) HLA-F (SEQ ID NO.:1), 2) HMCN2, 3) ADAMTSL1, 4) CD79B, 5) KEL, 6) KLHL14, 7) MPP2, 8) NMNAT2, 9) PLXDC1, 10) CAPN9, 11) TALDO1, 12) PLK4, 13) IGHV3-72, 14) IGKV1-9, and 15) CNTN4. In particular aspects, the classifier may contain 1, 2, 3, 4, 5, 6, 7, 8, or more additional genes. In other aspects, the classifier may omit 1, 2, 3, 4, 5, 6, 7, 8, or more, of these genes, while optionally including other genes.

In some embodiments, the present invention provides a method and/or system for differentiating UIP from non-UIP using a classifier that comprises or consists of the following sequences: 1) HLA-F (SEQ ID NO.:1), 2) HMCN2, 3) ADAMTSL1, 4) CD79B, 5) KEL, 6) KLHL14, 7) MPP2, 8) NMNAT2, 9) PLXDC1, 10) CAPN9, 11) TALDO1, 12) PLK4, 13) IGHV3-72, 14) IGKV1-9, and 15) CNTN4. In particular aspects, the classifier may contain 1, 2, 3, 4, 5, 6, 7, 8, or more additional genes. In other aspects, the classifier may omit 1, 2, 3, 4, 5, 6, 7, 8, or more, of these genes, while optionally including other genes.

In some particular embodiments, the present invention provides methods and/or systems for differentiating UIP from non-UIP using a classifier that comprises or consists of HLA-F (SEQ ID NO.:1) or fragments thereof. In one such embodiment, the method uses a classifier comprising 1) HLA-F (SEQ ID NO.:1) and at least one of 2) CDKL2 (SEQ ID NO.:2), 3) GPR98 (SEQ ID NO.:3), 4) PRKCQ (SEQ ID NO.:4), 5) HLA-G (SEQ ID NO.:5), 6) PFKFB3 (SEQ ID NO.:6), 7) CEACAM1 (SEQ ID NO.:7), 8) RABGAP1L (SEQ ID NO.:8), 9) CD274 (SEQ ID NO.:9), 10) PRUNE2 (SEQ ID NO.:10), 11) ARAP2 (SEQ ID NO.:11), 12) DZIP1 (SEQ ID NO.:12), 13) MXRA7 (SEQ ID NO.:13), 14) PTCHD4 (SEQ ID NO.:14), 15) PDLIM3 (SEQ ID NO.:15), 16) CNN1 (SEQ ID NO.:16), 17) NIPSNAP3B (SEQ ID NO.:17), 18) PAQR7 (SEQ ID NO.:18), 19) ACTG2 (SEQ ID NO.:19), 20) NA (SEQ ID NO.:20), 21) TIMP2 (SEQ ID NO.:21), and 22) DES (SEQ ID NO.:22). In particular aspects, the classifier contains 1, 2, 3, 4, 5, 6, 7, 8, or more additional genes.

In some particular embodiments, the present invention provides methods and/or systems for differentiating UIP from non-UIP using a classifier that comprises or consists of HMCN2 or fragments thereof. In one such embodiment, the method uses a classifier comprising HMCN2 and at least one of 1) HLA-F (SEQ ID NO.:1) 2) CDKL2 (SEQ ID NO.:2), 3) GPR98 (SEQ ID NO.:3), 4) PRKCQ (SEQ ID NO.:4), 5) HLA-G (SEQ ID NO.:5), 6) PFKFB3 (SEQ ID NO.:6), 7) CEACAM1 (SEQ ID NO.:7), 8) RABGAP1L (SEQ ID NO.:8), 9) CD274 (SEQ ID NO.:9), 10) PRUNE2 (SEQ ID NO.:10), 11) ARAP2 (SEQ ID NO.:11), 12) DZIP1 (SEQ ID NO.:12), 13) MXRA7 (SEQ ID NO.:13), 14) PTCHD4 (SEQ ID NO.:14), 15) PDLIM3 (SEQ ID NO.:15), 16) CNN1 (SEQ ID NO.:16), 17) NIPSNAP3B (SEQ ID NO.:17), 18) PAQR7 (SEQ ID NO.:18), 19) ACTG2 (SEQ ID NO.:19), 20) NA (SEQ ID NO.:20), 21) TIMP2 (SEQ ID NO.:21), and 22) DES (SEQ ID NO.:22). In particular aspects, the classifier contains 1, 2, 3, 4, 5, 6, 7, 8, or more additional genes.

In some particular embodiments, the present invention provides methods and/or systems for differentiating UIP from non-UIP using a classifier that comprises or consists of ADAMTSL1 or fragments thereof. In one such embodiment, the method uses a classifier comprising ADAMTSL1 and at least one of 1) HLA-F (SEQ ID NO.:1) 2) CDKL2 (SEQ ID NO.:2), 3) GPR98 (SEQ ID NO.:3), 4) PRKCQ (SEQ ID NO.:4), 5) HLA-G (SEQ ID NO.:5), 6) PFKFB3 (SEQ ID NO.:6), 7) CEACAM1 (SEQ ID NO.:7), 8) RABGAP1L (SEQ ID NO.:8), 9) CD274 (SEQ ID NO.:9), 10) PRUNE2 (SEQ ID NO.:10), 11) ARAP2 (SEQ ID NO.:11), 12) DZIP1 (SEQ ID NO.:12), 13) MXRA7 (SEQ ID NO.:13), 14) PTCHD4 (SEQ ID NO.:14), 15) PDLIM3 (SEQ ID NO.:15), 16) CNN1 (SEQ ID NO.:16), 17) NIPSNAP3B (SEQ ID NO.:17), 18) PAQR7 (SEQ ID NO.:18), 19) ACTG2 (SEQ ID NO.:19), 20) NA (SEQ ID NO.:20), 21) TIMP2 (SEQ ID NO.:21), and 22) DES (SEQ ID NO.:22). In particular aspects, the classifier contains 1, 2, 3, 4, 5, 6, 7, 8, or more additional genes.

In some particular embodiments, the present invention provides methods and/or systems for differentiating UIP from non-UIP using a classifier that comprises or consists of CD79B or fragments thereof. In one such embodiment, the method uses a classifier comprising CD79B and at least one of 1) HLA-F (SEQ ID NO.:1) 2) CDKL2 (SEQ ID NO.:2), 3) GPR98 (SEQ ID NO.:3), 4) PRKCQ (SEQ ID NO.:4), 5) HLA-G (SEQ ID NO.:5), 6) PFKFB3 (SEQ ID NO.:6), 7) CEACAM1 (SEQ ID NO.:7), 8) RABGAP1L (SEQ ID NO.:8), 9) CD274 (SEQ ID NO.:9), 10) PRUNE2 (SEQ ID NO.:10), 11) ARAP2 (SEQ ID NO.:11), 12) DZIP1 (SEQ ID NO.:12), 13) MXRA7 (SEQ ID NO.:13), 14) PTCHD4 (SEQ ID NO.:14), 15) PDLIM3 (SEQ ID NO.:15), 16) CNN1 (SEQ ID NO.:16), 17) NIPSNAP3B (SEQ ID NO.:17), 18) PAQR7 (SEQ ID NO.:18), 19) ACTG2 (SEQ ID NO.:19), 20) NA (SEQ ID NO.:20), 21) TIMP2 (SEQ ID NO.:21), and 22) DES (SEQ ID NO.:22). In particular aspects, the classifier contains 1, 2, 3, 4, 5, 6, 7, 8, or more additional genes.

In some particular embodiments, the present invention provides methods and/or systems for differentiating UIP from non-UIP using a classifier that comprises or consists of KEL or fragments thereof. In one such embodiment, the method uses a classifier comprising KEL and at least one of 1) HLA-F (SEQ ID NO.:1) 2) CDKL2 (SEQ ID NO.:2), 3) GPR98 (SEQ ID NO.:3), 4) PRKCQ (SEQ ID NO.:4), 5) HLA-G (SEQ ID NO.:5), 6) PFKFB3 (SEQ ID NO.:6), 7) CEACAM1 (SEQ ID NO.:7), 8) RABGAP1L (SEQ ID NO.:8), 9) CD274 (SEQ ID NO.:9), 10) PRUNE2 (SEQ ID NO.:10), 11) ARAP2 (SEQ ID NO.:11), 12) DZIP1 (SEQ ID NO.:12), 13) MXRA7 (SEQ ID NO.:13), 14) PTCHD4 (SEQ ID NO.:14), 15) PDLIM3 (SEQ ID NO.:15), 16) CNN1 (SEQ ID NO.:16), 17) NIPSNAP3B (SEQ ID NO.:17), 18) PAQR7 (SEQ ID NO.:18), 19) ACTG2 (SEQ ID NO.:19), 20) NA (SEQ ID NO.:20), 21) TIMP2 (SEQ ID NO.:21), and 22) DES (SEQ ID NO.:22). In particular aspects, the classifier contains 1, 2, 3, 4, 5, 6, 7, 8, or more additional genes.

In some particular embodiments, the present invention provides methods and/or systems for differentiating UIP from non-UIP using a classifier that comprises or consists of KLHL14 or fragments thereof. In one such embodiment, the method uses a classifier comprising KLHL14 and at least one of 1) HLA-F (SEQ ID NO.:1) 2) CDKL2 (SEQ ID NO.:2), 3) GPR98 (SEQ ID NO.:3), 4) PRKCQ (SEQ ID NO.:4), 5) HLA-G (SEQ ID NO.:5), 6) PFKFB3 (SEQ ID NO.:6), 7) CEACAM1 (SEQ ID NO.:7), 8) RABGAP1L (SEQ ID NO.:8), 9) CD274 (SEQ ID NO.:9), 10) PRUNE2 (SEQ ID NO.:10), 11) ARAP2 (SEQ ID NO.:11), 12) DZIP1 (SEQ ID NO.:12), 13) MXRA7 (SEQ ID NO.:13), 14) PTCHD4 (SEQ ID NO.:14), 15) PDLIM3 (SEQ ID NO.:15), 16) CNN1 (SEQ ID NO.:16), 17) NIPSNAP3B (SEQ ID NO.:17), 18) PAQR7 (SEQ ID NO.:18), 19) ACTG2 (SEQ ID NO.:19), 20) NA (SEQ ID NO.:20), 21) TIMP2 (SEQ ID NO.:21), and 22) DES (SEQ ID NO.:22). In particular aspects, the classifier contains 1, 2, 3, 4, 5, 6, 7, 8, or more additional genes.

In some particular embodiments, the present invention provides methods and/or systems for differentiating UIP from non-UIP using a classifier that comprises or consists of MPP2 or fragments thereof. In one such embodiment, the method uses a classifier comprising MPP2 and at least one of 1) HLA-F (SEQ ID NO.:1) 2) CDKL2 (SEQ ID NO.:2), 3) GPR98 (SEQ ID NO.:3), 4) PRKCQ (SEQ ID NO.:4), 5) HLA-G (SEQ ID NO.:5), 6) PFKFB3 (SEQ ID NO.:6), 7) CEACAM1 (SEQ ID NO.:7), 8) RABGAP1L (SEQ ID NO.:8), 9) CD274 (SEQ ID NO.:9), 10) PRUNE2 (SEQ ID NO.:10), 11) ARAP2 (SEQ ID NO.:11), 12) DZIP1 (SEQ ID NO.:12), 13) MXRA7 (SEQ ID NO.:13), 14) PTCHD4 (SEQ ID NO.:14), 15) PDLIM3 (SEQ ID NO.:15), 16) CNN1 (SEQ ID NO.:16), 17) NIPSNAP3B (SEQ ID NO.:17), 18) PAQR7 (SEQ ID NO.:18), 19) ACTG2 (SEQ ID NO.:19), 20) NA (SEQ ID NO.:20), 21) TIMP2 (SEQ ID NO.:21), and 22) DES (SEQ ID NO.:22). In particular aspects, the classifier contains 1, 2, 3, 4, 5, 6, 7, 8, or more additional genes.

In some particular embodiments, the present invention provides methods and/or systems for differentiating UIP from non-UIP using a classifier that comprises or consists of NMNAT2 or fragments thereof. In one such embodiment, the method uses a classifier comprising NMNAT2 and at least one of 1) HLA-F (SEQ ID NO.:1) 2) CDKL2 (SEQ ID NO.:2), 3) GPR98 (SEQ ID NO.:3), 4) PRKCQ (SEQ ID NO.:4), 5) HLA-G (SEQ ID NO.:5), 6) PFKFB3 (SEQ ID NO.:6), 7) CEACAM1 (SEQ ID NO.:7), 8) RABGAP1L (SEQ ID NO.:8), 9) CD274 (SEQ ID NO.:9), 10) PRUNE2 (SEQ ID NO.:10), 11) ARAP2 (SEQ ID NO.:11), 12) DZIP1 (SEQ ID NO.:12), 13) MXRA7 (SEQ ID NO.:13), 14) PTCHD4 (SEQ ID NO.:14), 15) PDLIM3 (SEQ ID NO.:15), 16) CNN1 (SEQ ID NO.:16), 17) NIPSNAP3B (SEQ ID NO.:17), 18) PAQR7 (SEQ ID NO.:18), 19) ACTG2 (SEQ ID NO.:19), 20) NA (SEQ ID NO.:20), 21) TIMP2 (SEQ ID NO.:21), and 22) DES (SEQ ID NO.:22). In particular aspects, the classifier contains 1, 2, 3, 4, 5, 6, 7, 8, or more additional genes.

In some particular embodiments, the present invention provides methods and/or systems for differentiating UIP from non-UIP using a classifier that comprises or consists of PLXDC1 or fragments thereof. In one such embodiment, the method uses a classifier comprising PLXDC1 and at least one of 1) HLA-F (SEQ ID NO.:1) 2) CDKL2 (SEQ ID NO.:2), 3) GPR98 (SEQ ID NO.:3), 4) PRKCQ (SEQ ID NO.:4), 5) HLA-G (SEQ ID NO.:5), 6) PFKFB3 (SEQ ID NO.:6), 7) CEACAM1 (SEQ ID NO.:7), 8) RABGAP1L (SEQ ID NO.:8), 9) CD274 (SEQ ID NO.:9), 10) PRUNE2 (SEQ ID NO.:10), 11) ARAP2 (SEQ ID NO.:11), 12) DZIP1 (SEQ ID NO.:12), 13) MXRA7 (SEQ ID NO.:13), 14) PTCHD4 (SEQ ID NO.:14), 15) PDLIM3 (SEQ ID NO.:15), 16) CNN1 (SEQ ID NO.:16), 17) NIPSNAP3B (SEQ ID NO.:17), 18) PAQR7 (SEQ ID NO.:18), 19) ACTG2 (SEQ ID NO.:19), 20) NA (SEQ ID NO.:20) 21) TIMP2 (SEQ ID NO.:21), and 22) DES (SEQ ID NO.:22). In particular aspects, the classifier contains 1, 2, 3, 4, 5, 6, 7, 8, or more additional genes.

In some particular embodiments, the present invention provides methods and/or systems for differentiating UIP from non-UIP using a classifier that comprises or consists of CAPN9 or fragments thereof. In one such embodiment, the method uses a classifier comprising CAPN9 and at least one of 1) HLA-F (SEQ ID NO.:1) 2) CDKL2 (SEQ ID NO.:2), 3) GPR98 (SEQ ID NO.:3), 4) PRKCQ (SEQ ID NO.:4), 5) HLA-G (SEQ ID NO.:5), 6) PFKFB3 (SEQ ID NO.:6), 7) CEACAM1 (SEQ ID NO.:7), 8) RABGAP1L (SEQ ID NO.:8), 9) CD274 (SEQ ID NO.:9), 10) PRUNE2 (SEQ ID NO.:10), 11) ARAP2 (SEQ ID NO.:11), 12) DZIP1 (SEQ ID NO.:12), 13) MXRA7 (SEQ ID NO.:13), 14) PTCHD4 (SEQ ID NO.:14), 15) PDLIM3 (SEQ ID NO.:15), 16) CNN1 (SEQ ID NO.:16), 17) NIPSNAP3B (SEQ ID NO.:17), 18) PAQR7 (SEQ ID NO.:18), 19) ACTG2 (SEQ ID NO.:19), 20) NA (SEQ ID NO.:20), 21) TIMP2 (SEQ ID NO.:21), and 22) DES (SEQ ID NO.:22). In particular aspects, the classifier contains 1, 2, 3, 4, 5, 6, 7, 8, or more additional genes.

In some particular embodiments, the present invention provides methods and/or systems for differentiating UIP from non-UIP using a classifier that comprises or consists of TALDO1 or fragments thereof. In one such embodiment, the method uses a classifier comprising TALDO1 and at least one of 1) HLA-F (SEQ ID NO.:1) 2) CDKL2 (SEQ ID NO.:2), 3) GPR98 (SEQ ID NO.:3), 4) PRKCQ (SEQ ID NO.:4), 5) HLA-G (SEQ ID NO.:5), 6) PFKFB3 (SEQ ID NO.:6), 7) CEACAM1 (SEQ ID NO.:7), 8) RABGAP1L (SEQ ID NO.:8), 9) CD274 (SEQ ID NO.:9), 10) PRUNE2 (SEQ ID NO.:10), 11) ARAP2 (SEQ ID NO.:11), 12) DZIP1 (SEQ ID NO.:12), 13) MXRA7 (SEQ ID NO.:13), 14) PTCHD4 (SEQ ID NO.:14), 15) PDLIM3 (SEQ ID NO.:15), 16) CNN1 (SEQ ID NO.:16), 17) NIPSNAP3B (SEQ ID NO.:17), 18) PAQR7 (SEQ ID NO.:18), 19) ACTG2 (SEQ ID NO.:19), 20) NA (SEQ ID NO.:20), 21) TIMP2 (SEQ ID NO.:21), and 22) DES (SEQ ID NO.:22). In particular aspects, the classifier contains 1, 2, 3, 4, 5, 6, 7, 8, or more additional genes.

In some particular embodiments, the present invention provides methods and/or systems for differentiating UIP from non-UIP using a classifier that comprises or consists of PLK4 or fragments thereof. In one such embodiment, the method uses a classifier comprising PLK4 and at least one of 1) HLA-F (SEQ ID NO.:1) 2) CDKL2 (SEQ ID NO.:2), 3) GPR98 (SEQ ID NO.:3), 4) PRKCQ (SEQ ID NO.:4), 5) HLA-G (SEQ ID NO.:5), 6) PFKFB3 (SEQ ID NO.:6), 7) CEACAM1 (SEQ ID NO.:7), 8) RABGAP1L (SEQ ID NO.:8), 9) CD274 (SEQ ID NO.:9), 10) PRUNE2 (SEQ ID NO.:10), 11) ARAP2 (SEQ ID NO.:11), 12) DZIP1 (SEQ ID NO.:12), 13) MXRA7 (SEQ ID NO.:13), 14) PTCHD4 (SEQ ID NO.:14), 15) PDLIM3 (SEQ ID NO.:15), 16) CNN1 (SEQ ID NO.:16), 17) NIPSNAP3B (SEQ ID NO.:17), 18) PAQR7 (SEQ ID NO.:18), 19) ACTG2 (SEQ ID NO.:19), 20) NA (SEQ ID NO.:20), 21) TIMP2 (SEQ ID NO.:21), and 22) DES (SEQ ID NO.:22). In particular aspects, the classifier contains 1, 2, 3, 4, 5, 6, 7, 8, or more additional genes.

In some particular embodiments, the present invention provides methods and/or systems for differentiating UIP from non-UIP using a classifier that comprises or consists of IGHV3-72 or fragments thereof. In one such embodiment, the method uses a classifier comprising IGHV3-72 and at least one of 1) HLA-F (SEQ ID NO.:1) 2) CDKL2 (SEQ ID NO.:2), 3) GPR98 (SEQ ID NO.:3), 4) PRKCQ (SEQ ID NO.:4), 5) HLA-G (SEQ ID NO.:5), 6) PFKFB3 (SEQ ID NO.:6), 7) CEACAM1 (SEQ ID NO.:7), 8) RABGAP1L (SEQ ID NO.:8), 9) CD274 (SEQ ID NO.:9), 10) PRUNE2 (SEQ ID NO.:10), 11) ARAP2 (SEQ ID NO.:11), 12) DZIP1 (SEQ ID NO.:12), 13) MXRA7 (SEQ ID NO.:13), 14) PTCHD4 (SEQ ID NO.:14), 15) PDLIM3 (SEQ ID NO.:15), 16) CNN1 (SEQ ID NO.:16), 17) NIPSNAP3B (SEQ ID NO.:17), 18) PAQR7 (SEQ ID NO.:18), 19) ACTG2 (SEQ ID NO.:19), 20) NA (SEQ ID NO.:20), 21) TIMP2 (SEQ ID NO.:21), and 22) DES (SEQ ID NO.:22). In particular aspects, the classifier contains 1, 2, 3, 4, 5, 6, 7, 8, or more additional genes.

In some particular embodiments, the present invention provides methods and/or systems for differentiating UIP from non-UIP using a classifier that comprises or consists of IGKV1-9 or fragments thereof. In one such embodiment, the method uses a classifier comprising IGKV1-9 and at least one of 1) HLA-F (SEQ ID NO.:1) 2) CDKL2 (SEQ ID NO.:2), 3) GPR98 (SEQ ID NO.:3), 4) PRKCQ (SEQ ID NO.:4), 5) HLA-G (SEQ ID NO.:5), 6) PFKFB3 (SEQ ID NO.:6), 7) CEACAM1 (SEQ ID NO.:7), 8) RABGAP1L (SEQ ID NO.:8), 9) CD274 (SEQ ID NO.:9), 10) PRUNE2 (SEQ ID NO.:10), 11) ARAP2 (SEQ ID NO.:11), 12) DZIP1 (SEQ ID NO.:12), 13) MXRA7 (SEQ ID NO.:13), 14) PTCHD4 (SEQ ID NO.:14), 15) PDLIM3 (SEQ ID NO.:15), 16) CNN1 (SEQ ID NO.:16), 17) NIPSNAP3B (SEQ ID NO.:17), 18) PAQR7 (SEQ ID NO.:18), 19) ACTG2 (SEQ ID NO.:19), 20) NA (SEQ ID NO.:20), 21) TIMP2 (SEQ ID NO.:21), and 22) DES (SEQ ID NO.:22). In particular aspects, the classifier contains 1, 2, 3, 4, 5, 6, 7, 8, or more additional genes.

In some particular embodiments, the present invention provides methods and/or systems for differentiating UIP from non-UIP using a classifier that comprises or consists of CNTN4 or fragments thereof. In one such embodiment, the method uses a classifier comprising CNTN4 and at least one of 1) HLA-F (SEQ ID NO.:1) 2) CDKL2 (SEQ ID NO.:2), 3) GPR98 (SEQ ID NO.:3), 4) PRKCQ (SEQ ID NO.:4), 5) HLA-G (SEQ ID NO.:5), 6) PFKFB3 (SEQ ID NO.:6), 7) CEACAM1 (SEQ ID NO.:7), 8) RABGAP1L (SEQ ID NO.:8), 9) CD274 (SEQ ID NO.:9), 10) PRUNE2 (SEQ ID NO.:10), 11) ARAP2 (SEQ ID NO.:11), 12) DZIP1 (SEQ ID NO.:12), 13) MXRA7 (SEQ ID NO.:13), 14) PTCHD4 (SEQ ID NO.:14), 15) PDLIM3 (SEQ ID NO.:15), 16) CNN1 (SEQ ID NO.:16), 17) NIPSNAP3B (SEQ ID NO.:17), 18) PAQR7 (SEQ ID NO.:18), 19) ACTG2 (SEQ ID NO.:19), 20) NA (SEQ ID NO.:20), 21) TIMP2 (SEQ ID NO.:21), and 22) DES (SEQ ID NO.:22). In particular aspects, the classifier contains 1, 2, 3, 4, 5, 6, 7, 8, or more additional genes.

In some embodiments, the present invention provides a method and/or system for differentiating UIP from non-UIP using a classifier that comprises or consists of 2; 3; 4; 5; 6; 7; 8; 9; 10; 11; 12; 13; 14; 15, 16, 17, 18, 19, 20, 21, 22, 23, 24, 25, 26, 27, 28, 29, 30, 31, 32, 33, 34, or 35 of the following sequences: 1) HLA-F (SEQ ID NO.:1), 2) CDKL2 (SEQ ID NO.:2), 3) GPR98 (SEQ ID NO.:3), 4) PRKCQ (SEQ ID NO.:4), 5) HLA-G (SEQ ID NO.:5), 6) PFKFB3 (SEQ ID NO.:6), 7) CEACAM1 (SEQ ID NO.:7), 8) RABGAP1L (SEQ ID NO.:8), 9) CD274 (SEQ ID NO.:9), 10) PRUNE2 (SEQ ID NO.:10), 11) ARAP2 (SEQ ID NO.:11), 12) DZIP1 (SEQ ID NO.:12), 13) MXRA7 (SEQ ID NO.:13), 14) PTCHD4 (SEQ ID NO.:14), 15) PDLIM3 (SEQ ID NO.:15), 16) CNN1 (SEQ ID NO.:16), 17) NIPSNAP3B (SEQ ID NO.:17), 18) PAQR7 (SEQ ID NO.:18), 19) ACTG2 (SEQ ID NO.:19), 20) NA (SEQ ID NO.:20), 21) TIMP2 (SEQ ID NO.:21), 22) DES (SEQ ID NO.:22), 23) HMCN2, 24) ADAMTSL1, 25) CD79B, 26) KEL, 27) KLHL14, 28) MPP2, 29) NMNAT2, 30) PLXDC1, 31) CAPN9, 32) TALDO1, 33) PLK4, 34) IGHV3-72, 35) IGKV1-9, and 36) CNTN4. In particular aspects, the classifier may contain 1, 2, 3, 4, 5, 6, 7, 8, or more additional genes. In other aspects, the classifier may omit 1, 2, 3, 4, 5, 6, 7, 8, or more, of these genes, while optionally including other genes.

In some embodiments, the present invention provides a method and/or system for differentiating UIP from non-UIP using a classifier that comprises or consists of all of the following sequences: 1) HLA-F (SEQ ID NO.:1), 2) CDKL2 (SEQ ID NO.:2), 3) GPR98 (SEQ ID NO.:3), 4) PRKCQ (SEQ ID NO.:4), 5) HLA-G (SEQ ID NO.:5), 6) PFKFB3 (SEQ ID NO.:6), 7) CEACAM1 (SEQ ID NO.:7), 8) RABGAP1L (SEQ ID NO.:8), 9) CD274 (SEQ ID NO.:9), 10) PRUNE2 (SEQ ID NO.:10), 11) ARAP2 (SEQ ID NO.:11), 12) DZIP1 (SEQ ID NO.:12), 13) MXRA7 (SEQ ID NO.:13), 14) PTCHD4 (SEQ ID NO.:14), 15) PDLIM3 (SEQ ID NO.:15), 16) CNN1 (SEQ ID NO.:16), 17) NIPSNAP3B (SEQ ID NO.:17), 18) PAQR7 (SEQ ID NO.:18), 19) ACTG2 (SEQ ID NO.:19), 20) NA (SEQ ID NO.:20), 21) TIMP2 (SEQ ID NO.:21), 22) DES (SEQ ID NO.:22), 23) HMCN2, 24) ADAMTSL1, 25) CD79B, 26) KEL, 27) KLHL14, 28) MPP2, 29) NMNAT2, 30) PLXDC1, 31) CAPN9, 32) TALDO1, 33) PLK4, 34) IGHV3-72, 35) IGKV1-9, and 36) CNTN4. In particular aspects, the classifier may contain 1, 2, 3, 4, 5, 6, 7, 8, or more additional genes. In other aspects, the classifier may omit 1, 2, 3, 4, 5, 6, 7, 8, or more, of these genes, while optionally including other genes. In some embodiments, the present invention provides a method and/or system for differentiating UIP from non-UIP using a classifier described herein, wherein the method further comprises implementing a classifier that classifies the subject as a smoker or non-smoker. Such a smoker status classification can optionally be implemented prior to implementing a UIP vs. Non-UIP classifier, or a smoker status classification step can be built in as a covariate used during the training (e.g., using a classifier training module) of a UIP vs. Non-UIP classifier of the present invention.

In some embodiments, alternatively, or additionally, the method of and/or system for differentiating UIP from non-UIP using a classifier described herein further comprises a step of excluding or assigning differential weight to certain genes or variants thereof that are susceptible to smoker-status bias during the training (e.g., using a classifier training module) or implementation of the UIP vs. Non-UIP classifier. As used herein, “smoker status bias” refers to genes or variants thereof, which in non-smoker patients are differentially expressed in UIP vs. non-UIP patients, but which are not detectably differentially expressed in UIP vs. non-UIP patients that are (or have been) smokers.

In some embodiments, the method of and/or system for the present invention comprises a tiered classifier comprising at least a first and a second classifier, wherein the first classifier is trained (e.g., using a classifier training module) to recognize gene signatures that distinguish smokers from non-smokers, and a second classifier is trained (e.g., using a classifier training module) to distinguish UIP vs. Non UIP in smokers or non-smokers, respectively.

In some embodiments, the method and/or systems of the present invention comprises:
extracting nucleic acids (e.g., RNA, such as, e.g., total RNA) from a test sample (e.g. lung tissue);
amplifying the nucleic acid to produce an expressed nucleic acid library (e.g., via polymerase chain reaction-mediated amplification of cDNAs (optionally labeled cDNAs), which cDNAs may be produced from one or more RNA sample by reverse transcription (RT-PCR));
detecting expression of one or more nucleic acid present in the nucleic acid library (e.g., detecting RNA expression profiles by measuring cDNA species produced via RT-PCR) via an array (e.g., a microarray) or via direct sequencing (e.g., RNAseq); and determining whether the test sample is UIP or non-UIP using a trained classifier described herein.

In some embodiments, the method and/or system of the present invention further comprises incorporating smoker status into the training exercise. In certain embodiments, smoker status is optionally incorporated in one of the following ways:
(i) by using smoking status as a covariate in a UIP or Non-UIP classifier during training (e.g., using a classifier training module).
(ii) by identifying a plurality of genes that are susceptible to smoker-status bias and excluding, or optionally weighing such genes differently than genes that are not susceptible to such bias, during UIP or Non-UIP classifier training (e.g., using a classifier training module).
(iii) by constructing a tiered classification in which an initial classifier that is trained (e.g., using a classifier training module) to recognize gene signatures that distinguish smokers from non-smokers is used to pre-classify a test sample as “smoker” or “non-smoker” based upon the gene signature of the test sample; and then, subsequent to pre-classification, a distinct classifier that was trained (e.g., using a classifier training module) to distinguish UIP vs. Non UIP in either smokers or non-smokers is implemented. For example, if the pre-classifier determines that the test sample is from a smoker, a UIP vs. Non-UIP classification is performed using a classifier trained (e.g., using a classifier training module) with UIP and Non-UIP samples from smokers. Conversely, if the pre-classifier determines that the test sample is from a non-smoker, a UIP vs. Non-UIP classification is performed using a classifier trained (e.g., using a classifier training module) with UIP and Non-UIP samples from non-smokers. In some embodiments, such smoker- or non-smoker-specific classifiers provide improved diagnostic performance due, at least in part, to a reduction in background noise caused by the inclusion of genes susceptible to smoker-status bias in the classifier training.

Accordingly, the present invention also provides suitable classifiers for use in methods of differentiating UIP from non-UIP, as disclosed herein. In various embodiments, the present invention provides a classifier suitable for differentiating UIP from non-UIP, wherein the classifier is trained (e.g., using a classifier training module) using microarray or sequencing data from a sample corresponding to one or more histopathology label determined by an expert pathologist. In some embodiments, the sample is labelled UIP or Non-UIP.

In some embodiments, the present invention presents a classifier comprising or consisting of one or more sequences or fragments thereof presented in any of Tables 5, 7, 8, 9, 10, 11, or 12, or at least one sequence or fragment thereof from each of Tables 5, 7, 8, 9, 10, 11, or 12. In some embodiments, the present invention provides a classifier comprising or consisting of at least 1, 2, 3, 4, 5, 6, 7, 8, 9, 10 or more of the sequences provided in any one or more or all of Tables 5, 7, 8, 9, 10, 11 and 12. For example, in some embodiments, the present invention provides a classifier comprising or consisting of at least 11, 12, 13, 14, 15, 20, 30, 50, 100, 150, 200, 250, 300, or more sequences provided in any one or more or all of Tables 5, 7, 8, 9, 10, 11, or 12, including all integers (e.g., 16, 17, 18, 19, 21, 22, 23, 24, 25 sequences, etc.) and ranges (e.g., from about 1-10 sequences from any one or more or all of Tables 5, 7, 8, 9, 10, 11, or 12, from about 10-15 sequences, 10-20 sequences, 5-30 sequences, 5-50 sequences, 10-100 sequences, 50-200 sequences from any one or more or all of Tables 5, 7, 8, 9, 10, 11, or 12, etc.) between. In one embodiment, the present invention provides a classifier that comprises or consists of all sequences provided in Table 5, all sequences provided in Table 7, all sequences provided in Table 8, all sequences provided in Table 9, all sequences provided in table 10, all sequences provided in Table 11, or all sequences provided in Table 12. In one embodiment, the present invention provides a classifier that comprises or consists of all sequences provided in each of Tables 5, 7, 8, 9, 10, 11, or 12.

In some particular embodiments, the present invention provides a classifier for differentiating UIP from non-UIP, wherein the classifier comprises or consists of one or more of the following sequences or fragments thereof: 1) HLA-F (SEQ ID NO.:1), 2) CDKL2 (SEQ ID NO.:2), 3) GPR98 (SEQ ID NO.:3), 4) PRKCQ (SEQ ID NO.:4), 5) HLA-G (SEQ ID NO.:5), 6) PFKFB3 (SEQ ID NO.:6), 7) CEACAM1 (SEQ ID NO.:7), 8) RABGAP1L (SEQ ID NO.:8), 9) CD274 (SEQ ID NO.:9), 10) PRUNE2 (SEQ ID NO.:10), 11) ARAP2 (SEQ ID NO.:11), 12) DZIP1 (SEQ ID NO.:12), 13) MXRA7 (SEQ ID NO.:13), 14) PTCHD4 (SEQ ID NO.:14), 15) PDLIM3 (SEQ ID NO.:15), 16) CNN1 (SEQ ID NO.:16), 17) NIPSNAP3B (SEQ ID NO.:17), 18) PAQR7 (SEQ ID NO.:18), 19) ACTG2 (SEQ ID NO.:19), 20) NA (SEQ ID NO.:20) 21) TIMP2 (SEQ ID NO.:21), and 22) DES (SEQ ID NO.:22). In one embodiment, the classifier comprises or consists of all 22 of the above mentioned sequences. In some embodiments, the present invention provides a classifier for differentiating UIP from non-UIP, wherein the classifier comprises or consists of 2; 3; 4; 5; 6; 7; 8; 9; 10; 11; 12; 13; 14; 15; 16; 17; 18; 19; 20; or 21 of the abovementioned 22 sequences. In particular aspects, the classifier contains 1, 2, 3, 4, 5, 6, 7, 8, or more additional genes or fragments thereof. In other aspects, the classifier omits 1, 2, 3, 4, 5, 6, 7, 8, or more, of the abovementioned 22 sequences, while optionally including other genes. In other aspects, each of the 22 genes may be used in combination with any 1 or more, up to 20 more, of the other genes.

Tissue Samples

A lung tissue sample for use in a subject analytical or diagnostic method can be a biopsy sample (e.g., a biopsy sample obtained by video-assisted thoracoscopic surgery; VATS); a bronchoalveolar lavage (BAL) sample; a transbronchial biopsy; a cryo-transbronchial biopsy; and the like.” Lung tissue samples for analysis can be provided in a suitable preservation solution.

Tissue samples can be obtained from a patient suspected of having a lung disease, e.g., an ILD, based on clinical signs and symptoms with which the patient presents (e.g., shortness of breath (generally aggravated by exertion), dry cough), and, optionally the results of one or more of an imaging test (e.g., chest X-ray, computerized tomography (CT)), a pulmonary function test (e.g., spirometry, oximetry, exercise stress test), lung tissue analysis (e.g., histological and/or cytological analysis of samples obtained by bronchoscopy, bronchoalveolar lavage, surgical biopsy).

The lung tissue sample can be processed in any of a variety of ways. For example, the lung tissue sample can be subjected to cell lysis. The lung tissue sample can be preserved in RNAprotect solution (a solution that inhibits RNA degradation, e.g., that inhibits nuclease digestion of RNA) and subsequently subjected to cell lysis. Components such as nucleic acids and/or proteins can be enriched or isolated from the lung tissue sample, and the enriched or isolated component can be used in a subject method. Methods of enriching for and isolating components such nucleic acids and proteins are known in the art; and any known method can be used. Methods of isolating RNA for expression analysis have been described in the art.

In Vitro Methods of Determining Expression Product Levels

Additional approaches to assess expression of the panel further demonstrated the genomic signal observed in UIP vs. non-UIP classification is robust across diverse biochemical assays and detection methods. Specifically we generated RNASeq data for a subset of the cohort and evaluated performance under CV. Performance comparisons with matched array data demonstrated that classification using RNASeq data achieves similar performance to data generated from the microarray platform.

The general methods for determining gene expression product levels are known to the art and may include but are not limited to one or more of the following: additional cytological assays, assays for specific proteins or enzyme activities, assays for specific expression products including protein or RNA or specific RNA splice variants, in situ hybridization, whole or partial genome expression analysis, microarray hybridization assays, serial analysis of gene expression (SAGE), enzyme linked immunoabsorbance assays, mass-spectrometry, immunohistochemistry, blotting, sequencing, RNA sequencing, DNA sequencing (e.g., sequencing of cDNA obtained from RNA); Next-Gen sequencing, nanopore sequencing, pyrosequencing, or Nanostring sequencing. For example, gene expression product levels can be determined according to the methods described in Kim, et. al. (Lancet Respir Med. 2015 June;3(6):473-82, incorporated herein in its entirety, including all supplements). As used herein, the terms “assaying” or “detecting” or “determining” are used interchangeably in reference to determining gene expression product levels, and in each case, it is contemplated that the above-mentioned methods of determining gene expression product levels are suitable for detecting or assaying gene expression product levels. Gene expression product levels may be normalized to an internal standard such as total mRNA or the expression level of a particular gene including but not limited to glyceraldehyde 3 phosphate dehydrogenase, or tubulin.

In various embodiments, a sample comprises cells harvested from a tissue sample (e.g., a lung tissue sample such as a TBB sample). Cells can be harvested from a sample using standard techniques known in the art or disclosed herein. For example, in one embodiment, cells are harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract nucleic acid, e.g., messenger RNA. All samples obtained from a subject, including those subjected to any sort of further processing, are considered to be obtained from the subject.

The sample, in one embodiment, is further processed before detection of the gene expression products is performed as described herein. For example, mRNA in a cell or tissue sample can be separated from other components of the sample. The sample can be concentrated and/or purified to isolate mRNA in its non-natural state, as the mRNA is not in its natural environment. For example, studies have indicated that the higher order structure of mRNA in vivo differs from the in vitro structure of the same sequence (see, e.g., Rouskin et al. (2014). Nature 505, pp. 701-705, incorporated herein in its entirety for all purposes).

mRNA from the sample in one embodiment, is hybridized to a synthetic DNA probe, which in some embodiments, includes a detection moiety (e.g., detectable label, capture sequence, barcode reporting sequence). Accordingly, in these embodiments, a non-natural mRNA-cDNA complex is ultimately made and used for detection of the gene expression product. In another embodiment, mRNA from the sample is directly labeled with a detectable label, e.g., a fluorophore. In a further embodiment, the non-natural labeled-mRNA molecule is hybridized to a cDNA probe and the complex is detected.

In one embodiment, once the mRNA is obtained from a sample, it is converted to complementary DNA (cDNA) in a hybridization reaction or is used in a hybridization reaction together with one or more cDNA probes. cDNA does not exist in vivo and therefore is a non-natural molecule. Furthermore, cDNA-mRNA hybrids are synthetic and do not exist in vivo. Besides cDNA not existing in vivo, cDNA is necessarily different than mRNA, as it includes deoxyribonucleic acid and not ribonucleic acid. The cDNA is then amplified, for example, by the polymerase chain reaction (PCR) or other amplification method known to those of ordinary skill in the art. For example, other amplification methods that may be employed include the ligase chain reaction (LCR) (Wu and Wallace, Genomics, 4:560 (1989), Landegren et al., Science, 241:1077 (1988), incorporated by reference in its entirety for all purposes, transcription amplification (Kwoh et al., Proc. Natl. Acad. Sci. USA, 86:1173 (1989), incorporated by reference in its entirety for all purposes), self-sustained sequence replication (Guatelli et al., Proc. Nat. Acad. Sci. USA, 87:1874 (1990), incorporated by reference in its entirety for all purposes), incorporated by reference in its entirety for all purposes, and nucleic acid based sequence amplification (NASBA). Guidelines for selecting primers for PCR amplification are known to those of ordinary skill in the art. See, e.g., McPherson et al., PCR Basics: From Background to Bench, Springer-Verlag, 2000, incorporated by reference in its entirety for all purposes. The product of this amplification reaction, i.e., amplified cDNA is also necessarily a non-natural product. First, as mentioned above, cDNA is a non-natural molecule. Second, in the case of PCR, the amplification process serves to create hundreds of millions of cDNA copies for every individual cDNA molecule of starting material. The number of copies generated are far removed from the number of copies of mRNA that are present in vivo.

In one embodiment, cDNA is amplified with primers that introduce an additional DNA sequence (e.g., adapter, reporter, capture sequence or moiety, barcode) onto the fragments (e.g., with the use of adapter-specific primers), or mRNA or cDNA gene expression product sequences are hybridized directly to a cDNA probe comprising the additional sequence (e.g., adapter, reporter, capture sequence or moiety, barcode). Amplification and/or hybridization of mRNA to a cDNA probe therefore serves to create non-natural double stranded molecules from the non-natural single stranded cDNA, or the mRNA, by introducing additional sequences and forming non-natural hybrids. Further, as known to those of ordinary skill in the art, amplification procedures have error rates associated with them. Therefore, amplification introduces further modifications into the cDNA molecules. In one embodiment, during amplification with the adapter-specific primers, a detectable label, e.g., a fluorophore, is added to single strand cDNA molecules. Amplification therefore also serves to create DNA complexes that do not occur in nature, at least because (i) cDNA does not exist in vivo, (i) adapter sequences are added to the ends of cDNA molecules to make DNA sequences that do not exist in vivo, (ii) the error rate associated with amplification further creates DNA sequences that do not exist in vivo, (iii) the disparate structure of the cDNA molecules as compared to what exists in nature and (iv) the chemical addition of a detectable label to the cDNA molecules.

In some embodiments, the expression of a gene expression product of interest is detected at the nucleic acid level via detection of non-natural cDNA molecules.

The gene expression products described herein include RNA comprising the entire or partial sequence of any of the nucleic acid sequences of interest, or their non-natural cDNA product, obtained synthetically in vitro in a reverse transcription reaction. The term “fragment” is intended to refer to a portion of the polynucleotide that generally comprise at least 10, 15, 20, 50, 75, 100, 150, 200, 250, 300, 350, 400, 450, 500, 550, 600, 650, 700, 800, 900, 1,000, 1,200, or 1,500 contiguous nucleotides, or up to the number of nucleotides present in a fulllength gene expression product polynucleotide disclosed herein. A fragment of a gene expression product polynucleotide will generally encode at least 15, 25, 30, 50, 100, 150, 200, or 250 contiguous amino acids, or up to the total number of amino acids present in a full-length gene expression product protein of the invention.

In certain embodiments, a gene expression profile may be obtained by whole transcriptome shotgun sequencing (“WTSS” or “RNAseq”; see, e.g., Ryan et al BioTechniques 45: 81-94), which makes the use of high-throughput sequencing technologies to sequence cDNA in order to about information about a sample's RNA content. In general terms, cDNA is made from RNA, the cDNA is amplified, and the amplification products are sequenced.

After amplification, the cDNA may be sequenced using any convenient method. For example, the fragments may be sequenced using Illumina's reversible terminator method, Roche's pyrosequencing method (454), Life Technologies' sequencing by ligation (the SOLiD platform) or Life Technologies' Ion Torrent platform. Examples of such methods are described in the following references: Margulies et al (Nature 2005 437: 376-80); Ronaghi et al (Analytical Biochemistry 1996 242: 84-9); Shendure (Science 2005 309: 1728); Imelfort et al (Brief Bioinform. 2009 10:609-18); Fox et al (Methods Mol Biol. 2009; 553:79-108); Appleby et al (Methods Mol Biol. 2009; 513: 19-39) and Morozova (Genomics. 2008 92:255-64), which are incorporated by reference for the general descriptions of the methods and the particular steps of the methods, including all starting products, reagents, and final products for each of the steps. As would be apparent, forward and reverse sequencing primer sites that compatible with a selected next generation sequencing platform can be added to the ends of the fragments during the amplification step.

In other embodiments, the products may be sequenced using nanopore sequencing (e.g. as described in Soni et al Clin Chem 53: 1996-2001 2007, or as described by Oxford Nanopore Technologies). Nanopore sequencing is a single-molecule sequencing technology whereby a single molecule of DNA is sequenced directly as it passes through a nanopore. A nanopore is a small hole, of the order of 1 nanometer in diameter. Immersion of a nanopore in a conducting fluid and application of a potential (voltage) across it results in a slight electrical current due to conduction of ions through the nanopore. The amount of current which flows is sensitive to the size and shape of the nanopore. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore represents a reading of the DNA sequence. Nanopore sequencing technology as disclosed in U.S. Pat. Nos. 5,795,782, 6,015,714, 6,627,067, 7,238,485 and 7,258,838 and U.S. patent application publications US2006003171 and US20090029477.

In some embodiments, the gene expression product of the subject methods is a protein, and the amount of protein in a particular biological sample is analyzed using a classifier derived from protein data obtained from cohorts of samples. The amount of protein can be determined by one or more of the following: enzyme-linked immunosorbent assay (ELISA), mass spectrometry, blotting, or immunohistochemistry.

In some embodiments, gene expression product markers and alternative splicing markers may be determined by microarray analysis using, for example, Affymetrix arrays, cDNA microarrays, oligonucleotide microarrays, spotted microarrays, or other microarray products from Biorad, Agilent, or Eppendorf. Microarrays provide particular advantages because they may contain a large number of genes or alternative splice variants that may be assayed in a single experiment. In some cases, the microarray device may contain the entire human genome or transcriptome or a substantial fraction thereof allowing a comprehensive evaluation of gene expression patterns, genomic sequence, or alternative splicing. Markers may be found using standard molecular biology and microarray analysis techniques as described in Sambrook Molecular Cloning a Laboratory Manual 2001 and Baldi, P., and Hatfield, W. G., DNA Microarrays and Gene Expression 2002.

Microarray analysis generally begins with extracting and purifying nucleic acid from a biological sample, (e.g. a biopsy or fine needle aspirate) using methods known to the art. For expression and alternative splicing analysis it may be advantageous to extract and/or purify RNA from DNA. It may further be advantageous to extract and/or purify niRNA from other forms of RNA such as tRNA and rRNA.

Purified nucleic acid may further be labeled with a fluorescent label, radionuclide, or chemical label such as biotin, digoxigenin, or digoxin for example by reverse transcription, polymerase chain reaction (PCR), ligation, chemical reaction or other techniques. The labeling can be direct or indirect which may further require a coupling stage. The coupling stage can occur before hybridization, for example, using aminoallyl-UTP and NHS amino-reactive dyes (like cyanine dyes) or after, for example, using biotin and labelled streptavidin. In one example, modified nucleotides (e.g. at a 1 aaUTP: 4 TTP ratio) are added enzymatically at a lower rate compared to normal nucleotides, typically resulting in 1 every 60 bases (measured with a spectrophotometer). The aaDNA may then be purified with, for example, a column or a diafiltration device. The aminoallyl group is an amine group on a long linker attached to the nucleobase, which reacts with a reactive label (e.g. a fluorescent dye).

The labeled samples may then be mixed with a hybridization solution which may contain sodium dodecyl sulfate (SDS), SSC, dextran sulfate, a blocking agent (such as COT1 DNA, salmon sperm DNA, calf thymus DNA, PolyA or PolyT), Denhardt's solution, formamine, or a combination thereof

A hybridization probe is a fragment of DNA or RNA of variable length, which is used to detect in DNA or RNA samples the presence of nucleotide sequences (the DNA target) that are complementary to the sequence in the probe. The probe thereby hybridizes to single-stranded nucleic acid (DNA or RNA) whose base sequence allows probe-target base pairing due to complementarity between the probe and target. The labeled probe is first denatured (by heating or under alkaline conditions) into single DNA strands and then hybridized to the target DNA.

To detect hybridization of the probe to its target sequence, the probe is tagged (or labeled) with a molecular marker; commonly used markers are 32P or Digoxigenin, which is nonradioactive antibody-based marker. DNA sequences or RNA transcripts that have moderate to high sequence complementarity (e.g. at least 70%, 80%, 90%, 95%, 96%, 97%, 98%, 99%, or more complementarity) to the probe are then detected by visualizing the hybridized probe via autoradiography or other imaging techniques. Detection of sequences with moderate or high complementarity depends on how stringent the hybridization conditions were applied; high stringency, such as high hybridization temperature and low salt in hybridization buffers, permits only hybridization between nucleic acid sequences that are highly similar, whereas low stringency, such as lower temperature and high salt, allows hybridization when the sequences are less similar. Hybridization probes used in DNA microarrays refer to DNA covalently attached to an inert surface, such as coated glass slides or gene chips, and to which a mobile cDNA target is hybridized.

A mix comprising target nucleic acid to be hybridized to probes on an array may be denatured by heat or chemical means and added to a port in a microarray. The holes may then be sealed and the microarray hybridized, for example, in a hybridization oven, where the microarray is mixed by rotation, or in a mixer. After an overnight hybridization, non-specific binding may be washed off (e.g. with SDS and SSC). The microarray may then be dried and scanned in a machine comprising a laser that excites the dye and a detector that measures emission by the dye. The image may be overlaid with a template grid and the intensities of the features (e.g. a feature comprising several pixels) may be quantified.

Various kits can be used for the amplification of nucleic acid and probe generation of the subject methods. Examples of kit that can be used in the present invention include but are not limited to Nugen WT-Ovation FFPE kit, cDNA amplification kit with Nugen Exon Module and Frag/Label module. The NuGEN WT-Ovation™. FFPE System V2 is a whole transcriptome amplification system that enables conducting global gene expression analysis on the vast archives of small and degraded RNA derived from FFPE samples. The system is comprised of reagents and a protocol required for amplification of as little as 50 ng of total FFPE RNA. The protocol can be used for qPCR, sample archiving, fragmentation, and labeling. The amplified cDNA can be fragmented and labeled in less than two hours for GeneChip™. 3′ expression array analysis using NuGEN's FL-Ovation™. cDNA Biotin Module V2. For analysis using Affymetrix GeneChip™. Exon and Gene ST arrays, the amplified cDNA can be used with the WT-Ovation Exon Module, then fragmented and labeled using the FL-Ovation™. cDNA Biotin Module V2. For analysis on Agilent arrays, the amplified cDNA can be fragmented and labeled using NuGEN's FL-Ovation™. cDNA Fluorescent Module.

In some embodiments, Ambion WT-expression kit can be used. Ambion WT-expression kit allows amplification of total RNA directly without a separate ribosomal RNA (rRNA) depletion step. With the Ambion™ WT Expression Kit, samples as small as 50 ng of total RNA can be analyzed on Affymetrix™. GeneChip™ Human, Mouse, and Rat Exon and Gene 1.0 ST Arrays. In addition to the lower input RNA requirement and high concordance between the Affymetrix™ method and TaqMan™ real-time PCR data, the Ambion™. WT Expression Kit provides a significant increase in sensitivity. For example, a greater number of probe sets detected above background can be obtained at the exon level with the Ambion™ WT Expression Kit as a result of an increased signal-to-noise ratio. Ambion™-expression kit may be used in combination with additional Affymetrix labeling kit. In some embodiments, AmpTec Trinucleotide Nano mRNA Amplification kit (6299-A15) can be used in the subject methods. The ExpressArt™ TRinucleotide mRNA amplification Nano kit is suitable for a wide range, from 1 ng to 700 ng of input total RNA. According to the amount of input total RNA and the required yields of aRNA, it can be used for 1-round (input >300 ng total RNA) or 2-rounds (minimal input amount 1 ng total RNA), with aRNA yields in the range of >10 μg. AmpTec's proprietary TRinucleotide priming technology results in preferential amplification of mRNAs (independent of the universal eukaryotic 3′-poly(A)-sequence), combined with selection against rRNAs. More information on AmpTec Trinucleotide Nano mRNA Amplification kit can be obtained at www.amp-tec.com/products.htm. This kit can be used in combination with cDNA conversion kit and Affymetrix labeling kit.

The raw data may then be normalized, for example, by subtracting the background intensity and then dividing the intensities making either the total intensity of the features on each channel equal or the intensities of a reference gene and then the t-value for all the intensities may be calculated. More sophisticated methods, include z-ratio, loess and lowess regression and RMA (robust multichip analysis), such as for Affymetrix chips.

In some embodiments, the above described methods may be used for determining transcript expression levels for training (e.g., using a classifier training module) a classifier to differentiate whether a subject is a smoker or non-smoker. In some embodiments, the above described methods may be used for determining transcript expression levels for training (e.g., using a classifier training module) a classifier to differentiate whether a subject has UIP or non-UIP.

Data Analysis

(i) Comparison of Sample to Normal

In some embodiments, results of molecular profiling performed on a sample from a subject (“test sample”) may be compared to a biological sample that is known or suspected to be normal (“normal sample”). In some embodiments, a normal sample is a sample that does not comprise or is expected to not comprise an ILD, or conditions under evaluation, or would test negative in the molecular profiling assay for the one or more ILDs under evaluation. In some embodiments, a normal sample is that which is or is expected to be free of any ILD, or a sample that would test negative for any ILD in the molecular profiling assay. The normal sample may be from a different subject from the subject being tested, or from the same subject. In some cases, the normal sample is a lung tissue sample obtained from a subject such as the subject being tested for example. The normal sample may be assayed at the same time, or at a different time from the test sample. In some embodiments, a normal sample is a sample that is known or suspected to be from a non-smoker. In particular embodiments, the normal sample is a sample that has been confirmed by at least two expert pathologists to be Non-UIP. In particular embodiments, the normal sample is a sample that has been confirmed by at least two expert pathologists to be Non-IPF.

The results of an assay on the test sample may be compared to the results of the same assay on a sample having a known disease state (e.g., normal, affected by a selected ILD (e.g., IPF, NSIP, etc.), smoker, non-smoker). In some cases the results of the assay on the normal sample are from a database, or a reference. In some cases, the results of the assay on the normal sample are a known or generally accepted value or range of values by those skilled in the art. In some cases the comparison is qualitative. In other cases the comparison is quantitative. In some cases, qualitative or quantitative comparisons may involve but are not limited to one or more of the following: comparing fluorescence values, spot intensities, absorbance values, chemiluminescent signals, histograms, critical threshold values, statistical significance values, gene product expression levels, gene product expression level changes, alternative exon usage, changes in alternative exon usage, protein levels, DNA polymorphisms, copy number variations, indications of the presence or absence of one or more DNA markers or regions, or nucleic acid sequences.

(ii) Evaluation of Results

In some embodiments, the molecular profiling results are evaluated using methods known to the art for correlating gene product expression levels or alternative exon usage with specific phenotypes such as a particular ILD, or normalcy (e.g. disease or condition free). In some cases, a specified statistical confidence level may be determined in order to provide a diagnostic confidence level. For example, it may be determined that a confidence level of greater than 90% may be a useful predictor of the presence of an ILD or of a smoker or non-smoker status. In other embodiments, more or less stringent confidence levels may be chosen. For example, a confidence level of about or at least about 50%, 60%, 70%, 75%, 80%, 85%, 90%, 95%, 97.5%, 99%, 99.5%, or 99.9% may be chosen as a useful phenotypic predictor. The confidence level provided may in some cases be related to the quality of the sample, the quality of the data, the quality of the analysis, the specific methods used, and/or the number of gene expression products analyzed. The specified confidence level for providing a diagnosis may be chosen on the basis of the expected number of false positives or false negatives and/or cost. Methods for choosing parameters for achieving a specified confidence level or for identifying markers with diagnostic power include but are not limited to Receiver Operating Characteristic (ROC) curve analysis, binormal ROC, principal component analysis, partial least squares analysis, singular value decomposition, least absolute shrinkage and selection operator analysis, least angle regression, and the threshold gradient directed regularization method.

(iii) Data analysis

Raw gene expression level and alternative splicing data may in some cases be improved through the application of methods and/or processes designed to normalize and or improve the reliability of the data. In some embodiments of the present disclosure the data analysis requires a computer or other device, machine or apparatus for application of the various methods and/or processes described herein due to the large number of individual data points that are processed. A “machine learning classifier” refers to a computational-based prediction data structure or method, employed for characterizing a gene expression profile. The signals corresponding to certain expression levels, which are obtained by, e.g., microarray-based hybridization assays, are typically subjected to the classifier to classify the expression profile. Supervised learning generally involves “training” a classifier to recognize the distinctions among classes and then “testing” the accuracy of the classifier on an independent test set. For new, unknown samples the classifier can be used to predict the class in which the samples belong. In various embodiments, such training is be achieved, e.g., using a classifier training module.

In some cases, the robust multi-array average (RMA) method may be used to normalize raw data. The RMA method begins by computing background-corrected intensities for each matched cell on a number of microarrays. The background corrected values are restricted to positive values as described by Irizarry et al. Biostatistics 2003 Apr. 4 (2): 249-64. After background correction, the base-2 logarithm of each background corrected matched-cell intensity is then obtained. The back-ground corrected, log-transformed, matched intensity on each microarray is then normalized using the quantile normalization method in which for each input array and each probe expression value, the array percentile probe value is replaced with the average of all array percentile points, this method is more completely described by Bolstad et al. Bioinformatics 2003. Following quantile normalization, the normalized data may then be fit to a linear model to obtain an expression measure for each probe on each microarray. Tukey's median polish algorithm (Tukey, J. W., Exploratory Data Analysis. 1977) may then be used to determine the log-scale expression level for the normalized probe set data.

Various other software and/or hardware modules or processes may be implemented. In certain methods, feature selection and model estimation may be performed by logistic regression with lasso penalty using glmnet (Friedman J, Hastie T, Tibshirani R. Regularization Paths for Generalized Linear Models via Coordinate Descent. Journal of statistical software 2010; 33(1): 1-22). Raw reads may be aligned using TopHat (Trapnell C, Pachter L, Salzberg SL. TopHat: discovering splice junctions with RNA-Seq. Bioinformatics 2009; 25(9): 1105-11). Gene counts may be obtained using HTS eq (Anders S, Pyl PT, Huber W. HTSeq-a Python framework to work with high-throughput sequencing data. Bioinformatics 2014) and normalized using DESeq (Love MI, Huber W, Anders S. Moderated estimation of fold change and dispersion for RNA-Seq data with DESeq2; 2014). In methods, top features (N ranging from 10 to 200) were used to train a linear support vector machine (SVM) (Suykens JAK, Vandewalle J. Least Squares Support Vector Machine Classifiers. Neural Processing Letters 1999; 9(3): 293-300) using the e1071 library (Meyer D. Support vector machines: the interface to libsvm in package e1071. 2014). Confidence intervals may be computed using the pROC package (Robin X, Turck N, Hainard A, et al. pROC: an open-source package for R and S+ to analyze and compare ROC curves. BMC bioinformatics 2011; 12: 77)

In addition, data may be filtered to remove data that may be considered suspect. In some embodiments, data deriving from microarray probes that have fewer than about 4, 5, 6, 7 or 8 guanosine+cytosine nucleotides may be considered to be unreliable due to their aberrant hybridization propensity or secondary structure issues. Similarly, data deriving from microarray probes that have more than about 12, 13, 14, 15, 16, 17, 18, 19, 20, 21, or 22 guanosine+cytosine nucleotides may be considered unreliable due to their aberrant hybridization propensity or secondary structure issues.

In some cases, unreliable probe sets may be selected for exclusion from data analysis by ranking probe-set reliability against a series of reference datasets. For example, RefSeq or Ensembl (EMBL) are considered very high quality reference datasets. Data from probe sets matching RefSeq or Ensembl sequences may in some cases be specifically included in microarray analysis experiments due to their expected high reliability. Similarly data from probe-sets matching less reliable reference datasets may be excluded from further analysis, or considered on a case by case basis for inclusion. In some cases, the Ensembl high throughput cDNA (HTC) and/or mRNA reference datasets may be used to determine the probe-set reliability separately or together. In other cases, probe-set reliability may be ranked. For example, probes and/or probe-sets that match perfectly to all reference datasets such as for example RefSeq, HTC, HTSeq, and mRNA, may be ranked as most reliable (1). Furthermore, probes and/or probe-sets that match two out of three reference datasets may be ranked as next most reliable (2), probes and/or probe-sets that match one out of three reference datasets may be ranked next (3) and probes and/or probe sets that match no reference datasets may be ranked last (4). Probes and or probe-sets may then be included or excluded from analysis based on their ranking. For example, one may choose to include data from category 1, 2, 3, and 4 probe-sets; category 1, 2, and 3 probe-sets; category 1 and 2 probe-sets; or category 1 probe-sets for further analysis. In another example, probe-sets may be ranked by the number of base pair mismatches to reference dataset entries. It is understood that there are many methods understood in the art for assessing the reliability of a given probe and/or probe-set for molecular profiling and the methods of the present disclosure encompass any of these methods and combinations thereof.

In some embodiments of the present invention, data from probe-sets may be excluded from analysis if they are not expressed or expressed at an undetectable level (not above background). A probe-set is judged to be expressed above background if for any group:
Integral from TO to Infinity of the standard normal distribution<Significance (0.01)
Where: TO=Sqr(GroupSize) (T-P)/Sqr(Pvar); GroupSize=Number of CEL files in the group, T=Average of probe scores in probe-set, P=Average of Background probes averages of GC content, and Pvar=Sum of Background probe variances/(Number of probes in probe-set) 2,

This allows including probe-sets in which the average of probe-sets in a group is greater than the average expression of background probes of similar GC content as the probe-set probes as the center of background for the probe-set and enables one to derive the probe-set dispersion from the background probe-set variance.

In some embodiments of the present disclosure, probe-sets that exhibit no, or low variance may be excluded from further analysis. Low-variance probe-sets are excluded from the analysis via a Chi-Square test. A probe-set is considered to be low-variance if its transformed variance is to the left of the 99 percent confidence interval of the Chi-Squared distribution with (N−1) degrees of freedom. (N−1)*Probe-set Variance/(Gene Probe-set Variance). about.Chi-Sq(N−1) where N is the number of input CEL files, (N−1) is the degrees of freedom for the Chi-Squared distribution, and the “probe-set variance for the gene” is the average of probe-set variances across the gene. In some embodiments of the present invention, probe-sets for a given gene or transcript cluster may be excluded from further analysis if they contain less than a minimum number of probes that pass through the previously described filter steps for GC content, reliability, variance and the like. For example in some embodiments, probe-sets for a given gene or transcript cluster may be excluded from further analysis if they contain less than about 1, 2, 3, 4, 5, 6, 7, 8, 9, 10, 11, 12, 13, 14, 15, or less than about 20 probes.

Methods of data analysis of gene expression levels or of alternative splicing may further include the use of a feature selection method and/or process as provided herein. In some embodiments of the present invention, feature selection is provided by use of the LIMMA software package (Smyth, G. K. (2005). Limma: linear models for microarray data. In: Bioinformatics and Computational Biology Solutions using R and Bioconductor, R. Gentleman, V. Carey, S. Dudoit, R. Irizarry, W. Huber (eds.), Springer, New York, pages 397-420).

Methods of data analysis of gene expression levels and or of alternative splicing may further include the use of a pre-classifier method and/or process (e.g., implemented by a pre-classifier analysis module). For example, a method and/or process may use a cell-specific molecular fingerprint to pre-classify the samples according to their composition and then apply a correction/normalization factor. This data/information may then be fed in to a final classification method and/or process which would incorporate that information to aid in the final diagnosis.

In certain embodiments, the methods of the present invention include the use of a pre-classifier method and/or process (e.g., implemented by a pre-classifier analysis module) that uses a molecular fingerprint to pre-classify the samples as smoker or non-smoker prior to application of a UIP/non-UIP classifier of the present invention.

Methods of data analysis of gene expression levels and/or of alternative splicing may further include the use of a classifier method and/or process (e.g., implemented by a classifier analysis module) as provided herein. In some embodiments of the present invention a diagonal linear discriminant analysis, k-nearest neighbor classifier, support vector machine (SVM) classifier, linear support vector machine, random forest classifier, or a probabilistic model-based method or a combination thereof is provided for classification of microarray data. In some embodiments, identified markers that distinguish samples (e.g. first ILD from second ILD, normal vs. ILD) or distinguish subtypes (e.g. IPF vs. NSIP) are selected based on statistical significance of the difference in expression levels between classes of interest. In some cases, the statistical significance is adjusted by applying a Benjamin Hochberg or another correction for false discovery rate (FDR).

In some cases, the classifier may be supplemented with a meta-analysis approach such as that described by Fishel and Kaufman et al. 2007 Bioinformatics 23(13): 1599-606. In some cases, the classifier may be supplemented with a meta-analysis approach such as a repeatability analysis. In some cases, the repeatability analysis selects markers that appear in at least one predictive expression product marker set.

Methods for deriving and applying posterior probabilities to the analysis of microarray data are known in the art and have been described for example in Smyth, G. K. 2004 Stat. Appi. Genet. Mol. Biol. 3: Article 3. In some cases, the posterior probabilities may be used to rank the markers provided by the classifier. In some cases, markers may be ranked according to their posterior probabilities and those that pass a chosen threshold may be chosen as markers whose differential expression is indicative of or diagnostic for samples that are for example IPF or NSIP. Illustrative threshold values include prior probabilities of 0.7, 0.75, 0.8, 0.85, 0.9, 0.925, 0.95, 0.975, 0.98, 0.985, 0.99, 0.995 or higher.

A statistical evaluation of the results of the molecular profiling may provide, but is not limited to providing, a quantitative value or values indicative of one or more of the following: the likelihood of diagnostic accuracy; the likelihood of an ILD; the likelihood of a particular ILD; the likelihood of the success of a particular therapeutic intervention, the likelihood the subject is a smoker, and the likelihood the subject is a non-smoker. Thus a physician, who is not likely to be trained in genetics or molecular biology, need not understand the raw data. Rather, the data is presented directly to the physician in its most useful form to guide patient care. The results of the molecular profiling can be statistically evaluated using a number of methods known to the art including, but not limited to: the students T test, the two sided T test, pearson rank sum analysis, hidden markov model analysis, analysis of q-q plots, principal component analysis, one way ANOVA, two way ANOVA, LIMMA and the like.

In some embodiments of the present invention, the use of molecular profiling alone or in combination with cytological analysis may provide a classification, identification, or diagnosis that is between about 85% accurate and about 99% or about 100% accurate. In some cases, the molecular profiling process and/or cytology provide a classification, identification, diagnosis of an ILD that is about, or at least about 85%, 86%, 87%, 88%, 90%, 91%, 92%, 93%, 94%, 95%, 96%, 97%, 97.5%, 98%, 98.5%, 99%, 99.5%, 99.75%, 99.8%, 99.85%, or 99.9% accurate. In some embodiments, the molecular profiling process and/or cytology provide a classification, identification, or diagnosis of the presence of a particular ILD type (e.g. IPF; NSIP; HP) that is about, or at least about 85%, 86%, 87%, 88%, 90%, 91%, 92%, 93%, 94%, 95%, 96%, 97%, 97.5%, 98%, 98.5%, 99%, 99.5%, 99.75%, 99.8%, 99.85%, or 99.9% accurate.

In some cases, accuracy may be determined by tracking the subject over time to determine the accuracy of the original diagnosis. In other cases, accuracy may be established in a deterministic manner or using statistical methods. For example, receiver operator characteristic (ROC) analysis may be used to determine the optimal assay parameters to achieve a specific level of accuracy, specificity, positive predictive value, negative predictive value, and/or false discovery rate.

In some embodiments of the present disclosure, gene expression products and compositions of nucleotides encoding for such products which are determined to exhibit the greatest difference in expression level or the greatest difference in alternative splicing between a first ILD and a second ILD (e.g., between IPF and NSIP), between ILD and normal, and/or between smoker and non-smoker may be chosen for use as molecular profiling reagents of the present disclosure. Such gene expression products may be particularly useful by providing a wider dynamic range, greater signal to noise, improved diagnostic power, lower likelihood of false positives or false negative, or a greater statistical confidence level than other methods known or used in the art.

In other embodiments of the present invention, the use of molecular profiling alone or in combination with cytological analysis may reduce the number of samples scored as non-diagnostic by about, or at least about 100%, 99%, 95%, 90%, 80%, 75%, 70%, 65%, or about 60% when compared to the use of standard cytological techniques known to the art. In some cases, the methods of the present invention may reduce the number of samples scored as intermediate or suspicious by about, or at least about 100%, 99%, 98%, 97%, 95%, 90%, 85%, 80%, 75%, 70%, 65%, or about 60%, when compared to the standard cytological methods used in the art.

In some cases the results of the molecular profiling assays, are entered into a database for access by representatives or agents of a molecular profiling business, the individual, a medical provider, or insurance provider. In some cases assay results include sample classification, identification, or diagnosis by a representative, agent or consultant of the business, such as a medical professional. In other cases, a computer analysis of the data is provided automatically. In some cases the molecular profiling business may bill the individual, insurance provider, medical provider, researcher, or government entity for one or more of the following: molecular profiling assays performed, consulting services, data analysis, reporting of results, or database access.

In some embodiments of the present invention, the results of the molecular profiling are presented as a report on a computer screen or as a paper record. In some cases, the report may include, but is not limited to, such information as one or more of the following: the number of genes differentially expressed, the suitability of the original sample, the number of genes showing differential alternative splicing, a diagnosis, a statistical confidence for the diagnosis, the likelihood the subject is a smoker, the likelihood of an ILD, and indicated therapies.

(iv) Categorization of Samples Based on Molecular Profiling Results

The results of the molecular profiling may be classified into one of the following: smoker, non-smoker, ILD, a particular type of ILD, a non-ILD, or non-diagnostic (providing inadequate information concerning the presence or absence of an ILD). In some cases, the results of the molecular profiling may be classified into IPF versus NSIP categories. In particular cases, the results may be classified as UIP or non-UIP.

In some embodiments of the present invention, results are classified using a trained classifier. Trained classifiers of the present invention implement methods and/or processes that have been developed using a reference set of known ILD and normal samples, known smoker and non-smoker samples, or combinations of known ILD and normal samples from smokers and/or non-smokers including, but not limited to, samples with one or more histopathologies. In some embodiments, training (e.g., using a classifier training module) comprises comparison of gene expression product levels in a first set biomarkers from a first ILD to gene expression product levels in a second set of biomarkers from a second ILD, where the first set of biomarkers includes at least one biomarker that is not in the second set. In some embodiments, training (e.g., using a classifier training module) comprises comparison of gene expression product levels in a first set biomarkers from a first ILD that is non-UIP to gene expression product levels in a second set of biomarkers from a second ILD that is UIP, where the first set of biomarkers includes at least one biomarker that is not in the second set. In some embodiments, training (e.g., using a classifier training module) further comprises comparison of gene expression product levels in a first set biomarkers from a first subject that is a smoker to gene expression product levels in a second set of biomarkers from a second subject that is a non-smoker, where the first set of biomarkers includes at least one biomarker that is not in the second set. In some embodiments, either the entire classifier or portions of the classifier can be trained (e.g., using a classifier training module) using comparisons of expression levels of biomarker panels within a classification panel against all other biomarker panels (or all other biomarker signatures) used in the classifier.

Classifiers suitable for categorization of samples include but are not limited to k-nearest neighbor classifiers, support vector machines, linear discriminant analysis, diagonal linear discriminant analysis, updown, naive Bayesian classifiers, neural network classifiers, hidden Markov model classifiers, genetic classifiers, or any combination thereof.

In some cases, trained classifiers of the present invention may incorporate data other than gene expression or alternative splicing data such as but not limited to DNA polymorphism data, sequencing data, scoring or diagnosis by cytologists or pathologists of the present invention, information provided by the pre-classifier method and/or process of the present disclosure, or information about the medical history of the subject of the present disclosure.

When classifying a biological sample for diagnosis of ILD, there are typically two possible outcomes from a binary classifier. Similarly, when classifying a biological sample for diagnosis of smoker, there are typically two possible outcomes from a binary classifier. When a binary classifier is compared with actual true values (e.g., values from a biological sample), there are typically four possible outcomes. If the outcome from a prediction is p (where “p” is a positive classifier output, such as a particular ILD) and the actual value is also p, then it is called a true positive (TP); however if the actual value is n then it is said to be a false positive (FP). Conversely, a true negative has occurred when both the prediction outcome and the actual value are n (where “n” is a negative classifier output, such as no ILD, or absence of a particular disease tissue as described herein), and false negative is when the prediction outcome is n while the actual value is p. In one embodiment, consider a diagnostic test that seeks to determine whether a person has a certain disease. A false positive in this case occurs when the person tests positive, but actually does not have the disease. A false negative, on the other hand, occurs when the person tests negative, suggesting they are healthy, when they actually do have the disease. In some embodiments, a Receiver Operator Characteristic (ROC) curve assuming real-world prevalence of subtypes can be generated by re-sampling errors achieved on available samples in relevant proportions.

The positive predictive value (PPV), or precision rate, or post-test probability of disease, is the proportion of patients with positive test results who are correctly diagnosed. It is the most important measure of a diagnostic method as it reflects the probability that a positive test reflects the underlying condition being tested for. Its value does however depend on the prevalence of the disease, which may vary. In one example, FP (false positive); TN (true negative); TP (true positive); FN (false negative). False positive rate (α)=FP/(FP+TN)-specificity; False negative rate (β)=FN/(TP+FN)-sensitivity; Power=sensitivity=1-β; Likelihood-ratio positive=sensitivity/(1-specificity); Likelihood-ratio negative=(1-sensitivity)/specificity.

The negative predictive value is the proportion of patients with negative test results who are correctly diagnosed. PPV and NPV measurements can be derived using appropriate disease subtype prevalence estimates. An estimate of the pooled disease prevalence can be calculated from the pool of indeterminates which roughly classify into B vs M by surgery. For subtype specific estimates, in some embodiments, disease prevalence may sometimes be incalculable because there are not any available samples. In these cases, the subtype disease prevalence can be substituted by the pooled disease prevalence estimate.

In some embodiments, the level of expression products or alternative exon usage is indicative of one or the following: IPF, NSIP, or HP.

In some embodiments, the level of expression products or alternative exon usage is indicative that the subject is a smoker or a non-smoker.

In some embodiments, the results of the expression analysis of the subject methods provide a statistical confidence level that a given diagnosis is correct. In some embodiments, such statistical confidence level is at least about, or more than about 85%, 90%, 91%, 92%, 93%, 94%, 95%, 96%, 97%, 98%, 99% 99.5%, or more.

Reports

A subject method and/or system may include generating a report that provides an indication that a sample (a lung tissue sample) is an ILD sample (e.g., using a report module). A subject diagnostic method can include generating a report that provides an indication as to whether an individual being tested has an ILD. A subject diagnostic method can include generating a report that provides an indication as to whether an individual being tested is, or is not a smoker. A subject method (or report module) can include generating a report that provides an indication as to whether an individual being tested has IPF (and not, e.g., an ILD other than IPF; e.g., the report can indicate that the individual has IPF and not NSIP).

In some embodiments, a subject method of diagnosing an ILD involves generating a report (e.g., using a report module). Such a report can include information such as a likelihood that the patient has an ILD; a likelihood that the patient is a smoker; a recommendation regarding further evaluation; a recommendation regarding therapeutic drug and/or device intervention; and the like.

For example, the methods disclosed herein can further include a step of generating or outputting a report providing the results of a subject diagnostic method, which report can be provided in the form of an electronic medium (e.g., an electronic display on a computer monitor), or in the form of a tangible medium (e.g., a report printed on paper or other tangible medium). An assessment as to the results of a subject diagnostic method (e.g., a likelihood that an individual has an ILD; a likelihood that an individual has IPF; a likelihood that an individual is a smoker) can be referred to as a “report” or, simply, a “score.” A person or entity that prepares a report (“report generator”) may also perform steps such as sample gathering, sample processing, and the like. Alternatively, an entity other than the report generator can perform steps such as sample gathering, sample processing, and the like. A diagnostic assessment report can be provided to a user. A “user” can be a health professional (e.g., a clinician, a laboratory technician, a physician (e.g., a cardiologist), etc.).

A subject report can further include one or more of: 1) service provider information; 2) patient data; 3) data regarding the expression level of a given gene product or set of gene products, a score or classifier decision; 4) follow-up evaluation recommendations; 5) therapeutic intervention or recommendations; and 6) other features.

Further Evaluation

Based on the expression level of a given gene product or set of gene products, and/or based on a report (as described above), a physician or other qualified medical personnel can determine whether further evaluation of the test subject (the patient) is required. Further evaluation can include, e.g., spirometry.

Therapeutic intervention

Based on the expression level of a given gene product or set of gene products, and/or based on a report (as described above), a physician or other qualified medical personnel can determine whether appropriate therapeutic intervention is advised.

Therapeutic intervention includes drug-based therapeutic intervention, device-based therapeutic intervention, and surgical intervention. Where a report indicates a likelihood that an individual has IPF, drug-based therapeutic intervention includes, e.g., administering to the individual an effective amount of pirfenidone, prednisone, azathioprine, or N-acetylcysteine. Surgical intervention includes, e.g., arterial bypass surgery.

Computer-Implemented Methods, Systems and Devices

Therapeutic Intervention

The methods of the present disclosure can be computer-implemented, such that method steps (e.g., assaying, comparing, calculating, and the like) are be automated in whole or in part.

Accordingly, the present disclosure provides methods, computer systems, devices and the like in connection with computer-implemented methods of facilitating a diagnosis of an interstitial lung disease (e.g., a diagnosis of IPF, NSIP, HP, etc.), including differential diagnosis.

The present disclosure further provides methods, computer systems, devices and the like in connection with computer-implemented methods of facilitating determination of smoker status (e.g., smoker vs. non-smoker).

The present disclosure further provides methods, computer systems, devices and the like in connection with computer-implemented methods of facilitating a diagnosis of an interstitial lung disease (e.g., a diagnosis of IPF, NSIP, HP, etc.), including differential diagnosis, wherein the methods further comprise determining a subjects smoker status (smoker vs. non-smoker) and incorporating smoker status into the determination of the subjects interstitial lung disease diagnosis. In some embodiments, (i) smoker status is incorporated into the interstitial lung disease diagnosis as a covariate in the model used during training (e.g., using a classifier training module). This approach boosts signal-to-noise ratio, particularly in data derived from smokers (were noise is higher) and allows data derived from smokers and non-smokers to be combined and used simultaneously. In some embodiments, (ii) smoker status is incorporated into the interstitial lung disease diagnosis by identifying one or more genes that are susceptible to smoker status bias and excluding such genes or weighing such genes differently than other genes that are not susceptible to smoker-status during interstitial lung disease diagnosis classifier training. In some embodiments, (iii) smoker status is incorporated into the interstitial lung disease diagnosis by constructing a tiered classification in which an initial classifier is trained to recognize the gene signatures that distinguish smokers from non-smokers (e.g., using a classifier training module). Once patient samples are pre-classified as “smoker” or “non-smoker” (e.g., using a pre-classifier analysis module), distinct classifiers that were each trained to distinguish UIP vs. Non UIP in smokers or non-smokers, respectively can be implemented to diagnose interstitial lung disease. In still further embodiments, such methods comprising the step of incorporating smoker status into the determination of the subjects interstitial lung disease diagnosis include a combination of one or more of the above mentioned means of such incorporation (i.e., a combination of two or more of embodiments (i) to (iii) in the instant paragraph.

For example, the method steps, including obtaining values for biomarker levels, comparing normalized biomarker (gene) expression levels to a control level, calculating the likelihood of an ILD (and optionally the likelihood a subject is a smoker), generating a report, and the like, can be completely or partially performed by a computer program product. Values obtained can be stored electronically, e.g., in a database, and can be subjected to a classifier executed by a programmed computer (e.g., using a classifier analysis module).

For example, the methods and/or systems of the present disclosure can involve inputting a biomarker level (e.g., a normalized expression level of a gene product) into a classifier analysis module to execute a method and/or process to perform the comparing and calculating step(s) described herein, and generate a report (e.g., using a report module) as described herein, e.g., by displaying or printing a report to an output device at a location local or remote to the computer. The output to the report can be a score (e.g., numerical score (representative of a numerical value) or a non-numerical score (e.g., non-numerical output (e.g., “IPF”, “No evidence of IPF”) representative of a numerical value or range of numerical values. In other aspects, the output may indicate “UIP” vs. “non-UIP.” In other aspects, the output may indicate “Smoker” vs. “Non-smoker”

The present disclosure thus provides a computer program product including a computer readable storage medium having software and/or hardware modules stored on it. The software and/or hardware modules can, when executed by a processor, execute relevant calculations based on values obtained from analysis of one or more biological sample (e.g., lung tissue sample) from an individual. The computer program product has stored therein a computer program for performing the calculation(s).

The present disclosure provides systems for executing the program described above, which system generally includes: a) a central computing environment or processor executing software and/or hardware modules; b) an input device, operatively connected to the computing environment, to receive patient data, wherein the patient data can include, for example, biomarker level or other value obtained from an assay using a biological sample from the patient, as described above; c) an output device, connected to the computing environment, to provide information to a user (e.g., medical personnel); and d) a method and/or process executed by the central computing environment (e.g., a processor), where the method and/or process is executed based on the data received by the input device, and wherein the method and/or process calculates a value, which value is indicative of the likelihood the subject has an ILD, as described herein.

The present disclosure also provides systems for executing the program described above, which system generally includes: a) a central computing environment or processor executing software and/or hardware modules; b) an input device, operatively connected to the computing environment, to receive patient data, wherein the patient data can include, for example, biomarker level or other value obtained from an assay using a biological sample from the patient, as described above; c) an output device, connected to the computing environment, to provide information to a user (e.g., medical personnel); and d) a method and/or process executed by the central computing environment (e.g., a processor), where the method and/or process is executed based on the data received by the input device, wherein the method and/or process calculates a value, which value is indicative of the likelihood the subject has an ILD, as described herein, and wherein the method and/or process uses smoking status (smoker vs. non-smoker) as a covariate in the model used during training. In some embodiments, the method and/or process excludes or weighs one or more gene that is susceptible to smoker status bias differently during classifier training to enrich the feature space used for training with genes that are not confounded or affected by smoking status.

In still further embodiments, the present disclosure provides systems for executing the program described above, which system generally includes: a) a central computing environment or processor executing software and/or hardware modules; b) an input device, operatively connected to the computing environment, to receive patient data, wherein the patient data can include, for example, biomarker level or other value obtained from an assay using a biological sample from the patient, as described above; c) an output device, connected to the computing environment, to provide information to a user (e.g., medical personnel); and d) a first method and/or process executed by the central computing environment (e.g., a processor), where the first method and/or process is executed based on the data received by the input device, wherein the first method and/or process calculates a value, which value is indicative of the likelihood a subject is a smoker or a non-smoker, as described herein, wherein the subject's status as a smoker or non-smoker causes the first method and/or process to apply a second method and/or process specifically trained (e.g., using a classifier training module) to distinguish UIP vs. Non UIP in smokers or non-smokers, respectively and e) wherein the second method and/or process is executed by the central computing environment (e.g., a processor), where the second method and/or process is executed based on the data received by the input device, and wherein the second method and/or process calculates a value, which value is indicative of the likelihood the subject has an ILD, as described herein,

Computer Systems

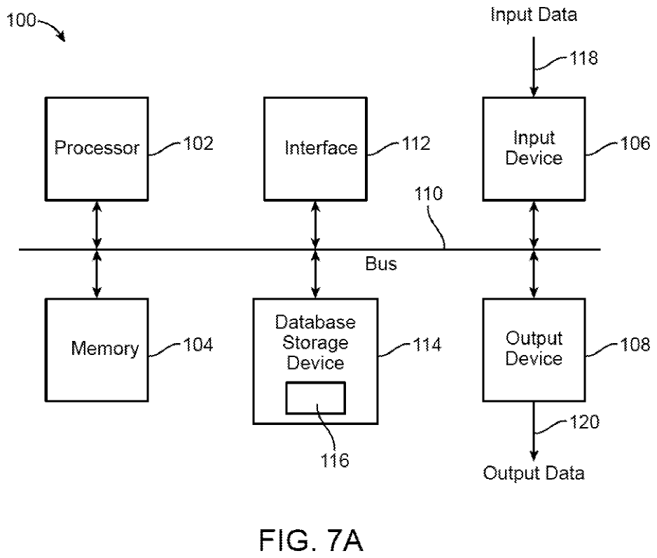

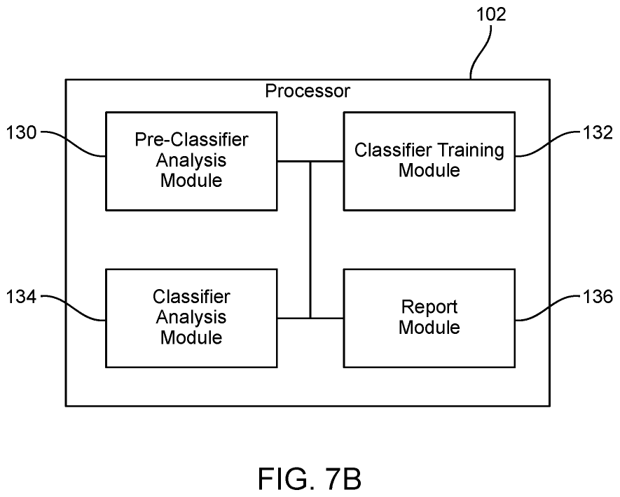

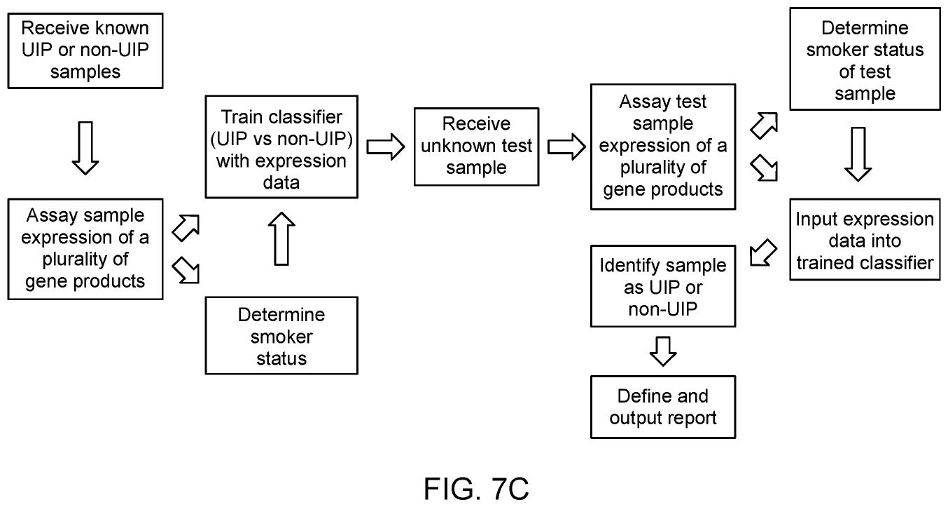

FIG. 7A illustrates a processing system 100 including at least one processor 102, or processing unit or plurality of processors, memory 104, at least one input device 106 and at least one output device 108, coupled together via a bus or group of buses 110. Processing system can be implemented on any suitable device, such as, for example, a host device, a personal computer, a handheld or laptop device, a personal digital assistant, a multiprocessor system, a microprocessor-based system, a programmable consumer electronic device, a minicomputer, a server computer, a web server computer, a mainframe computer, and/or a distributed computing environment that includes any of the above systems or devices

In certain embodiments, input device 106 and output device 108 can be the same device. An interface 112 can also be provided for coupling the processing system 100 to one or more peripheral devices, for example interface 112 can be a PCI card or PC card. At least one storage device 114 which houses at least one database 116 can also be provided.

The memory 104 can be any form of memory device, for example, volatile or nonvolatile memory, solid state storage devices, magnetic devices, etc. For example, in some embodiments, the memory 104 can be a random access memory (RAM), a memory buffer, a hard drive, a read-only memory (ROM), an erasable programmable read-only memory (EPROM), a database, and/or the like.

The processor 102 can include more than one distinct processing device, for example to handle different functions within the processing system 100. The processor 100 can be any suitable processing device configured to run or execute a set of instructions or code (e.g., stored in the memory) such as a general-purpose processor (GPP), a central processing unit (CPU), an accelerated processing unit (APU), a graphics processor unit (GPU), an Application Specific Integrated Circuit (ASIC), and/or the like. Such a processor 100 can run or execute a set of instructions or code stored in the memory associated with using a personal computer application, a mobile application, an internet web browser, a cellular and/or wireless communication (via a network), and/or the like. More specifically, the processor can execute a set of instructions or code stored in the memory 104 associated with analyzing and classifying data, as described herein.

Input device 106 receives input data 118 and can comprise, for example, a keyboard, a pointer device such as a pen-like device or a mouse, audio receiving device for voice controlled activation such as a microphone, data receiver or antenna such as a modem or wireless data adaptor, data acquisition card, etc. Input data 118 can come from different sources, for example keyboard instructions in conjunction with data received via a network.

Output device 108 produces or generates output data 120 and can comprise, for example, a display device or monitor in which case output data 120 is visual, a printer in which case output data 120 is printed, a port for example a USB port, a peripheral component adaptor, a data transmitter or antenna such as a modem or wireless network adaptor, etc. Output data 120 can be distinct and derived from different output devices, for example a visual display on a monitor in conjunction with data transmitted to a network. A user can view data output, or an interpretation of the data output, on, for example, a monitor or using a printer.

In some embodiments, the input device 106 and/or the output device 108 can be a communication interface configured to send and/or receive data via a network. More specifically, in such embodiments, the processing system 100 can act as a host device to one or more client devices (not shown in FIG. 7A). As such, the processing system 100 can send data to (e.g., output data 120) and receive data from (e.g., input data 118) the client devices. Such a communication interface can be any suitable module and/or device that can place the processing system 100 in communication with a client device such as one or more network interface cards or the like. Such a network interface card can include, for example, an Ethernet port, a WiFi® radio, a Bluetooth® radio, a near field communication (NFC) radio, and/or a cellular radio that can place the client device 150 in communication with the host device 110 via a network or the like.

The storage device 114 can be any form of data or information storage means, for example, volatile or non-volatile memory, solid state storage devices, magnetic devices, etc. For example, in some embodiments, the storage device 114 can be a random access memory (RAM), a memory buffer, a hard drive, a read-only memory (ROM), an erasable programmable read-only memory (EPROM), a database, and/or the like.

In use, the processing system 100 is adapted to allow data or information to be stored in and/or retrieved from, via wired or wireless communication means, at least one database 116. The interface 112 may allow wired and/or wireless communication between the processing unit 102 and peripheral components that may serve a specialized purpose. In general, the processor 102 can receive instructions as input data 118 via input device 106 and can display processed results or other output to a user by utilizing output device 108. More than one input device 106 and/or output device 108 can be provided. The processing system 100 may be any suitable form of terminal, server, specialized hardware, or the like. The processing system 100 may be a part of a networked communications system.

Processing system 100 can connect to a network, for example, a local area network (LAN), a virtual network such as a virtual local area network (VLAN), a wide area network (WAN), a metropolitan area network (MAN), a worldwide interoperability for microwave access network (WiMAX), a cellular network, the Internet, and/or any other suitable network implemented as a wired and/or wireless network. For instance, when used in a LAN networking environment, the computing system environment 100 is connected to the LAN through a network interface or adapter. When used in a WAN networking environment, the computing system environment typically includes a modem or other means for establishing communications over the WAN, such as the Internet. The modem, which may be internal or external, may be connected to a system bus via a user input interface, or via another appropriate mechanism. In a networked environment, program modules depicted relative to the computing system environment 100, or portions thereof, may be stored in a remote memory storage device. It is to be appreciated that the illustrated network connections of FIG. 7 are examples and other means of establishing a communications link between multiple computers may be used.

Input data 118 and output data 120 can be communicated to other devices via the network. The transfer of information and/or data over the network can be achieved using wired communications means or wireless communications means. A server can facilitate the transfer of data between the network and one or more databases. A server and one or more databases provide an example of an information source.

Thus, the processing computing system environment 100 illustrated in FIG. 7A may operate in a networked environment using logical connections to one or more remote computers. The remote computer may be a personal computer, a server, a router, a network PC, a peer device, or other common network node, and typically includes many or all of the elements described above.

FIG. 7B illustrates the processor 102 of FIG. 7A in greater detail. The processor 102 can be configured to execute specific modules. The modules can be, for example, hardware modules, software modules stored in the memory 104 and/or executed in the processor 102, and/or any combination thereof. For example, as shown in FIG. 7B, the processor 102 includes and/or executes a pre-classifier analysis module 130, a classifier training module 132, a classifier analysis module 134 and a report module 136. As shown in FIG. 7B, the pre-classifier analysis module 130, the classifier training module 132, the classifier analysis module 134 and the report module 136 can be connected and/or electrically coupled. As such, signals can be sent between the pre-classifier analysis module 130, the classifier training module 132, the classifier analysis module 134 and the report module 136.

The classifier training module 132 can be configured to receive a corpora of data (e.g. gene expression data, sequencing data) and train a classifier. For example, clinical annotation data from samples previously identified as UIP and non-UIP (e.g., by an expert) can be received by the input device 106 and used by the classifier training module 132 to identify correlations between the samples previously identified as UIP and non-UIP. For example, expert TBB histopathology labels (i.e., UIP or Non UIP), expert HRCT labels, and/or expert patient-level clinical outcome labels can be obtained and used alone or in combination to train the classifier using microarray and/or sequencing data. The feature space used can include gene expression, variants, mutations, fusions, loss of heterozygoxity (LOH), biological pathway effect and/or any other dimension of the data that can be extracted as a feature for the purposes of training a machine-learning algorithm. In some embodiments, the feature space used for training a UIP vs. Non-UIP classifier, a smoker vs. Non-smoker classifier, or a UIP vs. Non-UIP and smoker vs. Non-smoker classifier includes gene expression, variants, mutations, fusions, loss of heterozygoxity (LOH), and biological pathway effect. In some embodiments, the feature space used for training a UIP vs. Non-UIP classifier, a smoker vs. Non-smoker classifier, or a UIP vs. Non-UIP and smoker vs. Non-smoker classifier includes gene expression and variant dimensions.

In some embodiments, the classifier training module 132 can train a smoker classifier and a non-smoker classifier based on an indication associated with whether a received sample is associated with a smoker or non-smoker. In other embodiments, the smoker/non-smoker can be used as an attribute (a model covariate) to train a single classifier. After the classifier is trained, it can be used to identify and/or classify newly received and unknown samples as described herein.

The pre-classifier analysis module 130 can identify whether a sample is associated with a smoker or a non-smoker. Specifically, the pre-classifier analysis module 130 can use any suitable method to identify and/or classify a sample as coming from an individual that smokes (or has a past history of heavy smoking) versus an individual that does not smoke (or has no smoking history). The classification can be done in any suitable manner such as, receiving an indication from a user, identification of genes that are susceptible to smoker-status bias, using a machine-learning classifier, and/or any other suitable method described herein.

The classifier analysis module 134 can input the sample into the classifier to identify and/or classify the received sample as associated with UIP and non-UIP. Specifically, the classifier analysis module 134 can use a trained classifier to identify whether the sample indicates UIP or non-UIP. In some embodiments, the classifier analysis module 134 can indicate a percentage or confidence score of the sample being associated with UIP or non-UIP. In some embodiments, the classifier analysis module 134 can execute two separate classifiers: one for smoker samples and the other for non-smoker samples (as determined by the pre-classifier analysis module 130). In other embodiments, a single classifier is executed for both smoker and non-smoker samples with an input for smoker status.

The report module 136 can be configured to generate any suitable report based on the outcome of the classifier analysis module 134 as described in further detail herein. In some cases, the report may include, but is not limited to, such information as one or more of the following: the number of genes differentially expressed, the suitability of the original sample, the number of genes showing differential alternative splicing, a diagnosis, a statistical confidence for the diagnosis, the likelihood the subject is a smoker, the likelihood of an ILD, and indicated therapies.

FIG. 7C illustrates a flow chart of one non-limiting embodiment of the present invention wherein gene product expression data for known UIP and non-UIP samples are used to train (e.g., using a classifier training module) a classifier for differentiating UIP vs. non-UIP, wherein the classifier optionally considers smoker status as a covariant, and wherein gene product expression data from unknown samples are input into the trained classifier to identify the unknown samples as either UIP or non-UIP, and wherein the results of the classification via the classifier are defined and output via a report.

Certain embodiments may be described with reference to acts and symbolic representations of operations that are performed by one or more computing devices, such as the computing system environment 100 of FIG. 7A. As such, it will be understood that such acts and operations, which are at times referred to as being computer-executed, include the manipulation by the processor of the computer of electrical signals representing data in a structured form. This manipulation transforms the data or maintains them at locations in the memory system of the computer, which reconfigures or otherwise alters the operation of the computer in a manner understood by those skilled in the art. The data structures in which data is maintained are physical locations of the memory that have particular properties defined by the format of the data. However, while an embodiment is being described in the foregoing context, it is not meant to be limiting as those of skill in the art will appreciate that the acts and operations described hereinafter may also be implemented in hardware.

Embodiments may be implemented with numerous other general-purpose or special-purpose computing devices and computing system environments or configurations. Examples of other computing systems, environments, and configurations that may be suitable for use with an embodiment include, but are not limited to, personal computers, handheld or laptop devices, personal digital assistants, multiprocessor systems, microprocessor-based systems, programmable consumer electronics, network, minicomputers, server computers, web server computers, mainframe computers, and distributed computing environments that include any of the above systems or devices.

Embodiments may be described in a general context of computer-executable instructions, such as hardware and/or software modules. An embodiment may also be practiced in a distributed computing environment where tasks are performed by remote processing devices that are linked through a communications network. In a distributed computing environment, program modules may be located in both local and remote computer storage media including memory storage devices.

Computer program products

The present disclosure provides computer program products that, when executed on a programmable computer such as that described above with reference to FIG. 7, can carry out the methods of the present disclosure. As discussed above, the subject matter described herein may be embodied in systems, apparatus, methods, and/or articles depending on the desired configuration. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device (e.g. video camera, microphone, joystick, keyboard, and/or mouse), and at least one output device (e.g. display monitor, printer, etc.).

Computer programs (also known as programs, software, software applications, applications, components, or code) include instructions for a programmable processor, and may be implemented in a high-level procedural and/or object-oriented programming language, and/or in assembly/machine language. As used herein, the term “machine-readable medium” refers to any computer program product, apparatus and/or device (e.g., magnetic discs, optical disks, memory, etc.) used to provide machine instructions and/or data to a programmable processor, including a machine-readable medium that receives machine instructions as a machine-readable signal.

It will be apparent from this description that aspects of the present disclosure may be embodied, at least in part, in software, hardware, firmware, or any combination thereof. Thus, the techniques described herein are not limited to any specific combination of hardware circuitry and/or software, or to any particular source for the instructions executed by a computer or other data processing system. Rather, these techniques may be carried out in a computer system or other data processing system in response to one or more processors, such as a microprocessor, executing sequences of instructions stored in memory or other computer-readable medium including any type of ROM, RAM, cache memory, network memory, floppy disks, hard drive disk (HDD), solid-state devices (SSD), optical disk, CD-ROM, and magnetic-optical disk, EPROMs, EEPROMs, flash memory, or any other type of media suitable for storing instructions in electronic format.

In addition, the processor(s) may be, or may include, one or more programmable general-purpose or special-purpose microprocessors, digital signal processors (DSPs), programmable controllers, application specific integrated circuits (ASICs), programmable logic devices (PLDs), trusted platform modules (TPMs), or the like, or a combination of such devices. In alternative embodiments, special-purpose hardware such as logic circuits or other hardwired circuitry may be used in combination with software instructions to implement the techniques described herein.

Arrays and Kits

The present disclosure provides arrays and kits for use in carrying out a subject evaluating method or a subject diagnostic method.

Arrays

A subject array can comprise a plurality of nucleic acids, each of which hybridizes to a gene differentially expressed in a cell present in a tissue sample obtained from an individual being tested for an ILD.

A subject array can comprise a plurality of nucleic acids, each of which hybridizes to a gene differentially expressed in a cell present in a tissue sample obtained from an individual being tested for smoker status.

A subject array can comprise a plurality of nucleic acids, each of which hybridizes to a gene differentially expressed in a cell present in a tissue sample obtained from an individual being tested for both smoker status and an ILD.

A subject array can comprise a plurality of member nucleic acids, each of which member nucleic acids hybridizes to a different gene product. In some cases, two or more member nucleic acids hybridize to the same gene product; e.g., in some cases 2, 3, 4, 5, 6, 7, 8, 9, 10, or more member nucleic acids hybridize to the same gene product. A member nucleic acid can have a length of from about 5 nucleotides (nt) to about 100 nt, e.g., 5, 6, 7, 8, 9, 10, 11, 12, 13, 14, 15, 16, 17 18, 19, 20, 20-25, 25-30, 30-40, 40-50, 50-60, 60-70, 70-80, 80-90, or 90-100 nt. A nucleic acid can have one or more phosphate backbone modifications.

A subject array can include from about 10 to about 10⁵ unique member nucleic acids, or more than 10⁵ unique member nucleic acids. For example, a subject array can include from about 10 to about 10², from about 10² to about 10³, from about 10³ to about 10⁴, from about 10⁴ to about 10⁵, or more than 10⁵, unique member nucleic acids.

Abbreviations

| | |
|---|---|
| adj.P.Value.edgeR: | False discovery rate adjusted p value of RNAseq gene |
| | expression data using edgeR analysis. |
| adj.P.Value.microarray | False discovery rate adjusted p value of RNAseq gene |
| | expression data using microarray analysis |
| adj.P.Value.npSeq: | False discovery rate adjusted p value of RNAseq gene |
| | expression data using npSeq analysis |
| BRONCH: | Broncholitis |
| CIF-NOC | Chronic Interstitial Fibrosis Not Otherwise Classified |
| edgeR: | an R package for the significance analysis of sequencing data |
| Ensembl ID: | Gene Identifier from Ensembl Genome Browser database |
| FDR: | False Discovery Rate, an adjusted p value that limits the |
| | possibility that the results are random due to the large |
| | number of genes simultaneously evaluated. |
| Gene Symbol: | Gene Identifier from HUGO Gene Nomenclature Committee |
| logFC.edgeR: | Log2 fold change of RNAseq gene expression data using |
| | edgeR analysis |
| logFC.microarray: | Log2 fold change of RNAseq gene expression data using |
| | LIMMA microarray analysis |
| logFC.npSeq: | Log2 fold change of RNAseq gene expression data using |
| | npSeq analysis |
| microarray: | Gene expression analysis using gene arrays such as from |
| | Affymetrix. |
| NML: | Normal Lung, usually obtained from human lung donor |
| | tissue that was ultimately never transplanted |
| npSeq: | an R package for the significance analysis of sequencing data |
| NSIP: | Non Specific Interstitial Pneumonia |
| OP: | Organizing Pneumonia |
| P.value.edgeR: | p value of RNAseq gene expression data using edgeR analysis |
| P.value.microarray: | p value of RNAseq gene expression data using LIMMA microarray |
| | analysis |
| P.value.npSeqp: | value of RNAseq gene expression data using npSeq analysis |
| RB: | Respiratory Broncholitis |
| REST: | A combination of all other ILDs except the subtype it is being |
| | compared to. Usually HP and NSIP, BRONCH, CIF-NOC, OP, |
| | RB and SARC. |
| SARC: | Sarcoidosis |
| SQC: | Squamous Cell Carcinoma |
| TCID: | “TCID” or “Transcript Cluster Identifier” refers to a gene |
| | level identifier used by all Affymetrix microarrays. Each TCID is |
| | associated with a fixed reference number that identifies a set of |
| | specific probes having sequences for a specific gene. Such specific |
| | probes are present on a given array commercially available from |
| | Affymetrix. TCID numbers thus refer to a gene product(s) of a |
| | specific gene, and can be found, e.g., at the following world wide |
| | web address: affymetrix.com/the sequences of which probes and |
| | gene products are hereby incorporation herein in their entirety. |
| UIP: | Usual Interstitial Pneumonia; the HRCT or histopathology pattern |
| | observed in IPF |
| LIMMA: | Linear Models for Microarray Data; an R package for the |
| | significance analysis of microarray data. |

“ENSEMBL ID” refers to a gene identifier number from the Ensembl Genome Browser database (see world wide web address: ensembl.org/index.html, incorporate herein). Each identifier begins with the letters ENSG to denote “Ensembl Gene”. Each ENSEMBL ID number (i.e., each “gene” in the Ensembl database) refers to a gene defined by a specific start and stop position on a particular human chromosome, and therefore defines a specific locus of the human genome. As one of average skill in the art would fully appreciate, all of the gene symbols disclosed herein refer to gene sequences, which are readily available on publically available databases, e.g., UniGene database (Pontius J U, Wagner L, Schuler G D. UniGene: a unified view of the transcriptome. In: The NCBI Handbook. Bethesda (MD): National Center for Biotechnology Information; 2003, available at the world wide web address ncbi.nlm.nih.gov/unigene, incorporated herein), RefSeq (The NCBI handbook [Internet], Bethesda (MD): National Library of Medicine (US), National Center for Biotechnology Information; 2002 October Chapter 18, The Reference Sequence (RefSeq) Project, available at the world wide web address: ncbi.nlm.nih.gov/refseq/, incorporate herein), Ensembl (EMBL, available at the world wide web address: ensembl.org/index.html, incorporated herein), and the like. The sequences of the genes disclosed herein via their gene symbols, Ensembl IDs, and Entrez IDs are herein incorporated in their entirety.

All references, patents, and patent applications cited herein are incorporated in their entirety for all purposes.

### EXAMPLES

### Example 1

Sample Collection, Pathology Diagnosis, and Labeling

Video-assisted thoracoscopic surgery (VATS) specimens were prospectively collected as a part of an Institutional Review Board (IRB) approved ongoing multi-center clinical protocol, BRonchial sAmple collection for a noVel gEnomic test (BRAVE), sponsored by Veracyte, Inc. (South San Francisco, CA). Additional VATS and surgical lung biopsy specimens were obtained from banked sources.

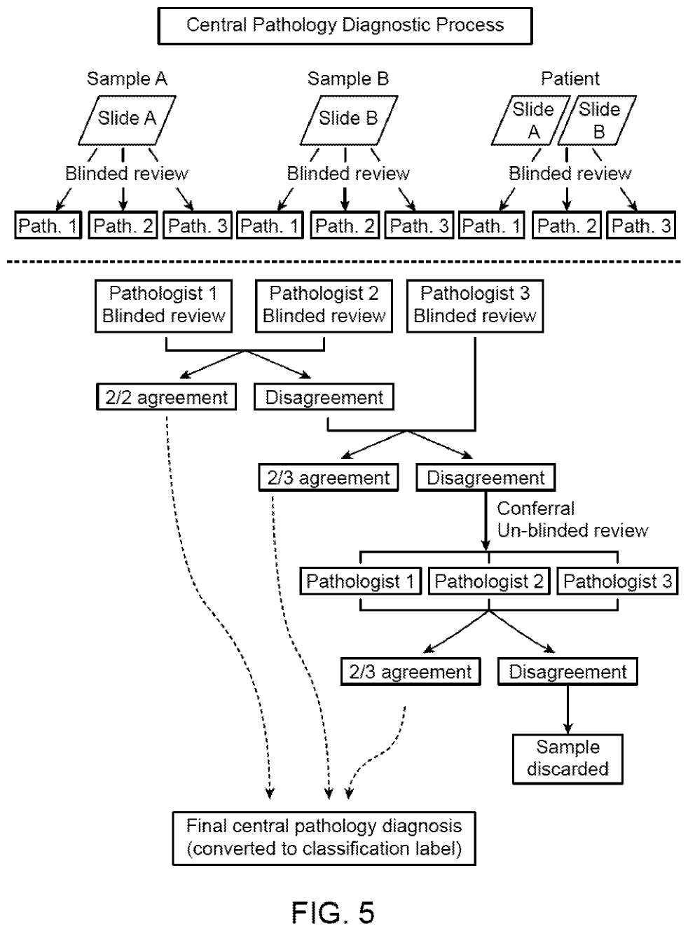

Following surgery, histology slides were collected, de-identified, and submitted to expert pathology review. Selected slides were scanned to construct a permanent digital file of microscopic images (Aperio, Vista, CA). Slides were evaluated according to the central pathology diagnostic process described in FIG. 5, resulting in both sample-level and patient-level pathology diagnoses. Pathology categories are summarized in Table 3. A patient can have more than one sample-level diagnosis (i.e. one per VATS sample per patient, most often one from each of the lower and upper lobes of the right lung), but can only have one patient-level diagnosis.

TABLE 3
List of all pathology diagnoses considered in
our central pathology diagnostic process.

| Diagnosis | Abbreviation |
|---|---|
| Classic Usual Interstitial Pneumonia | Classic UIP |
| Difficult Usual Interstitial Pneumonia | Difficult UIP |
| Favor Usual Interstitial Pneumonia | Favor UIP |
| Cellular Non-specific Interstitial Pneumonia | Cellular NSIP |
| Fibrotic Non-specific Interstitial Pneumonia | Fibrotic NSIP |
| Both cellular and fibrotic Non-specific Interstitial | Both cellular |
| | and fibrotic |
| Pneumonia | NSIP |
| Favor Non-specific Interstitial Pneumonia | Favor NSIP |
| Hypersensitivity Pneumonitis | HP |
| Favor Hypersensitivity Pneumonitis | Favor HP |
| Chronic Interstitial Fibrosis, Not Otherwise Classified | CIF/NOC |
| Organizing Pneumonia | OP |
| Diffuse Alveolar Damage | DAD |
| Respiratory Bronchiolitis | RB |
| Smoking-Related Interstitial Fibrosis | SRIF |
| Emphysema | Emphysema |
| Bronchiolitis | Bronchiolitis |
| Sarcoidosis | Sarcoidosis |
| Lymphangioleiomyomatosis | LAM |
| Langerhans cell histiocytosis | LCH |
| Eosinophilic Pneumonia | EP |
| Non-diagnostic | ND |
| Other | Other |

Most diagnostic terminologies follow American Thoracic Society (ATS) 2011 or 2013 guidelines^{5,6}, but a few changes were made by the expert pathologist panel to better characterize features at the lobe level. In particular, ‘Classic UIP’ and ‘Difficult UIP’ were included instead of ‘Definite UIP’ and ‘Probable UIP’ as described in the ATS 2011 guidelines. Chronic Interstitial Fibrosis, Not Otherwise Classified (CIF/NOC) corresponds to unclassifiable fibrotic ILD. Three subcategories of CIF/NOC, ‘Favor UIP’, ‘Favor NSIP’, and ‘Favor HP’, were defined to specify cases of unclassifiable fibrosis which, in the judgment of the expert pathology panel, exhibit features suggestive of UIP, non-specific interstitial pneumonia (NSIP), or hypersensitivity pneumonitis (HP). A diagnosis of Smoking-Related Interstitial Fibrosis (SRIF) is also included²⁰.

For classification, sample-level pathology diagnoses were converted into binary class labels (UIP and non-UIP). Among the pathology diagnosis categories (Table 3), the ‘UIP’ class includes (1) UIP, (2) Classic UIP, (3) Difficult UIP, and (4) CIF/NOC, Favor UIP. All other pathology diagnoses except Non-diagnostic (ND) were assigned to the ‘non-UIP’ class.

### Example 2

Sample Processing

Frozen tissue samples were mounted for sectioning using Tissue-Tek O.C.T. medium (Sakura Finetek U.S.A.) and 2×20 μm sections generated using a CM1800 cryostat (Leica Biosystems, Buffalo Grove, Illinois). Tissue curls were immediately immersed in RNAprotect (QIAGEN, Valencia, California), incubated overnight at 4° C. and stored at −80° C. until extraction. Whenever possible, adjacent 5 μm tissue curls were mounted onto glass slides and processed for hematoxylin and eosin (H&E) staining following standard procedures.

Nucleic acids were extracted using the AllPrep Micro Kit (QIAGEN) according to manufacturer's guidelines. Total RNA yield and quality was determined using Quant-it (Invitrogen) and Pico BioAnalyzer kits (Agilent). Fifteen nanograms of total RNA were amplified using Ovation FFPE WTA System (NuGEN, San Carlos, California), hybridized to GeneChip Gene ST 1.0 (Affymetrix, Santa Clara, California) microarrays, processed and scanned according to the manufacturer's protocols. Expression data was normalized by Robust Multi-array Average (RMA).

### Example 3

Next-Generation RNA Sequencing

Whole transcriptome RNA sequencing was performed on select samples at a targeted minimum read depth of 80 million paired-end reads per sample. Briefly, 10 ng of total RNA was amplified using the Ovation RNASeq System v2 (NuGEN, San Carlos, California) and TruSeq (Illumina, San Diego, California) sequencing libraries were prepared and sequenced on an Illumina HiSeq according to manufacturer's instructions. Raw reads were aligned to the hg19 genome assembly using TopHat2. Gene counts were obtained using HTS eq and normalized in Bioconductor using the varianceStabilizingTransformation function in the DESeq2 package. Raw counts and normalized expression levels were obtained for 55,097 transcripts.

### Example 4

Cohort Selection and Classifier Training

The study cohort initially included both banked (n=128) and prospectively collected BRAVE (n=38) tissues. Banked samples with poor cellularity on H&E staining (n=4 from a single patient) or normal lung tissue appearance (n=1) were excluded, as were samples diagnosed as ‘unclassifiable fibrotic ILD’ i.e. CIF/NOC (n=3) or samples that lacked pathology agreement by at least two pathologists (n=29). For BRAVE samples, CIF/NOC samples were not excluded. Only one BRAVE cohort sample was omitted, due to missing central pathology diagnosis. Processed RNA samples with residual genomic DNA contamination (n=2) or low RNA quality (RNA integrity number (RIN)<4) (n=1) were also excluded. After all exclusions, 125 samples from 86 patients remained for use in classification. The age, gender, smoking history and pathology diagnoses of included patients are summarized in Table 1.

TABLE 1
Cohort summary. Within each set of microarray data or RNASeq data, clinical factors such
as age, gender and smoking history are summarized across patients. In addition, samples
are summarized by sample-level pathology diagnosis (counts without parenthesis), and patients
are summarized by patient-level pathology diagnosis (counts within parenthesis). Zeros
in either case are due to discordance between sample-level and patient-level pathology;
counts will therefore not be additive. Of the 36 samples in the RNASeq training set, 22
overlap with the microarray training set and 14 overlap with the microarray test set.

| | | | | Microarray | | | | RNASeq | |
|---|---|---|---|---|---|---|---|---|---|
| Category | Sub-category | All | | Training | | Test | | Training | |
| Size | Patient | 86 | | 54 | | 32 | | 29 | |
| Age | Mean (range) | 57.9 | (25-83) | 58.9 | (25-83) | 56.3 | (32-76) | 60.5 | (32-80) |
| Gender | Male | 32 | (37.2%) | 22 | (40.7%) | 10 | (31.2%) | 16 | (55.2%) |
| | Female | 54 | (62.8%) | 32 | (59.3%) | 22 | (68.8%) | 13 | (44.8%) |
| Smoking | Yes | 45 | (52.3%) | 33 | (61.1%) | 12 | (37.5%) | 15 | (51.7%) |
| | No | 38 | (44.2%) | 19 | (35.2%) | 19 | (59.4%) | 14 | (48.3%) |
| | Unknown | 3 | (3.5%) | 2 | (3.7%) | 1 | (3.1%) | 0 | |
| Pathology | UIP | 45 | (30) | 28 | (18) | 17 | (12) | 14 | (14) |
| diagnosis | Classic UIP | 8 | (5) | 5 | (4) | 3 | (1) | 2 | (0) |
| | Difficult UIP | 5 | (2) | 3 | (1) | 2 | (1) | 1 | (0) |
| | Favor UIP | 3 | (1) | 3 | (1) | 0 | (0) | 0 | (0) |
| | Fibrotic NSIP | 2 | (0) | 0 | (0) | 2 | (0) | 0 | (0) |
| | Cellular NSIP | 7 | (0) | 5 | (0) | 2 | (0) | 2 | (0) |
| | Both cellular and fibrotic | 14 | (0) | 9 | (0) | 5 | (0) | 3 | (0) |
| | NSIP | | | | | | | | |
| | NSIP | 0 | (15) | 0 | (10) | 0 | (5) | 0 | (5) |
| | Favor HP | 2 | (1) | 0 | (0) | 2 | (1) | 0 | (0) |
| | HP | 16 | (14) | 11 | (10) | 5 | (4) | 3 | (2) |
| | Unclassifiable fibrotic | 4 | (5) | 2 | (3) | 2 | (2) | 0 | (0) |
| | ILD | | | | | | | | |
| | Sarcoidosis | 4 | (4) | 2 | (2) | 2 | (2) | 2 | (2) |
| | RB | 4 | (2) | 0 | (1) | 4 | (1) | 3 | (1) |
| | OP | 2 | (1) | 2 | (1) | 0 | (0) | 1 | (1) |
| | SRIF | 1 | (1) | 0 | (0) | 1 | (1) | 1 | (1) |
| | Bronchiolitis | 1 | (1) | 1 | (1) | 0 | (0) | 1 | (1) |
| | Emphysema | 2 | (0) | 2 | (0) | 0 | (0) | 0 | (0) |
| | DAD | 0 | (1) | 0 | (0) | 0 | (1) | 0 | (1) |
| | Other | 5 | (3) | 4 | (2) | 1 | (1) | 3 | (1) |
| | Total | 125 | (86) | 77 | (54) | 48 | (32) | 36 | (29) |

125 samples (86 patients) were available for microarray classification. The 86 patients were randomized into training and test sets while controlling for patient-level pathology subtype bias (Table 1). The microarray training set consists of 77 samples (39 UIP and 38 non-UIP) from 54 patients. The microarray test set consists of 48 samples (22 UIP vs. 26 non-UIP) from 32 patients.

RNASeq data was generated for a subset of 36 samples (17 UIP and 19 non-UIP) from 29 patients (Table 1), representing a spectrum of ILD subtypes. Among the 36 samples, 22 overlap with the microarray training set and 14 overlap with the microarray test set. Due to the small sample size of this dataset, classification performance was evaluated by cross-validation (CV) only.

### Example 5

Training Models, Classification, Feature Selection

All statistical analyses were carried out using R version 3.0.1²¹. For the microarray classifier, genes differentially expressed between UIP and non-UIP classes were ranked by limma, then the top 200 genes with lowest false discovery rate (FDR) (<0.0003) were carried forward as candidate genes for model building. Several models were built using different methods, and the one with the lowest error was chosen. Feature selection and model estimation were performed by logistic regression with lasso penalty using glmnet. For the RNASeq classifier, genes were ranked by FDR resulting from a Wald-style test implemented in the DESeq2 package on the raw count data. The top features (N ranging from 10 to 200) were used to train a linear support vector machine (SVM) using the e1071 library on the normalized expression data.

Classifier performance was evaluated by CV and, when available, by an independent test set. To minimize over-fitting, a single patient was maintained as the smallest unit when defining the training/test set and the CV partition; i.e. all samples belonging to the same patient were held together as a group in the training/test set or in CV partitions. The CV methods used include leave-one-patient-out (LOPO) and 10-fold patient-level CV.

Performance was reported as the area under the curve (AUC), and specificity (1·0— false positive rate) and sensitivity (1·0— false negative rate) at a given score threshold. We set the score threshold to require at least >90% specificity. For each performance measurement, 95% confidence intervals were computed using 2000 stratified bootstrap replicates and the pROC package and reported as [CI lower-upper].

### Example 6

Spatial Heterogeneity in Samplings From Explanted Lungs

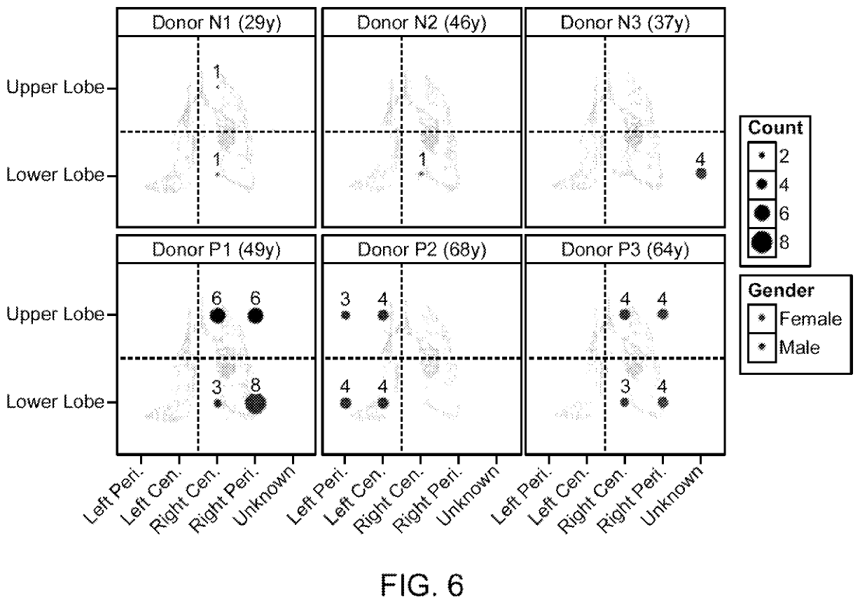

A total of 60 samplings from three normal lung donors (n=7) and three lungs from patients diagnosed with IPF (n=53) were analyzed using genome-wide microarray data. Intact normal and diseased lungs obtained during transplant procedures were collected following a protocol approved by the Institutional Review Board (IRB) of Inova Fairfax, Falls Church, Virginia. The upper and lower lobes of explanted lungs from three normal donors and three patients diagnosed with IPF were sampled centrally and peripherally. The location and number of the explant samples is illustrated in FIG. 6. Surgical pathology and final clinical diagnoses were provided by the originating institutions. Pathology over-reads by three expert pathologists unanimously confirmed UIP in all three IPF patient explant lungs.

Gene expression was evaluated in seven normal and 53 IPF explant lung samples. Genes differentially expressed between normal and IPF patient explant samples were identified and ranked by false discovery rate (FDR) using the R limma package (Smyth, G. K. (2005)). The top 200 genes differentially expressed between UIP and non-UIP classes in the microarray training set are shown in Table 12. Using the top 200 genes with the lowest FDR adjusted P-values (<1.45e-07), the Pearson correlation coefficient was calculated for all pairs of 53 UIP samples.

TABLE 12
Top 200 genes differentially expressed between UIP and non-UIP classes in the microarray
training set, with indication of 22 genes used by the microarray classifier.

| | | | | | FDR | | |
|---|---|---|---|---|---|---|---|
| | GENE | | | | adjusted | | Used by |
| TCID | SYMBOL | logFC | MedExpr.UIP | MedExpr.NonUIP | p-value | rank | Classifier |
| 8117760 | HLA-F | −0.48 | 9.02 | 9.53 | 2.35E−09 | 1 | Used |
| 8177717 | HLA-F | −0.50 | 9.35 | 9.94 | 2.35E−09 | 2 | |
| 8179019 | HLA-F | −0.50 | 9.31 | 9.83 | 2.35E−09 | 3 | |
| 8101031 | CDKL2 | −0.95 | 7.31 | 8.16 | 1.29E−07 | 4 | Used |
| 8106827 | GPR98 | −0.77 | 6.11 | 6.74 | 2.16E−07 | 5 | Used |
| 8100026 | ATP8A1 | −0.65 | 7.63 | 8.17 | 9.86E−07 | 6 | |
| 7931930 | PRKCQ | −0.64 | 6.78 | 7.40 | 1.81E−06 | 7 | Used |
| 8135661 | CFTR | −1.00 | 6.50 | 7.56 | 2.37E−06 | 8 | |
| 8177725 | HLA-G | −0.29 | 10.67 | 10.89 | 2.37E−06 | 9 | Used |
| 8179034 | HLA-G | −0.29 | 10.67 | 10.89 | 2.37E−06 | 10 | |
| 8123246 | SLC22A3 | −0.94 | 6.90 | 7.85 | 2.37E−06 | 11 | |
| 8118571 | PSMB9 | −0.53 | 10.04 | 10.68 | 2.37E−06 | 12 | |
| 8178211 | PSMB9 | −0.53 | 10.04 | 10.68 | 2.37E−06 | 13 | |
| 8179495 | PSMB9 | −0.53 | 10.04 | 10.68 | 2.37E−06 | 14 | |
| 8065719 | PXMP4 | −0.67 | 7.12 | 7.94 | 2.37E−06 | 15 | |
| 7926037 | PFKFB3 | −0.45 | 7.66 | 8.10 | 2.81E−06 | 16 | Used |
| 8037205 | CEACAM1 | −0.41 | 6.47 | 6.97 | 3.04E−06 | 17 | Used |
| 8178489 | HLA-C | −0.36 | 11.26 | 11.64 | 3.04E−06 | 18 | |
| 7917561 | GBP4 | −0.87 | 8.49 | 9.57 | 3.59E−06 | 19 | |
| 7968678 | FREM2 | −1.08 | 6.67 | 7.72 | 3.88E−06 | 20 | |
| 7907492 | RABGAP1L | −0.32 | 8.01 | 8.33 | 3.88E−06 | 21 | Used |
| 8124901 | HLA-C | −0.35 | 11.27 | 11.61 | 3.89E−06 | 22 | |
| 8096682 | ARHGEF38 | −0.78 | 6.52 | 7.21 | 3.89E−06 | 23 | |
| 8049187 | EFHD1 | 0.36 | 7.69 | 7.30 | 4.36E−06 | 24 | |
| 8117890 | HLA-B | −0.28 | 10.64 | 10.89 | 4.54E−06 | 25 | |
| 8179731 | HLA-B | −0.26 | 11.89 | 12.09 | 4.54E−06 | 26 | |
| 8154233 | CD274 | −0.84 | 6.35 | 7.14 | 4.54E−06 | 27 | Used |
| 8177788 | HLA-E | −0.29 | 10.68 | 10.94 | 4.54E−06 | 28 | |
| 8179103 | HLA-E | −0.29 | 10.68 | 10.94 | 4.54E−06 | 29 | |
| 8117777 | HLA-H | −0.24 | 9.99 | 10.21 | 4.89E−06 | 30 | |
| 7981290 | WARS | −0.55 | 9.39 | 9.97 | 5.08E−06 | 31 | |
| 8177732 | HLA-A | −0.27 | 11.72 | 12.01 | 5.30E−06 | 32 | |
| 7965565 | USP44 | −0.71 | 5.92 | 6.68 | 5.30E−06 | 33 | |
| 8125512 | TAP1 | −0.55 | 8.54 | 9.14 | 5.30E−06 | 34 | |
| 8178867 | TAP1 | −0.55 | 8.54 | 9.14 | 5.50E−06 | 35 | |
| 8180061 | TAP1 | −0.55 | 8.54 | 9.14 | 5.30E−06 | 36 | |
| 8022145 | L3MBTL4 | −0.37 | 6.74 | 7.07 | 5.37E−06 | 37 | |
| 8106098 | MAP1B | 0.68 | 8.77 | 8.00 | 5.37E−06 | 38 | |
| 7934719 | SFTPD | −0.71 | 9.02 | 9.63 | 5.75E−06 | 39 | |
| 7905929 | EFNA1 | −0.51 | 7.67 | 8.14 | 5.88E−06 | 40 | |
| 7917516 | GBP1 | −0.62 | 9.37 | 10.05 | 6.11E−06 | 41 | |
| 8161865 | PRUNE2 | 0.79 | 7.44 | 6.75 | 7.05E−06 | 42 | Used |
| 8044353 | ACOXL | −0.68 | 6.13 | 6.74 | 7.10E−06 | 43 | |
| 8057418 | ZNF385B | −0.71 | 6.95 | 7.71 | 7.15E−06 | 44 | |
| 8101131 | CXCL11 | −1.44 | 5.33 | 6.99 | 8.16E−06 | 45 | |
| 8058498 | FZD5 | −0.52 | 7.72 | 8.27 | 8.87E−06 | 46 | |
| 8082100 | PARP14 | −0.36 | 8.93 | 9.25 | 9.55E−06 | 47 | |
| 8001007 | PRSS8 | −0.51 | 7.39 | 7.89 | 9.85E−06 | 48 | |
| 8099760 | ARAP2 | −0.41 | 7.55 | 7.90 | 1.06E−05 | 49 | Used |
| 7914950 | CSF3R | −0.45 | 7.12 | 7.60 | 1.14E−05 | 50 | |
| 7972336 | DZIP1 | 0.33 | 7.72 | 7.47 | 1.16E−05 | 51 | Used |
| 8014591 | HNF1B | −0.53 | 6.88 | 7.33 | 1.20E−05 | 52 | |
| 8151423 | JPH1 | −0.52 | 6.68 | 7.17 | 1.21E−05 | 53 | |
| 8056217 | MXRA7 | 0.40 | 8.58 | 8.29 | 1.21E−05 | 54 | Used |
| 8117861 | HLA-L | −0.24 | 6.64 | 6.89 | 1.25E−05 | 55 | |
| 8179080 | HLA-L | −0.24 | 6.64 | 6.89 | 1.25E−05 | 56 | |
| 7976443 | IFI27 | −0.57 | 9.21 | 9.74 | 1.54E−05 | 57 | |
| 8022022 | LPIN2 | −0.30 | 8.06 | 8.37 | 1.55E−05 | 58 | |
| 7997593 | ATP2C2 | −0.46 | 6.73 | 7.13 | 1.62E−05 | 59 | |
| 8054846 | SCTR | −0.48 | 6.23 | 6.74 | 1.72E−05 | 60 | |
| 8178498 | HLA-B | −0.26 | 11.86 | 12.07 | 1.87E−05 | 61 | |
| 8140971 | SAMD9L | −0.48 | 8.09 | 8.52 | 2.12E−05 | 62 | |
| 7931728 | LARP4B | −0.28 | 8.76 | 9.07 | 2.15E−05 | 63 | |
| 8058857 | IGFBP5 | 0.62 | 10.41 | 9.73 | 2.15E−05 | 64 | |
| 7946504 | TMEM41B | −0.39 | 7.58 | 7.95 | 2.61E−05 | 65 | |
| 8057744 | STAT1 | −0.60 | 9.64 | 10.45 | 2.72E−05 | 66 | |
| 8107129 | SLCO4C1 | −1.08 | 7.96 | 8.93 | 2.72E−05 | 67 | |
| 8109938 | RANBP17 | −0.58 | 6.10 | 6.71 | 2.72E−05 | 68 | |
| 7934271 | PLA2G12B | −0.40 | 6.37 | 6.79 | 2.72E−05 | 69 | |
| 8126855 | PTCHD4 | 0.31 | 6.51 | 6.22 | 2.72E−05 | 70 | |
| 8097829 | FHDC1 | −0.54 | 6.39 | 6.91 | 2.80E−05 | 71 | |
| 8140478 | GSAP | −0.50 | 7.87 | 8.41 | 2.82E−05 | 72 | |
| 8079334 | LIMD1 | −0.34 | 7.37 | 7.66 | 2.83E−05 | 73 | |
| 7992828 | IL32 | −0.47 | 8.23 | 8.83 | 2.83E−05 | 74 | |
| 8103563 | DDX60 | −0.43 | 7.34 | 7.80 | 2.83E−05 | 75 | |
| 8082928 | CLDN18 | −1.10 | 8.77 | 9.84 | 2.83E−05 | 76 | |
| 7970716 | LNX2 | −0.46 | 8.40 | 8.79 | 3.01E−05 | 77 | |
| 7944739 | CRTAM | −0.62 | 5.05 | 5.69 | 3.07E−05 | 78 | |
| 8089026 | STX19 | −0.49 | 5.91 | 6.36 | 3.18E−05 | 79 | |
| 8079377 | CXCR6 | −0.46 | 5.75 | 6.17 | 3.22E−05 | 80 | |
| 7956120 | ERBB3 | −0.65 | 7.37 | 7.96 | 3.67E−05 | 81 | |
| 7981514 | AHNAK2 | 0.54 | 7.30 | 6.80 | 3.84E−05 | 82 | |
| 8134036 | STEAP2 | 0.66 | 9.18 | 8.42 | 3.87E−05 | 83 | |
| 8109639 | PTTG1 | −0.67 | 7.91 | 8.53 | 3.89E−05 | 84 | |
| 8101118 | CXCL9 | −1.55 | 7.35 | 9.08 | 4.07E−05 | 85 | |
| 7919984 | SELENBP1 | −0.44 | 8.87 | 9.25 | 4.25E−05 | 86 | |
| 8108724 | PCDHB10 | 0.61 | 5.78 | 5.12 | 4.27E−05 | 87 | |
| 8126853 | PTCHD4 | 0.75 | 7.02 | 6.07 | 4.27E−05 | 88 | Used |
| 8134384 | DYNG1I1 | 0.23 | 5.71 | 5.43 | 4.52E−05 | 89 | |
| 7935535 | CRTAC1 | −0.68 | 6.04 | 6.73 | 4.73E−05 | 90 | |
| 8097080 | SYNPO2 | 0.89 | 9.20 | 8.26 | 4.73E−05 | 91 | |
| 8129410 | THEMIS | −0.85 | 6.35 | 7.33 | 4.73E−05 | 92 | |
| 7968035 | SPATA13 | −0.24 | 6.85 | 7.12 | 4.83E−05 | 93 | |
| 8104022 | PDLIM3 | 0.58 | 8.33 | 7.63 | 5.03E−05 | 94 | Used |
| 8125545 | HLA-DOA | −0.48 | 8.31 | 8.98 | 5.03E−05 | 95 | |
| 8115147 | CD74 | −0.20 | 12.35 | 12.54 | 5.03E−05 | 96 | |
| 8144758 | ZDHHC2 | −0.31 | 7.85 | 8.21 | 5.03E−05 | 97 | |
| 7910466 | CAPN9 | −0.71 | 5.34 | 6.15 | 5.03E−05 | 98 | |
| 8124911 | HLA-B | −0.25 | 11.87 | 12.05 | 5.12E−05 | 99 | |
| 7938834 | NAV2 | −0.36 | 7.79 | 8.04 | 5.18E−05 | 100 | |
| 8146092 | IDO1 | −1.21 | 6.59 | 8.00 | 5.43E−05 | 101 | |
| 8117800 | HLA-A | −0.25 | 11.70 | 11.94 | 5.43E−05 | 102 | |
| 8025918 | CNN1 | 0.86 | 8.39 | 7.52 | 5.43E−05 | 103 | Used |
| 8020847 | DTNA | 0.42 | 7.10 | 6.70 | 5.52E−05 | 104 | |
| 7934411 | USP54 | −0.43 | 7.89 | 8.31 | 5.61E−05 | 105 | |
| 8101126 | CXCL10 | −1.70 | 6.30 | 8.19 | 5.61E−05 | 106 | |
| 7993458 | C16orf45 | 0.39 | 7.38 | 7.02 | 5.62E−05 | 107 | |
| 8157027 | NIPSNAP3B | 0.29 | 5.25 | 4.98 | 5.62E−05 | 108 | Used |
| 8007931 | ITGB3 | 0.40 | 6.54 | 6.10 | 5.62E−05 | 109 | |
| 7947248 | KIF18A | −0.48 | 5.00 | 5.51 | 6.16E−05 | 110 | |
| 7978360 | GZMH | −0.58 | 6.95 | 7.54 | 6.51E−05 | 111 | |
| 8142997 | PLXNA4 | 0.44 | 6.55 | 6.07 | 6.64E−05 | 112 | |
| 8125993 | ETV7 | −0.28 | 5.78 | 6.03 | 6.97E−05 | 113 | |
| 8149725 | PEBP4 | −1.05 | 8.77 | 9.75 | 7.36E−05 | 114 | |
| 8178295 | UBD | −0.74 | 7.87 | 8.57 | 7.36E−05 | 115 | |
| 8122986 | SNX9 | 0.28 | 8.54 | 8.22 | 7.50E−05 | 116 | |
| 8154981 | UXC13B | −0.49 | 8.16 | 8.62 | 7.50E−05 | 117 | |
| 8175369 | MAP7D3 | 0.55 | 8.76 | 8.26 | 8.13E−05 | 118 | |
| 8091600 | PLCH1 | −0.62 | 6.84 | 7.31 | 8.13E−05 | 119 | |
| 8124650 | UBD | −0.75 | 7.99 | 8.74 | 8.22E−05 | 120 | |
| 8161044 | TPM2 | 0.60 | 10.51 | 9.89 | 8.37E−05 | 121 | |
| 8002218 | ESRP2 | −0.41 | 7.21 | 7.56 | 8.37E−05 | 122 | |
| 8180093 | HLA-DOA | −0.45 | 8.07 | 8.59 | 8.37E−05 | 123 | |
| 8136473 | TRIM24 | −0.28 | 8.33 | 8.58 | 8.41E−05 | 124 | |
| 7956856 | MSRB3 | 0.44 | 7.99 | 7.55 | 8.49E−05 | 125 | |
| 8035304 | BST2 | −0.50 | 8.02 | 8.52 | 8.49E−05 | 126 | |
| 8072710 | APOL6 | −0.37 | 8.35 | 8.84 | 8.51E−05 | 127 | |
| 8052882 | ADD2 | 0.28 | 5.63 | 5.38 | 8.58E−05 | 128 | |
| 7958019 | DRAM1 | −0.52 | 8.60 | 9.13 | 9.18E−05 | 129 | |
| 8069565 | BTG3 | −0.38 | 8.02 | 8.40 | 9.23E−05 | 130 | |
| 8114010 | IRF1 | −0.53 | 7.62 | 8.14 | 9.28E−05 | 131 | |
| 7986446 | ALDH1A3 | 0.49 | 8.01 | 7.51 | 9.28E−05 | 132 | |
| 8068583 | KCNJ15 | −0.76 | 8.89 | 9.53 | 9.68E−05 | 133 | |
| 7909586 | PPP2R5A | −0.37 | 7.92 | 8.32 | 1.05E−04 | 134 | |
| 8178220 | HLA-DPB1 | −0.54 | 9.76 | 10.34 | 1.06E−04 | 135 | |
| 8116932 | PHACTR1 | −0.38 | 7.38 | 7.81 | 1.15E−04 | 136 | |
| 8136095 | AHCYL2 | −0.57 | 8.98 | 9.39 | 1.23E−04 | 137 | |
| 8073088 | APOBEC3G | −0.53 | 7.17 | 7.77 | 1.31E−04 | 138 | |
| 8109462 | CNOT8 | −0.26 | 8.54 | 8.78 | 1.33E−04 | 139 | |
| 8006608 | CCL4L1 | −0.64 | 6.44 | 7.12 | 1.39E−04 | 140 | |
| 8083709 | SMC4 | −0.29 | 8.48 | 8.78 | 1.41E−04 | 141 | |
| 8138489 | CDCA7L | −0.44 | 7.79 | 8.21 | 1.45E−04 | 142 | |
| 7913858 | PAQR7 | 0.22 | 6.58 | 6.38 | 1.46E−04 | 143 | |
| 8148070 | COL14A1 | 0.71 | 9.87 | 9.29 | 1.48E−04 | 144 | |
| 8096314 | PKD2 | 0.32 | 8.43 | 8.07 | 1.50E−04 | 145 | |
| 8014349 | CCL15-CCL14 | 0.69 | 9.75 | 9.08 | 1.57E−04 | 146 | |
| 7919314 | FMO5 | −0.68 | 7.05 | 7.69 | 1.58E−04 | 147 | |
| 8006621 | CCL4L1 | −0.75 | 7.10 | 7.93 | 1.61E−04 | 148 | |
| 8019651 | CCL4L1 | −0.75 | 7.10 | 7.93 | 1.61E−04 | 149 | |
| 8089299 | CD47 | −0.25 | 10.44 | 10.72 | 1.61E−04 | 150 | |
| 7904106 | MAGI3 | −0.42 | 7.60 | 7.99 | 1.71E−04 | 151 | |
| 8008321 | ACSF2 | −0.34 | 6.38 | 6.73 | 1.75E−04 | 152 | |
| 8005048 | MYOCD | 0.92 | 7.04 | 6.16 | 1.77E−04 | 153 | |
| 8042788 | ACTG2 | 0.98 | 10.11 | 9.21 | 1.79E−04 | 154 | Used |
| 7929466 | CYP2C18 | −0.35 | 5.09 | 5.39 | 1.84E−04 | 155 | |
| 8129888 | NHSL1 | −0.41 | 8.14 | 8.57 | 1.99E−04 | 156 | |
| 8173924 | NA | −0.95 | 5.22 | 6.25 | 2.03E−04 | 157 | Used |
| 7923958 | C1orf116 | −0.74 | 8.38 | 9.00 | 2.07E−04 | 158 | |
| 7979269 | GCH1 | −0.42 | 6.93 | 7.36 | 2.16E−04 | 159 | |
| 8020495 | CABLES1 | −0.37 | 7.82 | 8.21 | 2.17E−04 | 160 | |
| 7919645 | SV2A | 0.28 | 6.06 | 5.82 | 2.18E−04 | 161 | |
| 8077458 | EDEM1 | −0.33 | 7.97 | 8.32 | 2.18E−04 | 162 | |
| 8117476 | BTN3A3 | −0.40 | 8.58 | 9.00 | 2.24E−04 | 163 | |
| 8021376 | NEDD4L | −0.66 | 8.35 | 8.94 | 2.25E−04 | 164 | |
| 8056457 | SCN1A | −0.52 | 4.80 | 5.29 | 2.25E−04 | 165 | |
| 8150962 | TOX | −0.51 | 8.15 | 8.60 | 2.25E−04 | 166 | |
| 8058591 | ACADL | −1.01 | 6.63 | 7.62 | 2.26E−04 | 167 | |
| 8126653 | MRPL14 | −0.37 | 9.31 | 9.62 | 2.29E−04 | 168 | |
| 8098611 | TLR3 | −0.44 | 8.52 | 8.89 | 2.29E−04 | 169 | |
| 8066822 | SULF2 | 0.54 | 8.51 | 7.83 | 2.29E−04 | 170 | |
| 8109507 | ITK | −0.60 | 6.14 | 6.97 | 2.30E−04 | 171 | |
| 8099506 | TAPT1 | −0.32 | 7.42 | 7.70 | 2.32E−04 | 172 | |
| 7973564 | PSME1 | −0.19 | 9.85 | 10.07 | 2.34E−04 | 173 | |
| 7897044 | PRKCZ | −0.47 | 7.44 | 7.85 | 2.48E−04 | 174 | |
| 7974080 | MIA2 | −0.31 | 4.02 | 4.26 | 2.56E−04 | 175 | |
| 7917576 | GBP5 | −0.98 | 7.57 | 8.74 | 2.57E−04 | 176 | |
| 8085774 | ZNF385D | 0.99 | 7.66 | 6.39 | 2.58E−04 | 177 | |
| 7923386 | LMOD1 | 0.69 | 7.68 | 7.05 | 2.58E−04 | 178 | |
| 8073522 | SREBF2 | −0.28 | 8.20 | 8.44 | 2.59E−04 | 179 | |
| 7981460 | PPP1R13B | −0.25 | 7.83 | 8.04 | 2.62E−04 | 180 | |
| 8010454 | RNF213 | −0.36 | 8.63 | 9.01 | 2.63E−04 | 181 | |
| 8097903 | TLR2 | −0.37 | 7.77 | 8.26 | 2.68E−04 | 182 | |
| 8113369 | SLCO4C1 | −0.99 | 7.24 | 8.31 | 2.73E−04 | 183 | |
| 7950235 | STARD10 | −0.30 | 7.13 | 7.43 | 2.79E−04 | 184 | |
| 7910600 | KIAA1804 | −0.33 | 6.00 | 6.29 | 2.79E−04 | 185 | |
| 8117435 | BTN3A2 | −0.52 | 9.06 | 9.56 | 2.79E−04 | 186 | |
| 8143327 | PARP12 | −0.30 | 7.96 | 8.30 | 2.80E−04 | 187 | |
| 8087925 | TNNC1 | −0.90 | 8.53 | 9.30 | 2.82E−04 | 188 | |
| 8022045 | MYOM1 | 0.42 | 5.80 | 5.42 | 2.82E−04 | 189 | |
| 8096070 | BMP3 | −0.60 | 7.22 | 7.81 | 2.82E−04 | 190 | |
| 8075709 | APOL4 | −0.39 | 7.01 | 7.41 | 2.87E−04 | 191 | |
| 7915500 | C1orf210 | −0.32 | 7.17 | 7.59 | 2.91E−04 | 192 | |
| 7920297 | S100A14 | −0.56 | 8.42 | 8.87 | 2.94E−04 | 193 | |
| 7983630 | FGF7 | 0.67 | 7.64 | 6.98 | 2.94E−04 | 194 | |
| 8010287 | C1QTNF1 | 0.35 | 7.81 | 7.49 | 3.09E−04 | 195 | |
| 8018966 | TIMP2 | 0.18 | 10.53 | 10.34 | 3.09E−04 | 196 | Used |
| 7951593 | NA | −0.51 | 7.28 | 7.84 | 3.16E−04 | 197 | |
| 8048541 | DES | 0.71 | 8.81 | 8.19 | 3.20E−04 | 198 | Used |
| 7975361 | KIAA0247 | −0.22 | 8.17 | 8.37 | 3.20E−04 | 199 | |
| 8170428 | MTM1 | −0.31 | 7.23 | 7.52 | 3.22E−04 | 200 | |

Abbreviations: TCID = transcript-cluster identity; Symbol = gene symbol; logFC = log fold-change; MedExpr.UIP = median expression level across the UIP samples; MedExpr.NonUIP = median expression level across the UIP samples; FDR = false discovery rate; Used by classifier = indicator of whether the gene is used by the microarray classifier.

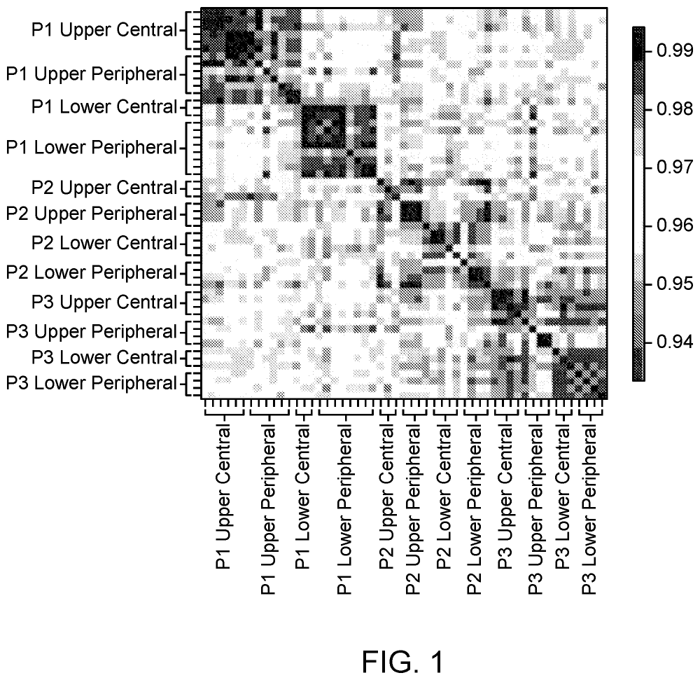

The number and location of the samplings (upper vs. lower and central vs. peripheral) are indicated in FIG. 6 and IPF patient clinical characteristics in Table 4. To identify genes useful in measuring spatial heterogeneity, we looked for differential expression in normal versus IPF samples. This comparison produced 5,000 significantly differentially expressed RNA transcripts with FDR <0.05 (data not shown). We selected the top 200 differentially expressed genes and measured pairwise correlation. The results for the three patients diagnosed with IPF are shown in FIG. 1. Although correlation across all IPF samples is high, three distinct patterns emerge in the correlation structure among IPF samples. One patient (P1) shows substantial differences in upper vs. lower lobe gene expression i.e. lower correlation in gene signals. One patient (P3) shows higher correlation between the upper and lower lobe samplings. The third patient (P2) shows an intermediate result between these two cases, with sometimes higher, and sometimes lower, correlation between samplings from the upper and lower lobes. These results, while on a small number of patients, suggest that samples with lobe-specific pathology may be more accurate during the training phase of classifier development. Based on this information we prepared a classifier using SLB tissues with truth labels assigned at the sample level, using lobe-derived pathology. Our results, which are presented in Example 7, demonstrate the presence of a molecular signature in SLB tissues that classifies UIP and non-UIP samples with high prospective accuracy.

TABLE 4
Clinical characteristics of three IPF explant patients.

| Patient | Gender | Age | Smoking Status | Clinical Remarks |
|---|---|---|---|---|
| P1 | M | 49 | non-smoker | Exertional dyspnea for ~2 years prior |
| | | | | to initial evaluation. |
| | | | | Pre-transplant SLB demonstrated |
| | | | | clear UIP pattern, no granuloma or |
| | | | | bronchiolocentricity. |
| | | | | Diagnosis of IPF at transplant |
| | | | | reported patchy subpleural and |
| | | | | paraseptal interstitial fibrosis, dense |
| | | | | scarring and honeycombing by |
| | | | | surgical pathology. No evidence of |
| | | | | granuloma or extensive |
| | | | | inflammation. |
| P2 | M | 68 | 50 pack years | Initial evaluation occurred almost |
| | | | | immediately after first presentation of |
| | | | | exertional dyspnea; progressive |
| | | | | worsening over ~2.5 years. |
| | | | | Pathology at transplant demonstrated |
| | | | | end-stage lung disease, diffuse |
| | | | | fibrosis, temporal heterogeneity and |
| | | | | fibroblastic foci suggestive of UIP. |
| P3 | F | 64 | 24 pack years | Exertional dyspnea for ~2 years prior |
| | | | | to initial evaluation, worsening over |
| | | | | the last year. Possible occupational |
| | | | | exposure. |
| | | | | Pre-transplant SLB demonstrated UIP |
| | | | | pattern with fibroblastic foci |
| | | | | consistent with IPF. Pathology at |
| | | | | transplant showed interstitial fibrosis, |
| | | | | giant cell reaction, reorganization, |
| | | | | bronchiectasis and reactive lymph |
| | | | | node. |

### Example 7

Performance of Microarray Classifier on Surgical Lung Biopsies

Using sample-specific pathology labels on biopsies obtained during VATS, a microarray classifier was trained by logistic regression on the top 200 genes separating UIP and non-UIP samples (see Table 12). A final model was built with 22 genes (Table 5).

Expression data was normalized by Robust Multi-array Average (RMA). Feature selection and model estimation were performed by logistic regression with lasso penalty using glmnet3. Raw reads were aligned using TopHat. Gene counts were obtained using HTSeq and normalized using DESeq. The top features (N ranging from 10 to 200) were used to train a linear support vector machine (SVM) using the e1071 library. Confidence intervals were computed using the pROC package.

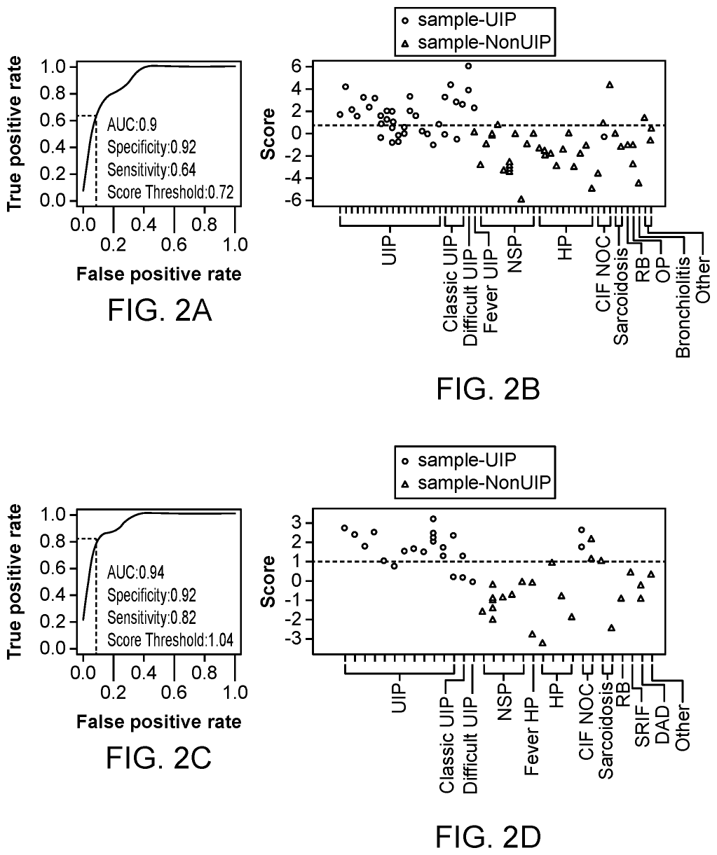

LOPO CV performance is summarized as a receiver operating characteristic (ROC) curve (FIG. 2A). The AUC is 0.9 [CI 0.82-0.96], with 92% [CI 84%400%] specificity and 64% [CI 49%-79%] sensitivity. Individual LOPO CV classification scores are shown for all patients (FIG. 2B). Among the three misclassified non-UIP samples, two have scores very close to the threshold (0.86 and 1.30), and one has a high score (421). The latter sample with the high score was diagnosed as an ‘unclassifiable fibrotic ILD’ at both the sample- and patient-level. Among the UIP samples, fifteen (36%) have a score below the threshold (false negatives) but none of those samples have a large negative score. Since LOPO CV in certain cases has the potential to overestimate performance, we also evaluated 10-fold patient-level CV (i.e., 10% of patients are left out in each loop) which gives very similar performance (the median AUC from five repeated 10-fold CVs is 0.88).

TABLE 5
Twenty two genes included in a preferred array classifier.

| | SYMBOL | | | | |
|---|---|---|---|---|---|
| | (SEQ ID | | | | |
| TCID | NO.) | logFC | MedExpr.UIP | MedExpr.NonUIP | FDR |
| 8117760 | HLA-F | −0.48 | 9.02 | 9.53 | 2.35E−09 |
| | (1) | | | | |
| 8101031 | CDKL2 | −0.95 | 7.31 | 8.16 | 1.29E−07 |
| | (2) | | | | |
| 8106827 | GPR98 | −0.77 | 6.11 | 6.74 | 2.16E−07 |
| | (3) | | | | |
| 7931930 | PRKCQ | −0.64 | 6.78 | 7.4 | 1.81E−06 |
| | (4) | | | | |
| 8177725 | HLA-G | −0.29 | 10.67 | 10.89 | 2.37E−06 |
| | (5) | | | | |
| 7926037 | PFKFB3 | −0.45 | 7.66 | 8.1 | 2.81E−06 |
| | (6) | | | | |
| 8037205 | CEACAM1 | −0.41 | 6.47 | 6.97 | 3.04E−06 |
| | (7) | | | | |
| 7907492 | RABGAP1L | −0.32 | 8.01 | 8.33 | 3.88E−06 |
| | (8) | | | | |
| 8154233 | CD274 | −0.84 | 6.35 | 7.14 | 4.54E−06 |
| | (9) | | | | |
| 8161865 | PRUNE2 | 0.79 | 7.44 | 6.75 | 7.05E−06 |
| | (10) | | | | |
| 8099760 | ARAP2 | −0.41 | 7.55 | 7.9 | 1.06E−05 |
| | (11) | | | | |
| 7972336 | DZIP1 | 0.33 | 7.72 | 7.47 | 1.16E−05 |
| | (12) | | | | |
| 8056217 | MXRA7 | 0.4 | 8.58 | 8.29 | 1.21E−05 |
| | (13) | | | | |
| 8126853 | PTCHD4 | 0.75 | 7.02 | 6.07 | 4.27E−05 |
| | (14) | | | | |
| 8104022 | PDLIM3 | 0.58 | 8.33 | 7.63 | 5.03E−05 |
| | (15) | | | | |
| 8025918 | CNN1 | 0.86 | 8.39 | 7.52 | 5.43E−05 |
| | (16) | | | | |
| 8157027 | NIPSNAP3B | 0.29 | 5.25 | 4.98 | 5.62E−05 |
| | (17) | | | | |
| 7913858 | PAQR7 | 0.22 | 6.58 | 6.38 | 1.46E−04 |
| | (18) | | | | |
| 8042788 | ACTG2 | 0.98 | 10.11 | 9.21 | 1.79E−04 |
| | (19) | | | | |
| 8173924 | NA | −0.95 | 5.22 | 6.25 | 2.03E−04 |
| | (20) | | | | |
| 8018966 | TIMP2 | 0.18 | 10.53 | 10.34 | 3.09E−04 |
| | (21) | | | | |
| 8048541 | DES | 0.71 | 8.81 | 8.19 | 3.20E−04 |
| | (22) | | | | |

Abbreviations:
TCID = transcript-cluster identify; Symbol = gene symbol; logFC = log fold-change; MedExpr.UIP = median expression level across the UIP samples; MedExpr.NonUIP = median expression level across the UIP samples; FDR = false discovery rate.

Independent test set performance is shown in FIG. 2C showing an AUC of 0.94 [CI 0.86-0.99], with 92% [CI 81%400%] specificity and 82% [CI 64%-95%] sensitivity. The individual classification score distribution shows good separation between UIP and non-UIP classes (FIG. 2D). The two misclassified non-UIP samples have both patient- and sample-level expert diagnoses of ‘unclassifiable fibrotic ILD’ indicating uncertainty in the diagnosis. The score range observed in the test set (FIG. 2D) is narrower than the range seen in LOPO CV scores (FIG. 2B), likely due to the larger variability inherent in applying a series of sub-classifiers within each CV loop, compared to scores obtained by applying a single model. Classification performance including 95% confidence intervals are summarized in Table 6.

TABLE 6
Classifier performance summary, including 95% confidence intervals (CI).

| | | | | | | | RNASeqSet- | |
|---|---|---|---|---|---|---|---|---|
| | Array | | Array | | RNASeq | | matched array | |
| | classifier | | classifier | | classifier | | classifier | |
| | (LOPO CV) | | (Testing) | | (LOPO CV) | | (LOPO CV) | |
| AUC [95% CI] | 0.90 | [0.82-0.96] | 0.94 | [0.86-0.99] | 0.90 | [0.77-1.00] | 0.86 | [0.73-0.96] |
| Specificity [95% | 92% | [84%-100%] | 92% | [81%-100%] | 95% | [84%-100%] | 95% | [84%-100%] |
| CI] | | | | | | | | |
| Sensitivity [95% | 64% | [49%-79%] | 82% | [64%-95%] | 59% | [35%-82%] | 47% | [24%-71%] |
| CI] | | | | | | | | |

| | | | | |
|---|---|---|---|---|
| Threshold | 0.72 | 1.04 | 0.64 | 1 |
| False positive | 3 | 2 | 1 | 1 |
| count | | | | |
| True negative | 35 | 24 | 18 | 18 |
| count | | | | |
| False negative | 14 | 4 | 7 | 9 |
| count | | | | |
| True negative | 25 | 18 | 10 | 8 |
| count | | | | |
| Total | 77 | 48 | 36 | 36 |

Our approach offers significant advantages. Earlier gene-expression profiling studies focused on comparing IPF versus a few non-IPF ILD subtypes such as HP or NSIP, or against subjects without ILD^{18,19,23,25}. The non-UIP cohort reported here represents a broad spectrum of pathology subtypes including HP, NSIP, sarcoidosis, RB, bronchiolitis, organizing pneumonia (OP), and others, thus approximating the diversity of ILDs encountered in clinical practice. In addition, the classifier was trained and tested using a combination of banked and prospectively collected SLBs to ensure robustness against potential differences in sample handling and collection. Finally, many earlier studies focused on differential gene expression analyses alone, without building a classification engine. In contrast, our approach is a rigorous method for the development of molecular tests which, when properly trained and validated, generalized well to independent data sets.

### Example 8

Performance of RNASeq classifier on surgical lung biopsies

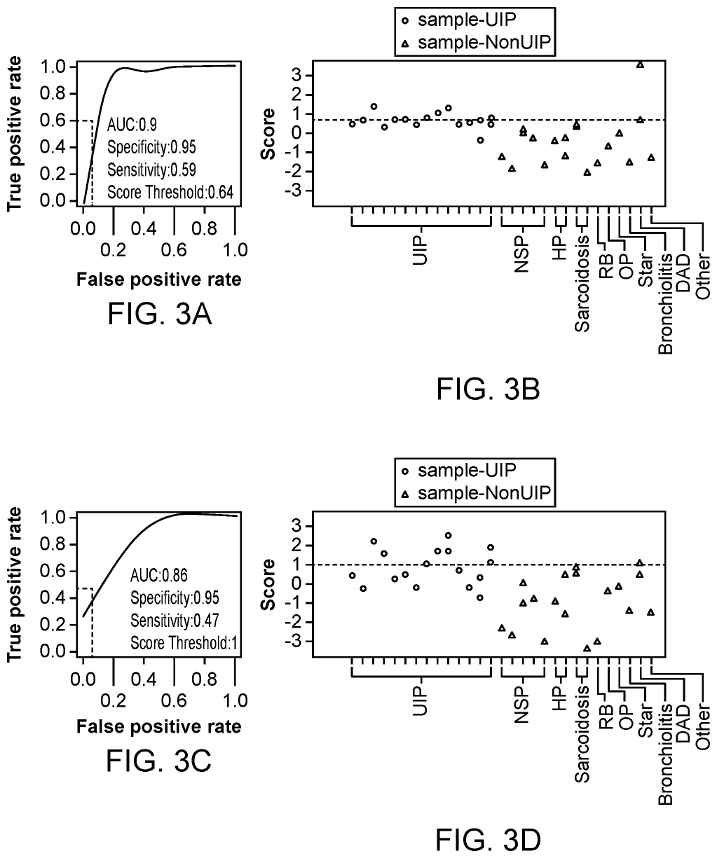

A subset of 36 samples with RNASeq data were used to train a linear SVM classifier and the performance evaluated by LOPO CV. AUCs are consistently above 0.80 for gene numbers spanning 10 to 200 (data not shown). We chose a model using 100 genes for further examination. The AUC is 0.9 [CI 0.77-1.00] (specificity=95% [CI 84%-100%], sensitivity=59% [CI 35%-82%]) (FIG. 3A). Only a single non-UIP sample is misclassified (FIG. 3B). The sample-level pathology for this sample is respiratory bronchiolitis (RB), and the patient-pathology is diffuse alveolar damage (DAD), two subtypes that may have been difficult to model because of their sparsity. We carried out a similar analysis using matching array data on the same set of samples; the array-based classifier achieves similar performance (AUC=0.86 [CI 0.73-0.96]) using 160 genes. Specificity is 95% [CI 84%400%] and sensitivity is 47% [CI 24%-71%] (FIG. 3C). Interestingly, the same non-UIP sample that was misclassified as a UIP by the RNASeq classifier is also misclassified by the microarray classifier (FIG. 3D). Overall, classification based on RNASeq achieves comparable performance to that of the array platform.

### Example 9

Biological Pathways Associated With Genes Used By The Classifiers

To determine if there are common biological underpinnings across the genes selected by the machine learning process, we used over-representation analysis (ORA) to identify statistically significant participation of genes in selected pathways. Over/under-representation analyses (ORA) were performed using GeneTrail software (genetrail.bioinf.uni-sb.de/) and the top 1,000 genes differentially expressed by limma between UIP and non-UIP samples (FDR <0.013) in the microarray testing set (n=77) as the ORA test sets. The ORA reference set included all human genes (n=44,829) and annotation in the KEGG pathways and gene ontology (GO) databases. Significance was evaluated via Fisher's exact test with a corrected FDR threshold of p<0.05.

In examining the top 1000 genes found in the UIP vs non-UIP comparison, distinct findings emerged (Table 2).

TABLE 2
Kyoto Encyclopedia of Genes and Genomes (KEGG) Pathways and Gene
Ontologies (GO) over-represented in UIP and non-UIP samples.
Categories in each sample cohort are ranked by FDR p value.

| | | | | | |
|---|---|---|---|---|---|
| | | No. | No. | ORA | FDR |
| Source | Category | expected | observed | Proportion | p value |

Over-represented in UIP

| | | | | | |
|---|---|---|---|---|---|
| GO | Extracellular matrix | 5 | 31 | 6.2 | 1.46E−12 |
| GO | Muscle system process | 5 | 23 | 4.6 | 1.21E−07 |
| GO | Cell migration/motility | 9 | 27 | 3.0 | 7.31E−06 |
| KEGG | Focal adhesion | 6 | 21 | 3.5 | 1.20E−05 |
| GO | Contractile fiber | 2 | 12 | 6.0 | 1.73E−05 |
| KEGG | Calcium signaling, pathway | 5 | 18 | 3.6 | 5.38E−05 |
| KEGG | Dilated cardiomyopathy | 3 | 13 | 4.3 | 5.38E−05 |
| KEGG | ECM-receptor interaction | 2 | 12 | 6.0 | 5.95E−05 |
| KEGG | Vascular Muscle contraction | 3 | 14 | 4.7 | 5.95E−05 |
| KEGG | Hypertropic cardiomyopathy (HCM) | 2 | 11 | 5.5 | 2.90E−04 |
| KEGG | Arrhythmogenic right ventricular | 2 | 9 | 4.5 | 2.82E−03 |
| | cardiomyopathy (ARVC) | | | | |
| KEGG | Regulation of Actin cytoskeleton | 6 | 16 | 2.7 | 3.39E−03 |
| KEGG | Melanoma | 2 | 8 | 4.0 | 6.26E−03 |
| KEGG | Cardiac muscle contraction | 2 | 7 | 3.5 | 4.53E−02 |
| KEGG | MAPK signaling pathway | 8 | 15 | 1.9 | 4.76E−02 |

Over-represented in non-UIP

| | | | | | |
|---|---|---|---|---|---|
| KEGG | Allograft rejection | 1 | 15 | 15.0 | 9.25E−12 |
| KEGG | Cell adhesion molecules | 4 | 25 | 6.3 | 9.25E−12 |
| KEGG | Antigen processing and presentation | 2 | 19 | 9.5 | 3.40E−11 |
| KEGG | Type I diabetes mellitus | 1 | 14 | 14.0 | 5.95E−10 |
| GO | Immune response | 13 | 42 | 3.2 | 6.52E−09 |
| KEGG | Autoimmune thyroid disorder | 2 | 14 | 7.0 | 6.72E−09 |
| KEGG | Viral myocarditis | 2 | 16 | 8.0 | 6.72E−09 |
| KEGG | Phagosome | 5 | 20 | 4.0 | 7.01E−07 |
| GO | MHC class I receptor activity | 1 | 7 | 7.0 | 2.66E−05 |
| KEGG | Systemic lupus erythematosus | 4 | 16 | 4.0 | 6.11E−05 |
| KEGG | Leishmaniasis | 2 | 11 | 5.5 | 1.14E−04 |
| GO | Innate immune response | 3 | 14 | 4.7 | 2.34E−04 |
| KEGG | Asthma | 1 | 7 | 7.0 | 2.83E−04 |
| GO | Signal transducer activity | 24 | 48 | 2 | 3.44E−04 |
| GO | Antigen processing and | 1 | 4 | 4 | 7.50E−04 |
| | presentation of endogenous | | | | |
| | antigen | | | | |
| KEGG | Intestinal immune network for | 2 | 8 | 4.0 | 8.40E−04 |
| | IgA production | | | | |
| KEGG | Malaria | 2 | 8 | 4.0 | 1.04E−03 |
| GO | T cell activation | 3 | 12 | 4.0 | 1.58E−03 |
| KEGG | Natural killer cell mediated | 4 | 13 | 3.3 | 1.91E−03 |
| | cytotoxicity | | | | |
| KEGG | Endocytosis | 6 | 16 | 2.7 | 3.42E−03 |
| KEGG | Steroid biosynthesis | 1 | 4 | 4.0 | 8.78E−03 |
| KEGG | Tight junction | 4 | 11 | 2.8 | 1.50E−02 |
| KEGG | Proteasome | 1 | 6 | 6.0 | 1.71E−02 |
| KEGG | Primary immunodeficiency | 1 | 5 | 5.0 | 1.89E−02 |
| KEGG | Toll-like receptor signaling | 3 | 9 | 3.0 | 1.89E−02 |

Abbreviations:
FDR = false discovery rate; GO = Gene Ontology; KEGG = Kyoto Encyclopedia of Genes and Genomes; ORA = over-representation analysis.

In UIP, genes involved in cell adhesion, muscle disease, cell migration and motility predominate. These results are consistent with previous reports of pathways differentially regulated in IPF18,19,22,23. In contrast, other non-UIP subtypes overexpress genes involved in immune processes, including both the adaptive and innate systems. This enrichment could be due to the RB and HP subtypes present in the non-UIP cohort; diseases known to exhibit immune components’. Genes over-represented in KEGG pathways and Gene Ontology groups are summarized in Tables 7 and 8.

TABLE 7
Genes over-represented in KEGG pathways and Gene Ontology groups of UIP samples.
OVER-REPRESENTED IN UIP

| | | No. | |
|---|---|---|---|
| Source | Category | observed | Genes Observed |
| GO | Extracellular matrix | 31 | ABI3BP, ADAMTS3, AEBP1, CLU, |
| | | | COL14A1, COL15A1, COL1A2, COL21A1, |
| | | | COL6A1, COL6A2, CPXM2, DST, FBLN1, |
| | | | FBN1, FGF10, FMOD, HTRA1, LAMA4, |
| | | | LAMB2, LAMC1, LTBP1, MGP, NID1, |
| | | | PLAT, POSTN, SERPINF1, SFRP2, SNCA, |
| | | | SPON1, TNC, VCAN |
| GO | Muscle system process | 23 | ACTA2, ACTG2, AGT, ATP1A2, BDKRB2, |
| | | | CALD1, CNN1, CRYAB, DES, DTNA, IGF1, |
| | | | LMOD1, MYH11, MYL9, MYLK, MYOCD, |
| | | | SLC6A8, SSPN, TNNI2, TNNT3, TPM1, |
| | | | TPM2, TPM4 |
| GO | Cell | 27 | ADRA2A, AGT, ANGPT2, CCL24, CXCL12, |
| | migration/motility | | ENPP2, F10, FGF10, FGF7, IGF1, IGFBP5, |
| | | | ITGB3, KRT2, LAMC1, NEXN, NR2F2, |
| | | | PDGFRB, PODN, PPAP2A, ROR2, S100A2, |
| | | | SCG2, SFRP2, SLIT3, THY1, TPM1, VCAN |
| KEGG | Focal adhesion | 21 | ACTN1, COLIA2, COL6A1, COL6A2, FLNC, |
| | | | HGF, IGF1, ITGA7, ITGB3, ITGB4, LAMA4, |
| | | | LAMB2, LAMC1, MYL9, MYLK, PARVA, |
| | | | PDGFD, PDGFRB, SHC4, SPP1, TNC |
| GO | Contractile fiber | 12 | DES, MYH11, MYL9, MYOM1, NEXN, |
| | | | PGM5, SVIL, TNNI2, TNNT3, TPM1, TPM2, |
| | | | TPM4 |
| KEGG | Calcium signaling, | 18 | ADCY3, AVPR1A, BDKRB2, CACNA1C, |
| | pathway | | GNAL, GRIN2A, HRH1, HTR2A, MYLK, |
| | | | P2RX1, PDE1A, PDGFRB, PLCB1, PLN, |
| | | | PTGER3, RYR3, TACR1, TRPC1 |
| KEGG | Dilated | 13 | ADCY3, ADCY5, CACNA1C, CACNA2D1, |
| | cardiomyopathy | | DES, IGF1, ITGA7, ITGB3, ITGB4, PLN, |
| | | | TPM1, TPM2, TPM4 |
| KEGG | ECM-receptor | 12 | COL1A2, COL6A1, COL6A2, ITGA7, ITGB3, |
| | interaction | | ITGB4, LAMA4, LAMB2, LAMC1, SPP1, |
| | | | SV2A, TNC |
| KEGG | Vascular Muscle | 14 | ACTA2, ACTG2, ADCY3, ADCY5, AVPR1A, |
| | contraction | | CACNA1C, CALD1, KCNMB1, MRVI1, |
| | | | MYH11, MYL9, MYLK, PLA2G2A, PLCB1, |
| KEGG | Hypertropic | 11 | CACNA1C, CACNA2D1, DES, IGF1, ITGA7, |
| | cardiomyopathy | | ITGB3, ITGB4, PRKAA2, TPM1, TPM2, |
| | (HCM) | | TPM4 |
| KEGG | Arrhythmogenic right | 9 | ACTN1, CACNA1C, CACNA2D1, CDH2, |
| | ventricular | | DES, ITGA7, ITGB3, ITGB4, PKP2 |
| | cardiomyopathy | | |
| | (ARVC) | | |
| KEGG | Regulation of Actin | 16 | ACTN1, BDKRB2, ENAH, FGF10, FGF14, |
| | cytoskeleton | | FGF7, FGFR1, GNG12, ITGA7, ITGB3, |
| | | | ITGB4, MRAS, MYL9, MYLK, PDGFD, |
| | | | PDGFRB |
| KEGG | Melanoma | 8 | FGF10, FGF14, FGF7, FGFR1, HGF, IGF1, |
| | | | PDGFD, PDGFRB |
| KEGG | Cardiac muscle | 7 | ATP1A2, ATP1B2, CACNA1C, CACNA2D1, |
| | contraction | | TPM1, TPM2, TPM4 |
| KEGG | MAPK signaling | 15 | CACNA1C, CACNA2D1, FGF10, FGF14, |
| | pathway | | FGF7, FGFR1, FLNC, GNG12, HSPA2, |
| | | | MAP4K4, MRAS, NFATC4, PDGFRB, |
| | | | PLA2G2A, ZAK |

TABLE 8
Genes over-represented in KEGG pathways and Gene Ontology groups of non-UIP samples,
OVER-REPRESENTED IN non-UIP

| | | No. | |
|---|---|---|---|
| Source | Category | observed | Genes Observed |
| KEGG | Allograft rejection | 15 | CD40, FASLG, HLA-A, HLA-B, HLA-C, HLA- |
| | | | DMA, HLA-DOA, HLA-DPA1, HLA-DPB1, |
| | | | HLA-DQA1, HLA-DRA, HLA-E, HLA-F, |
| | | | HLA-G, IFNG |
| KEGG | Cell adhesion | 25 | CADM1, CD2, CD274, CD40, CD8A, |
| | molecules | | CLDN18, CLDN4, F11R, HLA-A, HLA-B, |
| | | | HLA-C, HLA-, DMA, HLA-DOA, HLA-DPA1, |
| | | | HLA-DPB1, HLA-DQA1, HLA-DRA, HLA-E, |
| | | | HLA-F, HLA-G, ICAM1, ICOS, ITGAL, |
| | | | OCLN, SDC4 |
| KEGG | Antigen processing | 19 | CD74, CD8A, HLA-A, HLA-B, HLA-C, HLA- |
| | and presentation | | DMA, HLA-DOA, HLA-DPA1, HLA-DPB1, |
| | | | HLA-, DQA1, HLA-DRA, HLA-E, HLA-F, |
| | | | HLA-G, IFNG, PSME1, PSME2, TAP1, TAP2 |
| KEGG | Type I diabetes | 14 | FASLG, HLA-A, HLA-B, HLA-C, HLA-DMA, |
| | mellitus | | HLA-DOA, HLA-DPA1, HLA-DPB1, HLA-, |
| | | | DQA1, HLA-DRA, HLA-E, HLA-F, HLA-G, |
| | | | IFNG |
| GO | Immune response | 42 | APOBEC3G, AQP4, BST2, C2, CADM1, |
| | | | CCR5, CD274, CD74, CD8A, CRTAM, CTSC, |
| | | | CTSW, CXCL16, ERAP1, FCGR1A, FCGR1B, |
| | | | FCGR3A, GBP2, GCH1, GZMA, HLA-A, |
| | | | HLA-B, HLA-C, HLA-DPA1, HLA-DQA1, |
| | | | HLA-DRA, HLA-H, ICAM1, ICOS, IL32, |
| | | | ITGAL, MICB, NUB1, PLA2G1B, PSMB10, |
| | | | S100A14, SFTPD, SKAP1, THEMIS, TLR2, |
| | | | TLR3, UBD |
| KEGG | Autoimmune thyroid | 14 | CD40, FASLG, HLA-A, HLA-B, HLA-C, HLA- |
| | disorder | | DMA, HLA-DOA, HLA-DPA1, HLA-DPB1, |
| | | | HLADQA1, HLA-DRA, HLA-E, HLA-F, |
| | | | HLA-G |
| KEGG | Viral myocarditis | 16 | CD40, HLA-A, HLA-B, HLA-C, HLA-DMA, |
| | | | HLA-DOA, HLA-DPA1, HLA-DPB1, HLA- |
| | | | DQA1, HLA-DRA, HLA-E, HLA-F, HLA-G, |
| | | | ICAM1, ITGAL, MYH14 |
| KEGG | Phagosome | 20 | ATP6V0D1, FCGR1A, FCGR3A, HLA-A, |
| | | | HLA-B, HLA-C, HLA-DMA, HLA-DOA, |
| | | | HLA-, DPA1, HLA-DPB1, HLA-DQA1, HLA- |
| | | | DRA, HLA-E, HLA-F, HLA-G, SFTPA1, |
| | | | SFTPD, TAP1, TAP2, TLR2 |
| GO | MHC class I receptor | 7 | HLA-A, HLA-B, HLA-C, HLA-E, HLA-F, |
| | activity | | HLA-G, HLA-H |
| KEGG | Systemic lupus | 16 | C2, CD40, FCGR1A, FCGR3A, HIST1H2BJ, |
| | erythematosus | | HIST1H3F, HIST1H4E, HIST2H2BE, HLA-, |
| | | | DMA, HLA-DOA, HLA-DPA1, HLA-DPB1, |
| | | | HLA-DQA1, HLA-DRA, IFNG, TRIM21 |
| KEGG | Leishmaniasis | 11 | FCGR1A, FCGR3A, HLA-DMA, HLA-DOA, |
| | | | HLA-DPA1, HLA-DPB1, HLA-DQA1, HLA-, |
| | | | DRA, IFNG, STAT1, TLR2 |
| GO | Innate immune | 14 | APOBEC3G, AQP4, C2, CADM1, CRTAM, |
| | response | | CXCL16, ERAP1, GCH1, NUB1, S100A14, |
| | | | SFTPD, TLR2, TLR3, UBD |
| KEGG | Asthma | 7 | CD40, HLA-DMA, HLA-DOA, HLA-DPA1, |
| | | | HLA-DPB1, HLA-DQA1, HLA-DRA |
| GO | Signal transducer | 48 | AGER, BST2, CCL4, CCL5, CCR5, CD2, |
| | activity | | CD40, CD47, CD74, CD8A, CLDN4, CXCL16, |
| | | | CXCR6, EBP, ERBB3, FCGR1A, FCGR1B, |
| | | | FGFR2, FLRT3, FZD5, GPR98, HLA-A, HLA- |
| | | | B, HLA-C, HLA-DOA, HLA-DPA1, HLA- |
| | | | DQA1, HLA-DRA, HLA-E, HLA-F, HLA-G, |
| | | | HLA-H, ICAM1, IL2RB, KLRB1, LDLR, |
| | | | MAP3K13, PTPRJ, SCTR, SDC4, SH2D1A, |
| | | | SKAP1, STAT1, TAPT1, TLR2, TLR3, |
| | | | TP53BP2, UNC13B |
| GO | Antigen processing | 4 | CD74, ERAP1, TAP1, TAP2 |
| | and presentation of | | |
| | endogenous antigen | | |
| KEGG | Intestinal immune | 8 | CD40, HLA-DMA, HLA-DOA, HLA-DPA1, |
| | network for IgA | | HLA-DPB1, HLA-DQA1, HLA-DRA, ICOS |
| | production | | |
| KEGG | Malaria | 8 | CD40, ICAM1, IFNG, ITGAL, KLRB1, |
| | | | KLRK1, SDC4, TLR2 |
| GO | T cell activation | 12 | CADM1, CD2, CD47, CD74, CD8A, CRTAM, |
| | | | ICAM1, ITGAL, KLRK1, MICB, SFTPD, |
| | | | THEMIS |
| KEGG | Natural killer cell | 13 | FASLG, FCGR3A, HLA-A, HLA-B, HLA-C, |
| | mediated cytotoxicity | | HLA-E, HLA-G, ICAM1, IFNG, ITGAL, |
| | | | KLRK1, MICB, SH2D1A |
| KEGG | Endocytosis | 16 | ARAP2, CCR5, DNM2, ERBB3, FGFR2, HLA- |
| | | | A, HLA-B, HLA-C, HLA-E, HLA-F, HLA-G, |
| | | | IL2RB, LDLR, NEDD4L, PARD6B, PRKCZ |
| KEGG | Steroid biosynthesis | 4 | DHCR24, EBP, FDFT1, SQLE |
| KEGG | Tight junction | 11 | CLDN18, CLDN4, F11R, INADL, LLGL2, |
| | | | MAGI3, MYH14, OCLN, PARD6B, PRKCQ, |
| | | | PRKCZ |
| KEGG | Proteasome | 6 | IFNG, PSMB10, PSMB8, PSMB9, PSME1, |
| | | | PSME2 |
| KEGG | Primary | 5 | CD40, CD8A, ICOS, TAP1, TAP2 |
| | immunodeficiency | | |
| KEGG | Toll-like receptor | 9 | CCL4, CCL5, CD40, CXCL10, CXCL11, |
| | signaling | | CXCL9, STAT1, TLR2, TLR3 |

### Example 10

Mislabeling Simulation Study

A simulation study swapping binary classification labels (UIP or non-UIP) was performed on the microarray training set. Samples were selected at random for label permutation, at total proportions per simulation set ranging from 1% to 40%. The level of agreement in the blinded review of the three expert pathology diagnoses is 3/3 (n=44), 2/2 (n=8), 2/3 (n=24), and 1/3 (n=1). Sample labels were changed to the other class with a weight proportional to the probability accounting for the disagreement level in the blinded review of the three expert pathologists: 5% for 3/3 or 2/2 agreement, 50% for 2/3 agreement, and 90% for 1/3 agreement. Simulations were repeated 100 times at each proportion.

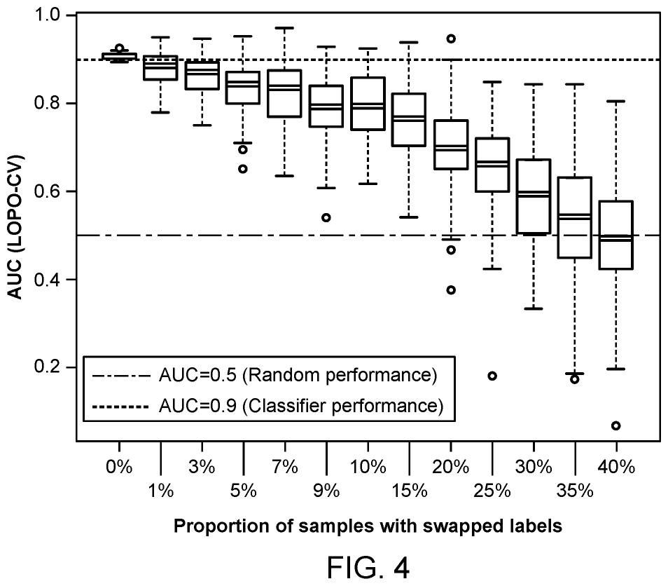

The LOPO CV performance (AUC) was evaluated over 100 repeated simulations across a range of proportion of swapped labels (FIG. 4). When there is no label swap, the median performance is very close to the array classifier performance shown in FIG. 2A (AUC=0.9). (Using the same set of samples and labels, model estimation can have slight variability). As the swap rate increases, the performance decreases monotonically. When 40% of the labels are swapped, the median performance approaches 0.5, indicating classification is no better than random chance.

### Example 11

Magnitude and direction of UIP/Non-UIP differential gene expression differs in smoker vs. non-smoker test subjects.

Interstitial lung diseases are more prevalent in persons that smoke, or have had a long history of smoking prior to quitting, than in persons who never smoked. We compared differential gene expression profiles of samples derived from smoker and non-smoker UIP or non-UIP subjects to determine if smoking status affects performance of UIP diagnostic classifiers.

Transbronchial biopsy samples were prepared [according to the methods described in Examples 1 and 2, and RNA sequencing analysis was performed according to the method described in Example 3]. RNASeq data was generated for a subset of 24 samples (9 UIP and 15 non-UIP), and differential gene expression was analyzed according to three binary comparisons: (i) UIP vs. non-UIP, n=9 and 15 samples, respectively; (ii) Non-smoker UIP vs. Non-smoker non-UIP, n=3 and 5 samples, respectively; and (iii) Smoker UIP vs. Smoker non-UIP, n=12 and 4 samples, respectively.

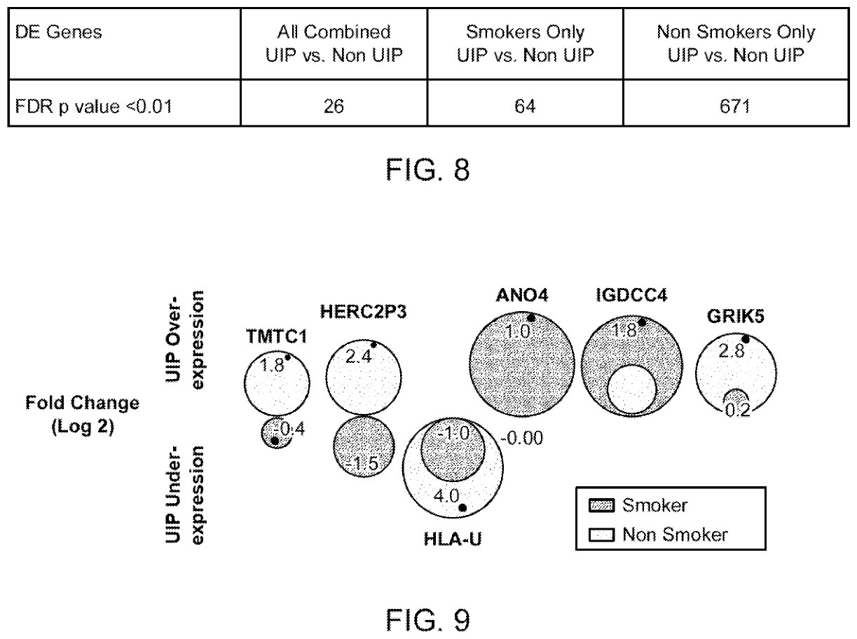

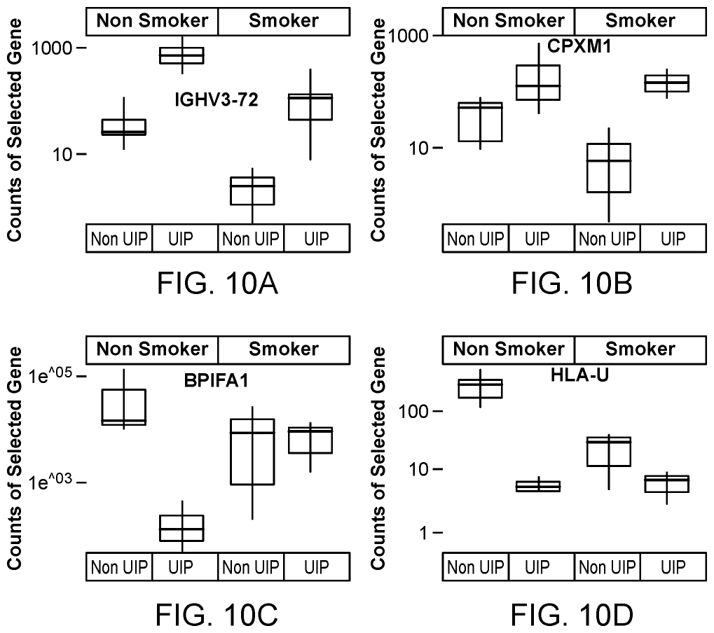

The results of the expression analysis for groups (i) to (iii) are shown in Tables 9 to 11, respectively, and are summarized in FIGS. 8-10. The number of genes differentially expressed between UIP and Non UIP samples differs drastically between smokers and non-smokers (64 differentially expressed in samples from smokers, 671 differentially expressed in samples from non-smokers) (FIG. 8). Moreover, certain genes that were differentially upregulated in non-smokers were downregulated on not differentially expressed in smokers (FIGS. 9 and 10). These data demonstrate that certain genes that are useful in UIP classification of samples from non-smokers are not informative, or can be contradictory in the diagnosis of the same disease, in smokers. Smoker-status differences in gene expression can reduce the performance of gene expression classifier predictions generated using traditional 2-class machine learning methods. We overcome this problem using three different techniques that are optionally combined or used individually in combination with the UIP vs. Non-UIP classifiers and methods of diagnosis UIP vs. Non-UIP via the diagnostic methods disclosed herein.

In a first approach, smoking status (smoker vs. non-smoker) is used as a covariate in the model during training. This simple approach boosts signal-to-noise ratio, particularly in data derived from smokers (were noise is higher) and allows data derived from smokers and non-smokers to be combined and used simultaneously.

In a second approach, genes that are susceptible to smoker-status bias are identified and excluded, or optionally weighted differently than genes that are not susceptible to such bias, during classifier training. This method enriches the feature space used for training with genes that are not confounded or affected by smoking status.

In a third approach, a tiered classification effort is utilized in which an initial classifier is trained to recognize gene signatures that distinguish smokers from non-smokers. Once patient samples are pre-classified as “smoker” or “non-smoker”, distinct classifiers that were each trained to distinguish UIP vs. Non UIP in smokers or non-smokers, respectively, are implemented. Such smoker or non-smoker-specific classifiers provide improved diagnostic performance.

TABLE 9
Differentially expressed genes in UIP vs. Non-UIP samples, irrespective of smoker status.

| | | | log2 Fold | | |
|---|---|---|---|---|---|
| Table 9. All samples | | | Change | | FDR |
| Ensembl ID | Entrez ID | Gene symbol | (UIP/NonUIP) | p value | p value |
| ENSG00000204256 | 6046 | BRD2 | −0.30 | 4.03E−11 | 6.40E−07 |
| ENSG00000129204 | 9098 | USP6 | 1.88 | 2.57E−10 | 2.04E−06 |
| ENSG00000148357 | 256158 | HMCN2 | 1.71 | 1.62E−08 | 8.58E−05 |
| ENSG00000178031 | 92949 | ADAMTSL1 | 1.69 | 1.23E−07 | 4.83E−04 |
| ENSG00000112130 | 9025 | RNF8 | −0.40 | 1.52E−07 | 4.83E−04 |
| ENSG00000197705 | 57565 | KLHL14 | 1.58 | 1.97E−07 | 5.21E−04 |
| ENSG00000204632 | 3135 | HLA-G | −1.65 | 4.51E−07 | 1.02E−03 |
| ENSG00000157064 | 23057 | NMNAT2 | 1.47 | 6.62E−07 | 1.31E−03 |
| ENSG00000112245 | 7803 | PTP4A1 | −0.59 | 1.38E−06 | 2.43E−03 |
| ENSG00000114115 | 5947 | RBP1 | 0.95 | 1.69E−06 | 2.68E−03 |
| ENSG00000152413 | 9456 | HOMER1 | −0.48 | 2.30E−06 | 3.32E−03 |
| ENSG00000143603 | 3782 | KCNN3 | 0.81 | 2.85E−06 | 3.54E−03 |
| ENSG00000198074 | 57016 | AKR1B10 | −1.48 | 2.90E−06 | 3.54E−03 |
| ENSG00000160007 | 2909 | ARHGAP35 | −0.28 | 4.24E−06 | 4.49E−03 |
| ENSG00000076053 | 10179 | RBM7 | −0.30 | 4.23E−06 | 4.49E−03 |
| ENSG00000085563 | 5243 | ABCB1 | 1.40 | 5.90E−06 | 5.55E−03 |
| ENSG00000047365 | 116984 | ARAP2 | −0.60 | 5.95E−06 | 5.55E−03 |
| ENSG00000130600 | 283120 | H19 | 1.45 | 7.10E−06 | 5.68E−03 |
| ENSG00000131355 | 84658 | ADGRE3 | 1.43 | 7.22E−06 | 5.68E−03 |
| ENSG00000135837 | 9857 | CEP350 | −0.23 | 6.63E−06 | 5.68E−03 |
| ENSG00000102221 | 9767 | JADE3 | −0.42 | 7.52E−06 | 5.68E−03 |
| ENSG00000105784 | 154661 | RUNDC3B | 1.31 | 1.02E−05 | 6.94E−03 |
| ENSG00000198734 | 2153 | F5 | 0.96 | 1.05E−05 | 6.94E−03 |
| ENSG00000124226 | 55905 | RNF114 | −0.39 | 1.04E−05 | 6.94E−03 |
| ENSG00000106852 | 26468 | LHX6 | 1.16 | 1.12E−05 | 7.10E−03 |
| ENSG00000183454 | 2903 | GRIN2A | 1.41 | 1.47E−05 | 8.93E−03 |
| ENSG00000170153 | 57484 | RNF150 | 1.04 | 1.54E−05 | 8.93E−03 |
| ENSG00000187527 | 344905 | ATP13A5 | −1.39 | 1.58E−05 | 8.93E−03 |
| ENSG00000244734 | 3043 | HBB | 1.40 | 1.71E−05 | 9.05E−03 |
| ENSG00000204936 | 57126 | CD177 | 1.40 | 1.67E−05 | 9.05E−03 |
| ENSG00000136560 | 10010 | TANK | −0.37 | 2.26E−05 | 1.16E−02 |
| ENSG00000196562 | 55959 | SULF2 | 0.98 | 2.87E−05 | 1.42E−02 |
| ENSG00000151789 | 79750 | ZNF385D | 1.36 | 3.17E−05 | 1.44E−02 |
| ENSG00000145808 | 171019 | ADAMTS19 | 1.07 | 3.14E−05 | 1.44E−02 |
| ENSG00000177666 | 57104 | PNPLA2 | −0.53 | 3.00E−05 | 1.44E−02 |
| ENSG00000007312 | 974 | CD79B | 1.31 | 3.51E−05 | 1.47E−02 |
| ENSG00000104213 | 5157 | PDGFRL | 1.24 | 3.53E−05 | 1.47E−02 |
| ENSG00000023171 | 57476 | GRAMD1B | −0.86 | 3.41E−05 | 1.47E−02 |
| ENSG00000117322 | 1380 | CR2 | 1.31 | 4.37E−05 | 1.78E−02 |
| ENSG00000171724 | 57687 | VAT1L | 1.33 | 4.51E−05 | 1.79E−02 |
| ENSG00000108852 | 4355 | MPP2 | 1.21 | 4.84E−05 | 1.87E−02 |
| ENSG00000136098 | 4752 | NEK3 | 0.51 | 5.00E−05 | 1.89E−02 |
| ENSG00000189056 | 5649 | RELN | 1.29 | 5.13E−05 | 1.89E−02 |
| ENSG00000054938 | 25884 | CHRDL2 | 1.30 | 6.70E−05 | 2.36E−02 |
| ENSG00000132704 | 79368 | FCRL2 | 1.30 | 6.62E−05 | 2.36E−02 |
| ENSG00000196628 | 6925 | TCF4 | 0.50 | 7.00E−05 | 2.41E−02 |
| ENSG00000142856 | 23421 | ITGB3BP | 0.55 | 7.32E−05 | 2.47E−02 |
| ENSG00000136153 | 4008 | LMO7 | −0.80 | 8.61E−05 | 2.85E−02 |
| ENSG00000203867 | 282996 | RBM20 | 1.20 | 9.01E−05 | 2.92E−02 |
| ENSG00000171049 | 2358 | FPR2 | 1.26 | 9.26E−05 | 2.94E−02 |
| ENSG00000152953 | 55351 | STK32B | 1.23 | 9.47E−05 | 2.95E−02 |
| ENSG00000099968 | 23786 | BCL2L13 | −0.37 | 9.73E−05 | 2.97E−02 |
| ENSG00000188536 | 3040 | HBA2 | 1.26 | 1.03E−04 | 3.00E−02 |
| ENSG00000198569 | 142680 | SLC34A3 | 0.94 | 1.04E−04 | 3.00E−02 |
| ENSG00000163701 | 132014 | IL17RE | −0.60 | 1.02E−04 | 3.00E−02 |
| ENSG00000197614 | 8076 | MFAP5 | 1.25 | 1.10E−04 | 3.12E−02 |
| ENSG00000146021 | 26249 | KLHL3 | 0.84 | 1.15E−04 | 3.19E−02 |
| ENSG00000156103 | 4325 | MMP16 | 1.25 | 1.21E−04 | 3.24E−02 |
| ENSG00000185022 | 23764 | MAFF | −1.05 | 1.20E−04 | 3.24E−02 |
| ENSG00000088882 | 56265 | CPXM1 | 1.22 | 1.32E−04 | 3.49E−02 |
| ENSG00000163032 | 7447 | VSNL1 | 0.78 | 1.38E−04 | 3.58E−02 |
| ENSG00000186184 | 51082 | POLR1D | −0.47 | 1.40E−04 | 3.58E−02 |
| ENSG00000177106 | 64787 | EPS8L2 | −0.52 | 1.42E−04 | 3.58E−02 |
| ENSG00000214711 | 440854 | CAPN14 | 1.23 | 1.44E−04 | 3.58E−02 |
| ENSG00000113212 | 56129 | PCDHB7 | 1.04 | 1.51E−04 | 3.64E−02 |
| ENSG00000001629 | 54467 | ANKIB1 | −0.22 | 1.52E−04 | 3.64E−02 |
| ENSG00000180871 | 3579 | CXCR2 | 1.20 | 1.79E−04 | 4.17E−02 |
| ENSG00000065618 | 1308 | COL17A1 | 1.04 | 1.79E−04 | 4.17E−02 |
| ENSG00000008394 | 4257 | MGST1 | −0.59 | 1.81E−04 | 4.17E−02 |
| ENSG00000185046 | 56899 | ANKS1B | 1.01 | 2.16E−04 | 4.82E−02 |
| ENSG00000165949 | 3429 | IFI27 | −1.05 | 2.14E−04 | 4.82E−02 |
| ENSG00000186847 | 3861 | KRT14 | 1.20 | 2.30E−04 | 5.04E−02 |
| ENSG00000136929 | 55363 | HEMGN | 1.19 | 2.35E−04 | 5.04E−02 |
| ENSG00000167107 | 80221 | ACSF2 | −0.55 | 2.35E−04 | 5.04E−02 |
| ENSG00000169129 | 84632 | AFAP1L2 | 0.87 | 2.45E−04 | 5.19E−02 |
| ENSG00000134762 | 1825 | DSC3 | 1.10 | 2.86E−04 | 5.88E−02 |
| ENSG00000212743 | NA | | 0.96 | 2.85E−04 | 5.88E−02 |
| ENSG00000149575 | 6327 | SCN2B | 1.13 | 3.00E−04 | 6.10E−02 |
| ENSG00000188257 | 5320 | PLA2G2A | 1.16 | 3.22E−04 | 6.15E−02 |
| ENSG00000102935 | 23090 | ZNF423 | 1.02 | 3.15E−04 | 6.15E−02 |
| ENSG00000143320 | 1382 | CRABP2 | 0.99 | 3.22E−04 | 6.15E−02 |
| ENSG00000138660 | 55435 | AP1AR | −0.49 | 3.07E−04 | 6.15E−02 |
| ENSG00000157601 | 4599 | MX1 | −0.88 | 3.15E−04 | 6.15E−02 |
| ENSG00000171847 | 55138 | FAM90A1 | 0.80 | 3.36E−04 | 6.22E−02 |
| ENSG00000115461 | 3488 | IGFBP5 | 0.71 | 3.37E−04 | 6.22E−02 |
| ENSG00000151632 | 1646 | AKR1C2 | −1.17 | 3.36E−04 | 6.22E−02 |
| ENSG00000189306 | 27341 | RRP7A | 0.44 | 3.42E−04 | 6.24E−02 |
| ENSG00000148541 | 220965 | FAM13C | 0.72 | 3.60E−04 | 6.49E−02 |
| ENSG00000111725 | 5564 | PRKAB1 | −0.49 | 3.65E−04 | 6.50E−02 |
| ENSG00000182885 | 222487 | ADGRG3 | 1.15 | 3.74E−04 | 6.54E−02 |
| ENSG00000206172 | 3039 | HBA1 | 1.15 | 3.80E−04 | 6.54E−02 |
| ENSG00000157502 | 139221 | MUM1L1 | 1.09 | 3.77E−04 | 6.54E−02 |
| ENSG00000169891 | 9185 | REPS2 | −0.46 | 3.88E−04 | 6.54E−02 |
| ENSG00000129437 | 43847 | KLK14 | −1.09 | 3.85E−04 | 6.54E−02 |
| ENSG00000211956 | NA | IGHV4-34 | 1.15 | 3.93E−04 | 6.57E−02 |
| ENSG00000106483 | 6424 | SFRP4 | 1.15 | 4.19E−04 | 6.75E−02 |
| ENSG00000175879 | 3234 | HOXD8 | 1.13 | 4.36E−04 | 6.75E−02 |
| ENSG00000166947 | 2038 | EPB42 | 1.04 | 4.35E−04 | 6.75E−02 |
| ENSG00000028310 | 65980 | BRD9 | 0.17 | 4.15E−04 | 6.75E−02 |
| ENSG00000134046 | 8932 | MBD2 | −0.18 | 4.39E−04 | 6.75E−02 |
| ENSG00000011007 | 6924 | TCEB3 | −0.29 | 4.10E−04 | 6.75E−02 |
| ENSG00000198722 | 10497 | UNC13B | −0.46 | 4.24E−04 | 6.75E−02 |
| ENSG00000182795 | 79098 | C1orf116 | −0.80 | 4.33E−04 | 6.75E−02 |
| ENSG00000110042 | 23220 | DTX4 | −0.40 | 4.44E−04 | 6.78E−02 |
| ENSG00000169612 | 83640 | FAM103A1 | −0.67 | 4.56E−04 | 6.88E−02 |
| ENSG00000139517 | 222484 | LNX2 | −0.38 | 4.66E−04 | 6.98E−02 |
| ENSG00000165966 | 29951 | PDZRN4 | 1.14 | 4.80E−04 | 7.01E−02 |
| ENSG00000196109 | 163223 | ZNF676 | 1.12 | 4.81E−04 | 7.01E−02 |
| ENSG00000188517 | 84570 | COL25A1 | 1.12 | 4.86E−04 | 7.01E−02 |
| ENSG00000121898 | 119587 | CPXM2 | 1.07 | 4.83E−04 | 7.01E−02 |
| ENSG00000185739 | 6345 | SRL | 1.13 | 5.01E−04 | 7.10E−02 |
| ENSG00000166033 | 5654 | HTRA1 | 0.82 | 4.98E−04 | 7.10E−02 |
| ENSG00000179772 | 2307 | FOXS1 | 1.13 | 5.20E−04 | 7.17E−02 |
| ENSG00000172137 | 794 | CALB2 | 1.13 | 5.19E−04 | 7.17E−02 |
| ENSG00000086544 | 80271 | ITPKC | −0.42 | 5.15E−04 | 7.17E−02 |
| ENSG00000130513 | 9518 | GDF15 | −1.04 | 5.29E−04 | 7.17E−02 |
| ENSG00000129451 | 5655 | KLK10 | −1.05 | 5.26E−04 | 7.17E−02 |
| ENSG00000109472 | 1363 | CPE | 1.03 | 5.40E−04 | 7.20E−02 |
| ENSG00000171206 | 81603 | TRIM8 | −0.30 | 5.38E−04 | 7.20E−02 |
| ENSG00000128285 | 2847 | MCHR1 | 1.09 | 5.60E−04 | 7.40E−02 |
| ENSG00000197993 | 3792 | KEL | 1.08 | 5.68E−04 | 7.45E−02 |
| ENSG00000138642 | 55008 | HERC6 | −0.82 | 5.78E−04 | 7.51E−02 |
| ENSG00000162729 | 93185 | IGSF8 | 0.32 | 5.93E−04 | 7.65E−02 |
| ENSG00000105369 | 973 | CD79A | 1.11 | 6.54E−04 | 7.67E−02 |
| ENSG00000146374 | 84870 | RSPO3 | 1.11 | 6.56E−04 | 7.67E−02 |
| ENSG00000167483 | 199786 | FAM129C | 1.11 | 6.75E−04 | 7.67E−02 |
| ENSG00000205038 | 93035 | PKHD1L1 | 1.07 | 6.67E−04 | 7.67E−02 |
| ENSG00000158560 | 1780 | DYNC1I1 | 1.07 | 6.63E−04 | 7.67E−02 |
| ENSG00000101000 | 10544 | PROCR | 1.04 | 6.65E−04 | 7.67E−02 |
| ENSG00000197410 | 54798 | DCHS2 | 1.03 | 6.66E−04 | 7.67E−02 |
| ENSG00000137573 | 23213 | SULF1 | 1.02 | 6.74E−04 | 7.67E−02 |
| ENSG00000091972 | 4345 | CD200 | 1.01 | 6.81E−04 | 7.67E−02 |
| ENSG00000161381 | 57125 | PLXDC1 | 0.97 | 6.23E−04 | 7.67E−02 |
| ENSG00000067840 | 57595 | PDZD4 | 0.94 | 6.64E−04 | 7.67E−02 |
| ENSG00000244363 | NA | RPL7P23 | 0.77 | 6.71E−04 | 7.67E−02 |
| ENSG00000141698 | 115024 | NT5C3B | 0.41 | 6.51E−04 | 7.67E−02 |
| ENSG00000184056 | 26276 | VPS33B | 0.24 | 6.73E−04 | 7.67E−02 |
| ENSG00000226742 | 440498 | HSBP1L1 | −0.37 | 6.66E−04 | 7.67E−02 |
| ENSG00000064666 | 1265 | CNN2 | −0.38 | 6.73E−04 | 7.67E−02 |
| ENSG00000198142 | 65124 | SOWAHC | −0.53 | 6.36E−04 | 7.67E−02 |
| ENSG00000198183 | 51297 | BPIFA1 | −1.09 | 6.79E−04 | 7.67E−02 |
| ENSG00000185271 | 123103 | KLHL33 | 1.10 | 7.36E−04 | 7.96E−02 |
| ENSG00000143248 | 8490 | RGS5 | 1.10 | 7.26E−04 | 7.96E−02 |
| ENSG00000106018 | 7434 | VIPR2 | 1.10 | 7.45E−04 | 7.96E−02 |
| ENSG00000168542 | 1281 | COL3A1 | 1.10 | 7.33E−04 | 7.96E−02 |
| ENSG00000186105 | 100130733 | LRRC70 | 0.93 | 7.44E−04 | 7.96E−02 |
| ENSG00000265150 | NA | NA | 0.87 | 7.23E−04 | 7.96E−02 |
| ENSG00000172840 | 57546 | PDP2 | −0.34 | 7.47E−04 | 7.96E−02 |
| ENSG00000156675 | 80223 | RAB11FIP1 | −0.61 | 7.27E−04 | 7.96E−02 |
| ENSG00000108001 | 253738 | EBF3 | 1.10 | 7.84E−04 | 8.11E−02 |
| ENSG00000114948 | 8745 | ADAM23 | 1.08 | 7.82E−04 | 8.11E−02 |
| ENSG00000106404 | 24146 | CLDN15 | 1.08 | 7.73E−04 | 8.11E−02 |
| ENSG00000139910 | 4857 | NOVA1 | 1.07 | 7.92E−04 | 8.11E−02 |
| ENSG00000146802 | 64418 | TMEM168 | 0.33 | 7.92E−04 | 8.11E−02 |
| ENSG00000183486 | 4600 | MX2 | −0.91 | 7.89E−04 | 8.11E−02 |
| ENSG00000157766 | 176 | ACAN | 1.08 | 8.06E−04 | 8.16E−02 |
| ENSG00000214174 | 201283 | AMZ2P1 | −0.54 | 8.08E−04 | 8.16E−02 |
| ENSG00000185305 | 54622 | ARL15 | 0.67 | 8.21E−04 | 8.19E−02 |
| ENSG00000137709 | 25833 | POU2F3 | −0.67 | 8.17E−04 | 8.19E−02 |
| ENSG00000117707 | 5629 | PROX1 | 0.99 | 8.39E−04 | 8.31E−02 |
| ENSG00000144619 | 152330 | CNTN4 | 0.77 | 8.54E−04 | 8.41E−02 |
| ENSG00000158578 | 212 | ALAS2 | 1.08 | 8.94E−04 | 8.58E−02 |
| ENSG00000228570 | NA | NUTM2E | 1.08 | 9.23E−04 | 8.58E−02 |
| ENSG00000163735 | 6374 | CXCL5 | 1.08 | 9.30E−04 | 8.58E−02 |
| ENSG00000167476 | 126306 | JSRP1 | 1.07 | 9.11E−04 | 8.58E−02 |
| ENSG00000116833 | 2494 | NR5A2 | 1.06 | 8.81E−04 | 8.58E−02 |
| ENSG00000120322 | 56128 | PCDHB8 | 1.05 | 9.31E−04 | 8.58E−02 |
| ENSG00000238741 | 677767 | SCARNA7 | 0.37 | 9.26E−04 | 8.58E−02 |
| ENSG00000169093 | 8623 | ASMTL | 0.23 | 9.12E−04 | 8.58E−02 |
| ENSG00000081026 | 260425 | MAGI3 | −0.61 | 9.06E−04 | 8.58E−02 |
| ENSG00000103528 | 51760 | SYT17 | −0.62 | 9.00E−04 | 8.58E−02 |
| ENSG00000100033 | 5625 | PRODH | −0.81 | 9.09E−04 | 8.58E−02 |
| ENSG00000165868 | 259217 | HSPA12A | 0.86 | 9.54E−04 | 8.75E−02 |
| ENSG00000163251 | 7855 | FZD5 | −0.74 | 9.87E−04 | 9.00E−02 |
| ENSG00000122140 | 51116 | MRPS2 | −0.38 | 9.93E−04 | 9.00E−02 |
| ENSG00000171792 | 83695 | RHNO1 | −0.48 | 1.00E−03 | 9.02E−02 |
| ENSG00000104369 | 56704 | JPH1 | −0.58 | 1.03E−03 | 9.20E−02 |
| ENSG00000168079 | 286133 | SCARA5 | 1.07 | 1.03E−03 | 9.21E−02 |
| ENSG00000092295 | 7051 | TGM1 | 1.00 | 1.04E−03 | 9.22E−02 |
| ENSG00000106809 | 4969 | OGN | 1.07 | 1.06E−03 | 9.35E−02 |
| ENSG00000163534 | 115350 | FCRL1 | 1.06 | 1.11E−03 | 9.44E−02 |
| ENSG00000091137 | 5172 | SLC26A4 | 1.00 | 1.10E−03 | 9.44E−02 |
| ENSG00000099958 | 91319 | DERL3 | 0.89 | 1.15E−03 | 9.44E−02 |
| ENSG00000153253 | 6328 | SCN3A | 0.82 | 1.16E−03 | 9.44E−02 |
| ENSG00000251402 | NA | FAM90A25P | 0.74 | 1.12E−03 | 9.44E−02 |
| ENSG00000120327 | 56122 | PCDHB14 | 0.72 | 1.09E−03 | 9.44E−02 |
| ENSG00000138795 | 51176 | LEF1 | 0.64 | 1.13E−03 | 9.44E−02 |
| ENSG00000144040 | 94097 | SFXN5 | 0.44 | 1.12E−03 | 9.44E−02 |
| ENSG00000155463 | 5018 | OXA1L | −0.20 | 1.15E−03 | 9.44E−02 |
| ENSG00000136830 | 64855 | FAM129B | −0.24 | 1.15E−03 | 9.44E−02 |
| ENSG00000141452 | 29919 | C18orf8 | −0.30 | 1.15E−03 | 9.44E−02 |
| ENSG00000148730 | 1979 | EIF4EBP2 | −0.33 | 1.12E−03 | 9.44E−02 |
| ENSG00000115415 | 6772 | STAT1 | −0.86 | 1.10E−03 | 9.44E−02 |
| ENSG00000126709 | 2537 | IFI6 | −1.03 | 1.15E−03 | 9.44E−02 |
| ENSG00000095951 | 3096 | HIVEP1 | −0.24 | 1.20E−03 | 9.76E−02 |
| ENSG00000187942 | 401944 | LDLRAD2 | 0.97 | 1.21E−03 | 9.80E−02 |
| ENSG00000105928 | 1687 | DFNA5 | 0.83 | 1.22E−03 | 9.81E−02 |
| ENSG00000224397 | NA | LINC01272 | 1.02 | 1.23E−03 | 9.83E−02 |
| ENSG00000170011 | 25924 | MYRIP | 0.86 | 1.32E−03 | 1.00E−01 |
| ENSG00000120784 | 22835 | ZFP30 | 0.37 | 1.30E−03 | 1.00E−01 |
| ENSG00000170185 | 84640 | USP38 | −0.26 | 1.31E−03 | 1.00E−01 |
| ENSG00000076513 | 88455 | ANKRD13A | −0.31 | 1.26E−03 | 1.00E−01 |
| ENSG00000135148 | 10906 | TRAFD1 | −0.32 | 1.28E−03 | 1.00E−01 |
| ENSG00000170085 | 375484 | SIMC1 | −0.43 | 1.32E−03 | 1.00E−01 |
| ENSG00000175324 | 27257 | LSM1 | −0.45 | 1.30E−03 | 1.00E−01 |
| ENSG00000141738 | 2886 | GRB7 | −0.54 | 1.28E−03 | 1.00E−01 |
| ENSG00000088002 | 6820 | SULT2B1 | −0.75 | 1.28E−03 | 1.00E−01 |
| ENSG00000010030 | 51513 | ETV7 | −0.86 | 1.29E−03 | 1.00E−01 |
| ENSG00000137959 | 10964 | IFI44L | −1.01 | 1.27E−03 | 1.00E−01 |

UIP (n = 9 samples); Non UIP (n = 15 samples). Positive log2 fold change value indicates over- expression in UIP relative to Non UIP; negative log2 value indicates under-expression in UIP relative to Non UIP. In this analysis the smoking history status of the patients involved was not evaluated, and the cohort harbored both smokers and non-smokers.

TABLE 10
Differentially expressed genes in non-smoker UIP vs. non-smoker Non-UIP samples.
Table 10. Non-smokers

| | | | Log2 Fold | | |
|---|---|---|---|---|---|
| | | | Change | | |
| Ensembl ID | Entrez ID | Gene symbol | (UIP/NonUIP) | P value | FDR P value |
| ENSG00000133687 | 83857 | TMTC1 | 1.72 | 4.52E−21 | 6.68E−17 |
| ENSG00000169129 | 84632 | AFAP1L2 | 1.63 | 1.62E−19 | 1.19E−15 |
| ENSG00000180229 | 283755 | HERC2P3 | 2.52 | 9.77E−15 | 4.81E−11 |
| ENSG00000107518 | 26033 | ATRNL1 | 3.00 | 2.49E−14 | 9.21E−11 |
| ENSG00000198380 | 2673 | GFPT1 | −0.62 | 9.55E−14 | 2.35E−10 |
| ENSG00000211976 | NA | IGHV3-73 | 2.21 | 9.47E−14 | 2.35E−10 |
| ENSG00000114902 | 28972 | SPCS1 | −0.64 | 1.99E−12 | 4.20E−09 |
| ENSG00000108001 | 253738 | EBF3 | 2.77 | 3.39E−12 | 6.27E−09 |
| ENSG00000154122 | 56172 | ANKH | 1.02 | 4.17E−12 | 6.85E−09 |
| ENSG00000012660 | 60481 | ELOVL5 | −0.66 | 6.94E−12 | 1.03E−08 |
| ENSG00000119888 | 4072 | EPCAM | −1.04 | 8.67E−12 | 1.16E−08 |
| ENSG00000147676 | 114569 | MAL2 | −1.08 | 1.83E−11 | 1.94E−08 |
| ENSG00000148357 | 256158 | HMCN2 | 1.99 | 1.62E−11 | 1.94E−08 |
| ENSG00000185499 | 4582 | MUC1 | −1.09 | 1.84E−11 | 1.94E−08 |
| ENSG00000157557 | 2114 | ETS2 | 0.73 | 8.18E−11 | 8.06E−08 |
| ENSG00000151789 | 79750 | ZNF385D | 2.73 | 1.04E−10 | 9.62E−08 |
| ENSG00000213088 | 2532 | ACKR1 | 2.18 | 3.53E−10 | 3.06E−07 |
| ENSG00000038210 | 55300 | PI4K2B | −0.77 | 4.14E−10 | 3.40E−07 |
| ENSG00000105426 | 5802 | PTPRS | 0.80 | 9.94E−10 | 7.73E−07 |
| ENSG00000100034 | 9647 | PPM1F | 1.27 | 1.17E−09 | 8.14E−07 |
| ENSG00000112562 | 64094 | SMOC2 | 1.70 | 1.21E−09 | 8.14E−07 |
| ENSG00000139211 | 347902 | AMIGO2 | 0.93 | 1.15E−09 | 8.14E−07 |
| ENSG00000130158 | 57572 | DOCK6 | 0.75 | 1.42E−09 | 9.10E−07 |
| ENSG00000124145 | 6385 | SDC4 | −0.67 | 2.44E−09 | 1.44E−06 |
| ENSG00000129255 | 9526 | MPDU1 | −0.54 | 2.44E−09 | 1.44E−06 |
| ENSG00000100219 | 7494 | XBP1 | −1.02 | 2.76E−09 | 1.48E−06 |
| ENSG00000120318 | 64411 | ARAP3 | 0.93 | 2.80E−09 | 1.48E−06 |
| ENSG00000144136 | 6574 | SLC20A1 | −0.65 | 2.63E−09 | 1.48E−06 |
| ENSG00000140873 | 170692 | ADAMTS18 | 2.27 | 3.32E−09 | 1.69E−06 |
| ENSG00000120693 | 4093 | SMAD9 | 1.43 | 4.04E−09 | 1.99E−06 |
| ENSG00000080007 | 55510 | DDX43 | −2.11 | 6.55E−09 | 3.12E−06 |
| ENSG00000105737 | 2901 | GRIK5 | 2.34 | 8.09E−09 | 3.54E−06 |
| ENSG00000157064 | 23057 | NMNAT2 | 2.14 | 8.14E−09 | 3.54E−06 |
| ENSG00000161381 | 57125 | PLXDC1 | 1.21 | 7.73E−09 | 3.54E−06 |
| ENSG00000154736 | 11096 | ADAMTS5 | 1.92 | 1.01E−08 | 4.13E−06 |
| ENSG00000185046 | 56899 | ANKS1B | 1.51 | 9.78E−09 | 4.13E−06 |
| ENSG00000104213 | 5157 | PDGFRL | 2.09 | 1.19E−08 | 4.52E−06 |
| ENSG00000166398 | 9710 | KIAA0355 | 0.82 | 1.19E−08 | 4.52E−06 |
| ENSG00000189058 | 347 | APOD | 1.55 | 1.14E−08 | 4.52E−06 |
| ENSG00000102935 | 23090 | ZNF423 | 1.47 | 1.34E−08 | 4.86E−06 |
| ENSG00000111145 | 2004 | ELK3 | 0.89 | 1.35E−08 | 4.86E−06 |
| ENSG00000138160 | 3832 | KIF11 | −1.35 | 1.44E−08 | 5.06E−06 |
| ENSG00000197147 | 23507 | LRRC8B | −0.89 | 1.80E−08 | 6.19E−06 |
| ENSG00000131386 | 117248 | GALNT15 | 2.41 | 1.95E−08 | 6.39E−06 |
| ENSG00000177839 | 56127 | PCDHB9 | 1.87 | 1.91E−08 | 6.39E−06 |
| ENSG00000114446 | 55081 | IFT57 | −0.65 | 2.20E−08 | 7.06E−06 |
| ENSG00000198932 | 9737 | GPRASP1 | 0.90 | 2.34E−08 | 7.36E−06 |
| ENSG00000178031 | 92949 | ADAMTSL1 | 2.30 | 2.50E−08 | 7.70E−06 |
| ENSG00000230989 | 3281 | HSBP1 | −0.74 | 2.59E−08 | 7.80E−06 |
| ENSG00000100065 | 29775 | CARD10 | 1.69 | 2.91E−08 | 8.60E−06 |
| ENSG00000142798 | 3339 | HSPG2 | 1.20 | 3.13E−08 | 9.06E−06 |
| ENSG00000134533 | 85004 | RERG | 1.68 | 3.70E−08 | 1.05E−05 |
| ENSG00000105784 | 154661 | RUNDC3B | 2.28 | 3.87E−08 | 1.08E−05 |
| ENSG00000039068 | 999 | CDH1 | −0.87 | 6.14E−08 | 1.55E−05 |
| ENSG00000104549 | 6713 | SQLE | −1.38 | 6.10E−08 | 1.55E−05 |
| ENSG00000105996 | 3199 | HOXA2 | 1.60 | 5.72E−08 | 1.55E−05 |
| ENSG00000130234 | 59272 | ACE2 | −1.52 | 6.19E−08 | 1.55E−05 |
| ENSG00000162881 | 165140 | OXER1 | 1.30 | 5.81E−08 | 1.55E−05 |
| ENSG00000177098 | 6330 | SCN4B | 1.12 | 6.10E−08 | 1.55E−05 |
| ENSG00000007312 | 974 | CD79B | 2.13 | 6.62E−08 | 1.58E−05 |
| ENSG00000163898 | 200879 | LIPH | −0.91 | 6.52E−08 | 1.58E−05 |
| ENSG00000189377 | 284340 | CXCL17 | −1.17 | 6.53E−08 | 1.58E−05 |
| ENSG00000198814 | 2710 | GK | −0.83 | 6.79E−08 | 1.59E−05 |
| ENSG00000076944 | 6813 | STXBP2 | −0.56 | 7.52E−08 | 1.71E−05 |
| ENSG00000153902 | 163175 | LGI4 | 1.74 | 7.53E−08 | 1.71E−05 |
| ENSG00000117448 | 10327 | AKR1A1 | −0.63 | 7.74E−08 | 1.73E−05 |
| ENSG00000164530 | 221476 | PI16 | 2.17 | 8.33E−08 | 1.84E−05 |
| ENSG00000233297 | NA | RASA4DP | 2.29 | 9.09E−08 | 1.97E−05 |
| ENSG00000182718 | 302 | ANXA2 | −0.62 | 9.95E−08 | 2.13E−05 |
| ENSG00000110455 | 84680 | ACCS | 0.98 | 1.03E−07 | 2.17E−05 |
| ENSG00000197253 | 64499 | TPSB2 | 2.07 | 1.10E−07 | 2.29E−05 |
| ENSG00000125999 | 92747 | BPIFB1 | −1.76 | 1.15E−07 | 2.37E−05 |
| ENSG00000211936 | NA | NA | 2.23 | 1.22E−07 | 2.47E−05 |
| ENSG00000108852 | 4355 | MPP2 | 1.68 | 1.28E−07 | 2.55E−05 |
| ENSG00000119514 | 79695 | GALNT12 | −0.60 | 1.64E−07 | 3.18E−05 |
| ENSG00000146021 | 26249 | KLHL3 | 1.36 | 1.63E−07 | 3.18E−05 |
| ENSG00000173702 | 56667 | MUC13 | −2.33 | 1.78E−07 | 3.41E−05 |
| ENSG00000116748 | 270 | AMPD1 | 1.80 | 1.85E−07 | 3.50E−05 |
| ENSG00000196411 | 2050 | EPHB4 | 0.86 | 2.09E−07 | 3.91E−05 |
| ENSG00000086061 | 3301 | DNAJA1 | −0.73 | 2.20E−07 | 4.07E−05 |
| ENSG00000074181 | 4854 | NOTCH3 | 0.92 | 2.24E−07 | 4.07E−05 |
| ENSG00000181234 | 92293 | TMEM132C | 2.08 | 2.26E−07 | 4.07E−05 |
| ENSG00000074590 | 9891 | NUAK1 | 1.27 | 2.47E−07 | 4.39E−05 |
| ENSG00000154263 | 10349 | ABCA10 | 1.44 | 2.92E−07 | 5.14E−05 |
| ENSG00000186322 | NA | NA | 1.99 | 3.30E−07 | 5.74E−05 |
| ENSG00000130164 | 3949 | LDLR | −1.00 | 3.36E−07 | 5.77E−05 |
| ENSG00000133935 | 11161 | C14orf1 | −0.76 | 3.54E−07 | 5.99E−05 |
| ENSG00000151892 | 2674 | GFRA1 | 1.96 | 3.57E−07 | 5.99E−05 |
| ENSG00000196628 | 6925 | TCF4 | 0.93 | 3.65E−07 | 6.06E−05 |
| ENSG00000106070 | 2887 | GRB10 | 1.22 | 3.94E−07 | 6.46E−05 |
| ENSG00000076555 | 32 | ACACB | 1.06 | 4.48E−07 | 7.27E−05 |
| ENSG00000173947 | 128344 | PIFO | −1.26 | 4.74E−07 | 7.61E−05 |
| ENSG00000004468 | 952 | CD38 | −1.40 | 5.09E−07 | 8.08E−05 |
| ENSG00000154330 | 5239 | PGM5 | 1.72 | 5.62E−07 | 8.83E−05 |
| ENSG00000085185 | 63035 | BCORL1 | 0.61 | 5.71E−07 | 8.88E−05 |
| ENSG00000157933 | 6497 | SKI | 0.57 | 5.77E−07 | 8.89E−05 |
| ENSG00000080854 | 22997 | IGSF9B | 1.56 | 6.01E−07 | 9.16E−05 |
| ENSG00000244734 | 3043 | HBB | 2.28 | 6.25E−07 | 9.42E−05 |
| ENSG00000080572 | 139212 | PIH1D3 | −1.44 | 6.80E−07 | 1.02E−04 |
| ENSG00000154529 | 728577 | CNTNAP3B | 1.83 | 7.23E−07 | 1.07E−04 |
| ENSG00000142731 | 10733 | PLK4 | −1.30 | 9.19E−07 | 1.34E−04 |
| ENSG00000146425 | 6993 | DYNLT1 | −1.05 | 9.34E−07 | 1.35E−04 |
| ENSG00000109861 | 1075 | CTSC | −0.92 | 9.89E−07 | 1.42E−04 |
| ENSG00000118640 | 8673 | VAMP8 | −0.83 | 1.00E−06 | 1.43E−04 |
| ENSG00000091262 | 368 | ABCC6 | −0.97 | 1.05E−06 | 1.48E−04 |
| ENSG00000012124 | 933 | CD22 | 1.94 | 1.08E−06 | 1.48E−04 |
| ENSG00000079337 | 10411 | RAPGEF3 | 1.63 | 1.09E−06 | 1.48E−04 |
| ENSG00000113161 | 3156 | HMGCR | −1.13 | 1.09E−06 | 1.48E−04 |
| ENSG00000183346 | 219621 | C10orf107 | −1.22 | 1.07E−06 | 1.48E−04 |
| ENSG00000197705 | 57565 | KLHL14 | 2.01 | 1.11E−06 | 1.49E−04 |
| ENSG00000215217 | 134121 | C5orf49 | −1.20 | 1.14E−06 | 1.52E−04 |
| ENSG00000070190 | 27071 | DAPP1 | −1.19 | 1.19E−06 | 1.55E−04 |
| ENSG00000165434 | 283209 | PGM2L1 | −0.92 | 1.18E−06 | 1.55E−04 |
| ENSG00000102738 | 10240 | MRPS31 | −0.97 | 1.22E−06 | 1.56E−04 |
| ENSG00000132698 | 57111 | RAB25 | −0.79 | 1.22E−06 | 1.56E−04 |
| ENSG00000186105 | 100130733 | LRRC70 | 1.77 | 1.24E−06 | 1.58E−04 |
| ENSG00000123983 | 2181 | ACSL3 | −0.87 | 1.38E−06 | 1.72E−04 |
| ENSG00000188010 | 729967 | MORN2 | −1.27 | 1.37E−06 | 1.72E−04 |
| ENSG00000138670 | 153020 | RASGEF1B | −0.83 | 1.56E−06 | 1.94E−04 |
| ENSG00000152104 | 5784 | PTPN14 | 0.94 | 1.62E−06 | 1.98E−04 |
| ENSG00000165995 | 783 | CACNB2 | 1.24 | 1.62E−06 | 1.98E−04 |
| ENSG00000109586 | 51809 | GALNT7 | −0.94 | 1.73E−06 | 2.10E−04 |
| ENSG00000129946 | 25759 | SHC2 | 1.66 | 1.82E−06 | 2.18E−04 |
| ENSG00000139910 | 4857 | NOVA1 | 2.15 | 1.99E−06 | 2.37E−04 |
| ENSG00000188175 | 253012 | HEPACAM2 | −1.75 | 2.01E−06 | 2.37E−04 |
| ENSG00000174405 | 3981 | LIG4 | −0.58 | 2.04E−06 | 2.37E−04 |
| ENSG00000181885 | 1366 | CLDN7 | −1.13 | 2.03E−06 | 2.37E−04 |
| ENSG00000187134 | 1645 | AKR1C1 | −0.86 | 2.07E−06 | 2.39E−04 |
| ENSG00000253731 | 56109 | PCDHGA6 | 1.19 | 2.09E−06 | 2.39E−04 |
| ENSG00000090006 | 8425 | LTBP4 | 1.38 | 2.13E−06 | 2.42E−04 |
| ENSG00000176438 | 161176 | SYNE3 | 0.98 | 2.18E−06 | 2.44E−04 |
| ENSG00000188536 | 3040 | HBA2 | 2.17 | 2.17E−06 | 2.44E−04 |
| ENSG00000141198 | 10040 | TOM1L1 | −0.57 | 2.27E−06 | 2.50E−04 |
| ENSG00000164124 | 55314 | TMEM144 | −0.88 | 2.26E−06 | 2.50E−04 |
| ENSG00000068912 | 27248 | ERLEC1 | −0.48 | 2.33E−06 | 2.55E−04 |
| ENSG00000167779 | 3489 | IGFBP6 | 1.85 | 2.44E−06 | 2.65E−04 |
| ENSG00000159212 | 54102 | CLIC6 | −0.90 | 2.50E−06 | 2.70E−04 |
| ENSG00000116833 | 2494 | NR5A2 | 1.94 | 2.54E−06 | 2.72E−04 |
| ENSG00000197614 | 8076 | MFAP5 | 2.15 | 2.70E−06 | 2.87E−04 |
| ENSG00000152518 | 678 | ZFP36L2 | 0.79 | 2.81E−06 | 2.96E−04 |
| ENSG00000177156 | 6888 | TALDO1 | −0.45 | 2.83E−06 | 2.96E−04 |
| ENSG00000144619 | 152330 | CNTN4 | 0.79 | 2.89E−06 | 3.01E−04 |
| ENSG00000124772 | 57699 | CPNE5 | 1.80 | 2.99E−06 | 3.04E−04 |
| ENSG00000166908 | 79837 | PIP4K2C | −0.49 | 2.99E−06 | 3.04E−04 |
| ENSG00000168765 | 2948 | GSTM4 | 0.72 | 2.95E−06 | 3.04E−04 |
| ENSG00000105928 | 1687 | DFNA5 | 1.47 | 3.02E−06 | 3.05E−04 |
| ENSG00000186471 | 158798 | AKAP14 | −1.21 | 3.04E−06 | 3.05E−04 |
| ENSG00000183604 | NA | SMG1P5 | 1.68 | 3.12E−06 | 3.11E−04 |
| ENSG00000182272 | 338707 | B4GALNT4 | 1.65 | 3.32E−06 | 3.29E−04 |
| ENSG00000170017 | 214 | ALCAM | −1.08 | 3.37E−06 | 3.32E−04 |
| ENSG00000163191 | 6282 | S100A11 | −0.92 | 3.77E−06 | 3.69E−04 |
| ENSG00000100243 | 1727 | CYB5R3 | 0.31 | 3.83E−06 | 3.70E−04 |
| ENSG00000119616 | 51077 | FCF1 | −0.57 | 3.81E−06 | 3.70E−04 |
| ENSG00000106852 | 26468 | LHX6 | 1.71 | 3.86E−06 | 3.70E−04 |
| ENSG00000164056 | 10252 | SPRY1 | 1.10 | 4.26E−06 | 4.06E−04 |
| ENSG00000256870 | 160728 | SLC5A8 | −1.68 | 4.31E−06 | 4.08E−04 |
| ENSG00000112110 | 29074 | MRPL18 | −0.74 | 4.36E−06 | 4.10E−04 |
| ENSG00000243716 | 440345 | NPIPB5 | 0.46 | 4.42E−06 | 4.13E−04 |
| ENSG00000182093 | 7485 | WRB | −0.58 | 4.63E−06 | 4.30E−04 |
| ENSG00000213398 | 3931 | LCAT | 0.87 | 4.66E−06 | 4.31E−04 |
| ENSG00000079459 | 2222 | FDFT1 | −0.89 | 4.75E−06 | 4.36E−04 |
| ENSG00000163082 | 130367 | SGPP2 | −0.75 | 4.82E−06 | 4.40E−04 |
| ENSG00000181722 | 26137 | ZBTB20 | 0.71 | 4.96E−06 | 4.50E−04 |
| ENSG00000152953 | 55351 | STK32B | 2.03 | 5.05E−06 | 4.55E−04 |
| ENSG00000070193 | 2255 | FGF10 | 1.89 | 5.24E−06 | 4.67E−04 |
| ENSG00000106404 | 24146 | CLDN15 | 2.03 | 5.27E−06 | 4.67E−04 |
| ENSG00000107281 | 56654 | NPDC1 | 1.21 | 5.22E−06 | 4.67E−04 |
| ENSG00000107949 | 56647 | BCCIP | −0.61 | 5.32E−06 | 4.68E−04 |
| ENSG00000161055 | 92304 | SCGB3A1 | −1.74 | 6.01E−06 | 5.25E−04 |
| ENSG00000087916 | NA | NA | −1.75 | 6.14E−06 | 5.34E−04 |
| ENSG00000119139 | 9414 | TJP2 | −0.47 | 6.21E−06 | 5.36E−04 |
| ENSG00000151468 | 83643 | CCDC3 | 1.74 | 6.27E−06 | 5.39E−04 |
| ENSG00000205809 | 3822 | KLRC2 | −1.67 | 6.68E−06 | 5.70E−04 |
| ENSG00000131374 | 9779 | TBC1D5 | 0.35 | 6.76E−06 | 5.71E−04 |
| ENSG00000178741 | 9377 | COX5A | −0.63 | 6.73E−06 | 5.71E−04 |
| ENSG00000075142 | 6717 | SRI | −0.78 | 6.86E−06 | 5.73E−04 |
| ENSG00000141720 | NA | NA | 0.40 | 6.87E−06 | 5.73E−04 |
| ENSG00000159167 | 6781 | STC1 | 1.91 | 6.99E−06 | 5.80E−04 |
| ENSG00000081818 | 56131 | PCDHB4 | 1.46 | 7.29E−06 | 5.95E−04 |
| ENSG00000135773 | 10753 | CAPN9 | −0.90 | 7.27E−06 | 5.95E−04 |
| ENSG00000165300 | 26050 | SLITRK5 | 1.88 | 7.26E−06 | 5.95E−04 |
| ENSG00000133321 | 5920 | RARRES3 | −1.58 | 7.92E−06 | 6.43E−04 |
| ENSG00000160447 | 29941 | PKN3 | 1.30 | 8.24E−06 | 6.66E−04 |
| ENSG00000136859 | 23452 | ANGPTL2 | 1.38 | 8.33E−06 | 6.69E−04 |
| ENSG00000222009 | 149478 | BTBD19 | 1.14 | 8.54E−06 | 6.82E−04 |
| ENSG00000185250 | 285755 | PPIL6 | −1.17 | 8.59E−06 | 6.83E−04 |
| ENSG00000164330 | 1879 | EBF1 | 1.73 | 8.71E−06 | 6.86E−04 |
| ENSG00000182771 | 2894 | GRID1 | 1.81 | 8.73E−06 | 6.86E−04 |
| ENSG00000137842 | 80021 | TMEM62 | −0.91 | 8.80E−06 | 6.88E−04 |
| ENSG00000088538 | 1795 | DOCK3 | 1.25 | 8.88E−06 | 6.91E−04 |
| ENSG00000178966 | 80010 | RMI1 | −0.93 | 9.01E−06 | 6.97E−04 |
| ENSG00000133665 | 84332 | DYDC2 | −1.18 | 9.18E−06 | 7.06E−04 |
| ENSG00000117322 | 1380 | CR2 | 2.00 | 9.44E−06 | 7.23E−04 |
| ENSG00000100170 | 6523 | SLC5A1 | −1.54 | 1.00E−05 | 7.61E−04 |
| ENSG00000101384 | 182 | JAG1 | 0.73 | 1.00E−05 | 7.61E−04 |
| ENSG00000120756 | 5357 | PLS1 | −1.01 | 1.10E−05 | 8.29E−04 |
| ENSG00000102098 | 10389 | SCML2 | 1.41 | 1.12E−05 | 8.41E−04 |
| ENSG00000114200 | 590 | BCHE | 1.93 | 1.13E−05 | 8.41E−04 |
| ENSG00000013275 | 5704 | PSMC4 | −0.67 | 1.15E−05 | 8.50E−04 |
| ENSG00000108953 | 7531 | YWHAE | −0.48 | 1.15E−05 | 8.50E−04 |
| ENSG00000140181 | NA | NA | 1.02 | 1.17E−05 | 8.53E−04 |
| ENSG00000152939 | 153562 | MARVELD2 | −0.86 | 1.16E−05 | 8.53E−04 |
| ENSG00000162882 | 23498 | HAAO | 1.22 | 1.28E−05 | 9.29E−04 |
| ENSG00000088448 | 55608 | ANKRD10 | 0.59 | 1.31E−05 | 9.49E−04 |
| ENSG00000272636 | 8447 | DOC2B | 1.36 | 1.34E−05 | 9.64E−04 |
| ENSG00000052802 | 6307 | MSMO1 | −1.29 | 1.36E−05 | 9.70E−04 |
| ENSG00000077063 | 83992 | CTTNBP2 | 1.25 | 1.37E−05 | 9.70E−04 |
| ENSG00000112972 | 3157 | HMGCS1 | −1.11 | 1.36E−05 | 9.70E−04 |
| ENSG00000133019 | 1131 | CHRM3 | 1.44 | 1.37E−05 | 9.70E−04 |
| ENSG00000069329 | 55737 | VPS35 | −0.48 | 1.40E−05 | 9.88E−04 |
| ENSG00000166265 | 116159 | CYYR1 | 1.63 | 1.42E−05 | 9.94E−04 |
| ENSG00000159399 | 3099 | HK2 | −1.04 | 1.44E−05 | 1.00E−03 |
| ENSG00000257057 | NA | C11orf97 | −1.40 | 1.47E−05 | 1.02E−03 |
| ENSG00000065618 | 1308 | COL17A1 | 1.62 | 1.52E−05 | 1.05E−03 |
| ENSG00000163624 | 1040 | CDS1 | −1.01 | 1.67E−05 | 1.14E−03 |
| ENSG00000164764 | 157869 | SBSPON | 1.82 | 1.67E−05 | 1.14E−03 |
| ENSG00000171444 | 4163 | MCC | 0.99 | 1.67E−05 | 1.14E−03 |
| ENSG00000183323 | 202243 | CCDC125 | −1.05 | 1.66E−05 | 1.14E−03 |
| ENSG00000152332 | 127933 | UHMK1 | −0.55 | 1.71E−05 | 1.16E−03 |
| ENSG00000205277 | 10071 | MUC12 | 1.47 | 1.75E−05 | 1.18E−03 |
| ENSG00000091592 | 22861 | NLRP1 | 0.94 | 1.77E−05 | 1.18E−03 |
| ENSG00000101230 | 140862 | ISM1 | 1.31 | 1.77E−05 | 1.18E−03 |
| ENSG00000112981 | 8382 | NME5 | −1.10 | 1.79E−05 | 1.18E−03 |
| ENSG00000196263 | 57573 | ZNF471 | 0.81 | 1.79E−05 | 1.18E−03 |
| ENSG00000149809 | 7108 | TM7SF2 | −0.74 | 1.81E−05 | 1.19E−03 |
| ENSG00000139644 | 7009 | TMBIM6 | −0.60 | 1.83E−05 | 1.20E−03 |
| ENSG00000116138 | 23341 | DNAJC16 | −0.51 | 1.88E−05 | 1.21E−03 |
| ENSG00000143248 | 8490 | RGS5 | 1.70 | 1.88E−05 | 1.21E−03 |
| ENSG00000183644 | 399949 | C11orf88 | −1.23 | 1.88E−05 | 1.21E−03 |
| ENSG00000109814 | 7358 | UGDH | −0.76 | 1.89E−05 | 1.22E−03 |
| ENSG00000131475 | 84313 | VPS25 | −0.57 | 1.94E−05 | 1.24E−03 |
| ENSG00000183454 | 2903 | GRIN2A | 1.94 | 1.98E−05 | 1.26E−03 |
| ENSG00000057252 | 6646 | SOAT1 | −0.77 | 2.07E−05 | 1.31E−03 |
| ENSG00000106123 | 2051 | EPHB6 | 1.12 | 2.11E−05 | 1.31E−03 |
| ENSG00000133067 | 59352 | LGR6 | 1.51 | 2.11E−05 | 1.31E−03 |
| ENSG00000138356 | 316 | AOX1 | 1.79 | 2.09E−05 | 1.31E−03 |
| ENSG00000173269 | 79812 | MMRN2 | 1.69 | 2.09E−05 | 1.31E−03 |
| ENSG00000186642 | 5138 | PDE2A | 1.94 | 2.12E−05 | 1.31E−03 |
| ENSG00000178053 | 4291 | MLF1 | −1.06 | 2.15E−05 | 1.33E−03 |
| ENSG00000087995 | 339175 | METTL2A | −0.57 | 2.18E−05 | 1.34E−03 |
| ENSG00000130600 | 283120 | H19 | 1.87 | 2.18E−05 | 1.34E−03 |
| ENSG00000114573 | 523 | ATP6V1A | −0.62 | 2.24E−05 | 1.35E−03 |
| ENSG00000131203 | 3620 | IDO1 | −1.91 | 2.23E−05 | 1.35E−03 |
| ENSG00000143036 | 126969 | SLC44A3 | −0.74 | 2.21E−05 | 1.35E−03 |
| ENSG00000262209 | 56102 | PCDHGB3 | 1.37 | 2.22E−05 | 1.35E−03 |
| ENSG00000168079 | 286133 | SCARA5 | 1.77 | 2.29E−05 | 1.37E−03 |
| ENSG00000163534 | 115350 | FCRL1 | 1.93 | 2.35E−05 | 1.40E−03 |
| ENSG00000165304 | 9833 | MELK | −1.58 | 2.36E−05 | 1.40E−03 |
| ENSG00000101421 | 128866 | CHMP4B | −0.48 | 2.37E−05 | 1.41E−03 |
| ENSG00000162961 | 84661 | DPY30 | −0.75 | 2.53E−05 | 1.49E−03 |
| ENSG00000188931 | 257177 | CFAP126 | −1.15 | 2.53E−05 | 1.49E−03 |
| ENSG00000129467 | 196883 | ADCY4 | 1.60 | 2.55E−05 | 1.49E−03 |
| ENSG00000173530 | 8793 | TNFRSF10D | 0.83 | 2.54E−05 | 1.49E−03 |
| ENSG00000158683 | 168507 | PKD1L1 | 1.68 | 2.68E−05 | 1.56E−03 |
| ENSG00000000003 | 7105 | TSPAN6 | −1.01 | 2.70E−05 | 1.56E−03 |
| ENSG00000135679 | 4193 | MDM2 | −0.59 | 2.70E−05 | 1.56E−03 |
| ENSG00000241244 | NA | IGKV1D-16 | 1.79 | 2.76E−05 | 1.58E−03 |
| ENSG00000086548 | 4680 | CEACAM6 | −1.11 | 2.86E−05 | 1.64E−03 |
| ENSG00000005108 | 221981 | THSD7A | 1.06 | 2.87E−05 | 1.64E−03 |
| ENSG00000163001 | 112942 | CFAP36 | −0.57 | 2.91E−05 | 1.65E−03 |
| ENSG00000178445 | 2731 | GLDC | 1.32 | 2.92E−05 | 1.65E−03 |
| ENSG00000196586 | 4646 | MYO6 | −0.56 | 2.94E−05 | 1.66E−03 |
| ENSG00000130300 | 83483 | PLVAP | 1.89 | 2.97E−05 | 1.66E−03 |
| ENSG00000173068 | 54796 | BNC2 | 1.44 | 2.98E−05 | 1.66E−03 |
| ENSG00000211972 | NA | IGHV3-66 | 1.82 | 2.98E−05 | 1.66E−03 |
| ENSG00000163531 | 23114 | NFASC | 0.75 | 3.10E−05 | 1.72E−03 |
| ENSG00000145287 | 51316 | PLAC8 | −1.30 | 3.13E−05 | 1.72E−03 |
| ENSG00000157764 | 673 | BRAF | 0.36 | 3.14E−05 | 1.72E−03 |
| ENSG00000197959 | 26052 | DNM3 | 1.09 | 3.12E−05 | 1.72E−03 |
| ENSG00000119138 | 687 | KLF9 | 1.20 | 3.18E−05 | 1.74E−03 |
| ENSG00000185760 | 56479 | KCNQ5 | 1.62 | 3.26E−05 | 1.78E−03 |
| ENSG00000066382 | 744 | MPPED2 | 1.28 | 3.35E−05 | 1.80E−03 |
| ENSG00000088986 | 8655 | DYNLL1 | −0.77 | 3.32E−05 | 1.80E−03 |
| ENSG00000104413 | 54845 | ESRP1 | −0.77 | 3.35E−05 | 1.80E−03 |
| ENSG00000116906 | 8443 | GNPAT | −0.33 | 3.32E−05 | 1.80E−03 |
| ENSG00000165716 | 138311 | FAM69B | 1.58 | 3.34E−05 | 1.80E−03 |
| ENSG00000163263 | 388701 | C1orf189 | −1.27 | 3.44E−05 | 1.83E−03 |
| ENSG00000163993 | 6286 | S100P | −1.73 | 3.46E−05 | 1.84E−03 |
| ENSG00000102287 | 2564 | GABRE | 1.02 | 3.59E−05 | 1.90E−03 |
| ENSG00000011523 | 23177 | CEP68 | 0.74 | 3.67E−05 | 1.94E−03 |
| ENSG00000149212 | 143686 | SESN3 | 0.83 | 3.74E−05 | 1.96E−03 |
| ENSG00000174059 | 947 | CD34 | 1.78 | 3.73E−05 | 1.96E−03 |
| ENSG00000178125 | 286187 | PPP1R42 | −1.05 | 3.74E−05 | 1.96E−03 |
| ENSG00000145494 | 4726 | NDUFS6 | −0.39 | 3.84E−05 | 2.00E−03 |
| ENSG00000166562 | 90701 | SEC11C | −0.65 | 3.97E−05 | 2.06E−03 |
| ENSG00000261934 | 56107 | PCDHGA9 | 0.90 | 3.99E−05 | 2.06E−03 |
| ENSG00000126895 | 554 | AVPR2 | 1.24 | 4.01E−05 | 2.06E−03 |
| ENSG00000131844 | 64087 | MCCC2 | −0.44 | 4.04E−05 | 2.07E−03 |
| ENSG00000166813 | 374654 | KIF7 | 1.34 | 4.12E−05 | 2.11E−03 |
| ENSG00000185245 | 2811 | GP1BA | 1.18 | 4.22E−05 | 2.15E−03 |
| ENSG00000057704 | 57458 | TMCC3 | 1.54 | 4.29E−05 | 2.18E−03 |
| ENSG00000148541 | 220965 | FAM13C | 1.17 | 4.38E−05 | 2.21E−03 |
| ENSG00000224114 | NA | | −1.66 | 4.44E−05 | 2.24E−03 |
| ENSG00000068796 | 3796 | KIF2A | −0.75 | 4.51E−05 | 2.26E−03 |
| ENSG00000254986 | 10072 | DPP3 | −0.57 | 4.52E−05 | 2.26E−03 |
| ENSG00000266714 | NA | MYO15B | 0.93 | 4.52E−05 | 2.26E−03 |
| ENSG00000145824 | 9547 | CXCL14 | 1.80 | 4.67E−05 | 2.32E−03 |
| ENSG00000262576 | 56111 | PCDHGA4 | 1.35 | 4.70E−05 | 2.33E−03 |
| ENSG00000204604 | 90333 | ZNF468 | −0.74 | 4.96E−05 | 2.45E−03 |
| ENSG00000111834 | 345895 | RSPH4A | −1.07 | 5.07E−05 | 2.46E−03 |
| ENSG00000134202 | 2947 | GSTM3 | 1.58 | 4.99E−05 | 2.46E−03 |
| ENSG00000139193 | 939 | CD27 | 1.61 | 5.06E−05 | 2.46E−03 |
| ENSG00000142687 | 79932 | KIAA0319L | −0.66 | 5.07E−05 | 2.46E−03 |
| ENSG00000143653 | 51097 | SCCPDH | −0.61 | 5.05E−05 | 2.46E−03 |
| ENSG00000187244 | 4059 | BCAM | 0.98 | 5.00E−05 | 2.46E−03 |
| ENSG00000225698 | NA | IGHV3-72 | 1.84 | 5.26E−05 | 2.54E−03 |
| ENSG00000162896 | 5284 | PIGR | −1.10 | 5.29E−05 | 2.54E−03 |
| ENSG00000253485 | 56110 | PCDHGA5 | 1.40 | 5.32E−05 | 2.55E−03 |
| ENSG00000103184 | 9717 | SEC14L5 | −1.57 | 5.39E−05 | 2.57E−03 |
| ENSG00000117399 | 991 | CDC20 | −1.23 | 5.38E−05 | 2.57E−03 |
| ENSG00000128591 | 2318 | FLNC | 1.71 | 5.43E−05 | 2.57E−03 |
| ENSG00000153291 | 9481 | SLC25A27 | 0.80 | 5.42E−05 | 2.57E−03 |
| ENSG00000143153 | 481 | ATP1B1 | −0.71 | 5.54E−05 | 2.62E−03 |
| ENSG00000161798 | 362 | AQP5 | −1.15 | 5.59E−05 | 2.63E−03 |
| ENSG00000138413 | 3417 | IDH1 | −0.56 | 5.62E−05 | 2.64E−03 |
| ENSG00000070182 | 6710 | SPTB | 1.24 | 5.67E−05 | 2.65E−03 |
| ENSG00000165325 | 159989 | CCDC67 | −1.20 | 5.68E−05 | 2.65E−03 |
| ENSG00000073060 | 949 | SCARB1 | 1.42 | 5.92E−05 | 2.75E−03 |
| ENSG00000120437 | 39 | ACAT2 | −1.08 | 5.99E−05 | 2.77E−03 |
| ENSG00000181789 | 22820 | COPG1 | −0.45 | 6.01E−05 | 2.77E−03 |
| ENSG00000265150 | NA | NA | 1.14 | 6.00E−05 | 2.77E−03 |
| ENSG00000211934 | NA | IGHV1-2 | 1.76 | 6.10E−05 | 2.80E−03 |
| ENSG00000143772 | 3707 | ITPKB | 0.62 | 6.17E−05 | 2.82E−03 |
| ENSG00000109846 | 1410 | CRYAB | 1.44 | 6.45E−05 | 2.93E−03 |
| ENSG00000138031 | 109 | ADCY3 | 1.04 | 6.49E−05 | 2.93E−03 |
| ENSG00000148180 | 2934 | GSN | 0.67 | 6.48E−05 | 2.93E−03 |
| ENSG00000253910 | 56103 | PCDHGB2 | 1.36 | 6.45E−05 | 2.93E−03 |
| ENSG00000156966 | 93010 | B3GNT7 | −0.86 | 6.54E−05 | 2.94E−03 |
| ENSG00000130770 | 93974 | ATPIF1 | −0.63 | 6.74E−05 | 3.02E−03 |
| ENSG00000170312 | 983 | CDK1 | −1.01 | 6.74E−05 | 3.02E−03 |
| ENSG00000140263 | 6652 | SORD | −1.46 | 6.79E−05 | 3.03E−03 |
| ENSG00000139625 | 7786 | MAP3K12 | 0.86 | 7.13E−05 | 3.17E−03 |
| ENSG00000089356 | 5349 | FXYD3 | −1.11 | 7.18E−05 | 3.19E−03 |
| ENSG00000172349 | 3603 | IL16 | 0.87 | 7.60E−05 | 3.36E−03 |
| ENSG00000103534 | 79838 | TMC5 | −1.14 | 7.68E−05 | 3.39E−03 |
| ENSG00000138175 | 403 | ARL3 | −0.75 | 7.75E−05 | 3.40E−03 |
| ENSG00000204632 | 3135 | HLA-G | −1.80 | 7.73E−05 | 3.40E−03 |
| ENSG00000156675 | 80223 | RAB11FIP1 | −0.72 | 7.88E−05 | 3.44E−03 |
| ENSG00000081870 | 51668 | HSPB11 | −0.85 | 7.95E−05 | 3.45E−03 |
| ENSG00000133056 | 5287 | PIK3C2B | 0.47 | 7.94E−05 | 3.45E−03 |
| ENSG00000133313 | 55748 | CNDP2 | −0.77 | 7.98E−05 | 3.46E−03 |
| ENSG00000184903 | 83943 | IMMP2L | 0.82 | 8.32E−05 | 3.60E−03 |
| ENSG00000068976 | 5837 | PYGM | 1.50 | 8.37E−05 | 3.60E−03 |
| ENSG00000037042 | 27175 | TUBG2 | 0.71 | 8.40E−05 | 3.61E−03 |
| ENSG00000168056 | 4054 | LTBP3 | 0.85 | 8.45E−05 | 3.61E−03 |
| ENSG00000168067 | 5871 | MAP4K2 | 0.60 | 8.44E−05 | 3.61E−03 |
| ENSG00000119630 | 5228 | PGF | 1.77 | 8.51E−05 | 3.62E−03 |
| ENSG00000116678 | 3953 | LEPR | 1.54 | 8.58E−05 | 3.64E−03 |
| ENSG00000166226 | 10576 | CCT2 | −0.59 | 8.78E−05 | 3.72E−03 |
| ENSG00000167088 | 6632 | SNRPD1 | −0.54 | 8.80E−05 | 3.72E−03 |
| ENSG00000105696 | 25789 | TMEM59L | 1.67 | 8.95E−05 | 3.73E−03 |
| ENSG00000105711 | 6324 | SCN1B | 1.26 | 8.96E−05 | 3.73E−03 |
| ENSG00000134339 | 6289 | SAA2 | −1.78 | 8.97E−05 | 3.73E−03 |
| ENSG00000139631 | 51380 | CSAD | 0.90 | 8.88E−05 | 3.73E−03 |
| ENSG00000162928 | 5194 | PEX13 | −0.59 | 8.91E−05 | 3.73E−03 |
| ENSG00000005189 | 81691 | | −0.88 | 9.05E−05 | 3.74E−03 |
| ENSG00000137947 | 2959 | GTF2B | −0.53 | 9.01E−05 | 3.74E−03 |
| ENSG00000166793 | 219539 | YPEL4 | 1.34 | 9.06E−05 | 3.74E−03 |
| ENSG00000156299 | 7074 | TIAM1 | 0.90 | 9.12E−05 | 3.75E−03 |
| ENSG00000138385 | 6741 | SSB | −0.66 | 9.17E−05 | 3.77E−03 |
| ENSG00000231259 | NA | | 0.99 | 9.22E−05 | 3.77E−03 |
| ENSG00000150773 | 120379 | PIH1D2 | −1.07 | 9.31E−05 | 3.80E−03 |
| ENSG00000172586 | 118487 | CHCHD1 | −0.80 | 9.32E−05 | 3.80E−03 |
| ENSG00000134058 | 1022 | CDK7 | −0.78 | 9.49E−05 | 3.84E−03 |
| ENSG00000196636 | 57001 | SDHAF3 | −0.92 | 9.47E−05 | 3.84E−03 |
| ENSG00000116288 | 11315 | PARK7 | −0.33 | 9.92E−05 | 4.00E−03 |
| ENSG00000078596 | 9452 | ITM2A | 1.13 | 1.01E−04 | 4.05E−03 |
| ENSG00000092096 | 51310 | SLC22A17 | 1.07 | 1.01E−04 | 4.05E−03 |
| ENSG00000233974 | NA | | 1.28 | 1.01E−04 | 4.05E−03 |
| ENSG00000104419 | 10397 | NDRG1 | 1.20 | 1.02E−04 | 4.07E−03 |
| ENSG00000197993 | 3792 | KEL | 1.38 | 1.02E−04 | 4.08E−03 |
| ENSG00000000419 | 8813 | DPM1 | −0.56 | 1.03E−04 | 4.08E−03 |
| ENSG00000075089 | 64431 | ACTR6 | −0.66 | 1.04E−04 | 4.08E−03 |
| ENSG00000101443 | 10406 | WFDC2 | −1.35 | 1.04E−04 | 4.08E−03 |
| ENSG00000140526 | 11057 | ABHD2 | −0.77 | 1.03E−04 | 4.08E−03 |
| ENSG00000198087 | 23607 | CD2AP | −0.57 | 1.04E−04 | 4.08E−03 |
| ENSG00000149150 | 8501 | SLC43A1 | 0.96 | 1.05E−04 | 4.11E−03 |
| ENSG00000251039 | NA | IGKV2D-40 | 1.53 | 1.05E−04 | 4.11E−03 |
| ENSG00000172935 | 116535 | MRGPRF | 1.56 | 1.06E−04 | 4.13E−03 |
| ENSG00000221716 | 677799 | SNORA11 | 1.44 | 1.06E−04 | 4.14E−03 |
| ENSG00000148400 | 4851 | NOTCH1 | 0.61 | 1.08E−04 | 4.16E−03 |
| ENSG00000224411 | NA | HSP90AA2P | −0.80 | 1.07E−04 | 4.16E−03 |
| ENSG00000103168 | 9013 | TAF1C | 0.98 | 1.11E−04 | 4.28E−03 |
| ENSG00000032742 | 8100 | IFT88 | −0.68 | 1.13E−04 | 4.34E−03 |
| ENSG00000112297 | 202 | AIM1 | −0.48 | 1.15E−04 | 4.42E−03 |
| ENSG00000172037 | 3913 | LAMB2 | 0.72 | 1.15E−04 | 4.42E−03 |
| ENSG00000119328 | 54942 | FAM206A | −0.63 | 1.17E−04 | 4.45E−03 |
| ENSG00000164114 | 79884 | MAP9 | −0.78 | 1.17E−04 | 4.45E−03 |
| ENSG00000100591 | 10598 | AHSA1 | −0.74 | 1.22E−04 | 4.62E−03 |
| ENSG00000127884 | 1892 | ECHS1 | −0.42 | 1.24E−04 | 4.62E−03 |
| ENSG00000134709 | 51361 | HOOK1 | −0.81 | 1.23E−04 | 4.62E−03 |
| ENSG00000153904 | 23576 | DDAH1 | −0.53 | 1.22E−04 | 4.62E−03 |
| ENSG00000165929 | 123036 | TC2N | −0.81 | 1.24E−04 | 4.62E−03 |
| ENSG00000169550 | 143662 | MUC15 | −1.09 | 1.23E−04 | 4.62E−03 |
| ENSG00000214776 | NA | | 1.28 | 1.23E−04 | 4.62E−03 |
| ENSG00000163406 | 6565 | SLC15A2 | −0.97 | 1.24E−04 | 4.63E−03 |
| ENSG00000177054 | 54503 | ZDHHC13 | −0.84 | 1.24E−04 | 4.63E−03 |
| ENSG00000100626 | 57452 | GALNT16 | 1.34 | 1.27E−04 | 4.70E−03 |
| ENSG00000129055 | 25847 | ANAPC13 | −0.81 | 1.27E−04 | 4.70E−03 |
| ENSG00000080824 | 3320 | HSP90AA1 | −0.67 | 1.28E−04 | 4.72E−03 |
| ENSG00000119912 | 3416 | IDE | −0.54 | 1.31E−04 | 4.81E−03 |
| ENSG00000148346 | 3934 | LCN2 | −1.55 | 1.31E−04 | 4.82E−03 |
| ENSG00000211459 | NA | MT-RNR1 | 0.83 | 1.32E−04 | 4.85E−03 |
| ENSG00000175309 | 85007 | PHYKPL | 0.77 | 1.33E−04 | 4.87E−03 |
| ENSG00000178460 | 157777 | MCMDC2 | −1.36 | 1.33E−04 | 4.87E−03 |
| ENSG00000159079 | 56683 | C21orf59 | −1.02 | 1.37E−04 | 4.99E−03 |
| ENSG00000137691 | 85016 | C11orf70 | −1.02 | 1.38E−04 | 5.02E−03 |
| ENSG00000185585 | 169611 | OLFML2A | 1.02 | 1.39E−04 | 5.02E−03 |
| ENSG00000127838 | 25953 | PNKD | −0.49 | 1.40E−04 | 5.06E−03 |
| ENSG00000143387 | 1513 | CTSK | 1.44 | 1.40E−04 | 5.06E−03 |
| ENSG00000142733 | 9064 | MAP3K6 | 0.60 | 1.42E−04 | 5.11E−03 |
| ENSG00000147862 | 4781 | NFIB | 0.61 | 1.46E−04 | 5.22E−03 |
| ENSG00000115339 | 2591 | GALNT3 | −0.90 | 1.46E−04 | 5.23E−03 |
| ENSG00000073969 | 4905 | NSF | −0.51 | 1.48E−04 | 5.28E−03 |
| ENSG00000109971 | 3312 | HSPA8 | −0.53 | 1.49E−04 | 5.29E−03 |
| ENSG00000116299 | 57535 | KIAA1324 | −0.76 | 1.51E−04 | 5.35E−03 |
| ENSG00000092421 | 57556 | SEMA6A | 1.00 | 1.52E−04 | 5.36E−03 |
| ENSG00000129493 | 25938 | HEATR5A | −0.39 | 1.52E−04 | 5.36E−03 |
| ENSG00000197279 | 7718 | ZNF165 | −0.98 | 1.51E−04 | 5.36E−03 |
| ENSG00000161544 | 114757 | CYGB | 0.93 | 1.54E−04 | 5.41E−03 |
| ENSG00000211890 | NA | IGHA2 | 1.70 | 1.54E−04 | 5.42E−03 |
| ENSG00000133063 | 1118 | CHIT1 | 1.69 | 1.57E−04 | 5.50E−03 |
| ENSG00000160469 | 84446 | BRSK1 | 1.36 | 1.62E−04 | 5.63E−03 |
| ENSG00000185681 | 254956 | MORN5 | −1.16 | 1.61E−04 | 5.63E−03 |
| ENSG00000122420 | 5737 | PTGFR | −1.33 | 1.63E−04 | 5.66E−03 |
| ENSG00000130066 | 6303 | SAT1 | −0.63 | 1.63E−04 | 5.66E−03 |
| ENSG00000179222 | 9500 | MAGED1 | −0.58 | 1.65E−04 | 5.72E−03 |
| ENSG00000095380 | 54187 | NANS | −0.75 | 1.68E−04 | 5.79E−03 |
| ENSG00000089127 | 4938 | OAS1 | −1.31 | 1.69E−04 | 5.83E−03 |
| ENSG00000066185 | 84217 | ZMYND12 | −1.04 | 1.70E−04 | 5.83E−03 |
| ENSG00000234745 | 3106 | HLA-B | −0.84 | 1.71E−04 | 5.85E−03 |
| ENSG00000175792 | 8607 | RUVBL1 | −0.80 | 1.72E−04 | 5.88E−03 |
| ENSG00000101846 | 412 | STS | −0.54 | 1.75E−04 | 5.97E−03 |
| ENSG00000206149 | 440248 | HERC2P9 | 0.90 | 1.78E−04 | 6.06E−03 |
| ENSG00000076685 | 22978 | NT5C2 | −0.42 | 1.79E−04 | 6.09E−03 |
| ENSG00000069702 | 7049 | TGFBR3 | 0.94 | 1.80E−04 | 6.10E−03 |
| ENSG00000153292 | 266977 | ADGRF1 | −1.45 | 1.81E−04 | 6.11E−03 |
| ENSG00000059145 | 64718 | UNKL | 0.91 | 1.83E−04 | 6.14E−03 |
| ENSG00000113212 | 56129 | PCDHB7 | 1.36 | 1.83E−04 | 6.14E−03 |
| ENSG00000177455 | 930 | CD19 | 1.71 | 1.82E−04 | 6.14E−03 |
| ENSG00000106483 | 6424 | SFRP4 | 1.62 | 1.83E−04 | 6.14E−03 |
| ENSG00000013016 | 30845 | EHD3 | 1.11 | 1.85E−04 | 6.18E−03 |
| ENSG00000168961 | 3965 | LGALS9 | −0.70 | 1.85E−04 | 6.18E−03 |
| ENSG00000163131 | 1520 | CTSS | −0.77 | 1.87E−04 | 6.24E−03 |
| ENSG00000147041 | 94122 | SYTL5 | −1.12 | 1.89E−04 | 6.25E−03 |
| ENSG00000214517 | 51400 | PPME1 | −0.60 | 1.89E−04 | 6.25E−03 |
| ENSG00000211956 | NA | IGHV4-34 | 1.68 | 1.89E−04 | 6.26E−03 |
| ENSG00000110675 | 55531 | ELMOD1 | 1.58 | 1.91E−04 | 6.29E−03 |
| ENSG00000128039 | 79644 | SRD5A3 | −0.97 | 1.93E−04 | 6.35E−03 |
| ENSG00000185813 | 5833 | PCYT2 | −0.92 | 1.95E−04 | 6.40E−03 |
| ENSG00000184254 | 220 | ALDH1A3 | −0.68 | 2.01E−04 | 6.58E−03 |
| ENSG00000084207 | 2950 | GSTP1 | −0.85 | 2.03E−04 | 6.59E−03 |
| ENSG00000123243 | 80760 | ITIH5 | 1.40 | 2.03E−04 | 6.59E−03 |
| ENSG00000143106 | 5686 | PSMA5 | −0.56 | 2.02E−04 | 6.59E−03 |
| ENSG00000167775 | 51293 | CD320 | 0.85 | 2.03E−04 | 6.59E−03 |
| ENSG00000187391 | 9863 | MAGI2 | 0.92 | 2.03E−04 | 6.59E−03 |
| ENSG00000168477 | 7148 | TNXB | 1.50 | 2.08E−04 | 6.74E−03 |
| ENSG00000241755 | NA | IGKV1-9 | 1.66 | 2.11E−04 | 6.81E−03 |
| ENSG00000136810 | 7295 | TXN | −0.77 | 2.13E−04 | 6.87E−03 |
| ENSG00000104332 | 6422 | SFRP1 | 1.68 | 2.14E−04 | 6.87E−03 |
| ENSG00000184076 | 29796 | UQCR10 | −0.78 | 2.14E−04 | 6.87E−03 |
| ENSG00000106537 | 27075 | TSPAN13 | −0.82 | 2.15E−04 | 6.88E−03 |
| ENSG00000127362 | 50831 | TAS2R3 | 1.02 | 2.16E−04 | 6.91E−03 |
| ENSG00000160213 | 1476 | CSTB | −0.51 | 2.17E−04 | 6.91E−03 |
| ENSG00000067064 | 3422 | IDI1 | −0.95 | 2.19E−04 | 6.95E−03 |
| ENSG00000143196 | 1805 | DPT | 1.57 | 2.21E−04 | 7.00E−03 |
| ENSG00000105929 | 50617 | ATP6V0A4 | −1.54 | 2.24E−04 | 7.09E−03 |
| ENSG00000145391 | 80854 | SETD7 | 0.69 | 2.27E−04 | 7.16E−03 |
| ENSG00000066735 | 26153 | KIF26A | 1.55 | 2.28E−04 | 7.19E−03 |
| ENSG00000108179 | 10105 | PPIF | −0.65 | 2.30E−04 | 7.22E−03 |
| ENSG00000124107 | 6590 | SLPI | −1.05 | 2.30E−04 | 7.22E−03 |
| ENSG00000163902 | 6184 | RPN1 | −0.42 | 2.31E−04 | 7.22E−03 |
| ENSG00000198919 | 9666 | DZIP3 | −0.79 | 2.31E−04 | 7.22E−03 |
| ENSG00000134363 | 10468 | FST | 1.30 | 2.32E−04 | 7.24E−03 |
| ENSG00000133328 | 54979 | HRASLS2 | −1.41 | 2.35E−04 | 7.32E−03 |
| ENSG00000100968 | 4776 | NFATC4 | 1.14 | 2.36E−04 | 7.33E−03 |
| ENSG00000097007 | 25 | ABL1 | 0.38 | 2.41E−04 | 7.46E−03 |
| ENSG00000147155 | 10682 | EBP | −0.75 | 2.41E−04 | 7.46E−03 |
| ENSG00000184432 | 9276 | COPB2 | −0.48 | 2.43E−04 | 7.50E−03 |
| ENSG00000135387 | 4076 | CAPRIN1 | −0.38 | 2.44E−04 | 7.52E−03 |
| ENSG00000177169 | 8408 | ULK1 | 0.49 | 2.47E−04 | 7.58E−03 |
| ENSG00000086232 | 27102 | EIF2AK1 | −0.46 | 2.49E−04 | 7.63E−03 |
| ENSG00000132141 | 10693 | CCT6B | −0.92 | 2.50E−04 | 7.66E−03 |
| ENSG00000102900 | 9688 | NUP93 | −0.43 | 2.51E−04 | 7.66E−03 |
| ENSG00000117528 | 5825 | ABCD3 | −0.57 | 2.52E−04 | 7.67E−03 |
| ENSG00000167523 | 124045 | SPATA33 | −0.90 | 2.54E−04 | 7.74E−03 |
| ENSG00000203734 | 345930 | ECT2L | −0.91 | 2.60E−04 | 7.88E−03 |
| ENSG00000044115 | 1495 | CTNNA1 | −0.40 | 2.65E−04 | 8.04E−03 |
| ENSG00000065361 | 2065 | ERBB3 | −0.86 | 2.67E−04 | 8.06E−03 |
| ENSG00000138294 | NA | NA | −1.54 | 2.67E−04 | 8.06E−03 |
| ENSG00000197697 | NA | NA | −0.77 | 2.68E−04 | 8.07E−03 |
| ENSG00000184983 | 4700 | NDUFA6 | −0.52 | 2.69E−04 | 8.07E−03 |
| ENSG00000155254 | 83742 | MARVELD1 | 1.25 | 2.73E−04 | 8.17E−03 |
| ENSG00000081760 | 65985 | AACS | −0.64 | 2.74E−04 | 8.17E−03 |
| ENSG00000132746 | 222 | ALDH3B2 | −1.46 | 2.74E−04 | 8.17E−03 |
| ENSG00000151364 | 65987 | KCTD14 | −1.25 | 2.73E−04 | 8.17E−03 |
| ENSG00000164347 | 84340 | GFM2 | −0.86 | 2.75E−04 | 8.17E−03 |
| ENSG00000105875 | 29062 | WDR91 | 0.65 | 2.77E−04 | 8.21E−03 |
| ENSG00000136153 | 4008 | LMO7 | −0.80 | 2.81E−04 | 8.31E−03 |
| ENSG00000116133 | 1718 | DHCR24 | −0.76 | 2.83E−04 | 8.35E−03 |
| ENSG00000124201 | 57169 | ZNFX1 | −0.52 | 2.84E−04 | 8.37E−03 |
| ENSG00000158234 | 55179 | FAIM | −0.82 | 2.85E−04 | 8.38E−03 |
| ENSG00000144834 | 29114 | TAGLN3 | −1.37 | 2.86E−04 | 8.41E−03 |
| ENSG00000099290 | 387680 | FAM21A | −1.43 | 2.87E−04 | 8.42E−03 |
| ENSG00000149021 | 7356 | SCGB1A1 | −1.45 | 2.90E−04 | 8.49E−03 |
| ENSG00000135378 | 79056 | PRRG4 | −0.92 | 2.92E−04 | 8.54E−03 |
| ENSG00000134744 | 23318 | ZCCHC11 | 0.50 | 2.99E−04 | 8.71E−03 |
| ENSG00000005175 | 79657 | RPAP3 | −0.38 | 3.01E−04 | 8.77E−03 |
| ENSG00000173467 | 155465 | AGR3 | −0.78 | 3.04E−04 | 8.81E−03 |
| ENSG00000164251 | 2150 | F2RL1 | −0.87 | 3.12E−04 | 9.03E−03 |
| ENSG00000253250 | 100127983 | C8orf88 | 1.46 | 3.14E−04 | 9.09E−03 |
| ENSG00000090061 | 8812 | CCNK | −0.34 | 3.23E−04 | 9.32E−03 |
| ENSG00000101204 | 1137 | CHRNA4 | 1.64 | 3.23E−04 | 9.32E−03 |
| ENSG00000143127 | 8515 | ITGA10 | 1.00 | 3.26E−04 | 9.36E−03 |
| ENSG00000187800 | 375033 | PEAR1 | 1.54 | 3.29E−04 | 9.45E−03 |
| ENSG00000162909 | 824 | CAPN2 | −0.49 | 3.32E−04 | 9.51E−03 |
| ENSG00000103316 | 1428 | CRYM | −1.06 | 3.34E−04 | 9.55E−03 |
| ENSG00000069869 | 4734 | NEDD4 | 0.92 | 3.37E−04 | 9.58E−03 |
| ENSG00000162407 | 8613 | PPAP2B | 1.12 | 3.37E−04 | 9.58E−03 |
| ENSG00000143933 | 805 | CALM2 | −0.58 | 3.40E−04 | 9.66E−03 |
| ENSG00000125827 | 56255 | TMX4 | 0.60 | 3.41E−04 | 9.67E−03 |
| ENSG00000185055 | NA | EFCAB10 | −1.13 | 3.47E−04 | 9.82E−03 |
| ENSG00000029993 | 3149 | HMGB3 | −0.96 | 3.54E−04 | 9.94E−03 |
| ENSG00000135424 | 3679 | ITGA7 | 1.29 | 3.53E−04 | 9.94E−03 |
| ENSG00000158373 | 3017 | HIST1H2BD | −0.54 | 3.52E−04 | 9.94E−03 |
| ENSG00000165097 | 221656 | KDM1B | −0.71 | 3.53E−04 | 9.94E−03 |
| ENSG00000173432 | 6288 | SAA1 | −1.61 | 3.55E−04 | 9.95E−03 |

UIP (n = 3 samples); Non-UIP (n = 5 samples). Positive log2 fold change value indicates over-expression in UIP relative to Non UIP; negative log2 value indicates under-expression in UIP relative to Non UIP. In this analysis only patients without any smoking history were evaluated, hence this subset harbored only non-smokers.

TABLE 11
Differentially expressed genes in UIP samples
from smokers vs. Non-UIP samples from smokers.
Table 11. Smokers

| | | | Log2 Fold | | |
|---|---|---|---|---|---|
| | | | Change | | |
| Ensembl ID | Entrez ID | Gene symbol | (UIP/NonUIP) | p value | FDR P value |
| ENSG00000137968 | 204962 | SLC44A5 | 3.58 | 2.38E−20 | 3.68E−16 |
| ENSG00000099968 | 23786 | BCL2L13 | −0.68 | 3.12E−18 | 2.41E−14 |
| ENSG00000168329 | 1524 | CX3CR1 | 2.27 | 8.45E−14 | 4.35E−10 |
| ENSG00000088882 | 56265 | CPXM1 | 2.72 | 1.16E−11 | 4.49E−08 |
| ENSG00000152672 | 165530 | CLEC4F | 2.63 | 1.78E−11 | 5.49E−08 |
| ENSG00000129204 | 9098 | USP6 | 2.74 | 5.40E−11 | 1.39E−07 |
| ENSG00000132823 | 51526 | OSER1 | −0.56 | 3.27E−10 | 7.21E−07 |
| ENSG00000177666 | 57104 | PNPLA2 | −0.84 | 9.60E−10 | 1.85E−06 |
| ENSG00000125730 | 718 | C3 | 1.60 | 1.11E−09 | 1.90E−06 |
| ENSG00000198074 | 57016 | AKR1B10 | −2.89 | 1.97E−09 | 3.04E−06 |
| ENSG00000198142 | 65124 | SOWAHC | −1.00 | 3.09E−09 | 4.34E−06 |
| ENSG00000112130 | 9025 | RNF8 | −0.56 | 4.53E−09 | 5.83E−06 |
| ENSG00000255112 | 57132 | CHMP1B | −0.86 | 1.94E−08 | 2.31E−05 |
| ENSG00000145888 | 2741 | GLRA1 | 2.19 | 2.15E−08 | 2.37E−05 |
| ENSG00000151632 | 1646 | AKR1C2 | −2.37 | 2.60E−08 | 2.68E−05 |
| ENSG00000238741 | 677767 | SCARNA7 | 0.58 | 2.90E−08 | 2.80E−05 |
| ENSG00000148948 | 57689 | LRRC4C | 2.14 | 3.13E−08 | 2.84E−05 |
| ENSG00000179344 | 3119 | HLA-DQB1 | 2.30 | 2.89E−07 | 2.35E−04 |
| ENSG00000211789 | NA | TRAV12-2 | 2.44 | 2.75E−07 | 2.35E−04 |
| ENSG00000106565 | 28959 | TMEM176B | 1.55 | 3.23E−07 | 2.44E−04 |
| ENSG00000204338 | NA | CYP21A1P | 2.23 | 3.32E−07 | 2.44E−04 |
| ENSG00000010932 | 2326 | FMO1 | 2.17 | 4.30E−07 | 3.01E−04 |
| ENSG00000103742 | 57722 | IGDCC4 | 2.37 | 4.82E−07 | 3.24E−04 |
| ENSG00000162692 | 7412 | VCAM1 | 2.12 | 5.91E−07 | 3.80E−04 |
| ENSG00000158481 | 911 | CD1C | 1.96 | 6.26E−07 | 3.87E−04 |
| ENSG00000136098 | 4752 | NEK3 | 0.67 | 6.66E−07 | 3.95E−04 |
| ENSG00000134375 | 10440 | TIMM17A | −0.48 | 7.00E−07 | 4.00E−04 |
| ENSG00000127951 | 10875 | FGL2 | 1.23 | 1.18E−06 | 6.49E−04 |
| ENSG00000185022 | 23764 | MAFF | −1.68 | 1.22E−06 | 6.49E−04 |
| ENSG00000100079 | 3957 | LGALS2 | 1.93 | 1.51E−06 | 7.56E−04 |
| ENSG00000151572 | 121601 | ANO4 | 2.28 | 1.57E−06 | 7.56E−04 |
| ENSG00000238460 | NA | | 1.65 | 1.52E−06 | 7.56E−04 |
| ENSG00000187527 | 344905 | ATP13A5 | −2.18 | 1.99E−06 | 9.29E−04 |
| ENSG00000176153 | 2877 | GPX2 | −2.11 | 2.29E−06 | 1.04E−03 |
| ENSG00000105559 | 57664 | PLEKHA4 | 1.30 | 2.99E−06 | 1.32E−03 |
| ENSG00000178115 | NA | GOLGA8Q | 2.05 | 3.27E−06 | 1.40E−03 |
| ENSG00000137033 | 90865 | IL33 | 1.41 | 4.10E−06 | 1.68E−03 |
| ENSG00000196735 | 3117 | HLA-DQA1 | 2.10 | 4.14E−06 | 1.68E−03 |
| ENSG00000007944 | 29116 | MYLIP | −0.46 | 4.53E−06 | 1.79E−03 |
| ENSG00000130695 | 64793 | CEP85 | −0.76 | 4.86E−06 | 1.88E−03 |
| ENSG00000262539 | NA | | −2.01 | 5.01E−06 | 1.89E−03 |
| ENSG00000174194 | NA | NA | 1.27 | 5.42E−06 | 1.99E−03 |
| ENSG00000178187 | 285676 | ZNF454 | 1.08 | 6.40E−06 | 2.25E−03 |
| ENSG00000204256 | 6046 | BRD2 | −0.33 | 6.28E−06 | 2.25E−03 |
| ENSG00000002933 | 55365 | TMEM176A | 1.30 | 6.91E−06 | 2.36E−03 |
| ENSG00000196139 | 8644 | AKR1C3 | −1.45 | 7.02E−06 | 2.36E−03 |
| ENSG00000186529 | 4051 | CYP4F3 | −1.80 | 8.32E−06 | 2.66E−03 |
| ENSG00000227097 | NA | RPS28P7 | 1.59 | 8.45E−06 | 2.66E−03 |
| ENSG00000244486 | 91179 | SCARF2 | 1.00 | 8.18E−06 | 2.66E−03 |
| ENSG00000172985 | 344558 | SH3RF3 | 0.84 | 9.51E−06 | 2.94E−03 |
| ENSG00000023171 | 57476 | GRAMD1B | −1.22 | 1.13E−05 | 3.21E−03 |
| ENSG00000065613 | 9748 | SLK | −0.49 | 1.09E−05 | 3.21E−03 |
| ENSG00000143603 | 3782 | KCNN3 | 1.09 | 1.07E−05 | 3.21E−03 |
| ENSG00000154096 | 7070 | THY1 | 2.05 | 1.14E−05 | 3.21E−03 |
| ENSG00000178562 | 940 | CD28 | 2.08 | 1.11E−05 | 3.21E−03 |
| ENSG00000196839 | 100 | ADA | 1.10 | 1.16E−05 | 3.21E−03 |
| ENSG00000159618 | 221188 | ADGRG5 | 1.59 | 1.21E−05 | 3.27E−03 |
| ENSG00000128309 | 4357 | MPST | −0.73 | 1.36E−05 | 3.61E−03 |
| ENSG00000168229 | 5729 | PTGDR | 1.61 | 1.39E−05 | 3.62E−03 |
| ENSG00000125510 | 4987 | OPRL1 | 1.56 | 1.45E−05 | 3.73E−03 |
| ENSG00000017427 | 3479 | IGF1 | 1.86 | 1.54E−05 | 3.83E−03 |
| ENSG00000159228 | 873 | CBR1 | −1.00 | 1.53E−05 | 3.83E−03 |
| ENSG00000136490 | 80774 | LIMD2 | 1.68 | 1.76E−05 | 4.19E−03 |
| ENSG00000177156 | 6888 | TALDO1 | −0.78 | 1.76E−05 | 4.19E−03 |
| ENSG00000225614 | 84627 | ZNF469 | 0.98 | 1.72E−05 | 4.19E−03 |
| ENSG00000218336 | 55714 | TENM3 | 1.69 | 1.88E−05 | 4.40E−03 |
| ENSG00000151693 | 8853 | ASAP2 | −0.60 | 1.97E−05 | 4.54E−03 |
| ENSG00000211941 | NA | IGHV3-11 | 2.04 | 2.00E−05 | 4.54E−03 |
| ENSG00000140961 | 29948 | OSGIN1 | −1.25 | 2.11E−05 | 4.73E−03 |
| ENSG00000124782 | 6239 | RREB1 | −0.41 | 2.30E−05 | 5.08E−03 |
| ENSG00000103222 | 4363 | ABCC1 | −0.81 | 2.55E−05 | 5.47E−03 |
| ENSG00000196664 | 51284 | TLR7 | 1.53 | 2.54E−05 | 5.47E−03 |
| ENSG00000148357 | 256158 | HMCN2 | 1.92 | 2.61E−05 | 5.53E−03 |
| ENSG00000124151 | 8202 | NCOA3 | −0.39 | 2.87E−05 | 6.00E−03 |
| ENSG00000211653 | NA | IGLV1-40 | 1.99 | 2.98E−05 | 6.06E−03 |
| ENSG00000230006 | 645784 | ANKRD36BP2 | 1.82 | 2.97E−05 | 6.06E−03 |
| ENSG00000106809 | 4969 | OGN | 1.85 | 3.11E−05 | 6.23E−03 |
| ENSG00000162877 | 148811 | PM20D1 | 1.70 | 3.17E−05 | 6.28E−03 |
| ENSG00000128016 | 7538 | ZFP36 | −1.41 | 3.51E−05 | 6.77E−03 |
| ENSG00000196345 | 55888 | ZKSCAN7 | 0.83 | 3.49E−05 | 6.77E−03 |
| ENSG00000108821 | 1277 | COL1A1 | 1.88 | 3.56E−05 | 6.78E−03 |
| ENSG00000137573 | 23213 | SULF1 | 1.63 | 3.68E−05 | 6.93E−03 |
| ENSG00000197993 | 3792 | KEL | 1.86 | 3.93E−05 | 7.32E−03 |
| ENSG00000170153 | 57484 | RNF150 | 1.44 | 4.36E−05 | 8.00E−03 |
| ENSG00000130513 | 9518 | GDF15 | −1.73 | 4.55E−05 | 8.20E−03 |
| ENSG00000174123 | 81793 | TLR10 | 1.81 | 4.57E−05 | 8.20E−03 |
| ENSG00000110076 | 9379 | NRXN2 | 1.95 | 4.68E−05 | 8.30E−03 |
| ENSG00000182551 | 55256 | ADI1 | −0.52 | 5.20E−05 | 9.08E−03 |
| ENSG00000182557 | 201305 | SPNS3 | 1.65 | 5.23E−05 | 9.08E−03 |
| ENSG00000117215 | 26279 | PLA2G2D | 1.94 | 5.54E−05 | 9.50E−03 |
| ENSG00000128285 | 2847 | MCHR1 | 1.85 | 5.80E−05 | 9.83E−03 |
| ENSG00000183813 | 1233 | CCR4 | 1.90 | 6.00E−05 | 1.01E−02 |
| ENSG00000007312 | 974 | CD79B | 1.81 | 6.30E−05 | 1.04E−02 |
| ENSG00000163817 | 54716 | SLC6A20 | 1.85 | 6.43E−05 | 1.06E−02 |
| ENSG00000102802 | 84935 | MEDAG | 1.85 | 6.69E−05 | 1.09E−02 |
| ENSG00000101134 | 55816 | DOK5 | 1.90 | 7.00E−05 | 1.10E−02 |
| ENSG00000102362 | 94121 | SYTL4 | −0.80 | 6.89E−05 | 1.10E−02 |
| ENSG00000128000 | 163131 | ZNF780B | 0.66 | 7.03E−05 | 1.10E−02 |
| ENSG00000256229 | 90649 | ZNF486 | 1.00 | 7.04E−05 | 1.10E−02 |
| ENSG00000086102 | 4799 | NFX1 | −0.36 | 7.46E−05 | 1.14E−02 |
| ENSG00000099875 | 2872 | MKNK2 | −0.58 | 7.58E−05 | 1.14E−02 |
| ENSG00000171502 | 255631 | COL24A1 | 1.24 | 7.69E−05 | 1.14E−02 |
| ENSG00000211637 | NA | IGLV4-69 | 1.91 | 7.66E−05 | 1.14E−02 |
| ENSG00000244731 | 720 | C4A | 1.50 | 7.70E−05 | 1.14E−02 |
| ENSG00000082641 | 4779 | NFE2L1 | −0.32 | 7.79E−05 | 1.14E−02 |
| ENSG00000136802 | 56262 | LRRC8A | −0.91 | 8.12E−05 | 1.18E−02 |
| ENSG00000225784 | NA | NA | 1.81 | 8.26E−05 | 1.19E−02 |
| ENSG00000181631 | 53829 | P2RY13 | 1.57 | 8.40E−05 | 1.20E−02 |
| ENSG00000116031 | 50489 | CD207 | 1.47 | 8.80E−05 | 1.25E−02 |
| ENSG00000108106 | 27338 | UBE2S | −0.71 | 9.40E−05 | 1.32E−02 |
| ENSG00000105639 | 3718 | JAK3 | 1.60 | 1.06E−04 | 1.43E−02 |
| ENSG00000125804 | 284800 | FAM182A | 1.82 | 1.07E−04 | 1.43E−02 |
| ENSG00000139722 | 79720 | VPS37B | −0.67 | 1.07E−04 | 1.43E−02 |
| ENSG00000157557 | 2114 | ETS2 | −0.85 | 1.03E−04 | 1.43E−02 |
| ENSG00000165841 | 1557 | CYP2C19 | −1.37 | 1.07E−04 | 1.43E−02 |
| ENSG00000182487 | 654816 | NCF1B | 1.59 | 1.05E−04 | 1.43E−02 |
| ENSG00000171847 | 55138 | FAM90A1 | 1.18 | 1.08E−04 | 1.43E−02 |
| ENSG00000162804 | 25992 | SNED1 | 0.71 | 1.09E−04 | 1.43E−02 |
| ENSG00000186184 | 51082 | POLR1D | −0.67 | 1.11E−04 | 1.43E−02 |
| ENSG00000172493 | 4299 | AFF1 | −0.34 | 1.14E−04 | 1.47E−02 |
| ENSG00000179954 | 284297 | SSC5D | 1.43 | 1.15E−04 | 1.47E−02 |
| ENSG00000244682 | NA | FCGR2C | 1.54 | 1.17E−04 | 1.48E−02 |
| ENSG00000081148 | 50939 | IMPG2 | 0.85 | 1.23E−04 | 1.54E−02 |
| ENSG00000126353 | 1236 | CCR7 | 1.51 | 1.26E−04 | 1.57E−02 |
| ENSG00000197705 | 57565 | KLHL14 | 1.75 | 1.27E−04 | 1.57E−02 |
| ENSG00000232268 | 390037 | OR52I1 | 1.53 | 1.30E−04 | 1.59E−02 |
| ENSG00000099204 | 3983 | ABLIM1 | −0.69 | 1.32E−04 | 1.61E−02 |
| ENSG00000158477 | 909 | CD1A | 1.84 | 1.39E−04 | 1.68E−02 |
| ENSG00000172336 | 10248 | POP7 | −0.45 | 1.40E−04 | 1.68E−02 |
| ENSG00000138109 | 1559 | CYP2C9 | −1.47 | 1.44E−04 | 1.71E−02 |
| ENSG00000124766 | 6659 | SOX4 | −0.89 | 1.50E−04 | 1.77E−02 |
| ENSG00000114115 | 5947 | RBP1 | 0.96 | 1.57E−04 | 1.84E−02 |
| ENSG00000156463 | 153769 | SH3RF2 | −1.28 | 1.62E−04 | 1.88E−02 |
| ENSG00000038358 | 23644 | EDC4 | −0.26 | 1.64E−04 | 1.88E−02 |
| ENSG00000211899 | NA | IGHM | 1.79 | 1.64E−04 | 1.88E−02 |
| ENSG00000263503 | NA | | −1.70 | 1.66E−04 | 1.88E−02 |
| ENSG00000005955 | NA | NA | −0.32 | 1.71E−04 | 1.91E−02 |
| ENSG00000144040 | 94097 | SFXN5 | 0.69 | 1.71E−04 | 1.91E−02 |
| ENSG00000105058 | 26017 | FAM32A | −0.37 | 1.90E−04 | 2.11E−02 |
| ENSG00000135837 | 9857 | CEP350 | −0.29 | 1.92E−04 | 2.12E−02 |
| ENSG00000205148 | NA | NA | 1.63 | 1.93E−04 | 2.12E−02 |
| ENSG00000028277 | 5452 | POU2F2 | 1.61 | 1.97E−04 | 2.15E−02 |
| ENSG00000211747 | NA | TRBV20-1 | 1.78 | 2.04E−04 | 2.20E−02 |
| ENSG00000148730 | 1979 | EIF4EBP2 | −0.51 | 2.25E−04 | 2.40E−02 |
| ENSG00000181036 | 343413 | FCRL6 | 1.52 | 2.26E−04 | 2.40E−02 |
| ENSG00000138660 | 55435 | AP1AR | −0.61 | 2.29E−04 | 2.41E−02 |
| ENSG00000144619 | 152330 | CNTN4 | 1.25 | 2.28E−04 | 2.41E−02 |
| ENSG00000000971 | 3075 | CFH | 0.81 | 2.42E−04 | 2.46E−02 |
| ENSG00000099251 | 158160 | HSD17B7P2 | 0.95 | 2.44E−04 | 2.46E−02 |
| ENSG00000156140 | 9508 | ADAMTS3 | 1.24 | 2.42E−04 | 2.46E−02 |
| ENSG00000163113 | NA | NA | −0.63 | 2.41E−04 | 2.46E−02 |
| ENSG00000181458 | 55076 | TMEM45A | 1.40 | 2.37E−04 | 2.46E−02 |
| ENSG00000251287 | 644974 | ALG1L2 | 1.42 | 2.44E−04 | 2.46E−02 |
| ENSG00000086544 | 80271 | ITPKC | −0.57 | 2.52E−04 | 2.52E−02 |
| ENSG00000167984 | 197358 | NLRC3 | 1.07 | 2.55E−04 | 2.54E−02 |
| ENSG00000149633 | 85449 | KIAA1755 | 1.69 | 2.59E−04 | 2.56E−02 |
| ENSG00000102271 | 56062 | KLHL4 | 1.75 | 2.62E−04 | 2.58E−02 |
| ENSG00000137815 | 23168 | RTF1 | −0.36 | 2.81E−04 | 2.72E−02 |
| ENSG00000141682 | 5366 | PMAIP1 | −1.36 | 2.81E−04 | 2.72E−02 |
| ENSG00000179909 | 7710 | ZNF154 | 1.14 | 2.80E−04 | 2.72E−02 |
| ENSG00000110427 | 25758 | KIAA1549L | 1.52 | 2.89E−04 | 2.77E−02 |
| ENSG00000144567 | 79137 | FAM134A | −0.43 | 2.97E−04 | 2.83E−02 |
| ENSG00000206561 | 8292 | COLQ | 0.81 | 3.01E−04 | 2.85E−02 |
| ENSG00000100304 | 23170 | TTLL12 | −0.92 | 3.11E−04 | 2.86E−02 |
| ENSG00000108852 | 4355 | MPP2 | 1.61 | 3.07E−04 | 2.86E−02 |
| ENSG00000181350 | 388341 | LRRC75A | 1.56 | 3.11E−04 | 2.86E−02 |
| ENSG00000186350 | 6256 | RXRA | −0.53 | 3.08E−04 | 2.86E−02 |
| ENSG00000253816 | NA | | 0.93 | 3.05E−04 | 2.86E−02 |
| ENSG00000026751 | 57823 | SLAMF7 | 1.36 | 3.13E−04 | 2.86E−02 |
| ENSG00000142178 | 150094 | SIK1 | −1.46 | 3.17E−04 | 2.86E−02 |
| ENSG00000148848 | 8038 | ADAM12 | 1.19 | 3.16E−04 | 2.86E−02 |
| ENSG00000148339 | 114789 | SLC25A25 | −0.71 | 3.20E−04 | 2.88E−02 |
| ENSG00000137747 | 84000 | TMPRSS13 | −0.89 | 3.23E−04 | 2.88E−02 |
| ENSG00000164061 | 8927 | BSN | 1.16 | 3.29E−04 | 2.92E−02 |
| ENSG00000102221 | 9767 | JADE3 | −0.48 | 3.34E−04 | 2.95E−02 |
| ENSG00000134291 | 79022 | TMEM106C | −0.84 | 3.67E−04 | 3.20E−02 |
| ENSG00000160007 | 2909 | ARHGAP35 | −0.32 | 3.69E−04 | 3.20E−02 |
| ENSG00000198060 | 54708 | MARCH5 | −0.39 | 3.67E−04 | 3.20E−02 |
| ENSG00000111725 | 5564 | PRKAB1 | −0.72 | 3.71E−04 | 3.20E−02 |
| ENSG00000211598 | NA | IGKV4-1 | 1.72 | 3.76E−04 | 3.23E−02 |
| ENSG00000101096 | 4773 | NFATC2 | 0.89 | 3.82E−04 | 3.25E−02 |
| ENSG00000215156 | 646652 | | 1.07 | 3.83E−04 | 3.25E−02 |
| ENSG00000117090 | 6504 | SLAMF1 | 1.64 | 3.89E−04 | 3.26E−02 |
| ENSG00000211892 | NA | IGHG4 | 1.71 | 3.88E−04 | 3.26E−02 |
| ENSG00000129245 | 9513 | FXR2 | −0.35 | 4.00E−04 | 3.33E−02 |
| ENSG00000211685 | NA | IGLC7 | 1.71 | 4.06E−04 | 3.37E−02 |
| ENSG00000085265 | 2219 | FCN1 | 1.67 | 4.11E−04 | 3.38E−02 |
| ENSG00000108691 | 6347 | CCL2 | 1.64 | 4.16E−04 | 3.38E−02 |
| ENSG00000109787 | 51274 | KLF3 | −0.67 | 4.15E−04 | 3.38E−02 |
| ENSG00000243264 | NA | IGKV2D-29 | 1.71 | 4.13E−04 | 3.38E−02 |
| ENSG00000178199 | 340152 | ZC3H12D | 1.65 | 4.19E−04 | 3.38E−02 |
| ENSG00000152413 | 9456 | HOMER1 | −0.58 | 4.24E−04 | 3.39E−02 |
| ENSG00000211893 | NA | IGHG2 | 1.70 | 4.25E−04 | 3.39E−02 |
| ENSG00000241755 | NA | IGKV1-9 | 1.70 | 4.21E−04 | 3.39E−02 |
| ENSG00000017797 | 10928 | RALBP1 | −0.39 | 4.36E−04 | 3.44E−02 |
| ENSG00000136826 | 9314 | KLF4 | −1.16 | 4.37E−04 | 3.44E−02 |
| ENSG00000102245 | 959 | CD40LG | 1.65 | 4.53E−04 | 3.49E−02 |
| ENSG00000144792 | 285349 | ZNF660 | 0.91 | 4.51E−04 | 3.49E−02 |
| ENSG00000151012 | 23657 | SLC7A11 | −1.55 | 4.45E−04 | 3.49E−02 |
| ENSG00000182218 | 84439 | HHIPL1 | 1.55 | 4.48E−04 | 3.49E−02 |
| ENSG00000204961 | 9752 | PCDHA9 | 1.11 | 4.54E−04 | 3.49E−02 |
| ENSG00000160229 | NA | ZNF66 | 0.77 | 4.59E−04 | 3.51E−02 |
| ENSG00000087842 | 8544 | PIR | −0.94 | 4.67E−04 | 3.55E−02 |
| ENSG00000108344 | 5709 | PSMD3 | −0.35 | 4.76E−04 | 3.60E−02 |
| ENSG00000167077 | 150365 | MEI1 | 1.53 | 4.87E−04 | 3.67E−02 |
| ENSG00000211625 | NA | IGKV3D-20 | 1.68 | 4.91E−04 | 3.68E−02 |
| ENSG00000087152 | 56970 | ATXN7L3 | −0.27 | 5.09E−04 | 3.80E−02 |
| ENSG00000138166 | 1847 | DUSP5 | −1.57 | 5.15E−04 | 3.82E−02 |
| ENSG00000187987 | 222696 | ZSCAN23 | 1.19 | 5.20E−04 | 3.84E−02 |
| ENSG00000166676 | 780776 | TVP23A | 1.60 | 5.24E−04 | 3.85E−02 |
| ENSG00000107736 | 64072 | CDH23 | 1.28 | 5.27E−04 | 3.86E−02 |
| ENSG00000109685 | 7468 | WHSC1 | −0.43 | 5.41E−04 | 3.93E−02 |
| ENSG00000240382 | NA | IGKV1-17 | 1.67 | 5.43E−04 | 3.93E−02 |
| ENSG00000196724 | 147686 | ZNF418 | 0.90 | 5.47E−04 | 3.95E−02 |
| ENSG00000178031 | 92949 | ADAMTSL1 | 1.64 | 5.54E−04 | 3.98E−02 |
| ENSG00000109854 | 10553 | HTATIP2 | −0.72 | 5.69E−04 | 4.03E−02 |
| ENSG00000128606 | 10234 | LRRC17 | 1.37 | 5.69E−04 | 4.03E−02 |
| ENSG00000211974 | NA | | 1.66 | 5.69E−04 | 4.03E−02 |
| ENSG00000145002 | 653333 | FAM86B2 | 1.42 | 5.90E−04 | 4.14E−02 |
| ENSG00000185271 | 123103 | KLHL33 | 1.66 | 5.88E−04 | 4.14E−02 |
| ENSG00000147394 | 7739 | ZNF185 | −0.95 | 6.04E−04 | 4.22E−02 |
| ENSG00000105369 | 973 | CD79A | 1.65 | 6.09E−04 | 4.23E−02 |
| ENSG00000205403 | 3426 | CFI | 0.87 | 6.11E−04 | 4.23E−02 |
| ENSG00000242732 | 340526 | RGAG4 | 0.69 | 6.16E−04 | 4.24E−02 |
| ENSG00000074410 | 771 | CA12 | −1.59 | 6.25E−04 | 4.29E−02 |
| ENSG00000107020 | 55848 | PLGRKT | 0.66 | 6.34E−04 | 4.31E−02 |
| ENSG00000145555 | 4651 | MYO10 | −0.45 | 6.31E−04 | 4.31E−02 |
| ENSG00000183486 | 4600 | MX2 | −1.26 | 6.46E−04 | 4.37E−02 |
| ENSG00000066827 | 57623 | ZFAT | 0.33 | 6.62E−04 | 4.46E−02 |
| ENSG00000225217 | NA | HSPA7 | 1.46 | 6.65E−04 | 4.46E−02 |
| ENSG00000143851 | 5778 | PTPN7 | 1.35 | 6.77E−04 | 4.50E−02 |
| ENSG00000161381 | 57125 | PLXDC1 | 1.44 | 6.76E−04 | 4.50E−02 |
| ENSG00000134775 | 80206 | FHOD3 | 1.24 | 6.89E−04 | 4.55E−02 |
| ENSG00000154040 | 26256 | CABYR | −1.18 | 6.89E−04 | 4.55E−02 |
| ENSG00000239951 | NA | IGKV3-20 | 1.64 | 6.97E−04 | 4.58E−02 |
| ENSG00000137709 | 25833 | POU2F3 | −0.82 | 7.04E−04 | 4.60E−02 |
| ENSG00000123159 | 10755 | GIPC1 | −0.48 | 7.11E−04 | 4.61E−02 |
| ENSG00000211939 | NA | NA | 1.63 | 7.10E−04 | 4.61E−02 |
| ENSG00000211666 | NA | IGLV2-14 | 1.63 | 7.17E−04 | 4.63E−02 |
| ENSG00000227507 | 4050 | LTB | 1.60 | 7.24E−04 | 4.66E−02 |
| ENSG00000171714 | 203859 | ANO5 | 1.12 | 7.41E−04 | 4.75E−02 |
| ENSG00000078589 | 27334 | P2RY10 | 1.38 | 7.53E−04 | 4.76E−02 |
| ENSG00000108381 | 443 | ASPA | 1.44 | 7.50E−04 | 4.76E−02 |
| ENSG00000186310 | 4675 | NAP1L3 | 1.58 | 7.47E−04 | 4.76E−02 |
| ENSG00000164398 | 23305 | ACSL6 | 1.19 | 7.61E−04 | 4.80E−02 |
| ENSG00000117122 | 4237 | MFAP2 | 1.28 | 7.65E−04 | 4.80E−02 |
| ENSG00000145536 | 170690 | ADAMTS16 | 1.62 | 7.71E−04 | 4.80E−02 |
| ENSG00000251402 | NA | FAM90A25P | 1.02 | 7.70E−04 | 4.80E−02 |
| ENSG00000161681 | 50944 | SHANK1 | 1.62 | 7.95E−04 | 4.93E−02 |
| ENSG00000130584 | 140685 | ZBTB46 | 0.99 | 8.00E−04 | 4.93E−02 |
| ENSG00000204839 | 642475 | MROH6 | −1.17 | 8.02E−04 | 4.93E−02 |
| ENSG00000204544 | 394263 | MUC21 | −1.52 | 8.06E−04 | 4.94E−02 |
| ENSG00000112245 | 7803 | PTP4A1 | −0.59 | 8.14E−04 | 4.97E−02 |
| ENSG00000137265 | 3662 | IRF4 | 1.61 | 8.28E−04 | 5.04E−02 |
| ENSG00000105364 | 51073 | MRPL4 | −0.56 | 8.39E−04 | 5.06E−02 |
| ENSG00000178922 | 81888 | HYI | 0.84 | 8.36E−04 | 5.06E−02 |
| ENSG00000118849 | 5918 | RARRES1 | 1.24 | 8.47E−04 | 5.09E−02 |
| ENSG00000212743 | NA | | 1.23 | 8.58E−04 | 5.13E−02 |
| ENSG00000135074 | 8728 | ADAM19 | 1.42 | 8.76E−04 | 5.16E−02 |
| ENSG00000152689 | 25780 | RASGRP3 | 1.42 | 8.72E−04 | 5.16E−02 |
| ENSG00000154640 | 10950 | BTG3 | −0.77 | 8.74E−04 | 5.16E−02 |
| ENSG00000196581 | 55966 | AJAP1 | −1.59 | 8.74E−04 | 5.16E−02 |
| ENSG00000211950 | NA | IGHV1-24 | 1.60 | 9.04E−04 | 5.30E−02 |
| ENSG00000188171 | 199777 | ZNF626 | 0.72 | 9.17E−04 | 5.36E−02 |
| ENSG00000134490 | 85019 | TMEM241 | 0.50 | 9.27E−04 | 5.40E−02 |
| ENSG00000186854 | 129293 | TRABD2A | 1.13 | 9.34E−04 | 5.42E−02 |
| ENSG00000173198 | 10800 | CYSLTR1 | 1.16 | 9.39E−04 | 5.43E−02 |
| ENSG00000132669 | 54453 | RIN2 | −0.47 | 9.54E−04 | 5.48E−02 |
| ENSG00000211640 | NA | IGLV6-57 | 1.59 | 9.52E−04 | 5.48E−02 |
| ENSG00000159450 | 7062 | TCHH | −1.34 | 9.59E−04 | 5.48E−02 |
| ENSG00000143515 | 57198 | ATP8B2 | 0.79 | 9.73E−04 | 5.54E−02 |
| ENSG00000198734 | 2153 | F5 | 1.10 | 9.90E−04 | 5.62E−02 |
| ENSG00000229645 | NA | NA | 1.32 | 9.99E−04 | 5.65E−02 |
| ENSG00000106333 | 5118 | PCOLCE | 1.21 | 1.02E−03 | 5.66E−02 |
| ENSG00000166869 | 63928 | CHP2 | 1.49 | 1.01E−03 | 5.66E−02 |
| ENSG00000243238 | NA | IGKV2-30 | 1.59 | 1.02E−03 | 5.66E−02 |
| ENSG00000259236 | NA | GOLGA8VP | 1.52 | 1.01E−03 | 5.66E−02 |
| ENSG00000240864 | NA | IGKV1-16 | 1.59 | 1.02E−03 | 5.67E−02 |
| ENSG00000170509 | 345275 | HSD17B13 | 1.49 | 1.03E−03 | 5.68E−02 |
| ENSG00000235602 | 642559 | POU5F1P3 | 1.51 | 1.06E−03 | 5.84E−02 |
| ENSG00000125813 | 5075 | PAX1 | 1.57 | 1.10E−03 | 6.04E−02 |
| ENSG00000138413 | 3417 | IDH1 | −0.69 | 1.11E−03 | 6.04E−02 |
| ENSG00000161896 | 117283 | IP6K3 | −1.20 | 1.11E−03 | 6.04E−02 |
| ENSG00000183624 | 56941 | HMCES | −0.49 | 1.11E−03 | 6.04E−02 |
| ENSG00000196834 | 653269 | POTEI | 1.56 | 1.11E−03 | 6.04E−02 |
| ENSG00000110777 | 5450 | POU2AF1 | 1.42 | 1.14E−03 | 6.10E−02 |
| ENSG00000183773 | 150209 | AIFM3 | 1.17 | 1.14E−03 | 6.10E−02 |
| ENSG00000225698 | NA | IGHV3-72 | 1.57 | 1.13E−03 | 6.10E−02 |
| ENSG00000023445 | 330 | BIRC3 | 0.84 | 1.18E−03 | 6.27E−02 |
| ENSG00000100078 | 50487 | PLA2G3 | 1.42 | 1.18E−03 | 6.27E−02 |
| ENSG00000116690 | 10216 | PRG4 | 1.56 | 1.19E−03 | 6.27E−02 |
| ENSG00000136011 | 55576 | STAB2 | 1.54 | 1.19E−03 | 6.27E−02 |
| ENSG00000172986 | 727936 | GXYLT2 | 0.82 | 1.19E−03 | 6.27E−02 |
| ENSG00000196344 | 131 | ADH7 | −1.54 | 1.19E−03 | 6.27E−02 |
| ENSG00000197006 | 51108 | METTL9 | −0.35 | 1.21E−03 | 6.33E−02 |
| ENSG00000087303 | 22795 | NID2 | 1.37 | 1.22E−03 | 6.34E−02 |
| ENSG00000211947 | NA | IGHV3-21 | 1.56 | 1.23E−03 | 6.38E−02 |
| ENSG00000170471 | 57148 | RALGAPB | −0.27 | 1.23E−03 | 6.39E−02 |
| ENSG00000146374 | 84870 | RSPO3 | 1.55 | 1.25E−03 | 6.45E−02 |
| ENSG00000221970 | 346528 | OR2A1 | 1.49 | 1.26E−03 | 6.48E−02 |
| ENSG00000163354 | 127579 | DCST2 | 1.15 | 1.27E−03 | 6.50E−02 |
| ENSG00000008277 | 53616 | ADAM22 | 0.80 | 1.28E−03 | 6.51E−02 |
| ENSG00000198829 | 56670 | SUCNR1 | 1.51 | 1.27E−03 | 6.51E−02 |
| ENSG00000145808 | 171019 | ADAMTS19 | 1.13 | 1.29E−03 | 6.51E−02 |
| ENSG00000197102 | 1778 | DYNC1H1 | −0.25 | 1.29E−03 | 6.51E−02 |
| ENSG00000198821 | 919 | CD247 | 1.40 | 1.30E−03 | 6.58E−02 |
| ENSG00000221914 | 5520 | PPP2R2A | −0.36 | 1.32E−03 | 6.64E−02 |
| ENSG00000078898 | 80341 | BPIFB2 | −1.47 | 1.36E−03 | 6.69E−02 |
| ENSG00000122140 | 51116 | MRPS2 | −0.56 | 1.35E−03 | 6.69E−02 |
| ENSG00000123595 | 9367 | RAB9A | −0.50 | 1.35E−03 | 6.69E−02 |
| ENSG00000145782 | 9140 | ATG12 | 0.46 | 1.35E−03 | 6.69E−02 |
| ENSG00000163297 | 118429 | ANTXR2 | 0.84 | 1.35E−03 | 6.69E−02 |
| ENSG00000180479 | 51276 | ZNF571 | 0.65 | 1.36E−03 | 6.69E−02 |
| ENSG00000182578 | 1436 | CSF1R | 1.23 | 1.35E−03 | 6.69E−02 |
| ENSG00000185518 | 9899 | SV2B | 1.22 | 1.37E−03 | 6.71E−02 |
| ENSG00000056586 | 54542 | RC3H2 | −0.24 | 1.38E−03 | 6.74E−02 |
| ENSG00000021574 | 6683 | SPAST | −0.28 | 1.39E−03 | 6.76E−02 |
| ENSG00000008394 | 4257 | MGST1 | −0.73 | 1.40E−03 | 6.77E−02 |
| ENSG00000112715 | 7422 | VEGFA | −1.09 | 1.40E−03 | 6.77E−02 |
| ENSG00000171724 | 57687 | VAT1L | 1.54 | 1.41E−03 | 6.81E−02 |
| ENSG00000124226 | 55905 | RNF114 | −0.35 | 1.43E−03 | 6.87E−02 |
| ENSG00000147459 | 80005 | DOCK5 | −0.44 | 1.45E−03 | 6.93E−02 |
| ENSG00000147140 | 4841 | NONO | −0.27 | 1.46E−03 | 6.99E−02 |
| ENSG00000118922 | 11278 | KLF12 | 0.79 | 1.49E−03 | 7.08E−02 |
| ENSG00000142731 | 10733 | PLK4 | −1.30 | 1.50E−03 | 7.12E−02 |
| ENSG00000159388 | 7832 | BTG2 | −1.23 | 1.50E−03 | 7.12E−02 |
| ENSG00000168904 | 123355 | LRRC28 | −0.39 | 1.53E−03 | 7.20E−02 |
| ENSG00000234231 | NA | | 1.34 | 1.53E−03 | 7.20E−02 |
| ENSG00000187446 | 11261 | CHP1 | −0.52 | 1.54E−03 | 7.22E−02 |
| ENSG00000180251 | 389015 | SLC9A4 | −1.51 | 1.55E−03 | 7.25E−02 |
| ENSG00000165912 | 29763 | PACSIN3 | −0.86 | 1.55E−03 | 7.25E−02 |
| ENSG00000165178 | 654817 | NCF1C | 1.35 | 1.56E−03 | 7.25E−02 |
| ENSG00000128383 | 200315 | APOBEC3A | 1.48 | 1.59E−03 | 7.39E−02 |
| ENSG00000155158 | 158219 | TTC39B | −0.49 | 1.60E−03 | 7.40E−02 |
| ENSG00000224041 | NA | IGKV3D-15 | 1.51 | 1.62E−03 | 7.45E−02 |
| ENSG00000101782 | 8780 | RIOK3 | −0.40 | 1.65E−03 | 7.55E−02 |
| ENSG00000108671 | 5717 | PSMD11 | −0.30 | 1.65E−03 | 7.55E−02 |
| ENSG00000135821 | 2752 | GLUL | −0.54 | 1.65E−03 | 7.55E−02 |
| ENSG00000076053 | 10179 | RBM7 | −0.35 | 1.68E−03 | 7.62E−02 |
| ENSG00000122966 | 11113 | CIT | −1.03 | 1.68E−03 | 7.62E−02 |
| ENSG00000253755 | NA | IGHGP | 1.52 | 1.68E−03 | 7.62E−02 |
| ENSG00000120129 | 1843 | DUSP1 | −1.38 | 1.73E−03 | 7.64E−02 |
| ENSG00000121807 | 729230 | CCR2 | 1.29 | 1.72E−03 | 7.64E−02 |
| ENSG00000134046 | 8932 | MBD2 | −0.24 | 1.73E−03 | 7.64E−02 |
| ENSG00000135773 | 10753 | CAPN9 | −1.11 | 1.73E−03 | 7.64E−02 |
| ENSG00000136830 | 64855 | FAM129B | −0.31 | 1.72E−03 | 7.64E−02 |
| ENSG00000157601 | 4599 | MX1 | −1.06 | 1.72E−03 | 7.64E−02 |
| ENSG00000162949 | 92291 | CAPN13 | 0.99 | 1.69E−03 | 7.64E−02 |
| ENSG00000173653 | 9986 | RCE1 | −0.43 | 1.73E−03 | 7.64E−02 |
| ENSG00000177106 | 64787 | EPS8L2 | −0.65 | 1.73E−03 | 7.64E−02 |
| ENSG00000115590 | 7850 | IL1R2 | −1.50 | 1.74E−03 | 7.65E−02 |
| ENSG00000136816 | 27348 | TOR1B | −0.45 | 1.75E−03 | 7.68E−02 |
| ENSG00000091831 | 2099 | ESR1 | 1.43 | 1.78E−03 | 7.76E−02 |
| ENSG00000101544 | 22850 | ADNP2 | −0.34 | 1.78E−03 | 7.76E−02 |
| ENSG00000086548 | 4680 | CEACAM6 | −1.10 | 1.79E−03 | 7.78E−02 |
| ENSG00000141738 | 2886 | GRB7 | −0.76 | 1.79E−03 | 7.78E−02 |
| ENSG00000224078 | NA | SNHG14 | 0.61 | 1.80E−03 | 7.78E−02 |
| ENSG00000131408 | 7376 | NR1H2 | −0.42 | 1.81E−03 | 7.80E−02 |
| ENSG00000111412 | 79794 | C12orf49 | −0.81 | 1.82E−03 | 7.84E−02 |
| ENSG00000124356 | 10617 | STAMBP | −0.33 | 1.85E−03 | 7.95E−02 |
| ENSG00000232216 | NA | IGHV3-43 | 1.49 | 1.86E−03 | 7.96E−02 |
| ENSG00000115963 | 390 | RND3 | −0.89 | 1.90E−03 | 8.05E−02 |
| ENSG00000120647 | 84318 | CCDC77 | −0.46 | 1.90E−03 | 8.05E−02 |
| ENSG00000141540 | 94015 | TTYH2 | 1.04 | 1.90E−03 | 8.05E−02 |
| ENSG00000147234 | 84443 | FRMPD3 | 1.24 | 1.90E−03 | 8.05E−02 |
| ENSG00000189221 | 4128 | MAOA | −1.08 | 1.91E−03 | 8.08E−02 |
| ENSG00000125430 | 9953 | HS3ST3B1 | −0.95 | 1.94E−03 | 8.15E−02 |
| ENSG00000144191 | 1261 | CNGA3 | 1.43 | 1.94E−03 | 8.15E−02 |
| ENSG00000132405 | 57533 | TBC1D14 | −0.40 | 1.95E−03 | 8.16E−02 |
| ENSG00000179840 | 644997 | PIK3CD-AS1 | 1.50 | 1.96E−03 | 8.16E−02 |
| ENSG00000259261 | NA | IGHV4OR15-8 | 1.49 | 1.96E−03 | 8.16E−02 |
| ENSG00000147168 | 3561 | IL2RG | 1.31 | 1.98E−03 | 8.21E−02 |
| ENSG00000164692 | 1278 | COL1A2 | 1.46 | 2.03E−03 | 8.40E−02 |
| ENSG00000013563 | 1774 | DNASE1L1 | −0.82 | 2.04E−03 | 8.42E−02 |
| ENSG00000103522 | 50615 | IL21R | 1.46 | 2.04E−03 | 8.42E−02 |
| ENSG00000108091 | 8030 | CCDC6 | −0.30 | 2.11E−03 | 8.64E−02 |
| ENSG00000157954 | 26100 | WIPI2 | −0.40 | 2.11E−03 | 8.64E−02 |
| ENSG00000070214 | 23446 | SLC44A1 | −0.42 | 2.13E−03 | 8.70E−02 |
| ENSG00000156671 | 142891 | SAMD8 | −0.39 | 2.17E−03 | 8.80E−02 |
| ENSG00000174807 | 57124 | CD248 | 1.46 | 2.17E−03 | 8.80E−02 |
| ENSG00000154277 | 7345 | UCHL1 | −1.35 | 2.19E−03 | 8.82E−02 |
| ENSG00000157766 | 176 | ACAN | 1.48 | 2.18E−03 | 8.82E−02 |
| ENSG00000171475 | 147179 | WIPF2 | −0.44 | 2.19E−03 | 8.82E−02 |
| ENSG00000211945 | NA | IGHV1-18 | 1.48 | 2.19E−03 | 8.82E−02 |
| ENSG00000186407 | 342510 | CD300E | 1.46 | 2.21E−03 | 8.86E−02 |
| ENSG00000211659 | NA | IGLV3-25 | 1.48 | 2.21E−03 | 8.86E−02 |
| ENSG00000167797 | 10263 | CDK2AP2 | −0.53 | 2.24E−03 | 8.92E−02 |
| ENSG00000132465 | 3512 | JCHAIN | 1.47 | 2.25E−03 | 8.95E−02 |
| ENSG00000201643 | 677801 | SNORA14A | 0.93 | 2.25E−03 | 8.95E−02 |
| ENSG00000036530 | 10858 | CYP46A1 | 1.15 | 2.26E−03 | 8.96E−02 |
| ENSG00000162782 | 163589 | TDRD5 | 1.42 | 2.27E−03 | 8.96E−02 |
| ENSG00000224373 | NA | IGHV4-59 | 1.47 | 2.28E−03 | 8.96E−02 |
| ENSG00000132938 | 23281 | MTUS2 | 1.44 | 2.28E−03 | 8.96E−02 |
| ENSG00000112299 | 8876 | VNN1 | 1.37 | 2.35E−03 | 9.19E−02 |
| ENSG00000164100 | 9348 | NDST3 | 1.12 | 2.36E−03 | 9.22E−02 |
| ENSG00000165949 | 3429 | IFI27 | −1.30 | 2.36E−03 | 9.22E−02 |
| ENSG00000107643 | 5599 | MAPK8 | −0.33 | 2.39E−03 | 9.29E−02 |
| ENSG00000184481 | 4303 | FOXO4 | −0.55 | 2.40E−03 | 9.29E−02 |
| ENSG00000105122 | 64926 | RASAL3 | 1.32 | 2.43E−03 | 9.40E−02 |
| ENSG00000167100 | 201191 | SAMD14 | 1.44 | 2.44E−03 | 9.42E−02 |
| ENSG00000198848 | 1066 | CES1 | −1.10 | 2.51E−03 | 9.63E−02 |
| ENSG00000211964 | NA | IGHV3-48 | 1.45 | 2.50E−03 | 9.63E−02 |
| ENSG00000099958 | 91319 | DERL3 | 0.90 | 2.51E−03 | 9.63E−02 |
| ENSG00000010017 | 10048 | RANBP9 | −0.43 | 2.54E−03 | 9.69E−02 |
| ENSG00000189056 | 5649 | RELN | 1.38 | 2.54E−03 | 9.69E−02 |
| ENSG00000213988 | 7643 | ZNF90 | 0.83 | 2.57E−03 | 9.74E−02 |
| ENSG00000232810 | 7124 | TNF | 1.30 | 2.57E−03 | 9.74E−02 |
| ENSG00000011295 | 54902 | TTC19 | −0.30 | 2.58E−03 | 9.75E−02 |
| ENSG00000126062 | 11070 | TMEM115 | −0.34 | 2.61E−03 | 9.82E−02 |
| ENSG00000211933 | NA | IGHV6-1 | 1.44 | 2.61E−03 | 9.82E−02 |
| ENSG00000181588 | 399664 | MEX3D | −0.62 | 2.62E−03 | 9.84E−02 |
| ENSG00000122188 | 54900 | LAX1 | 1.37 | 2.64E−03 | 9.89E−02 |
| ENSG00000162772 | 467 | ATF3 | −1.35 | 2.65E−03 | 9.90E−02 |
| ENSG00000157064 | 23057 | NMNAT2 | 1.33 | 2.67E−03 | 9.95E−02 |

UIP (n = 12 samples); Non UIP (n = 4 samples). Positive log2 fold change value indicates over-expression in UIP relative to Non UIP; negative log2 value indicates under-expression in UIP relative to Non UIP. In this analysis only patients with a smoking history were evaluated, hence this subset harbored only smokers.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

Some embodiments and/or methods described herein can be performed by software (executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including C, C++, Java™, Ruby, Visual Basic™, R, and/or other object-oriented, procedural, statistical, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (e.g., Haskell, Erlang, etc.), logical programming languages (e.g., Prolog), object-oriented programming languages (e.g., Java, C++, etc.), statistical programming languages and/or environments (e.g., R, etc.; or other suitable programming languages and/or development tools. Additional examples of computer code include, but are not limited to, control signals, encrypted code, and compressed code.

### REFERENCES

All of the following references are incorporated herein in their entirety.
1. du Bois R M. Strategies for treating idiopathic pulmonary fibrosis. Nature reviews Drug discovery 2010; 9(2): 129-40.
2. Hodnett P A, Naidich D P. Fibrosing Interstitial Lung Disease: A Practical HRCT Based Approach to Diagnosis and Management and Review of the Literature. American Journal of Respiratory Critical Care Medicine 2013.
3. American Thoracic Society. Idiopathic pulmonary fibrosis: diagnosis and treatment. International consensus statement. American Thoracic Society (ATS), and the European Respiratory Society (ERS). American journal of respiratory and critical care medicine 2000; 161(2 Pt 1): 646-64.
4. King T E, Jr., Pardo A, Selman M. Idiopathic pulmonary fibrosis. Lancet 2011; 378(9807): 1949-61.
5. Raghu G, Collard H R, Egan J J, et al. An official ATS/ERS/JRS/ALAT statement: idiopathic pulmonary fibrosis: evidence-based guidelines for diagnosis and management. American journal of respiratory and critical care medicine 2011; 183(6): 788-824.
6. Wells A U. The revised ATS/ERS/JRS/ALAT diagnostic criteria for idiopathic pulmonary fibrosis (IPF)—practical implications. Respiratory research 2013; 14 Suppl 1: S2.
7. Fernandez Perez E R, Daniels C E, Schroeder D R, et al. Incidence, prevalence, and clinical course of idiopathic pulmonary fibrosis: a population-based study. Chest 2010; 137(1): 129-37.
8. du Bois R M, Weycker D, Albera C, et al. Ascertainment of individual risk of mortality for patients with idiopathic pulmonary fibrosis. American journal of respiratory and critical care medicine 2011; 184(4): 459-66.
9. King T E, Jr., Bradford W Z, Castro-Bemardini S, et al. A phase 3 trial of pirfenidone in patients with idiopathic pulmonary fibrosis. N Engl J Med 2014; 370(22): 2083-92.
10. Richeldi L, du Bois R M, Raghu G, et al. Efficacy and safety of nintedanib in idiopathic pulmonary fibrosis. N Engl J Med 2014; 370(22): 2071-82.
11. Woodcock H V, Maher™. The treatment of idiopathic pulmonary fibrosis.

F1000prime reports 2014; 6: 16.
12. Coffin V, Richeldi L. Neglected evidence in idiopathic pulmonary fibrosis and the importance of early diagnosis and treatment. European respiratory review: an official journal of the European Respiratory Society 2014; 23(131): 106-10.
13. Sumikawa H, Johkoh T, Colby T V, et al. Computed tomography findings in pathological usual interstitial pneumonia: relationship to survival. American journal of respiratory and critical care medicine 2008; 177(4): 433-9.
14. Wells A U. Managing diagnostic procedures in idiopathic pulmonary fibrosis. European respiratory review: an official journal of the European Respiratory Society 2013; 22(128): 158-62.
15. Collard H R, King T E, Jr., Bartelson B B, Vourlekis J S, Schwarz M I, Brown K K. Changes in clinical and physiologic variables predict survival in idiopathic pulmonary fibrosis. American journal of respiratory and critical care medicine 2003; 168(5): 538-42.
16. Nicholson A G, Addis B J, Bharucha H, et al. Inter-observer variation between pathologists in diffuse parenchymal lung disease. Thorax 2004; 59(6): 500-5.
17. Flaherty K R, King T E, Jr., Raghu G, et al. Idiopathic interstitial pneumonia: what is the effect of a multidisciplinary approach to diagnosis? American journal of respiratory and critical care medicine 2004; 170(8): 904-10.
18. Selman M, Pardo A, Barrera L, et al. Gene expression profiles distinguish idiopathic pulmonary fibrosis from hypersensitivity pneumonitis. American journal of respiratory and critical care medicine 2006; 173(2): 188-98.
19. Lockstone H E, Sanderson S, Kulakova N, et al. Gene set analysis of lung samples provides insight into pathogenesis of progressive, fibrotic pulmonary sarcoidosis. American journal of respiratory and critical care medicine 2010; 181(12): 1367-75.
20. Katzenstein A L. Smoking-related interstitial fibrosis (SRIF), pathogenesis and treatment of usual interstitial pneumonia (UIP), and transbronchial biopsy in UIP. Modern pathology: an official journal of the United States and Canadian Academy of Pathology, Inc 2012; 25 Suppl 1: S68-78.
21. Team R C. R: A language and environment for statistical computing. R Foundation for Statistical Computing, Vienna, Austria wwwR-projectorg/2014.
22. Pardo A, Gibson K, Cisneros J, et al. Up-regulation and profibrotic role of osteopontin in human idiopathic pulmonary fibrosis. PLoS medicine 2005; 2(9): e251.
23. DePianto D J, Chandriani S, Abbas A R, et al. Heterogeneous gene expression signatures correspond to distinct lung pathologies and biomarkers of disease severity in idiopathic pulmonary fibrosis. Thorax 2014.
24. Selman M, Pardo A, King T E, Jr. Hypersensitivity pneumonitis: insights in diagnosis and pathobiology. American journal of respiratory and critical care medicine 2012; 186(4): 314-24.
25. Yang I V, Coldren C D, Leach S M, et al. Expression of cilium-associated genes defines novel molecular subtypes of idiopathic pulmonary fibrosis. Thorax 2013.
26. Garcia-Alvarez J, Ramirez R, Checa M, et al. Tissue inhibitor of metalloproteinase-3 is up-regulated by transforming growth factor-betal in vitro and expressed in fibroblastic foci in vivo in idiopathic pulmonary fibrosis. Experimental lung research 2006; 32(5): 201-14.
27. Piotrowski W J, Gorski P, Pietras T, Fendler W, Szemraj J. The selected genetic polymorphisms of metalloproteinases MMP2, 7, 9 and MMP inhibitor TIMP2 in sarcoidosis. Medical science monitor: international medical journal of experimental and clinical research 2011; 17(10): CR598-607.
28. Chaudhuri R, McSharry C, Brady J, et al. Low sputum MMP-9/TIMP ratio is associated with airway narrowing in smokers with asthma. The European respiratory journal: official journal of the European Society for Clinical Respiratory Physiology 2014; 44(4): 895-904.
29. Hviid T V, Milman N, Hylenius S, Jakobsen K, Jensen M S, Larsen L G. HLA-G polymorphisms and HLA-G expression in sarcoidosis. Sarcoidosis, vasculitis, and diffuse lung diseases: official journal of WASOG/World Association of Sarcoidosis and Other Granulomatous Disorders 2006; 23(1): 30-7.
30. Li G Y, Kim M, Kim J H, Lee M O, Chung J H, Lee B H. Gene expression profiling in human lung fibroblast following cadmium exposure. Food and chemical toxicology: an international journal published for the British Industrial Biological Research Association 2008; 46(3): 1131-7.
31. Ozsolak F, Milos P M. RNA sequencing: advances, challenges and opportunities. Nature reviews Genetics 2011; 12(2): 87-98.
32. Mutz K O, Heilkenbrinker A, Lonne M, Walter J G, Stahl F. Transcriptome analysis using next-generation sequencing. Current opinion in biotechnology 2013; 24(1): 22-30.

1.-5. (canceled)

6. A method for generating a classification of a biological sample as being positive for usual interstitial pneumonia (UIP) or non-UIP, comprising:
(a) assaying nucleic acid molecules derived from said biological sample to determine a level of expression of a plurality of genes; and
(b) processing with a classifier said level of expression of said plurality of genes to generate a classification of said biological sample as being positive for UIP or non-UIP, wherein said classifier differentiates between said UIP and a plurality of non-UIP subtypes, wherein said plurality of non-UIP subtypes comprises hypersensitivity pneumonitis (HP), non-specific interstitial pneumonia (NSIP), sarcoidosis, respiratory bronchiolitis (RB), bronchiolitis, diffuse alveolar damage (DAD), and organizing pneumonia (OP).

7. The method of claim 6, wherein said assaying comprises sequencing.

8. The method of claim 7, wherein said sequencing comprises whole transcriptome shotgun sequencing.

9. The method of claim 6, wherein said assaying comprises RT-PCR or DNA microarray hybridization.

10. The method of claim 6, wherein said nucleic acid molecules comprise ribonucleic acids (RNA) molecules, and wherein said assaying comprises generating complementary deoxyribonucleic acid (cDNA) molecules from said RNA molecules.

11. The method of claim 10, wherein said assaying further comprises amplifying said cDNA molecules.

12. The method of claim 6, wherein said assaying further comprises identifying a level of expression of at least one control nucleic acid molecule in said biological sample.

13. The method of claim 6, wherein said classifier comprises a machine learning algorithm that is trained to identify said UIP from said plurality of non-UIP subtypes.

14. The method of claim 13, wherein said machine learning algorithm is trained with a training set that is independent of said biological sample.

15. The method of claim 13, wherein said machine learning algorithm is trained using features comprising gene expression variants, gene fusions, loss of heterozygosity, or biological pathway effect.

16. The method of claim 6, wherein said classifier comprises a machine learning algorithm that is trained to recognize gene signatures that distinguish non-smokers from smokers.

17. The method of claim 6, wherein said biological sample is a transbronchial biopsy sample or a bronchoalveolar lavage sample.

18. The method of claim 6, wherein said biological sample is fresh-frozen or fixed.

19. The method of claim 6, wherein said biological sample is derived from a subject that is suspected of having an interstitial lung disease.

20. The method of claim 19, wherein said subject is suspected of having said interstitial lung disease based at least in part on one or more clinical signs or one or more symptoms.

21. The method of claim 20, wherein said one or more symptoms comprises shortness of breath or dry cough.

22. The method of claim 20, wherein said one or more clinical signs comprises a result of a pulmonary function test or a lung tissue analysis.

23. The method of claim 22, wherein said pulmonary function test is spirometry, oximetry, or an exercise stress test.

24. The method of claim 22, wherein said lung tissue analysis comprises histological or cytological analysis of a lung tissue sample of said subject.

25. The method of claim 6, wherein said biological sample is derived from a subject that is a smoker or has a past history of smoking.

26. The method of claim 6, further comprising providing a therapeutic intervention to said subject based at least in part on said classification generated in (b).


SEQUENCE LISTING (22 sequences)

SEQ ID NO: 1 - DNA, 1041 bp, Homo sapiens
  source 1..1041: mol_type = unassigned DNA
atggcgccccgaagcctcctcctgctgctctcaggggccctggccctgaccgatacttgggcaggctcccactccttgaggtatttcagcaccgctgtgtcgcggcccggccgcggggagccccgctacatcgccgtggagtacgtagacgacacgcaattcctgcggttcgacagcgacgccgcgattccgaggatggagccgcgggagccgtgggtggagcaagaggggccgcagtattgggagtggaccacagggtacgccaaggccaacgcacagactgaccgagtggccctgaggaacctgctccgccgctacaaccagagcgaggctgggtctcacaccctccagggaatgaatggctgcgacatggggcccgacggacgcctcctccgcgggtatcaccagcacgcgtacgacggcaaggattacatctccctgaacgaggacctgcgctcctggaccgcggcggacaccgtggctcagatcacccagcgcttctatgaggcagaggaatatgcagaggagttcaggacctacctggagggcgagtgcctggagttgctccgcagatacttggagaatgggaaggagacgctacagcgcgcagatcctccaaaggcacacgttgcccaccaccccatctctgaccatgaggccaccctgaggtgctgggccctgggcttctaccctgcggagatcacgctgacctggcagcgggatggggaggaacagacccaggacacagagcttgtggagaccaggcctgcaggggatggaaccttccagaagtgggccgctgtggtggtgccttctggagaggaacagagatacacatgccatgtgcagcacgaggggctgccccagcccctcatcctgagatgggagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagtgtga

SEQ ID NO: 2 - DNA, 1993 bp, Homo sapiens
  source 1..1993: mol_type = unassigned DNA
caggtgttggtgcctgccgtgaacgcattctgacctgggccgtatctgtctcccaagactttgtgcctatggttggggacagagtgaggtcgttgccttgacgacgacagcatgcggcccgtggtcctcctaagtgtgagcttgcggcggaccgaggcccacctgcctccctgcctgcttcgccctggactcgtgactgcgtccgcagaagaaatcacaacagcgctggaattgctagtttgctaggcagcatcttttggacctgcgaaccatatgcatttcacctcaaatttgtttccaagttgaaaacctttgggtctttctatgcgaacggattgaagaaacgcaaaaagtttctacggactttaaattaaaatggaaaaatatgaaaacctgggtttggttggagaagggagttatggaatggtgatgaagtgtaggaataaagatactggaagaattgtggccataaagaagttcttagaaagtgacgatgacaaaatggttaaaaagattgcaatgcgagaaatcaagttactaaagcaacttaggcatgaaaacttggtgaatctcttggaagtgtgtaagaaaaaaaaacgatggtacctagtctttgaatttgttgaccacacaattcttgatgacttggagctctttccaaatggactagactaccaagtagttcaaaagtatttgtttcagattattaatggaattggattttgtcacagtcacaatatcatacacagagatataaagccagagaatatattagtctcccagtctggcgttgtcaagctatgcgattttggatttgcgcgaacattggcagctcctggggaggtttatactgattatgtggcaacccgatggtacagagctccagaactattggttggtgatgtcaagtatggcaaggctgttgatgtgtgggccattggttgtctggtaactgaaatgttcatgggggaacccctatttcctggagattctgatattgatcagctatatcatattatgatgtgtttaggtaatctaattccaaggcatcaggagctttttaataaaaatcctgtgtttgctggagtaaggttgcctgaaatcaaggaaagagaacctcttgaaagacgctatcctaagctctctgaagtggtgatagatttagcaaagaaatgcttacatattgaccccgacaaaagacccttctgtgctgagctcctacaccatgatttctttcaaatggatggatttgctgagaggttttcccaagaactacagttaaaagtacagaaagatgccagaaatgtttctttatctaaaaaatcccaaaacagaaagaaggaaaaagaaaaagatgattccttagttgaagaaagaaaaacacttgtggtacaggataccaatgctgatcccaaaattaaggattataaactatttaaaataaaaggctcaaaaattgatggagaaaaagctgaaaaaggcaatagagcttcaaatgccagctgtctccatgacagtaggacaagccacaacaaaatagtgccttcaacaagcctcaaagactgcagcaatgtcagcgtggaccacacaaggaatccaagcgtggcaattcccccacttacacacaatctttctgcagttgctcccagcattaattctggaatggggactgagactataccaattcagggttacagagtggatgagaaaactaagaagtgttctattccatttgttaaaccgaacagacattccccatcaggcatttataacattaatgtgaccacattagtatcaggacctcccctgtcagatgattcaggggctgatttgcctcaaatggaacaccagcactgagaaccattttggttctgaactggatgatgctcttgcacttgagatgacatcttcttgcagcaagaaaaaaaaaaaaaaaaaaaaaaaaaaaacaaaaaaaaaaaaaaaaaaaaaaaaaaaaaaaaaaaaaaaaaaa

SEQ ID NO: 3 - DNA, 3918 bp, Homo sapiens
  source 1..3918: mol_type = unassigned DNA
gttaattggagcccacgcctgaatctagatttcagtgttgcagtgattacaatattggataatgatgacctggcaggaatggatatttccttccccgagacaactgtggctgtagcagttgacacaactctcattcctgtagaaactgaatccaccacatacctcagcacaagcaagacgactaccattctgcagccaaccaacgtggttgccattgttactgaggcaactggtgtatctgccatccctgagaaacttgtcacccttcatggcacacctgctgtgtctgaaaagcctgatgtggccactgtaactgccaatgtttccattcatggaacattcagccttgggccatccattgtttatattgaagaggagatgaagaatggcacattcaacactgcagaagttcttatccgaagaactggtgggtttactggcaatgtcagcataacagttaaaactttcggtgaaagatgtgctcagatggaaccaaatgcattgccctttcgtggtatctatgggatttccaacctaacatgggcagttgaagaagaagactttgaagaacaaactcttacccttatattcctagatggagaaagagaacgtaaagtatcagttcaaattttggatgatgatgagcctgaggggcaggaattcttctacgtgtttctcacaaaccctcaagggggagcacagattgtggaggggaaggatgatactggatttgcagcttttgccatggttattattacagggagtgaccttcacaatggcatcataggattcagtgaggagtcccagagtggactagaactcagggaaggagctgttatgagaagattgcaccttattgtcacaagacagccaaacagggcctttgaagatgtcaaggtcttttggcgagtcacacttaacaaaacagtcgtcgtgctccagaaggatggggtaaacctgatggaggaacttcagtctgtgtcagggaccacaacctgtacaatgggtcaaacaaaatgctttatcagcattgaactcaaaccagaaaaggtaccacaggttgaagtgtatttttttgtggaactatatgaagctactgctggagcagcaataaacaacagtgccagattcgcacagattaaaatcttagaaagtgatgaatctcaaagccttgtgtatttttctgtgggttctcggctggcagtggctcacaagaaggccactttaatcagtctgcaggtggccagagattctgggacaggactaatgatgtctgttaactttagtacccaggagttgaggagtgctgaaacaattggtcgtaccatcatatctccagctatttctggaaaggattttgtgataactgaaggcacattggtctttgaacctggccagagaagcactgtattggatgtcatcctaacgccagagacaggatctttaaattcatttcctaaacgcttccagattgtcctttttgacccaaaaggtggtgccagaattgataaagtgtatgggactgccaacatcactcttgtctcagatgcagattcgcaggccatttgggggcttgcagatcagctacatcagcctgtgaatgatgatattctcaacagagtgctccataccatcagcatgaaagtggccacagaaaacacagatgaacaactcagtgccatgatgcatttaatagaaaagataactactgaaggaaaaattcaagctttcagtgttgccagccgaactcttttctatgagattctttgttctcttattaacccaaagcgcaaggacactaggggattcagtcactttgctgaagtgactgagaattttgccttttctctgctgactaatgttacttgcggctctcctggtgaaaaaagcaaaaccatccttgatagttgcccatatttgtcaatattggctcttcactggtatcctcagcaaatcaatggacacaagtttgaaggaaaggaaggagattacattcgaattccagagaggctactggatgtccaggatgcagaaataatggctgggaaaagtacatgtaaattagtccagtttacagagtatagcagccaacagtggtttataagtggaaacaatcttcctaccctaaaaaataaggtattatctttgagtgtgaaaggtcagagttcacaactcctgactaatgacaatgaggttctctacaggatttatgctgctgagcctagaattattcctcagacatctctgtgtctcctttggaatcaggctgctgcaagctggttgtctgacagtcagttttgcaaagtgattgaggaaactgcagactatgtggaatgtgcctgttcacacatgtctgtgtatgctgtctatgctcggactgacaacttgtcttcatacaatgaagccttcttcacttctggatttatatgtatctcaggtctttgcttggctgttctttcccatatcttctgtgccaggtactccatgtttgcagctaaacttctgactcacatgatggcagccagcttaggtacacagattctgtttctggcgtctgcatacgcaagtccccaactcgctgaggagagctgttcagctatggctgctgtcacacattacctgtatctttgccagtttagctggatgctcattcagtctgtgaatttctggtacgtgctggtgatgaatgatgagcacacagagaggcgatatctgctgtttttccttctgagttggggactaccagcttttgtggtgattctcctcatagttattttgaaaggaatctatcatcagagcatgtcacagatctatggactcattcatggtgacctgtgttttattccaaacgtctatgctgctttgttcactgcagctcttgttcctttgacgtgcctcgtggtggtgttcgtggtgttcatccatgcctaccaggtgaagccacagtggaaagcatatgatgatgtcttcagaggaaggacaaatgctgcagaaattccactgattttatatctctttgctctgatttccgtgacatggctttggggaggactacacatggcctacagacacttctggatgttggttctctttgtcattttcaacagtctgcagggactttatgttttcatggtttatttcattttacacaaccaaatgtgttgccctatgaaggccagttacactgtggaaatgaatgggcatcctggacccagcacagcctttttcacgcccgggagtggaatgcctcctgctggaggggaaatcagcaagtccacccagaatctcatcggtgctatggaggaggtgccacctgactgggagagagcatccttccaacagggcagtcaggccagccctgatttaaagccaagtccacaaaatggagccacgttcccgtcctctggaggatatggccaggggtcactgatagccgatgaggagtcccaggagtttgatgatttaatatttgcattaaaaactggtgctggtctcagtgtcagtgataatgaatctggtcaaggcagccaggaggggggcaccttgactgactcccagatcgtggagctcaggaggatacccatcgccgacactcacctgtagcacctcactaaccattcgactgagcacactttcatatttgtatcagcttttgtgctaaaactctctaagtacatccacctgtgtaataggaacctgtgaattgtactggatgattaatacaaacgtgattgttgtatttggagtataaattactgattgtatgtgacctgaaaattcactgctataagaaaggtggagtcagtttgtatcagttaataggatgttcatattccaaggatattagttgtttttttaatcatcctatatggctaacattgtttaatgaaagtaataatcaataaagcaatagaatct

SEQ ID NO: 4 - DNA, 2705 bp, Homo sapiens
  source 1..2705: mol_type = unassigned DNA
tgctcgctccagggcgcaaccatgtcgccatttcttcggattggcttgtccaactttgactgcgggtcctgccagtcttgtcagggcgaggctgttaacccttactgtgctgtgctcgtcaaagagtatgtcgaatcagagaacgggcagatgtatatccagaaaaagcctaccatgtacccaccctgggacagcacttttgatgcccatatcaacaagggaagagtcatgcagatcattgtgaaaggcaaaaacgtggacctcatctctgaaaccaccgtggagctctactcgctggctgagaggtgcaggaagaacaacgggaagacagaaatatggttagagctgaaacctcaaggccgaatgctaatgaatgcaagatactttctggaaatgagtgacacaaaggacatgaatgaatttgagacggaaggcttctttgctttgcatcagcgccggggtgccatcaagcaggcaaaggtccaccacgtcaagtgccacgagttcactgccaccttcttcccacagcccacattttgctctgtctgccacgagtttgtctggggcctgaacaaacagggctaccagtgccgacaatgcaatgcagcaattcacaagaagtgtattgataaagttatagcaaagtgcacaggatcagctatcaatagccgagaaaccatgttccacaaggagagattcaaaattgacatgccacacagatttaaagtctacaattacaagagcccgaccttctgtgaacactgtgggaccctgctgtggggactggcacggcaaggactcaagtgtgatgcatgtggcatgaatgtgcatcatagatgccagacaaaggtggccaacctttgtggcataaaccagaagctaatggctgaagcgctggccatgattgagagcactcaacaggctcgctgcttaagagatactgaacagatcttcagagaaggtccggttgaaattggtctcccatgctccatcaaaaatgaagcaaggccgccatgtttaccgacaccgggaaaaagagagcctcagggcatttcctgggagtctccgttggatgaggtggataaaatgtgccatcttccagaacctgaactgaacaaagaaagaccatctctgcagattaaactaaaaattgaggattttatcttgcacaaaatgttggggaaaggaagttttggcaaggtcttcctggcagaattcaagaaaaccaatcaatttttcgcaataaaggccttaaagaaagatgtggtcttgatggacgatgatgttgagtgcacgatggtagagaagagagttctttccttggcctgggagcatccgtttctgacgcacatgttttgtacattccagaccaaggaaaacctcttttttgtgatggagtacctcaacggaggggacttaatgtaccacatccaaagctgccacaagttcgacctttccagagcgacgttttatgctgctgaaatcattcttggtctgcagttccttcattccaaaggaatagtctacagggacctgaagctagataacatcctgttagacaaagatggacatatcaagatcgcggattttggaatgtgcaaggagaacatgttaggagatgccaagacgaataccttctgtgggacacctgactacatcgccccagagatcttgctgggtcagaaatacaaccactctgtggactggtggtccttcggggttctcctttatgaaatgctgattggtcagtcgcctttccacgggcaggatgaggaggagctcttccactccatccgcatggacaatcccttttacccacggtggctggagaaggaagcaaaggaccttctggtgaagctcttcgtgcgagaacctgagaagaggctgggcgtgaggggagacatccgccagcaccctttgtttcgggagatcaactgggaggaacttgaacggaaggagattgacccaccgttccggccgaaagtgaaatcaccatttgactgcagcaatttcgacaaagaattcttaaacgagaagccccggctgtcatttgccgacagagcactgatcaacagcatggaccagaatatgttcaggaacttttccttcatgaaccccgggatggagcggctgatatcctgaatcttgcccctccagagacaggaaagaatttgccttctccctgggaactggttcaagagacactgcttgggttcctttttcaacttggaaaaagaaagaaacactcaacaataaagactgagacccgttcgcccccatgtgactttatctgtagcagaaaccaagtctacttcactaatgacgatgccgtgtgtctcgtctcctgacatgtctcacagacgctcctgaagttaggtcattactaaccatagttatttacttgaaagatgggtctccgcacttggaaaggtttcaagacttgatactgcaataaattatggctcttcacctgggcgccaactgctgatcaacgaaatgcttgttgaatcaggggcaaacggagtacagacgtctcaagactgaaacggccccattgcctggtctagtagcggatctcactcagccgcagacaagtaatcactaacccgttttattctatcctatctgtggatgtataaatgctgggggccagccctggataggtttttatgggaattctttacaataaacatagcttgtacttg

SEQ ID NO: 5 - DNA, 1578 bp, Homo sapiens
  source 1..1578: mol_type = unassigned DNA
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaatttactttttcaaattatttccaagagagattgatgggttaattaaaggagaagattcctgaaatttgagagacaaaataaatggaagacatgagaacttt

SEQ ID NO: 6 - DNA, 4553 bp, Homo sapiens
  source 1..4553: mol_type = unassigned DNA
acgcgtctgcggccagcccggactctttaaaagccggcggtgcgcggggcatcccagccaagccggagaggaggcgagcagcagggcctggtggcgagagcgcggctgtcactgcgcccgagcatcccagagctttccgagcggacgagccggccgtgccgggcatccccagcctcgctaccctcgcagcacacgtcgagccccgcacaggcgagggtccggaacttagcccaaagcacgtttcccctggcagcgcaggaaacgcccggccgcgcgccggcgcacgcccccctctcctcctttgttccgggggtcggcggccgctctcctgccagcgtcgggatctcggccccgggaggcgggccgtcgggcgcagccgcgaagatgccgttggaactgacgcagagccgagtgcagaagatctgggtgcccgtggaccacaggccctcgttgcccagatcctgtgggccaaagctgaccaactcccccaccgtcatcgtcatggtgggcctccccgcccggggcaagacctacatctccaagaagctgactcgctacctcaactggattggcgtccccacaaaagtgttcaacgtcggggagtatcgccgggaggctgtgaagcagtacagctcctacaacttcttccgccccgacaatgaggaagccatgaaagtccggaagcaatgtgccttagctgccttgagagatgtcaaaagctacctggcgaaagaagggggacaaattgcggttttcgatgccaccaatactactagagagaggagacacatgatccttcattttgccaaagaaaatgactttaaggcgtttttcatcgagtcggtgtgcgacgaccctacagttgtggcctccaatatcatggaagttaaaatctccagcccggattacaaagactgcaactcggcagaagccatggacgacttcatgaagaggatcagttgctatgaagccagctaccagcccctcgaccccgacaaatgcgacagggacttgtcgctgatcaaggtgattgacgtgggccggaggttcctggtgaaccgggtgcaggaccacatccagagccgcatcgtgtactacctgatgaacatccacgtgcagccgcgtaccatctacctgtgccggcacggcgagaacgagcacaacctccagggccgcatcgggggcgactcaggcctgtccagccggggcaagaagtttgccagtgctctgagcaagttcgtggaggagcagaacctgaaggacctgcgcgtgtggaccagccagctgaagagcaccatccagacggccgaggcgctgcggctgccctacgagcagtggaaggcgctcaatgagatcgacgcgggcgtctgtgaggagctgacctacgaggagatcagggacacctaccctgaggagtatgcgctgcgggagcaggacaagtactattaccgctaccccaccggggagtcctaccaggacctggtccagcgcttggagccagtgatcatggagctggagcggcaggagaatgtgctggtcatctgccaccaggccgtcctgcgctgcctgcttgcctacttcctggataagagtgcagaggagatgccctacctgaaatgccctcttcacaccgtcctgaaactgacgcctgtcgcttatggctgccgtgtggaatccatctacctgaacgtggagtccgtctgcacacaccgggagaggtcagaggatgcaaagaagggacctaacccgctcatgagacgcaatagtgtcaccccgctagccagccccgaacccaccaaaaagcctcgcatcaacagctttgaggagcatgtggcctccacctcggccgccctgcccagctgcctgcccccggaggtgcccacgcagctgcctggacaaaacatgaaaggctcccggagcagcgctgactcctccaggaaacactgaggcagacgtgtcggttccattccatttccatttctgcagcttagcttgtgtcctgccctccgcccgaggcaaaacgtatcctgaggacttcttccggagagggtggggtggagcagcgggggagccttggccgaagagaaccatgcttggcaccgtctgtgtcccctcggccgctggacaccagaaagccacgtgggtccctggcgccctgcctttagccgtggggcccccacctccactctctgggtttcctaggaatgtccagcctcggagaccttcacaaagccttgggagggtgatgagtgctggtcctgacaggaggccgctggggacactgtgctgttttgtttcgtttctgtgatctcccggcacgtttggagctgggaagaccacactggtggcagaatcctaaaattaaaggaggcaggctcctagttgctgaaagttaaggaatgtgtaaaacctccacgtgactgtttggtgcatcttgacctgggaagacgcctcatgggaacgaacttggacaggtgttgggttgaggcctcttctgcaggaagtccctgagctgagacgcaagttggctgggtggtccgcaccctggctctcctgcaggtccacacaccttccaggcctgtggcctgcctccaaagatgtgcaagggcaggctggctgcacggggagagggaagtattttgccgaaatatgagaactggggcctcctgctcccagggagctccagggcccctctctcctcccacctggacttggggggaactgagaaacactttcctggagctgctggcttttgcacttttttgatggcagaagtgtgacctgagagtcccaccttctcttcaggaacgtagatgttggggtgtcttgccctggggggcttggaacctctgaaggtggggagcggaacacctggcatccttccccagcacttgcattaccgtccctgctcttcccaggtggggacagtggcccaagcaaggcctcactcgcagccacttcttcaagagctgcctgcacactgtcttggagcatctgccttgtgcctggcactctgccggtgccttgggaaggtcggaagagtggactttgtcctggccttcccttcatggcgtctatgacacttttgtggtgatggaaagcatgggacctgtcgtctcagcctgttggtttctcctcattgcctcaaaccctggggtaggtgggacggggggtctcgtgcccagatgaaaccatttggaaactcggcagcagagtttgtccaaatgacccttttcaggatgtctcaaagcttgtgccaaaggtcacttttctttcctgccttctgctgtgagccctgagatcctcctcccagctcaagggacaggtcctgggtgagggtgggagatttagacacctgaaactgggcgtggagagaagagccgttgctgtttgttttttgggaagagcttttaaagaatgcatgtttttttcctggttggaattgagtaggaactgaggctgtgcttcaggtatggtacaatcaagtgggggattttcatgctgaaccattcaagccctccccgcccgttgcacccactttggctggcgtctgctggagaggatgtctctgtccgcattcccgtgcagctccaggctcgcgcagttttctctctctccctggatgttgagtctcatcagaatatgtgggtagggggtggacgtgcacgggtgcatgattgtgcttaacttggttgtatttttcgatttgacatggaaggcctgttgctttgctcttgagaatagtttctcgtgtccccctcgcaggcctcattctttgaacatcgactctgaagtttgatacagataggggcttgatagctgtggtcccctctcccctctgactacctaaaatcaatacctaaatacagaagccttggtctaacacgggacttttagtttgcgaagggcctagatagggagagaggtaacatgaatctggacagggagggagatactatagaaaggagaacactgcctactttgcaagccagtgacctgccttttgaggggacattggacgggggccgggggcgggggttgggtttgagctacagtcatgaacttttggcgtctactgattcctccaactctccaccccacaaaataacggggaccaatatttttaactttgcctatttgtttttgggtgagtttcccccctccttattctgtcctgagaccacgggcaaagctcttcattttgagagagaagaaaaactgtttggaaccacaccaatgatatttttctttgtaatacttgaaatttatttttttattattttgatagcagatgtgctatttatttatttaatatgtataaggagcctaaacaatagaaagctgtagagattgggtttcattgttaattggtttgggagcctcctatgtgtgacttatgacttctctgtgttctgtgtatttgtctgaattaatgacctgggatataaagctatgctagctttcaaacaggagatgcctttcagaaatttgtatattttgcagttgccagaccaataaaatacctggttgaaatacatggacgaagtaaaaaa

SEQ ID NO: 7 - DNA, 1773 bp, Homo sapiens
  source 1..1773: mol_type = unassigned DNA
ggaaaacagcagaggtgacagagcagccgtgctcgaagcgttcctggagcccaagctctcctccacaggtgaagacagggccagcaggagacaccatggggcacctctcagccccacttcacagagtgcgtgtaccctggcaggggcttctgctcacagcctcacttctaaccttctggaacccgcccaccactgcccagctcactactgaatccatgccattcaatgttgcagaggggaaggaggttcttctccttgtccacaatctgccccagcaactttttggctacagctggtacaaaggggaaagagtggatggcaaccgtcaaattgtaggatatgcaataggaactcaacaagctaccccagggcccgcaaacagcggtcgagagacaatataccccaatgcatccctgctgatccagaacgtcacccagaatgacacaggattctacaccctacaagtcataaagtcagatcttgtgaatgaagaagcaactggacagttccatgtatacccggagctgcccaagccctccatctccagcaacaactccaaccctgtggaggacaaggatgctgtggccttcacctgtgaacctgagactcaggacacaacctacctgtggtggataaacaatcagagcctcccggtcagtcccaggctgcagctgtccaatggcaacaggaccctcactctactcagtgtcacaaggaatgacacaggaccctatgagtgtgaaatacagaacccagtgagtgcgaaccgcagtgacccagtcaccttgaatgtcacctatggcccggacacccccaccatttccccttcagacacctattaccgtccaggggcaaacctcagcctctcctgctatgcagcctctaacccacctgcacagtactcctggcttatcaatggaacattccagcaaagcacacaagagctctttatccctaacatcactgtgaataatagtggatcctatacctgccacgccaataactcagtcactggctgcaacaggaccacagtcaagacgatcatagtcactgagctaagtccagtagtagcaaagccccaaatcaaagccagcaagaccacagtcacaggagataaggactctgtgaacctgacctgctccacaaatgacactggaatctccatccgttggttcttcaaaaaccagagtctcccgtcctcggagaggatgaagctgtcccagggcaacaccaccctcagcataaaccctgtcaagagggaggatgctgggacgtattggtgtgaggtcttcaacccaatcagtaagaaccaaagcgaccccatcatgctgaacgtaaactgtaagtgactcctcaccccttcctatatgtccctctaggattactctgtcaatggtgtgcaaaatggataaaactcacaggaggcagaatatcaatgaagagaccattatagcaaacagaattgcaaagtggttaagagctcagctcaggccgggcacagtggctcacgcctgtgatcccagcagtttgggaggccaaggcgggcggatcacgagggcaggagatcgaggccatcctggctaatatggtgaaaccccgtgtctactagaaatacaaaaaaaaattagccgggcatggtggcgggcgcctgtggtcccagctactcgggaggctgaggcgggagaatggcgtgaacctgggaggcggagctttcagtgagccgagatggtgccactgcactccagtctgggcaacagggcaagactctgtctc

SEQ ID NO: 8 - DNA, 1956 bp, Homo sapiens
  source 1..1956: mol_type = unassigned DNA
gtgatgtgtttaccttcagtgtctccttggaggtaaaagaagacgatggaaaaggaaactttagccctgtgcctaggatagagataaattttatttcaaattaaagcaaggaatagagaagaaggttgtgattacagtgcagcaactttctaacaaagaattagctattgaaagatgttttggaatgttattaagcccaggtcgaaacgtgaagaacagtgacatgcatttactggatatggaatccatgggaaagagctatgatgggagagcttatgtcatcactggcatgtggaaccccaatgcaccagtatttctggcacttaacgaggaaaccccaaaagataagcaagtatacatgactgtggcagtggatatggtagtcacagaggtggtggagcctgttcgctttctcctggagacagtagtccgtgtgtaccctgcaaatgagcgattttggtatttcagcagaaagactttcacagagactttcttcatgagattgaaacagtctgagggaaaaggccataccaatgctggagatgcaatatatgaggtggtgagtctacagcgagagtctgacaaggaggaaccagtcactcctactagtggagggggtccaatgtcaccccaggatgatgaagcagaagaggagagtgataatgaactctcaagtggaacaggtgatgtgtctaaggattgtcctgagaagatcctgtattcttggggagagttgctaggaaaatggcacagtaaccttggtgcacgaccgaaagggctgtctactctggtgaagagtggtgtccctgaagcattgagggcagaggtatggcagttattggcaggctgccatgacaaccaggcaatgctggatagataccgaattcttatcacaaaggtctgttggagtttgctggaggtccactccagaccctgtttgcctgggtatcaccagtggaagctgcagaacagcaaatattgcagaatggcaaatgttgctgcctgatccttcctctggaagcttcatctcagaagggcacctggctgtatgaggtgtcggttggcccctcctgggaggtgtctcccaattagactactcaggtgtcagggacccacttgaggaggcagtctgtccattctcaggtcccaaactacatgctgggagaaccactactctcttcaaagctgtcagacagggacatttaagtctgcagaagtttctgctgccgtttgttcaactatgccctgcccccagtactggagtccaggaaggcaggcaggcctccttgagctttggtgggctccacccagttcagtcttcccggctgctttgtttacctactcaagcttcagcaatggcggatgcccctccctcagccttgctgacacttgcggcttgatctcagactgctgtgttagcagtgagtgaggctccgtgggtgtgggactctccgagccaggtgcgggatataatctcctggtgttccgtttgctaagaccattggaaaagcgcagtattagggtgggagggtcccgattttccaggtactatctgtcatggcttcccttggctaggaaagggaattccccaaccccttgcacttcccaggtgaggcaatgccccacgctgctccttgggctgcacccactgtgtgacaagccccagtgagatgaacccggtacctctgttggaaatgcagaaatcacctgtcttctgcgtcgctcacgctgggagctgtagactggagctgttcctttttggccatcttggaacctcggttcaaacctgagttgtaatactcactcttcctgttgctgcctatgtaattttgtagaagttacctaattgcttccaagcttctgttggattttttgagaactgaatgacatagtacattttgagtgcttaatgtattgctttgtgcatggtattatttaataaatattagctttggt

SEQ ID NO: 9 - DNA, 873 bp, Homo sapiens
  source 1..873: mol_type = unassigned DNA
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 10 - DNA, 1264 bp, Homo sapiens
  source 1..1264: mol_type = unassigned DNA
ggagggagcagtcggtcgctgcgccccggcgggccactttcccgggaccccgctcgtcttccttgggccgagattttccactgcgcccctccgagtacccgggttccaaacccctagccacgacatggaagaatttttgcaacgcgccaaatctaaactgaatcgaagcaaacgcttggagaaggtccatgtggttattgggcctaaatcgtgtgacttggattctctcatttctaccttcacatatgcttactttctagacaaggtcagtccaccaggggttctgtgtttaccagtgctgaacataccaagaactgaattcaactacttcaccgagacgaggtttattttagaagagctaaatatttccgaatcattccacatattccgggatgaaattaacctgcatcagctaaatgatgaagggaagttatcgataacacttgttggcagcagtgtgctggcgagtgaagacaaaactttagaatcagcagttgtcaaagtcattaatccggttgagcagagcgatgccaacgttgagttccgagagtcttcctcttctctcgtgctaaaggagattctccaagaggctcctgagctcatcaccgagcaactggctcatcgcctcagaggtagcattcttttcaagtggatgaccatggaatcagagaagatctcagagaagcaggaggaaattctttctatcctggaagaaaaatttcctaacttgcctccaagagaggacatcatcaacgtcctacaggagacccagttcagtgctcagggtttaagtattgaacagacaatgttgaaagatctaaaggagctgtcagatggagaaataaaagtggccattagtactgtgagcatgaaccttgaggtaagggtgggaatgcttttttagcattgattgatttcccacaattgcagtctgagcaactggaatgtaactctctccattggataagtccatgatagtcctttgcttctttgtgatacatttgacttggaatatagtggcaggttattatttgggtgaaaacactatgctaagtcaatgaaaaatgccaaacctggattctcaagatgaatgctctttcattcatctcagtaaaacaaagcctaaaacaaacactcagatgtgggtgtatatttaacctgtttaatagcaattatgatatgtgtgattaggttcacttccatattttctgtgtaccaccctgtattgttcatagaacttttcttaaaaaaaaaaaaaaaaaaa

SEQ ID NO: 11 - DNA, 2592 bp, Homo sapiens
  source 1..2592: mol_type = unassigned DNA
atggatgctagcatttggagcaatgaactcatcgagctttttattgtcattggaaacaaaagagcaaatgacttttgggctggtaatcttcaaaaggatgaagaattacatatggactcaccagtagaaaagagaaaaaactttattactcagaaatataaagaaggaaaattcagaaaaactcttttggcatctctcaccaaagaagaattaaataaggctctatgtgctgctgtagtgaaaccggatgttctagaaacaatggctttgctgttcagtggagcagatgtcatgtgtgccaccggagaccccgtgcatagcaccccctatctgctagccaagaaagctgggcaaagtctgcaaatggaatttctctaccataacaaattctcagatttccctcaacatgatattcattccgagggtgtattaagtcaagagtcttcccagtccacattcctctgtgactttttatatcaagctccttctgctgcttctaaactctcttcagagaaaaaactgcttgaagagacaaataaaaaatggtgtgttttggaaggaggcttcttgagttactatgaaaatgataagtctaccacacctaatggcaccattaatatcaatgaagttatctgcctggctatacacaaagaggacttctatttaaatactgggcccatctttatctttgagatctacttaccctccgaacgtgtgtttttatttggagctgaaacatctcaagctcaaagaaaatggacagaggcaatagccaagcattttgttcccttatttgctgaaaacttaacagaagctgactatgatttgattggtcaactcttctacaaagactgccatgccctggatcagtggagaaaaggctggtttgctatggacaaatccagcttgcatttttgccttcaaatgcaagaagttcagggagatagaatgcacttaagaagactgcaagagctaacaatcagcacaatggttcaaaatggggaaaaactggatgttttactcttggtagaaaaagggagaacattatacatccatgggcataccaagttggatttcacagtctggcatactgcaattgaaaaagcagcaggtacagatggtaatgctttacaagatcagcagctcagcaaaaatgacgttcccattatagtgaacagctgtatagcatttgttacacagtatggtttaggatgcaaatatatctatcaaaagaatggtgatcctttgcatataagtgaactcctggagagtttcaaaaaggatgcaagaagctttaaattgagggctggaaaacatcagcttgaagatgtgacggctgtgttgaaaagttttctctctgacattgatgatgcactgcttactaaggagctctacccatattggatctctgctttagatacgcaagatgacaaggaaagaattaaaaaatatggagcatttatacgttctcttccaggggtcaaccgagcaacactagcagctatcattgaacacctgtatagggttcagaaatgctcagaaatcaatcacatgaatgcccataatttggccttggtcttttcatcctgtttgtttcaaacgaagggacaaactagtgaagaagtgaatgtaattgaggacctaattaataattatgtagaaatatttgaggttaaagaagatcaagtcaaacaaatggacatagaaaatagctttattaccaagtggaaagacacccaagtttcccaggctggagatttgttaattgaagtatatgtagaaaggaaggaacccgactgtagtattataattcggatatctcctgtgatggaagcagaagaattaactaatgatatattagcgataaaaaatattattcctacaaaaggtgatatttgggccacatttgaagtcattgaaaatgaagagctagagcgtcctcttcactacaaggaaaatgtactggagcaggtgcttcggtggagttcattagctgaacctggctctgcttacctggtggtgaagagattcttaaccgctgacacaattaaacactgcagtgaccggagtacactgggaagcatcaaagaaggaatcttgaaaatcaaagaagaaccatccaaaatactatctggaaataagtttcaagaccggtattttgttttacgagatgggtttctctttctttacaaggatgtgaagagtagtaaacatgacaagatgttttctctcagttccatgaagttttatcgtggagtgaaaaagaaaatgaagcctccaacaagctggggattgaccgcatattctgagaaacatcactggcacctgtgttgtgatagttcacgaactcagacggagtggatgaccagtatctttattgcccagcatgaatatgatatatggccaccagctggaaaggaacgaaaacgttcaataaccaaaaatcccaaaattggaggtttgcctctgattcctatacagcatgaggggaatgcaaccttggcccggaaaatattgagagtgcaagagcagaacttgaaaggctgcggctcagtgaaaagtgtgataaagagtccgtggactctagcttaa

SEQ ID NO: 12 - DNA, 4502 bp, Homo sapiens
  source 1..4502: mol_type = unassigned DNA
cgagggaagaggccggagggagatcgcggaggggagggggcgggaggggggtggggtatcctgagtcgtccgtggtccggagtctggaaggccctagagcggcgccagtcgacccgcctcgccacaacttgcccagaccagacacgtttcatcctgcgccctgcaagaaggagccggcctgcctctctccgctttgctgctcctcggcctccgcgggcccggcccgcgtcagcagcgaccctggggtctgggtcccctgtgtcgcccccgcccgcctgcagcgcccggcacccgcccaggagcgcgcagctggggttctagggacgtatacttgagcaagagagaccacagctcttgttcccgctgatcctgcagcccagtggatggagtccagaatctacagacctgccaggaaagaaaaaaaattcctgatgtctggtccaagcaggaattccccggtggattggggaatgtctggcttttcctcagcttattgatctctgtggtaaccactggaggcccccagaagacccatagaaatgagaggcctgaggtctacaagtcgctgctagaaatattttagcctctccaaagcccagaatgcagccccgacccaagtttgtaagggttctgggtgcacgctgaccctgcgcgggcagacgcgccctttgctccaggtccggacctgggcgctgctatagcaacgtcctggacgcccagaccttaggccgccgccgccgcggaagcgaggaacccggccttctcccgctcctgagggctgtggcggcggcggcccgggaggcggcccaggctgggtaaagaccgcccggctcctcctatgcaagctgaggcagcggattggttttcaagcatgcccttccagaagcatgtctactacccgctcgccagcggcccagaggggcccgacgtcgctgtggccgccgccgccgcgggtgcggcctccatggcctgtgcgccccccagcgcggcttcggggcccctgcccttcttccagttcaggccgcggctggagagtgtggactggcggcggctgagcgccatcgacgtggacaaggtggcgggggctgtggacgtgctgacgctgcaggagaacatcatgaacatcaccttctgcaagctggaagacgagaagtgcccacactgccagtcgggggtggacccggtgctgctgaagctcatccgtctggcgcagttcaccatcgagtacttgctgcactcacaagagttcctcacctcgcagctgcacaccctggaggagcggctgcgcctgagccactgcgacggcgagcagagcaagaagctgctcaccaagcaggcgggggagatcaagacgctcaaggaagagtgcaaacgccggaagaagatgatctccacccagcagctgatgatcgaggccaaagccaactattaccagtgccatttttgtgacaaggcctttatgaaccaagcttttctacaaagtcacattcaacgccgccacactgaagaaaattctcattttgagtatcagaaaaatgcacagattgagaagctccggagtgagatcgtcgtattgaaggaagagctgcagctcaccaggtctgagctagaggctgcacaccatgccagtgcagtcagattctccaaggaatatgaaatgcagaaaacaaaagaggaagactttttgaagttatttgacaggtggaaagaagaagaaaaggagaaactagttgatgaaatggaaaaagtcaaggagatgtttatgaaggaatttaaagaattaacttcgaagaattcagcattagaatatcaactgtcagaaatccagaagtccaatatgcagatcaagtccaacataggcacattaaaagatgcacacgagtttaaagaagaccgttctccatatccccaggatttccataatgtcatgcagcttcttgatagtcaggaaagcaaatggacagctcgagttcaagctattcatcaagaacacaagaaagagaagggtcggctcctgtcacatatagagaaacttcgaacctcaatgatagatgatctaaatgcaagcaatgttttctataagaaaaggatagaagagctagggcagagactccaggagcagaatgagctgattataactcagagacagcagattaaagactttacctgtaatccattaaacagtatcagtgaacccaaagtgaatgccccagccctgcacactttggaaactaaatcaagtctgccaatggtgcatgaacaggcattctcgtcgcacatactggaaccaatagaagaactttcagaggaagaaaaaggaagggaaaatgaacagaaattaaataacaacaaaatgcatttaaggaaagctttgaagagtaactcctccctcactaagggactaagaacaatggtggagcagaacttgatggagaaactggaaaccttggggattaatgcagatatacgtggcatttcaagtgatcagttgcatagagtactaaaaagtgtggaatcagaaagacataagcaagaaagagaaatacctaactttcatcaaattcgagaattccttgaacatcaagtcagctgtaaaattgaggagaaagcactactctcttcagatcagtgcagtgtttctcaaatggataccctttcaactggagaagtacccaaaatgatacaacttccttccaaaaacagacaactgattagacaaaaagctgtttctactgataggacatctgttccaaaaattaagaaaaatgtcatggaagatccttttcccagaaagtcttcaactattacgacccctccttttagttcagaggaggagcaggaggacgacgacctcatccgggcatacgcatccccaggcccacttcctgtgccgccaccacaaaacaagggcagcttcgggaagaacacagtgaaaagtgacgcggacgggaccgagggaagcgaaatcgaggacactgatgattctcccaagcccgcaggagtcgccgttaaaacacctactgaaaaagttgaaaagatgtttccacatcgcaaaaatgtgaacaaaccagtcggtggaactaatgtccctgagatgtttatcaaaaaagaagaattacaagaactaaagtgtgcggatgtggaggatgaagactgggacatatcatccctagaggaagagatatctttgggaaaaaaatctgggaaagaacagaaggaacctccacctgcgaaaaatgaaccacattttgctcatgtgctaaatgcctggggcgcatttaatcctaaggggccaaagggagaaggacttcaagaaaatgaatcaagcacattaaaaagcagcttagtaactgtgactgattggagcgacacttcagatgtctaattccacatgtcagaagattattccagaagccagcagtatttcagtatcacagtgtttcagtaatttgcctccatgattctagtgcttctgccttaccgtgtttcccacagcaacacagagactgattcaaagaacaatggtctctttaatggcacccaatacagtattgaaaatcagatcatcaacagtatttcgaagcatgtaaaggtgtttaagacttccgctgctgcttaaaaataacatgtcattgaagtcataaaaagttttttcttcagaaaggtactctagtgttaagtgtatttttttcaactaattttttagtgaattttttttaaacttacagcatgttttggtttgaattactaaaactttaaaaaatatttttcttatgtatgctgtcgtatcgtaggcgtttatattataaaattctgttagtagtcttaaaattgaattggtggaaccactaatccttaaaagttagtctggttatttttcatatagaagtaagtttaatccgagtgtggtggtgttcacctttaatcccagctacttgggaggctgaggtgggaggataacttgagcacaggagttcaagaccagcgtgggcaatatagcaagactccacccctccacaccccaaaaaagtaagtttaggattagaatatagctaggtccaatgttaaatacattttcctggagtacatttgtcacattcagctttgagccactgtaagcatgttactattaaatggttggttattttatatagcatattctttatcttggatattttatgaataaagtatagttattttaagtgccaattaatttatcagactaaatagaaaatatttgagccattactgaattcacatatgtatgtttttttttactatttaaaatacccaacatgtattatgaaatacctcaaaagtaatttagttacattcttaaacaatgacattgtcgaaagaaagttcttataagctgttttttgcatttttataacttggttatactatattctgtttccaagtaaccttttaactaaaagatttgttgggttttagatctcttttcatttgtcaaccttttcagtaaagccctctgttacatc

SEQ ID NO: 13 - DNA, 1887 bp, Homo sapiens
  source 1..1887: mol_type = unassigned DNA
cggggcgcggcgggctccctcggggtcccagctggccggcactcggcggccgcggcgcgatggaggcgccggccgagctactggccgcgctgcctgcgctggccaccgcgctggcccttctgctcgcctggctactggtgcggcgtggggcggccgcgagcccggagcctgcccgcgcgcccccggaacccgcgcccccggccgaggccaccggggccccggcgccgtcccgcccctgcgcccccgagccggcggcctcgcccgcggggccggaggagcctggagagcccgcggggctgggggagctcggggagcctgcgggaccgggggagcccgaagggccaggggatcccgcggcggcgccagcggaggcggaggagcaggcggtggaggcgaggcaggaagaggagcaggacttggatggtgagaaggggccatcatcggaagggcctgaggaggaggacggagaaggcttctccttcaaatacagccccgggaagctgaggggaaaccagtacaagaagatgatgaccaaagaggagctggaggaggagcagagaactgaagaataacgaagttatccttagcgtcctcctaaaggcttttccttttggcatcttaaaagcttgagagataaaacggaaaccccagagaggagtctgggcaggctcccagggtgcatgctgcctccataaatctgctgagctctagaccctcaatcaggacttgtcccttggctagcaggatcctgggaacacctttggccctgccctgtgtagagatgttcatgtctgttcctgtgggtcactttgttaagctgaagagttttaagaggtagagctcagaccctggactgggatttttcttaccactcaaacttgctatccacacaccctgcacaccttagataaaaagaacattttaaaagcagagttcactttcactccagtctcccctcttttgccctcactgaagccaaaccacagaagactttgaggaatgagagacaaatgaggtagagctcacctgtgctcaccagctccgtcagggtggtcagccgacccctttccctgggaaccccacttctctctgtggctggcttggttgtcgggggtgagatgccatattgattacagggcagcaaagaaccagtaccaggaatttacttgaccattccccttatttttcatctagaggaatctcggattcagccctttcattgctaagacaccttttcactgaggttcttaccagctcagccaaatctccactctgctatagcagaagcaataatgtttgctttaaaaagatttcttgacctatgccttttcttagaaagtttgatagattagttagaacttcagatcatcagatcagtctcaaatgggtttcttggaattttatatttgacaatatttatactataccaaactcatttgcagttcttaggtttgttggttaaaacatttttttaaagcagtaagtttatagaaaatgttttcatttaatggaaggctggggaatgtccagcatcaacccctatggcatgcattcccagtggccttctcatctgggcctggaacctttggttcagggcttaggggagaacaggccacatggcaacagccacacagtcattgccttcaacacagagccacgtgtccccaaacagcaatagtcatgcccttgtccaggctgggatctaattgatacaataggtcgttgactccctcctagtagagctatctaggtttgtctggaaagtttccgaccctggcttataggcaccacacctcatgtactcctcatggcttggatctctgtattcagcctttgttcagtccaataaactttgagtagatgatctcaaaaaaaaaaaaaaaaaaa

SEQ ID NO: 14 - DNA, 2850 bp, Homo sapiens
  source 1..2850: mol_type = unassigned DNA
gagaacggggtagcccggcgcttacacatgtcacatgtgctttttaagacggccgggagcgcctgcgagctggatctggtggaggatgctgcggcaggtgcttcgcagagggctccagtcgttctgccacaggctgggtttgtgcgtgagccggcacccggtctttttcctcaccgtgcccgcagtcctgacaatcaccttcggcctcagcgcgctcaaccgcttccagcccgagggcgacctggagcgcctggtcgctcccagccacagcctggccaagatcgagcgcagcctggccagcagccttttccccctggaccagtccaaaagccagctctattcggacttacacacccctgggaggtatggcagggtgatcctcctctccccaaccggggacaatattttgctccaggctgaggggatcctgcagacccaccgagccgtgctggaaatgaaggatgggaggaacagttttattggacaccaactgggcggggtagtggaagtgccaaacagcaaagatcagcgggtcaagtcagccagagccattcaaatcacctactacctccagacctatggctctgccacccaagacctcataggggagaagtgggagaatgagttctgtaagcttataaggaagctccaggaggagcatcaagaactccagctctactctttagcatcctttagcctctggagggactttcataagaccagcatcctggccagaagcaaggtcctggtgagcctcgtgctgatcctgaccacagccaccctctccagctccatgaaggactgcttgcgcagtaagcccttcctgggcctcctgggggtgctcacagtatgcatctccatcatcacagcagcagggatcttcttcatcaccgatggaaagtacaactccaccctgctgggaatcccgttcttcgccatgggtcatggaactaaaggagtgtttgagcttctgtccggatggcggagaaccaaagagaacttgcccttcaaagacaggatagcagatgcctattctgatgtgatggtcacctataccatgaccagctccctgtacttcatcacttttggcatgggtgccagcccattcacaaacatagaggctgtgaaggtcttctgtcaaaacatgtgtgtctctattctgttgaactacttctacattttctccttctttggctcctgtctggtctttgctggccaactagagcaaaaccgctaccacagcatcttttgctgtaagatcccttctgcagaatacctggatcgcaaacctgtgtggttccagacagtgatgagtgatgggcatcaacagacgtcccatcatgagacgaacccctaccagcaccacttcattcagcacttcctccgtgaacattataatgaatggattaccaatatatatgtgaagccatttgttgtcatcctctatctcatttatgcctccttctccttcatggggtgcttacagatcagtgacggagccaacatcatcaatctactagccagtgattcgccaagtgtttcctatgccatggttcagcagaaatatttcagcaactatagccctgtgataggattctacgtctatgagcccctagagtactggaacagcagcgtccaggatgacctaagaagactctgtagtggattcactgcagtgtcctgggtggagcagtactaccagttcctgaaagtcagcaacgtcagtgccaataacaaaagtgacttcatcagtgtcctgcaaagctcatttttaaaaaagccagaattccagcattttcgaaatgatatcatcttctccaaggcaggggatgaaagcaatatcattgcttctcgcttgtatctggtggccaggactagcagagacaagcagaaagaaatcacagaagtgttggaaaagctgaggcccctatccctctcaaagagcatccgattcatcgtgttcaacccctcctttgtcttcatggaccattacagcttgtctgtcacagtgcctgttctgattgcaggctttggtgttctcctggtgttaatcctgacttttttcctagtgatccaccctctgggaaacttctggctaattcttagcgtcacctcaattgagctgggcgttctgggcttaatgacattatggaacgtcgacatggattgcatttctatcttgtgccttatctacaccttgaatttcgccattgaccactgtgcaccactgcttttcacatttgtattagcaactgagcacacccgaacacaatgtataaaaagctccttgcaagaccatgggacagccattttgcaaaatgttacttcttttcttattgggttagtcccccttctatttgtgccttcgaacctgaccttcacactgttcaaatgcttgctgctcactgggggttgcacacttctgcactgttttgttattttacctgtgttcctaacgtttttccccccttccaaaaagcaccacaagaaaaagaaacgtgccaagcgaaaggagagagaggaaattgaatgcatagaaattcaagagaacccggatcacgtcaccacagtatgaggggtatagaccagtggattatttttcttttccagtattgcacaatgatgcagggcaagtaaagctcagacctcagctgcttgggctggccaggggtaacaaggcaagtcagatcaagagtgcattattcatgacacttcaaggtgcctgcttcttggggggaagagggaataaaaaaagaggaaaaagttatttgcaaccttgttctcctctaaaaacaagtttctggatgtaatctgagagctcttccaaggaatggatgaatcaatggagtg

SEQ ID NO: 15 - DNA, 1491 bp, Homo sapiens
  source 1..1491: mol_type = unassigned DNA
gccctgcgcggggacactcagagcccggtggggggaggaaggcggcatgccccagacggtgatcctcccgggccctgcggcctggggcttcaggctctcagggggcatagacttcaaccagcctttggtcatcaccaggattacaccaggaagcaaggcggcaggtgccaacctgtgtcctggagatgtcatcctggctattgacggctttgggacagagtccatgactcatgctgatgcgcaggacaggattaaagcagcagctcaccagctgtgtctcaaaattgacaggggagaaactcacttatggtctccacaagtatctgaagatgggaaagcccatcctttcaaaatcaacttagaatcagaaccacaggacgggaactactttgaacacaagcataatattcggcccaaacctttcgtgatcccgggccgaagcagtggatgcagcactccctccgggattgactgtggcagtggacgcagcaccccttcttctgtcagtactgttagtaccatttgcccaggtgacttgaaagttgcggctaagctggcccctaacattcctttggaaatggaacttcctggtgtgaagattgtacatgctcagtttaatacacctatgcagttgtactcagatgacaatattatggaaacactccagggtcaggtttcaacagccctaggggaaatacctttgatgagcgagcccacagcctcggtgccccccgagtcggacgtgtaccggatgctccacgacaatcggaatgagcccacacagcctcgccagtcgggctccttcagagtgctccagggaatggtggacgatggctctgatgaccgtccggctggaacgcggagtgtgagagctccggtgacgaaagtccatggcggttcaggcggggcacagaggatgccgctctgtgacaaatgtggcagtggcatagtcggtgctgtggtgaaggcgcgggataagtaccggcaccctgagtgcttcgtgtgtgccgactgcaacctcaacctcaagcaaaagggctacttcttcatagaaggggagctgtactgcgaaacccacgcaagagcccgcacaaaacccccaagaggctatgacacggtcactctgtatcccaaagcttaagtctctgcaggcgtggcacacgcacgcacccacccacgcgcacttacacgagaagacattcatggctttgggcagaaggattgtgcagattgtcaactccaaatctaaagtcaaggctttagacctttatcctattgtttattgaggaaaaggaatgggaggcaaatgcctgctatgtgaaaaaaacatacacttagctatgttttgcaactctttttggggctagcaataatgatatttaaagcaataattttttgtatgtcatactccacaatttacatgtatattacagccatcaaacacataaacatcaagatatttgaaggactctaattgtctttccttgacaa

SEQ ID NO: 16 - DNA, 1504 bp, Homo sapiens
  source 1..1504: mol_type = unassigned DNA
tgcagacggaacttcagccgctgcctctgttctcagcgtcagtgccgccactgcccccgccagagcccaccggccagcatgtcctctgctcacttcaaccgaggccctgcctacgggctgtcagccgaggttaagaacaagctggcccagaagtatgaccaccagcgggagcaggagctgagagagtggatcgagggggtgacaggccgtcgcatcggcaacaacttcatggacggcctcaaagatggcatcattctttgcgaattcatcaataagctgcagccaggctccgtgaagaagatcaatgagtcaacccaaaattggcaccagctggagaacatcggcaacttcatcaaggccatcaccaagtatggggtgaagccccacgacatttttgaggccaacgacctgtttgagaacaccaaccatacacaggtgcagtccaccctcctggctttggccagcatggcgaagacgaaaggaaacaaggtgaacgtgggagtgaagtacgcagagaagcaggagcggaaattcgagccggggaagctaagagaagggcggaacatcattgggcttcagatgggcagcaacaagtttgccagccagcagggcatgacggcctatggcacccggcgccacctctacgaccccaagctgggcacagaccagcctctggaccaggcgaccatcagcctgcagatgggcaccaacaaaggagccagccaggctggcatgactgcgccagggaccaagcggcagatcttcgagccggggctgggcatggagcactgcgacacgctcaatgtcagcctgcagatgggcagcaacaagggcgcctcgcagcggggcatgacggtgtatgggctgccacgccaggtctacgaccccaagtactgtctgactcccgagtacccagagctgggtgagcccgcccacaaccaccacgcacacaactactacaattccgcctaggtccacaaggccttcactgttttccccccaagggaggctgctgctgctcttggctggagccagccagggccagccgaccccctctccctgcatggcatcctccagcccctgtagaactcaacctctacagggttagagtttggagagagcagactggcggggggcccattggggggaaggggaccctccgctctgtagtgctacagggtccaacatagaacagggtgtccccaacagcgcccaaaggacgcactgagcaacgctattccagctgtccccccactccctcacaagtgggtacccccaggaccagaagctcccccagcaaagcccccagagcccaggctcggcctgcccccaccccattcccgcagtgggagcaaactgcatgcccagagacccagcggacacacgcggtttggtttgcagcgactggcatactatgtggatgtgacagtggcgtttgtaatgagagcactttcttttttttctatttcactggagcacaataaatggctgtaaaatctacacg

SEQ ID NO: 17 - DNA, 1781 bp, Homo sapiens
  source 1..1781: mol_type = unassigned DNA
actcgggaagacttcagagaagtctcacaaaggactcggctggctgcttttctcagtgccgaagccgcgccatgctcgttctcagaagcggcctgaccaaggcgcttgcctcacggacgctcgcgcctcaggtgtgttcatcttttgctacgggccctagacaatacgatggaacgttctatgaatttcgtacttattaccttaaaccttcaaatatgaatgcgttcatggaaaatcttaagaaaaacattcatcttcggacctcttactctgaattggttggattctggagtgtagaatttggaggcagaacgaataaagtgtttcatatttggaagtatgataattttgctcatcgagctgaagttcggaaagccttagccaactgtaaggaatggcaagaacaatctatcattccaaatttggctcgcattgataaacaagagacggaaattacttacctgataccatggtccaaattagaaaagcctccaaaagaaggagtctatgaactagctgtttttcagatgaaacctggtgggccagctctgtggggtgatgcatttgaaagagcaattaatgcccatgtcaatttaggctacacaaaagtagttggtgttttccacacagaatatggagaactcaacagagttcatgttctttggtggaatgagagtgcagatagtcgtgcagctgggagacataagtcccatgaggatcccagagttgtggcggctgttcgggaaagtgtcaactacctagtttctcagcagaatatgcttctgattcctgcatcattttcaccattgaaatagttttctactgaaatacaaaacatttcattaactgctctaagatgtgtctgctaatggtgcttaaattctcccaagaggttctcgcttttatttgaaggaggtggtaagttaattagttaatttgctgtgcttcttgcatttttgaaagttacatattctccactgctttaagaaataattcagttcactttcaccttggcatttcagtatctgttacacattagaagtagttgtcactatttcatcatcttggtttttcatttgttttagaatacctcttctgtattttgataactcattgctttatagcattttcttttactcaaataaggattttacatttccttgcctgacagtatttttgaattatttatataaaatatctatcttttcatcatgtctatagttcctgagattttaaaaaaatttgcttagtaaaggttattttgtgatataaaatgggatttataaaaatattagattgtttatttctttactgtggaaaagtagaatgtcatctgtattaattattgcttttacattcattgattattagtcattctaacttggaaaataatgcaattgggtcacagtgttaaaaatctagaaaagacttgttggtttatatgctgaaattgttcatttataattaattttactaatttctccttagtttggatcactaacagagatcttgggacatttatttgttttaaagaaatatttatggttatggaaacgcttgccctaataaaaatcctgcatattcattgtttttttaaattcacattttatacttatatgatctctaaagctcttgctatgttgctataagacagtaatatagtgataatttaccaactttattgaaaatgttgttacatcaataaaatagcatgctgggaacctgagaaggaaggtttctttagtactgccaaaaaaaaaaaaaaaaaaaaaaaaaaaaaa

SEQ ID NO: 18 - DNA, 3023 bp, Homo sapiens
  source 1..3023: mol_type = unassigned DNA
tgaattcaaaacagttactctgaatggtctttgctaagaacaatttaatgattaagtaaggtcagtgtccttggaagtccaaactctagccagatttccctggtctacacccctagggataaggtaaatgtttaagcacacagtgaacttcctgaggcccccaaatctaatggaactagctattgagggctaaaagaggatggtttttttagaaaactcgaagcaaatctctcaggctggggatatttcaaagactactactattattattaataacaattgcaatatttgttgagtccctaaatgaagctaaaactttgttctaataaatttaatctttacagcaacctatgaggtagataatattgtcattcccatgagggagctaaggatcagagaaggtaagtcacttgtctaaggtcacatagctagcatgttatgcaatcaggagtcaaacctggtttgtctgaatctgaagtccatctgctctgtgcacttttataccgtctgctttttcctttattcctaaccttcttccattctgattcccactgagtagtggacaggaaccactgaagtttgcctgacaccatcaaccaggccctagtcacctggctttgcctttgccctgctgtgtgatcttagctccctgcccaggcccacagccatggccatggcccagaaactcagccacctcctgccgagtctgcggcaggtcatccaggagcctcagctatctctgcagccagagcctgtcttcacggtggatcgagctgaggtgccgccgctcttctggaagccgtacatctatgcgggctaccggccgctgcatcagacctggcgcttctatttccgcacgctgttccagcagcacaacgaggccgtgaatgtctggacccacctgctggcggccctggtactgctgctgcggctggccctctttgtggagaccgtggacttctggggagacccacacgccctgcccctcttcatcattgtccttgcctctttcacctacctctccttcagtgccttggctcacctcctgcaggccaagtctgagttctggcattacagcttcttcttcctggactatgtgggggtggccgtgtaccagtttggcagtgccttggcacacttctactatgctatcgagcccgcctggcatgcccaggtgcaggctgtttttctgcccatggctgcctttctcgcctggctttcctgcattggctcctgctataacaagtacatccagaaaccaggcctgctgggccgcacatgccaggaggtgccctccgtcctggcctacgcactggacattagtcctgtggtgcatcgtatcttcgtgtcctccgaccccaccacggatgatccagctcttctctaccacaagtgccaggtggtcttctttctgctggctgctgccttcttctctaccttcatgcccgagcgctggttccctggcagctgccatgtcttcgggcagggccaccaacttttccacatcttcttggtgctgtgcacgctggctcagctggaggctgtggcactggactatgaggcccgacggcccatctatgagcctctgcacacgcactggcctcacaacttttctggcctcttcctgctcacggtgggcagcagcatcctcactgcattcctcctgagccagctggtacagcgcaaacttgatcagaagaccaagtgaagggggatggcatctggtagggagggaggtatagttgggggacaggggtctgggtttggctccaggtgggaacaaggcctggtaaagttgtttgtgtctggcccacagtgactctctgtgcacgactcaactgccaagggcatcactggccaattcttggatttagggattggctaggagttgctggggtccactcctgggcctgccccagctccttgcccagggagagggaaagagttaacggtgtgggccactccagcttgcccttccactgccactcactggggtgaggctgggggtcagcttggtgaggattggggcttctagattgtctaggcaggaggtgaaacttaggccagagtcagatttgagctgagccaggggaggccttggcaacctacttctactcagatttcattgctggatgcggaaggggtaggcccaaaatatatacaggatcttactgtcccttgaagcccagccacaagtgttggagctgcagagagaccccaaaggtagtagattgtgccagatacaaatgggtcccatccagtgcttcatactccttcagtcactatcccagacagtgagccccagatctcctagctctggcttctgtgtcccacacggcctgttcccagcttctctcctggttcccttgttacggattcatttatccattcagtgtttcctgggcctctgctcagaggcaggtcaccactgggccctgtggatcaatgcaagatgacaaaggctttttttttttttttttttttttttttttttgaggagtttcgctcttgttggctaggctggagtaaaatggtgcgatctcggctcactgcacctccgcctcccaggttcaagcgattttcctgcctcagcctcccgagtagctggggttacaggcatgcaccaccatgcctggctaattttctgtatttttagtagagacggggtttctccatgttggtcaggctggtcttgaactcctgacctcaggtgatctgcccgtctcggcctcccaaagtgctgggattaccggcatgagccactgcgcctggccgacaaaggctttgatatcagaatgaactgtcaagggaggtgctggagagggattaacctgtgctgcctgggaccctcagggtcttaggttggggagtgtgaataggagtttgcagatggagaataggaagggcattccaggcagagggaaacctgtgcagagaccaagaggtgtggaaggaaaagtggggttggggctgggtggtctggattatggcctggatgcaataaagtactgtgacagtagcca

SEQ ID NO: 19 - DNA, 1345 bp, Homo sapiens
  source 1..1345: mol_type = unassigned DNA
gcctctggggttttatattgctctggtattcatgccaaagacacaccagccctcagtcactgggagaagaacctctcataccctcggtgctccagtccccagctcactcagccacacacaccatgtgtgaagaggagaccaccgcgctcgtgtgtgacaatggctctggcctgtgcaaggcaggcttcgcaggagatgatgccccccgggctgtcttcccctccattgtgggccgccctcgccaccagggtgtgatggtgggaatgggccagaaagacagctatgtgggggatgaggctcagagcaagcgagggatcctaactctcaaataccccattgaacacggcatcatcaccaactgggatgacatggagaagatctggcaccactccttctacaatgagctgcgtgtagcacctgaagagcaccccaccctgctcacagaggctcccctaaatcccaaggccaacagggaaaagatgacccagatcatgtttgaaaccttcaatgtccctgccatgtacgtcgccattcaagctgtgctctccctctatgcctctggccgcacgacaggcatcgtcctggattcaggtgatggcgtcacccacaatgtccccatctatgaaggctatgccctgccccatgccatcatgcgcctggacttggctggccgtgacctcacggactacctcatgaagatcctcacagagagaggctattcctttgtgaccacagctgagagagaaattgtgcgagacatcaaggagaagctgtgctatgtggccctggattttgagaatgagatggccacagcagcttcctcttcctccctggagaagagctatgagctgccagatgggcaggttatcaccattggcaatgagcgcttccgctgccctgagaccctcttccagccttcctttattggcatggagtccgctggaattcatgagacaacctacaattccatcatgaagtgtgacattgacatccgtaaggacttatatgccaacaatgtcctctctgggggcaccaccatgtaccctggcattgctgacaggatgcagaaggagatcacagccctggcccccagcaccatgaagatcaagattattgctcccccagagcggaagtactcagtctggatcgggggctctatcctggcctctctctccaccttccagcagatgtggatcagcaagcctgagtatgatgaggcagggccctccattgtccacaggaagtgcttctaaagtcagaacaggttctccaaggatcccctcgagactactctgttaccagtcatgaaacattaaaacctacaagccttaaaaaaaaaaaaaaa

SEQ ID NO: 20 - DNA, 109 bp, Homo sapiens
  source 1..109: mol_type = unassigned DNA
gaaagggtggcattatagggttccacaaccatgtctaacaccttggacaagagcagcacactgaatgggtttatgatcctatgtgggcacttctcctggatattgttga

SEQ ID NO: 21 - DNA, 3670 bp, Homo sapiens
  source 1..3670: mol_type = unassigned DNA
cgcagcaaacacatccgtagaaggcagcgcggccgccgagaaccgcagcgccgctcgcccgccgccccccaccccgccgccccgcccggcgaattgcgccccgcgcccctcccctcgcgcccccgagacaaagaggagagaaagtttgcgcggccgagcggggcaggtgaggagggtgagccgcgcgggaggggcccgcctcggccccggctcagcccccgcccgcgcccccagcccgccgccgcgagcagcgcccggaccccccagcggcggcccccgcccgcccagccccccggcccgccatgggcgccgcggcccgcaccctgcggctggcgctcggcctcctgctgctggcgacgctgcttcgcccggccgacgcctgcagctgctccccggtgcacccgcaacaggcgttttgcaatgcagatgtagtgatcagggccaaagcggtcagtgagaaggaagtggactctggaaacgacatttatggcaaccctatcaagaggatccagtatgagatcaagcagataaagatgttcaaagggcctgagaaggatatagagtttatctacacggccccctcctcggcagtgtgtggggtctcgctggacgttggaggaaagaaggaatatctcattgcaggaaaggccgagggggacggcaagatgcacatcaccctctgtgacttcatcgtgccctgggacaccctgagcaccacccagaagaagagcctgaaccacaggtaccagatgggctgcgagtgcaagatcacgcgctgccccatgatcccgtgctacatctcctccccggacgagtgcctctggatggactgggtcacagagaagaacatcaacgggcaccaggccaagttcttcgcctgcatcaagagaagtgacggctcctgtgcgtggtaccgcggcgcggcgccccccaagcaggagtttctcgacatcgaggacccataagcaggcctccaacgcccctgtggccaactgcaaaaaaagcctccaagggtttcgactggtccagctctgacatcccttcctggaaacagcatgaataaaacactcatcccatgggtccaaattaatatgattctgctccccccttctccttttagacatggttgtgggtctggagggagacgtgggtccaaggtcctcatcccatcctccctctgccaggcactatgtgtctggggcttcgatccttgggtgcaggcagggctgggacacgcggcttccctcccagtccctgccttggcaccgtcacagatgccaagcaggcagcacttagggatctcccagctgggttagggcagggcctggaaatgtgcattttgcagaaacttttgagggtcgttgcaagactgtgtagcaggcctaccaggtccctttcatcttgagagggacatggcccttgttttctgcagcttccacgcctctgcactccctgcccctggcaagtgctcccatcgccccggtgcccaccatgagctcccagcacctgactccccccacatccaagggcagcctggaaccagtggctagttcttgaaggagccccatcaatcctattaatcctcagaattccagtgggagcctccctctgagccttgtagaaatgggagcgagaaaccccagctgagctgcgttccagcctcagctgagtctttttggtctgcacccacccccccacccccccccccccgcccacatgctccccagcttgcaggaggaatcggtgaggtcctgtcctgaggctgctgtccggggccggtggctgccctcaaggtcccttccctagctgctgcggttgccattgcttcttgcctgttctggcatcaggcacctggattgagttgcacagctttgctttatccgggcttgtgtgcagggcccggctgggctccccatctgcacatcctgaggacagaaaaagctgggtcttgctgtgccctcccaggcttagtgttccctccctcaaagactgacagccatcgttctgcacggggctttctgcatgtgacgccagctaagcatagtaagaagtccagcctaggaagggaaggattttggaggtaggtggctttggtgacacactcacttctttctcagcctccaggacactatggcctgttttaagagacatcttatttttctaaaggtgaattctcagatgataggtgaacctgagttgcagatataccaacttctgcttgtatttcttaaatgacaaagattacctagctaagaaacttcctagggaactagggaacctatgtgttccctcagtgtggtttcctgaagccagtgatatgggggttaggataggaagaactttctcggtaatgataaggagaatctcttgtttcctcccacctgtgttgtaaagataaactgacgatatacaggcacattatgtaaacatacacacgcaatgaaaccgaagcttggcggcctgggcgtggtcttgcaaaatgcttccaaagccaccttagcctgttctattcagcggcaaccccaaagcacctgttaagactcctgacccccaagtggcatgcagcccccatgcccaccgggacctggtcagcacagatcttgatgacttccctttctagggcagactgggagggtatccaggaatcggcccctgccccacgggcgttttcatgctgtacagtgacctaaagttggtaagatgtcataatggaccagtccatgtgatttcagtatatacaactccaccagacccctccaacccatataacaccccacccctgttcgcttcctgtatggtgatatcatatgtaacatttactcctgtttctgctgattgtttttttaatgttttggtttgtttttgacatcagctgtaatcattcctgtgctgtgttttttattacccttggtaggtattagacttgcacttttttaaaaaaaggtttctgcatcgtggaagcatttgacccagagtggaacgcgtggcctatgcaggtggattccttcaggtctttcctttggttctttgagcatctttgctttcattcgtctcccgtctttggttctccagttcaaattattgcaaagtaaaggatctttgagtaggttcggtctgaaaggtgtggcctttatatttgatccacacacgttggtcttttaaccgtgctgagcagaaaacaaaacaggttaagaagagccgggtggcagctgacagaggaagccgctcaaataccttcacaataaatagtggcaatatatatatagtttaagaaggctctccatttggcatcgtttaatttatatgttatgttctaagcacagctctcttctcctattttcatcctgcaagcaactcaaaatatttaaaataaagtttacattgtagttattttcaaatctttgcttgataagtattaagaaatattggacttgctgccgtaatttaaagctctgttgattttgtttccgtttggatttttgggggaggggagcactgtgtttatgctggaatatgaagtctgagaccttccggtgctgggaacacacaagagttgttgaaagttgacaagcagactgcgcatgtctctgatgctttgtatcattcttgagcaatcgctcggtccgtggacaataaacagtattatcaaagagaaaaaaaaaaaaaaaaa

SEQ ID NO: 22 - DNA, 1426 bp, Homo sapiens
  source 1..1426: mol_type = unassigned DNA
atgagccaggcctactcgtccagccagcgcgtgtcctcctaccgccgcaccttcggcggggccccgggcttcccgctcggctccccgctgagctcgcccgtgttcccgcgggcgggtttcggctctaagggctcctccagctcggtgacgtcccgcgtgtaccaggtgtcgcgcacgtcgggcggggccgggggcctggggtcgctgcgggccagccggctggggaccacccgcacgccctcctcctacggcgcaggcgagctgctggacttctcactggccgacgcggtgaaccaggagtttctgaccacgcgcaccaacgagaaggtggagctgcaggagctcaatgaccgcttcgccaactacatcgagaaggtgcgcttcctggagcagcagaacgcggcgctcgccgccgaagtgaaccggctcaagggccgcgagccgacgcgagtggccgagctctacgaggaggagctgcgggagctgcggcgccaggtggaggtgctcactaaccagcgcgcgcgcgtcgacgtcgagcgcgacaacctgctcgacgacctgcagcggctcaaggccaagctgcaggaggagattcagttgaaggaagaagcagagaacaatttggctgccttccgagcggacgtggatgcagctactctagctcgcattgacctggagcgcagaattgaatctctcaacgaggagatcgcgttccttaagaaagtgcatgaagaggagatccgtgagttgcaggctcagcttcaggaacagcaggtccaggtggagatggacatgtctaagccagacctcactgccgccctcagggacatccgggctcagtatgagaccatcgcggctaagaacatttctgaagctgaggagtggtacaagtcgaaggtgtcagacctgacccaggcagccaacaagaacaacgacgccctgcgccaggccaagcaggagatgatggaataccgacaccagatccagtcctacacctgcgagattgacgccctgaagggcactaacgattccctgatgaggcagatgcgggaattggaggaccgatttgccagtgaggccagtggctaccaggacattgcgcgcctggaggaggaaatccggcacctcaaggatgagatggcccgccatctgcgcgagtaccaggacctgctcaacgtgaagatggccctggatgtggagattgccacctaccggaagctgctggagggagaggagagccggatcaatctccccatccagacctactctgccctcaacttccgagaaaccagccctgagcaaaggggttctgaggtccataccaagaagacggtgatgatcaagaccatcgagacacgggatggggaggtcgtcagtgaggccacacagcagcagcatgaagtgctctaaagacgagagaccctct